SEQUENCE LISTING (213 sequences)

SEQ ID NO: 1 - DNA, 1248 bp, Glycine max
  source 1..1248: mol_type = other DNA
atgggctctggaaatatactaccttcttctggcagctctgacatgtctatctccgagctccgcgagaagcaccattgggagctagaaaatctgactctgaccacacaacctctcaaaacgttgaaattcttcacattagctgttattcaatacatcaagaaaacagcaatatatctattggcaaaaggtggatgggttatgcttttcagtgttgcagttgggactcttggcatagtgctgatgacccttggttgcctccatgagaagcatcttgaggagcttcttgaatattttcgctttggactgtggtgggttgcccttggggttgcttcttcaattggtctaggatctggtttgcacacatttgtcctatatttgggtccccacatagcactgtttacaataggagcagtgcaatgtggccgagttgatttgaaaagtgctccttatgatacgatacaattaaaaagaggtccttcttggcttgataaagactgttctgagtttgggccaccattattccagtcgcaggttccgcttagcagcattttgcctcaagttcagctggaggctattctatggggtattggaacagctataggggagcttcctccttactttatctccagggcagcacgcttgtctggtgggagagtagatgccatggaagaattagacagcgaagataaaagagtcttgagtcgaataaagtgctggtttctatcacactcgcaacatttgaatttctttactattctagtgcttgcttcggttccaaatcctctatttgaccttgccggcatcatgtgtggacaatttggcattccattttggaaattttttcttgcaaccttgattggaaaggcaattattaaaactcacatacagacgatattcataatctcagtttgcaacaatcaacttcttgattggattgagaatgaatttatttgggttctcagccacatacctggttttgcatctgtcttgcctagagtgactgctagtctccatgcaatgaaagataagtatctgaaagcaccccatccactttccccaaataaacaggggaaaaaatgggatttttcttttgcttcagtctggaacactgttgtgtggctcatgcttatgaacttctttgtcaagatagtgaatgcaacttcccagaggtatctgaagaaacagcaggagacacagcttgctgcattaacggaaaagtctaccccaacagactcagacgcacaatga

SEQ ID NO: 2 - DNA, 1362 bp, Glycine max
  source 1..1362: mol_type = other DNA
atgagacacgtggttattttttctttcaatttcttgctgctttgtgtcttcatttctgctataacaagcacaaaaagtgaagtatctagcacaaatgaagaacaatcattttcttaccatatcaaagctttggaattcatatggaaacatttgggctatcaacatgtgtggccagaaatggaatttagctggagaattgttgttggaaccttgattggaatcttgggagcagcatttggaagtgtagggggagttggtggtggtggcatctttgtgccaatgctaatcctcattattgggtttgatccaaaatcagcagttgctatttcaaagtgtatggtcacaggtgcagccatctcagcagtattcttctgcatgaagcaaaggcatcccacacttgatgaacccgttatcgactacgatttgatgttgctaatacaaccaaccctcatgcttggaatcagcataggagttattttgagtgtaatatttgctgattggatggtcacaattcttctaattattctatgtatagtgacatcaatcagggcattcttcatgggtgctgacacatggaaaaaggaaaccaaaatgaaagaggtgagcatccttggcaacatgtattggaaggagtttgtacttattttcattgtctggctcgcatttgtgaactatacagtttcctgttcagtcacatactggatacttattttgtcacagataccaattactgtaggattttatttgtaccaagcaagagccctatatcaggggagagctgcaggatctcaacacacacattggccattgcaccatctatttctagctagcatttgttctctgttagctggaattgttggtggacttcttggtacaggttctggatttgttatgggtcctttatttctagaagtgggaattgctccacaggtagcaagtgccacagccacttttggaatgatgtattcatcatctttatctgtcatacaatattacctgttgaatcgttttcctgttccttatggtaaaattaaacttaattcacattgtacccttctattatttctgaatggttttcaaaggcaacataaacttattcaagtaatttttatgcatgtttcaacctgttttgcagctctcttccttactcttgtggctgcaattgcagcattcctaggacagtatctcattgacaagcttgttaatatcttccaaagggcttctttaattatttttgtcttggccttcacaatatttgttagttcaattgcattaggtggagtcggcatatcaaacatgatcttgaagattcaaaggaatgaatacatgggatttgataatttttgcaggaatgatacatag

SEQ ID NO: 3 - DNA, 1392 bp, Glycine max
  source 1..1392: mol_type = other DNA
atggcttcttcgtttctatccgcagcttcgcacgctgtctcaccctcttgttctctgtccaccacgcacaagggaaagcccatgcttggaggcaacactttgagatttcacaaaggacccaattccttctctagttcaaggtctagaggtcggatctctatggctgttgcagttaatgtatctcggtttgaaggcatacctatggctcctcctgatccaattctcggagtttccgaggcgtttaaggcagacaatagtgatgtcaagctcaatcttggagttggggcatacagaacagaagaactacagccatatgtgcttaatgttgttaagaaggcagagaatcttatgctggagagaggggataacaaagagtatctcccaattgagggtttggcagcatttaataaggcaactgcagagttgttactcggagcagacaacccagcaatcaaacagcaaagagttgccactgtccaaggtctttctggaactggttctctgcgactaggtgcagctctgatagagcgatattttcctggggcaaaagttttgatatcagctcctacctggggtaatcacaagaatattttcaatgatgctagtgtcccatggtctgagtaccgatattatgatcccaagacagttgggttggattttgagggcatgattgaagatataaagtcagctccggaaggatcttttatcttacttcatggatgtgctcataatcctactggtattgatccaacacctgaacagtgggaaaaaatagctgatttaattgaagaaaagaaccacattcccttttttgatgttgcttaccaggggtttgctagtggaagccttgatgaagatgcagcttctgtgagactgtttgtggcacgtggcatcgaggttcttgtagctcaatcttacagtaaaaatctcggtctctatgctgaaaggattggagcaatcaatgtgatttcatcgtcaccagaatctgcagcaagggtaaagagccaactgaaaaggattgcccgaccaatgtactctaatccaccggtacacggggctaggatagttgccgatgttgttggaaacccagttctctttaatgaatggaaagcagagatggaaatgatggctggaaggataaagaatgttagacagcagctatatgatagtattacttcaaaagacaaaagtggaaaggattggtcattcatacttaagcagataggcatgttctcattcactggcttgaacaagaaccagagtgacaacatgacaaacaagtggcacgtatacatgacaaaggatggaaggatttccctggcaggattgtcgttggctaaatgcgaataccttgcggatgctattattgattcatatcataatgtcagctga

SEQ ID NO: 4 - DNA, 90 bp, Glycine max
  source 1..90: mol_type = other DNA
atggatataataagtcttgcttgggctactttaatggtagctttttcattttctcattccctcgtagtatggggaagaagtggactatag

SEQ ID NO: 5 - DNA, 798 bp, Glycine max
  source 1..798: mol_type = other DNA
atgtctaagaacaagctctgcacacccctttcaacccttctgagacgcactttaacttcatttccccaacacttcacttccaatttctctcagatcaaactctctcttcatgactcatcactaccgtttgcacccccttctcataaccccacctgcgattactattattctctcggatttttcaggaaaagcctatgctcaaaatctgctgaggaaatccaacgtggatgttggaattgccacactgtccctcattcggaacctttccttgtttgtgagtcatgtcggtgcattcagcctgtggatagctccgttgactattttgacatatttggggtggagaggaagtatgacatagagggtgagaatttggagggcaagtacaaagattggcaaaagaaactgcatcctgatttagtacattcaaaatctcagaaagaaagagattttgctgctgaacaatctgcaagggtcattgatgcataccgtactcttagcaagcctttgtcaagaggaatttacttgctaaagctttatggagaagaagttgatgaagaacagacaatttcagattcagaattacttgcagagattctggaaatcagggaagcagttgaagaagcaactaactctgagactttgaatcacattctctctgagatgcaggagaagctacataattggtccaatgcctttggtcatgcttttcaaagccaaaactttgaagaagcaaaaatggcaattaggagaatgacttactatagtcgtgtaattgatgaagttgtaaagaagctttga

SEQ ID NO: 6 - DNA, 2895 bp, Glycine max
  source 1..2895: mol_type = other DNA
atgccggaaaataaaagcgagaatcgtgctaaaatagaaagtttctgggaaggtgctttaaaggatatttacgaagaaacactaatgtatgataataatgatgtggtccacagtccggatgaatgcgttgatcctgaaacacgaaaaataaaagtccctaaatcagagtttgttaagacatataaaaatagcgtcttttctcttctggaccgttatagatatgaaaataatgatgcacaaacattagaatcgcttcagagagaaattgaggatctgactttgaacttctctgaagatagtatttataaaactagtcctaacattatggtaatgttaactaaaaacaacttgccaagtgctaaggaattccttcgtggaaagtatcttatggataaagaatatcccggcagaatgaccatttatgcagaaagtcgaagtggtgatagtctagaaaccagcggctcagagctatccgatgaggagaataaaaatctaagatgtgtactatctgaaagtgataagtcgttgttggtagagttaggcgagcactccattgagtgcctactggttcacaccctaggccatctgtttaatctggaaaatattgtcagtcttgctagtttggttgatcgtatagaacgcaatgtaagggattatgcctcatcgtcaacattgtattataatagtagggtatgtaatctaagtatggatgctgttgataaaggtactaaagcatctaaaactagagcttatccctttggtacagcattagttgaatttttagtgagtcgtggtctattgcgcttagtaacatataatgttgatttattaaatttctctgatgatagtggtccaaagactgttgtcaaaaatgttgtcaaaaagggccataattactataggagctctttagtatatgccgaatgcttgtttaaccccgcattacttcctataaagttaaacctacctatggtatttccacctaaagattgggagccccacaaacctgaacagaaacgtaagttgttacatatatctgatatatatggtggttatttaagcaaccctacgggccaaatctacagcactcaacggagtatgagttctcccgatgcgaagaatttcttcatctatttcggtgaaaataagtatcaagaaagtcataataagcaaaataagttttgtactagcatcagtagcttacagcgtcaatcctttaaaataaatagtaatttccttaattttatattagaaaatagggaattattagaagaatgtggctacttgatgcccgaatttctttcaaaggtaatactagcccatgcctccaggaaacttagaagtcattttgaaaagaataaggatattcaaacaatatacaaatttagtgatgtgtacgctctattaataaagaatatgcagcgggcacgttatgagtgtacgattttagatctggcaaaggcctactctggatattctttatattttccagcatttattgacttcaaaggtcgaatctatcgcagcggcatctttcacttccacgagcgggatttagcccgaagtctactattaatagattgtaaagataatatagaatattcttcccctgaaattaataattatctaaatatgtatctaaccgcaactgcattcctctatcaatcatataaatatgaggaggatgctctaaatgatataaataataaattattaccattacctaatctagagtgtaaagagaactttaaaacgtatttaaagactttagtgatagaatcacggatggctaaacggccattccaatatttatctaatatttaccttttattattatcattatcatcatcggatttaatgtctttcgttgatctgtataattgtgttcctgtaactcaggatgctagcgctagcgcatatcaactgatggcttatttcctcctggataagaattttgcgaaactaacaaatctttttgataccggcagcggtgagatatttgacatctattctcacatgagaattgaattaatttcattcatcaaagactctcttagtgaggagaatcctgagctatgcgcaattttggatagggtcctaactaggagtatagttaagcaaatatatatgccaattatatatggtaaaacagctaatagcacaactaaggatcttatagtgagtctttgccaagatctcttgccaaaagaatgtactatactgtcggttctatgcttcaaattttggcaagaaaaatacagttttatgtattcgtttatccagctgatcagcctggtaggtcgggtatgctcttacctagatcgaccagtcttgtacagcacagaatattttagcacatcccaggattataaaaagatggaaaaacactcagttagagtcttcaactcttacttgaaaaaggcccataatgtcacactctcgtttccctcaaaagagcgggataagaggaaaagtggggtatccacattcattaacttcattcatcaaaaggacgcgcaaattgctatgtctattgccttctatgagaactcatataatatacctctgtacacagttcacgacaacttcataacaaatatgcattattgtaaatatatatctaaaatctatctacatgtcataaaggaaatggggccccctcttaaaatcattaaccgctttatttataaaaatattatggagccggctatagcgaagggcctctataataatgtaaaaggctacaatttgaagtcttttgatgaaaggatcataccagagcctattttagatgagttcttaaaaattggactcatttctccagaagatgtcacgaatgcagctggtttaaaaaaaataaacaataagttgaaacaaagatatcaactcatataa

SEQ ID NO: 7 - DNA, 1014 bp, Glycine max
  source 1..1014: mol_type = other DNA
atgaatccttctctcacttcttcctcaaccttgtcgtcgtcttcttcgtcctggttctccggaatcgttcgagttggccgttccaacagtgtcaaaatgtccaataactccgctgctgcaccctcctccgacactgccggccccgtcgtccgcaaaaatcagttccgaggtgttcttttcaagtacggtcccaatcctattcaggttgcatttaagactggtgatttcaagcggcaagtcatttttattggtggattgaccgatggttttctagctactccatacttggaacctctggcaattgctctggaccatgagaattggtcgttagttcaattccttatgtcatcttcatacagtggatatggcacctccagcttgcaacaagatgctaaggagctggatcagctgattaattatttaattaacaaagaagactctgaaggtgtggcattacttgggcatagtactggctgtcaggatattgtgcattacatgcgcacaaattttgcttgctcccgagctgttcgtgctgccatctttcaggctccagtcagtgatcgggaatatcaagctacactccctcatacagcttctatgattgacttggctgctaagatgataagtgaaggccgaggtctagagttaatgccaagggaagcagatcctagtgccccaataactgcctataggtatcactccctttgttcatataatggcgatgatgacatgttcagttctgacctgagtgatgaccagttgaagatgagacttgggcatatgtctagcacacattgtcaggttatattttcaatggctgacgaatatgtgccagattatgttgataagaaagctctagttgagcggttatgcagagcgatgggaggagcagagaaagtagagattgaatatggaaatcattccctctctaacagagttgaagaagctgttgatgctattattgacttcttaaaaagagagggacccaagggatgggacgatccatggagttaa

SEQ ID NO: 8 - DNA, 780 bp, Glycine max
  source 1..780: mol_type = other DNA
atggctgaattcacagaaaatttgcaaaacatttcctcatcatctccattcttagacattgatccaagcatggaactcctaaaccaattcatagggatgaaccaattatatgtgctagacaactcaaacttgatgccttatttctcctgtgataccttcttgggcccccaagaacctgagtttccaggaaacttggaagaagattttccttttctcttccatcatgtcaaccacaatgcacctcctgtttccctccccattttccaagcagaaaatgagatccatgaaggtaataagaggaaatcaatggatcttcttgagaccagttttgctaattcaacttctgcagtttctgagactggaagcaagatcaaacatagttctggaagaggaaagagactgaaaaacaatgttacagaagaagaggagaaagcaaaggaagtggttaatgccagggctagaagaggtcaagctactgatagtcacaatttagcagaaagggttagaagagggaaaatcaatgagaaattaaggtacttacaaaatattgttccaggatgttacaagacaatgagtatggcagtaatgttagatgaaatcataaactatttcctttctttggagcttacagcagcaagtaccttttatgacttcaattccgagatagatgcttttgaaacaatgcagagatcaagggcatacgaggcaaaagagttaggcaagtataaaagagaaggacatggaggagtttctctccttcaaccaacatggcacctttga

SEQ ID NO: 9 - DNA, 1569 bp, Glycine max
  source 1..1569: mol_type = other DNA
atggaaagaagggttgtaattattggagcaggaatcagtggccttggtgcctgcaaataccttctagagtttggatttaacccaattgtctttgaagttgatgatggtgttggaggactatggagacataccatggactccaccaagctccaaaacaataaacaaatgttccagtttatggattttccatggccttcttctgtgaaagaagataatccaagtcacaagcaagtgctagattatgttaactcctatgctgaacatttttccctcattccttacattagattcaacttcaaagtcattgacatagattatgttggtggagagtctagtgaagaaatgaagtcatgggaattgtggggtggtaatggcaggcccttttgctccaagggaacttggcatattgctgtgcaacataccaaaaatttatccatagagatgcatgaggccgagtttgttgttctttgcattgggaaatatagtggttttccaaacattcctgaattccccccaggaaaaggcccagaagttttcaatggcaaggttatgcactccatggactactcgaatttggacaatgagactgctgctgaactgatcaaaggaaaaagagttacaataataggctcacagaaatctggtcttgatctagcagctgaatgtgcgaatgcaaatggagtgaagcatccttgcacagttatccaaagaaccgcacactggtttcttccagattttaacttttggggtgttattgctggattcttgtacttcaatcgctttgcggagcttttagttcacaagccaggagagtccttcctacttggccttgttgccactctgctttcaccatggagatggggaatttctaaacttgttgaaactactctaaaatggaagctgccattgaagaagtatggaatggcacccaatcacagctttcttcaggatctctccacatgtctgtttgcggtgtatcctgataacttttttgacaaactaaaagaaggatccatcattatgaagggatcgcaaaactttagcttttgtagagaaggtgtaatcattgacggagaagctaagcccctggaatcagatattgtattttttgccactggatacaaaggtgaccaaaaaatcaaaaacatattcaaatctccactcttccaaaagtacatcattgggcaagcaacctcaacagttcctctctacagacaaataattcaccctcagatcccccagttggcaataataggatatgccgagagcccatcaaatatatttgcctcggaaatgaaaagcttgtggctatcacatttcttggatggaaacatagagttgcctagtataagagagatggaaaaggacgtgaaattgtgggaagacaatttgaagcaatatggtgggaaatattattggaaaacatgcattgctcattgtggcatatggtatcatgatcaattgtgcaaagacatgaagcatgaccctagaaggaagaatggccttttttcggagttgtttgaaccatatggccacgctgattacgcaggtcttactcgtaaaagataa

SEQ ID NO: 10 - DNA, 528 bp, Glycine max
  source 1..528: mol_type = other DNA
atgggagacgaagcaagtgaagagcatcaaaggtcgctagcagctcctttcatatttttcgtggttctcgcatttcagtttgcttctcattggattgatcactttaagaagagtggatctgacaaagaaaaagaaactaagttgcgtggagaaataaaagagcttttgaaggaggcaagctcactgtcacagccatcaacctttgcacaagcagcgaaactcaagagactggcagctgcaaaggagagggaactggcaaagtgtcaaaatttacatcacaaggatgatgctttgtattcaaaagttctgctcatctcaaaggttttaacatatttaattctgcttatctggttttggagtgttcctgtctctagcatatctcaacaacttgtgcaaccatttgggagattattatcttggaggactggaggggttcaaaatagcaatattatggttgggataataccttggctgatagtgtctaccagggttagcagatttatttgtagactcacctatggcaaatag

SEQ ID NO: 11 - DNA, 1062 bp, Glycine max
  source 1..1062: mol_type = other DNA
atgcaaatgtctcaaaaacataaaatgtcctatgactctagcaggttcagcattgagcctgtgcaaaatctggggtcatgttgcttcctgcaaagtgggaacctagactactcctcatcctctgataacggtagccatgcaacctacccttctgtttgtattttcgaacaatattgcaccctggaatcttctacaaacaacaatatcccttccctaaactctccatctactgtcagtttctcacctaacaatagtccagtctcaaagctgcattcaaaagcatatgtgttgagttcacaaaatttgcttgaaattgttaatgaatcgctagaaaacaaatctttcttgacacttaacgatgatgagctgaggcacaagataagagagcttgaaagtgctatgttgggacatgatacagatattctggatacctatgataccataattccaaaagaatctgattcgtttttgaaagaggcagaaaggtggaagaggatggtggcaaagatatctagaggggatttaaaagagatgctttgtacatgtgcaaaagcagtggcaggaaatgatatggagacaactgaatggttgatgtcagagttgcgtaaaatggtctccgtctccggcaatccaatccaacgattgggagcatacatgttggaggctcttgttgcaaggcttgcctcttcaggaagcacaatcttcaaagtcttgaaatgtaaagagcctactagtagtgaactcctttcacacatgcatctactttatgaaatctgtccatatctcaaatttggttacatgtcagcaaatggagctattgctgaagtcatgaaggaggaaagtgaagtccacattattcattttcagattaaccagggaattcagtgggtaagcctaatccaagctgttgctggcagacctggagcaccccctaagattagaataacaagttttgatgactccacttcagcttatgccatggaaggaggccttgaaattgttggagcaaggttatcaaggcttgcacaatcatataatgtaccctttgagtctaattgctaa

SEQ ID NO: 12 - DNA, 1143 bp, Glycine max
  source 1..1143: mol_type = other DNA
atgtttacaaatgtaaaagaagaagaaaaaaaaaaacaaaaaaagaaagagctaacgcaacccaaacccaaagtatcaatctcctctacttccatttccagtggtcactcactcagccactgtgcttcttcttcttctctttctccctctctcctcttcccaatgccctctctccaccttctccccggcgccaccaccgcccccaactccacattcctcctccgcccccattcccctctcctcccctccaaaaccctaacccccaaaaaacccaaacccaaaccccccagtgccctcctccagtggaaccgcaagcccgagctctccggctccatcccgcgcgtcaccgtcattacctccggaaagggcggcgtgggcaagaccaccaccaccgccaacatcggcctctccctcgcccgcctgggcttctccgtggtcgccatcgacgccgacgtcggcctccgcaacctcgacctcctcctcggcctcgagaaccgcgtcaactacaccgtcatcgaggtcctcaacggcgactgccgcctcgaccaagccctcgtccgcgacaagcgctggtccaacttcgaactcctctgcatctccaaaccccgctccaagctccccctcggcttcggcggtaaagccctcacctggctcgtggacgcgctgaaagcgcgtccacagggctcccctgacttcatcctcatcgattgccccgccggcatcgacgccggcttcatcaccgccatcactcccgccaacgaggccgtcctcatcaccacccccgacatcaccagcctccgcgacgccgaccgcgtcaccggcctcctcgaatgcgacggaatccgggacatcaagatgatcgtcaaccgggttcgaaccgatatgataaaaggagaagacatgctgtcggtgttggacgtgcaagaaatgttagggttgcccttgcttggggctattcctgaggacactgaggttattagaagcaccaatagaggctaccctcttgtgctcaacaagcctcccactctggctggcttggcgttcgaacaagccgcttggaggctcgtggagcaggatagcatgcaggccgtggtggtggaagaacaacccaaacgagggtttttctccttttttggtgggtag

SEQ ID NO: 13 - DNA, 1038 bp, Glycine max
  source 1..1038: mol_type = other DNA
atgggcgaacaaccaaacccttggaacatactacagcctcaggtggcgaagagccgacatcgaggtgccaaaccttcccatccatgtgagatcttagggaagatcagaaacctttgcacgcctccgttacctttagggaggcttacgccccatagaaactatctacctgagactatccctcggctcgtaggtcctggaacaaggaaaagcccaaagccaatcccaggaaatagtgaagcttcatatggtctttctgtccaagtgcaggtagtccgcatcttcacaaacatgtctatttcaccgagtctctctccgaggcagtgcccaaatcattatgcctttcatgcggacgttatagttacggtcgccgttcaccgaggcttcggtcatcggatcccctgtcatctggtcactaacttccttgaccttccggcacttggcaggtgtcagcccccatacatggtcttacgactttgcggagacatgtgtttttgcctcctccatccctcgagtccaataaagggtagtacaagaatattcaccttttgtccatcgactacgcccttcggccggatcttaggccttgactcaccctccgtggatgaaccttacgaaggaacccttgggttttcgaggcattggattcttaccaacgtttatggtacactcccctaccgattcatttttacaccccacagctttggcagatcgcttagccccgttcatcttcggcacaagagcgctcgatcagtgagctattatgcattctttcaagggtggctgcttctaggaaaacctcctggttgtctctgcacccctacctcctttatcactgagcggtcatttaggggccttagctgtattagagtttgcctcgatttggtaccgctctcatggcccgcaccgaaacagtgttttacccctagatgtctagtaaactactgcacctcaacggattttagggagaaccagctagctctgggttcgagtggcatttcacccctaaccacaacttatccactgattcttcaacattag

SEQ ID NO: 14 - DNA, 1905 bp, Glycine max
  source 1..1905: mol_type = other DNA
atgtcatcatcaaccacaagcagcagcagcagcaacaacaacatgagcgacgtcgatcgtttgttcgcatcttttaagtgcggtttaacacctccaaaatctgctacgagggaaagaaaacgaagcaagaggagactggagcaggtgagttcaaaccacgagactccatcggaatctccgggtccaagatcgcctgaagaggaactgagaacccccttatcacgcatcagaaaaaccaacattgaatcttctattgagaaacttaaatcagcagtaaccagagcacaaagcattggttgtgggaaacagttctctccaattgtattttatggatctccctgtggcatccctcccaaaaagccaacccgtatgttatggcgaatgttacgtgagatatgccccaatgtctctcagcaaaatatattgagcataaggaaggaagtttggatgacattcccaaggcaggatgaagcaatgaagtttgccaaaggacaagaagatgttcatgttttcagctatcaagaccactttaatggccaaagaaggttccttgtgtctacatatacagaattctggcgaaggtacaataatatgaatcccaaattccgtcatcattatgaagtcattcaagcgggtttaccatgtcacctttattttgatttggagtttaataagaaagtcaacacaggaaagaatggagaagaaatggttgatctcttgatatcagtggttctagaagccttccatgaaaaatatgcaattcaaggagaccacgattgggtagtagaacttgattcttcaaatgaagataagttctctcgtcatataatagttcgcataccaaagactgcatttaaggacaactctcatgcaggtgcatttgtttcagaaatatgctcaagaattctaaatgcaagggagaaagataaaagttttgaaaaattgtttgtaatgaaagactctagcaacaatgaatctgcaggccaactttttgtggatactgctgtatattcacggaatcgttgttttcgtctccctctttcttcaaaagcaggaaaaagatcagttcttctgccaacaaagcgatttaagtgcaaggacttgggtgaagaagacatattcatggcttccttgatctgcaatatggatgttgattgcaaaacgtatctggtctgcaaaacagacttagattgtgtgaaaactctgcattttgatacagaggagaatagtaatgttagtaattccacccaaattcctccagaatttacattgggtactagcacaagtgatgtttcaactacatacttcatgggaaagtcaccattcccatttctggacaaatttatactgtctgttgcctctgtcgggaatataccaggaaacattcatagctggtattatttctcggaatttggattgatggtctacagcatgacaaaaaatagatactgtgagcgaattggcagacagcataaaagcaataatgtgatatatgtggttgatctacgaatggcagtgtattatcagaaatgtcatgatcctgactgcagaggttatcgatctccctcacgtcaaatcccagttcatgtgttctccaatccttcagatgtaattggttcctttgggttgttagatgatgaacagccagtagatgataagttgagacatcaacttgatgacaacaaggagcaaaatcttttacaattcaaagacactgttgaggacaactccaatgattcatggtggctagaagccataagagttgtcgaggatatggaaaataagcaaacaaagacagagcaggaggtaattgatgaagatgaggagtggtggcaggctgtagaaagtactgcatcccaggttgaacttacctgctccagtcaacaagaactctgcgcaatctaa

SEQ ID NO: 15 - DNA, 2142 bp, Glycine max
  source 1..2142: mol_type = other DNA
atggaattgaaaaagggcaagcttgccaggttctacacggatggtaaaaagcataagcaatcgttttggggagcaagagcaactgagcctccagcacttaaacctagcaatttgctaccactagggaaaaatagaattgctgaaacatttagaattggaggatcgaaggtgttcccggaggaccatgagccgtggcgtaaaagaatacttgacccgggtagcgatattgtgttgaaatggaaccgggtcttcatagtttcatgcctggtggctctttttgttgatccattgtacttttatttgcctagtgtaatagaaaacacagggtctacatgtgtaaggacagacctaacactgcgcattgtcgtgacctttcttcgcactattgcagaccttttttatctgttgcacctgataattaagtttaggacggcatatgtggcaccaagctcacgggtatttggcaggggtgaacttgtcatggacccaaagaagattgcaaggagatatataagatctgatttcttcattgattttattgccacactccctctacctcagatggtcatttggtttattataccagcaacaagaacccctcaaactgatcacaagaataatgcgcttgcattgattgttttgcttcaatatgttccgagattatatttgatttttccattaagttctcaaataatcaaagcaacaggagtggttacgaagactgcttgggcaggagctgcatataatttgctgttgtatatgttagctagccatgttttaggggcagcttggtatcttctatccgtggaccgatacactacttgttggaaatccttttgcaaaaaggaacatgatcctgaaaattgctttctttatctagactgcacttcgttgaatattaagctgcgcgagatatgggcaaatagtacaagtgtgtttagcagctgtgatcccagtaatgataatattaatttcaagtatggaatatttgaaaatgcagtaaagaaacatgttgtctcttcaaactttatcccaaagtacctttattgtttatggtggggattgcaacaattaagttcttatgggcagaatctagaaactagcacgtttattggagaaacatcatttgctattgtcattgccatcttgggtcttgtattattttcccatttaattgggaacatgcagacttatttgcaatcaattactataaggcttgaggagtggaggcttaagcgaagagacacagaggagtggatgaggcatcgccaactccctgaagatttgagaagtcgtgttagacgatttgttcaatataaatggcttgctactcgtggagttgatgaggaaaccattctacgtgccttacctgctgatcttcgccgtgatatacagcggcatttgtgcttagaccttgttagaagagttcccttcttctcacagatggatgatcagcttctggatgcaatatgtgagcggctggtatcgtctctgagcactcaaggcacctacattgttcgtgagggtgacccggtgactgaaatgctttttatcattagaggtagactggatagttctactacaaatggcggccgtagtggtttcttcaactcaatcatattgagacctggagatttttgtggggaggaacttctttcttgggctttactccccaaatccaccattaatttgccttcttcaactaggacagttaaagccctaagtgaggttgaagcttttgctcttcgggccgaagacctcaagtttgttgctaatcaatttaggcgccttcacagtaagaagctgcagcacacttttcgattttattcccaccattggaggacttgggcagcttgctttatacaggctgcttggcgccgatataagaaaaggatgactatgaaagacctcagtttgagggaaaccattcctttagacgaagcagtagctggtgagagaaaacatggggactattctgctggttcaaattcaactcagactaaattgaaccttggggctacaattcttgcttcgagatttgctgctaacacacgaagaggggctctgaagatgaaagatgacatgccccagttacagaagcctgaagaacctgacttttccactgaagctgacgatgattag

SEQ ID NO: 16 - DNA, 1068 bp, Glycine max
  source 1..1068: mol_type = other DNA
atgaaacggtttttcaaagtggtggagaaagattcagatgggtctgtgaagaagccaagagagaatgattcggaggctgaagaaagtaatgagaagaaggagccgttgaagtttatgacttggaatgccaacagctttctccttcgagtcaaaaacaactggcccgacttcaccaacctcatcaccacttttgatcctgatctcattgccatacaggaagtgcggatgcctgctgctggtgccaagggcgcgtctaaaattcagggagaaataaaagacgatacaagcgcggccagggaagaaaagaagattttgacacgtgcactttctgctccaccttttgggaactatcacgtttggtggtctcttgcagattcaaagtatgcagggacagcactactggtgaaaaaatgcttcaagccaaaaagtgttgttttcaatcttgataaattagcttcaaagcatgaaccagatggtcgagtaatcctagttgaatttgaaacacttcgtttattgaatacatatgtaccaaataatggatggaaagaggaggcaaactcatttcaaaggagaagaaaatgggacaaaaggatactggaatttgttacacaaagttcagacaagcctttaatatggtgtggggatctgaatgtcagccatgaagagatcgatgtgagtcatccagactttttcagtgtggcaaaactcaatggttatattcctccaaataaagaggactgtgggcagcctgggtttaccttgtctgaaagaaaacggtttggtaccattttaagagaggggaagctggtggatgcctacagatttctgcatgaagataaggacatggagcgaggtttctcatggtctggaaacccagtgggaaggtatcgtggaaaacgaatgagaatagactactttttagtttcagagaagctaaaagaaaggattgctgcatgtgaaatgcgtggacatgggatagagttagaaggtttctatggaagtgatcattgtccagttaccttggagctctctcctagttctaactctcaaaatgaggatccaatataa

SEQ ID NO: 17 - DNA, 1089 bp, Glycine max
  source 1..1089: mol_type = other DNA
atggctattcgtgtaaccttttccttttcgggctatgtcgcccagagcctcgcctcctccgccggcgtgcgcgtcgccaattcgcgttgcgtccaggaatgctggatccgtacgcgcctctctggcgccacccagaagacggatctagattcctccgccggcggagtccgcaattttgccgggcccaagcccaactgctgggcccagtccacttactccaccctcaccggggagttcctcggcgacggctgcaagagcccaatcatattgggcctgatttcgatcatgaagtcaacggcgggtgtgtctgggtcatccgcggcggctgcgggtattttcggtatttcgcccttcaaaaccacttcgatcgttcccttcttgcctggttcgaagtggcttccttgcaacgagtccgttcccgaccccaccacttcctgggaggtcgacaaaggtggcacgcgacgcgtcgtttcggacaccgaatccaattttgccaagactagttggctttcgcggttgatgaacgtgtgttccgaagacgcgaaggctgcgttcactgctgttactgttagtttgctctttaaatcgtccttggctgaaccaagatcaattccctcttcttctatgtaccctactcttgaagtaggtgaccgtgttctaaccgagaaggtttcgttcttctttagaaagcctgatgtttcggatatagtcatttttaaagcgcctccttgtttagaggagtttggttttagttcgtctgatgtgtttataaagaggattgtggctaaggctggggacactgttgaggtgcgtgatgggaaactactggtaaatggtgctgctgaagaacggcaatttgtagtagagcctctagcttatgagatggatccaatggttgtgccagaaggatatgtgtttgtaatgggagacaatcggaacaacagctttgattctcataactggggccctctcccagttgagaacattgttggtaggtccatgtttcgctactggcctccatccaaagtatctgacactgataccctaagtaaactcccacctgggaacaaacctgtggcaatttcttga

SEQ ID NO: 18 - DNA, 2115 bp, Glycine max
  source 1..2115: mol_type = other DNA
atgttgacaattctacgtcatgccacacgcaaaagagtaagagcaacacttattaaacccccaccgaatctactccaaatctcatccacttcagtaaacagtcacacttacactatcacacagcattactctcactcaacaacaatgttgccgccagcgggaaccatcgtattcaacaccgtcggaagagtccaatacggtttcgacgttttctctgtcgacctcaatcaccaacagcctccgccccacgccaccgcagaccaccgcctcaccgacggtgtctcgattaacttcaacgcacaattcattaacccccaaaacgacgtcgtcttcgtatccgaaagaaccgggtcgccccgcttctacattacaaaacccgacactaaaccccagcctttaccctttctgcccaacgctctcttccacgatcgcccaatcgttaaaaacggaaagctctacttcgtttccacgcacgaagaaaacgacgccgttttcactagctggagcgccgtttactccaccgccatagatgactccaccgccaccgtcaagagactcaccccgcgcggcgaggtggactacagccccgccgtctccctcaccgggaaattcctcggcgtcgcttcttaccgctctcgccgctggcaaaccaacgatttccgcgaactccaaacggagatcgtcgttttccccgagtccgatccaaaaaaccgagtcacggtgagcgagcgcggtggctggccatcgtggtcccaagattcaaccattttcttccaccgaatcgccgaagatggatggtggagcatcttccgcgtcgatttaaccgattccgaactttcagaatctcgaaactcgccgattcgcgtcacgccgccggggttgcactgtttcactccggcagctttccacgacggaaagcgcgtcgcggtcgcaactcgccggagagaaagcaatttaaggcacgtcgaaatcttcgatctcgaatcgcaaacgttccaacgaatcaccgaagacataaaccctaactttaaccattacaacccgttcgtctcgttggactctcgccacctcgggtaccaccgattccgaggcgagtcgattcagggcgagccaatatacccgcacctcgacaaagtcctctctccggttcgcgacctacagttactcagactcaacggttcgtttcccaccttctcccccgacggcgatttcatcgctttcaaccacggcttagacggcaacgttaacggcggcgttaaaatcgttaaatcgaacggttccaaacgctggacgttgctcaaaggacgaacgtgcttccacaacgcgtggagcccaacggagaagaacgtgatatacacctccgtcggtccaatcttcgagtcggtttataaaacggttcaaatcgcgcgcatcgagttcgacccggttcatttaactaacgaccgtgaagaggttccgtttaagttaacgattctcacgagagaagacacagggaacaatgcttttccgtcatgctcccctgacgggaagagcattgtgtttcggtctggaagggaaggatacaagaacttgtacatagttgacgctgttaaaggggagtttgacggcgggataaggaggttgacggagggggagtggatcgatacgatgccgtgttggtcgccgaaaggggatttgatagcgttttcatcgaacaggcatgaccctaagaatagtgaggtgtttgggatatatttggtggggccaaatgggagtgggttgaggagggttgaggtggcaaagggggtcgaaggaatgaaagagaggttgaaccacgtgtgcttcagccgcgacggagagtggttgctgttcacggcgaacttgggtggcgtgagtgcggagccggtggggctgccgaaccagtttcagccgtatggggatttgtatgtggtgaggttggatgggagtgggttgaggaggctgacgtgtaatgcgtatgagaatgggacgcccacgtggcaccatggggatttggcgctgtgtgctcctagagaagatgatggcgaaggaggtcaggattgggataagttgaagggtgagtttcaagaaccactttggatcacgtgtgatatttga

SEQ ID NO: 19 - DNA, 2463 bp, Glycine max
  source 1..2463: mol_type = other DNA
atgagcacaagtgtgcttccctctctcctgttcttcctagtcctcatcctcttgctacttgacccttctgaacccgaaaacgtagccatttactggggtcaaaacgccgacgagggaactcttaatgaaacatgtgcaacagggacatactcgcatgtaatcatagccttcctttccacctttgggaatggccagacccctcaactcagcctggctgatcattgtgacccctctactaatgggtgcactaagattgggagagagattaagaattgtcaagagcaagggatcacggtgatgctctctattggaggaggatctggtaattactctataacttccgatgaggatgccaacaatgtgtccaattatttgtgggataatttcttcggtggcttctcctcatcacgcccatttggtgatgcagttttagatggccttgactttgacatcgcgctgggtgataatacatctttcatggcaaatcttgcacaatatctcaaatcaaattcagattcacagacgatacaacaaaaacaactacctgcatctttgtacttgtcagcagcacctcagtgtccgtttcctgatgctaggctgggttcggccattggtaccggcatttttgactatgtctgggtacaattctataacaacccttcgtgcagttatagccaaaacaatttagataactttctaaaatcatggagagagtgggccacatcattgaaagtaggtaaactgtttctggggttaccagcagatgaagctgcagctccggccggaggttatgttccggctgatgtattaatgtctaagatcttacctgaaataaacaagtcaacaaattatggaggtctcatgttgtggtcaaggtactacgataaaattagtggatacagtacccgcattcaaaatcctctaagaaatgggacagcaaaccctctatgcacacggaaaagtcaagcatgtaggagccatgagggcggtttcgcggagcttcttggttacatgtccacgctcggtatcaaagtttatgaagacgacaacaatggaacacagtgctgtgagatcatatgtaggaacaactgttcctgcgacgcgtttgctcccttaaatcacattaacaatactagtactggatgtcagatatggctcaaaggaaccaaatttgttagagcttctggaaatatagctctgccaatcaacgtttctgttgcattgctggaacacaaagtaaacagctggtggatttggctcatcgttggggtgggagcagcttttgtaatacctgtgatattctatttatcgcgtgccttcctgagaaaatacaaagcaaaagtggagagaaaaaagatgcagaagaagttattacatgacattggaggtaatgcaatgcttgcaatggtgtacggcaaaaccataaaaagtaataacaagggaaagactaacaatgaagttgaactatttgcttttgataccatcgttgttgccacaaacaatttctcagctgcaaataagttaggagagggtggttttggtcctgtttataagggaaatctaagtgatcagcaagaagtagcaataaagcgactttctaaaagttcgggacaggggctaatagagtttacgaatgaagccaaactcatggccaaactccagcacactaatctggtaaagcttttagggttctgcatccagagagatgaaagaatattggtctatgagtacatgtctaacaagagcttagacttctacctatttgattcggctagaaaggatttgctagactgggaaaaacggttgaacatcattggaggaattgctcaaggacttctataccttcataagtattcaagactaaaggtgatacatcgggacctaaaggcaagcaatatattgcttgatcacgagatgaatgctaaaatatctgactttggcatggctcgaatatttggagtaagagtatcagaagaaaacacaaatagagttgttggtacatatggatatatggctccagagtatgcaatgaaaggtgttgtgtccatcaaaacagatgtgttcagctttggtgtgttgctactagagatacttagtagcaaaaagaataatagtcgctatcattcggatcacccactcaacctcataggatacgcgtggcagctctggaatgcaggcagagctctggagcttatagactcaacgttaaatggattgtgttctcagaatgaagttttcagatgtattcatattggcctcttgtgtgtacaagatcaagcaacggatagacctacaatggtagatattgtttcatttctatcaaatgatacaatccaacttccccaaccaatgcagccagcatacttcatcaatgaggttgtggaagagtcagagcttccttacaaccagcaagaatttcattccgaaaatgacgtaacaatttctagtacgcgtgcaagataa

SEQ ID NO: 20 - DNA, 552 bp, Glycine max
  source 1..552: mol_type = other DNA
atgccaattgttcagaaattgtatgatacctgcaaggcatctttgtctcctgagggacccatttcagaggaagctctggaaaaagttcgaatcattttagctgagttgaagccttctaatgtgggtcttgaacaagaggcgcaattgatggaaatcttctgtaggtctccaggttctgttatcccgcttcataatcaccccggaatgactgttttgagcaagcttcttattacttgtaaggtcatatgctgggtttactctgggttcgatgatccatctcaagcaagaccagcaaaacttatgaaagattgtctgatgagtgcaccctgcaacacaacagttcttcatccaagcaaaggtggcaacattcattgtttcaaagccttaaccccatccgctctctttgacattctttcccctccttactcatcagaagatggaagacactgctcatatttcaggaagtcaactaggaaagatcttccaggtgttgagcatgatcagctgagtggggtaaagccatctgaaataacctggtcagaagagatctaa

SEQ ID NO: 21 - DNA, 3180 bp, Glycine max
  source 1..3180: mol_type = other DNA
atgataaagtatttcaatttcaatcgaggaagggacaatgccgccgatgcctcaccggcggcgacgccttcttcgtccccggtgaccggtccggcgaggccaattcgccttgtctattgcgatgagaatggacggttccgcatggaccccgaagccgtcgccacgcttcagctcgtcaaggaacccgtcggcgtggtctctgtgtgcggccgtgctcgccagggcaagagtttcattctgaatcagcttttggggaggactagtggatttcaggtagcatcaacacatcgaccatgtaccaaaggactatggttgtggagtgcaccgttgaagaggactgcccttgatggaacagagtacaatctcctgctattagacagtgaaggaatagatgcttatgatcaaacgggaacatacagcacccagatcttctccttagctgtccttttgtctagcatgttcatttataaccagatgggtggtattgatgaggctgcccttgatcgactttctcttgtcactcagatgacaaagcacattcgtgttagagcatctggaggaaggagttctacatctgaactgggacagttttcccctatttttgtttggcttctaagagacttctatttggacctcgtagaagataacagaaaaataacaccccgtgactatctggaaattgctttgaggcctttccaagggagtggaaaagatataactgctaaaaacgagattcgagattccattcgggctttattcccagatagagaatgcttcactcttgtgcgacctctgaacgatgaaaatgatctacagcgacttgatcagatatcgttggaaaaattacggcctgaatttcgatctagcctggatactctgacgaagtttgtttttgagagagcaaggccaaaacaagttggggcaactatgatgacaggtcctgtgttgattggaatcactgaatcttatctggatgctctcaatcatggtgctgtgcctacaatttcttcctcctggcagagtgttgaagaagctgagtgtcgtaaagcttatgattctgctgccgagatttatatgtcctcttttgactgtactaagcctcctgaggaggctgctttgagggaagcacatgaaaaggcagttcgaatttcaatggcagcctttactgctagtgctgtaggggttggttcagtaagaacaaaatatgaaggcatgctgcagaaattcctcaaaaaggcatttgaggattataagaggaatgcgtatatggaagctgatctacaatgctctaatgccatacagtctatggaaaagaggctaagggcagcttgtaatgcttccgatgcaaagatagataatgttgccaaggttcttgatgctcttttatgtgaatatgaaaaatccattcaagctccagagaagtggcaaaaacttgctgtcttcttacaacaaagttttgaaggcccggtactagacctaaccaggagactgataaataaagttgaatctgacaagagttctctcagtttaaactatcgattgactgaagataagattgccctgcttaataagagactggaaaccagtgaaagtgaaaagtctgagtatattaaacgctatgaggatgccatcaatgataagaagcaactaactgatgaatatatgaatcgcattactgaactgagggctagctgtcgctcattggatgagagatattctagcttatcaaaaacattggattctaccaagcaagaatccatggattggaaaagaaaatatgaacaagttttatcaaggcataaatctgaggaagaccaagctagttctgaaatagcagccctcaagtcacatagtagtgctgctgaagcaaggttggctgcagcaagggagcaatcccagtctgctcaggaggaggctgaagagtggaagcggaagtatgaaattgctgttagagaagcaaaagctgctctagagaaggcagcaattgtgcaggagtacacaaacaagcaatcacaattgagggaagatgctttaagagaggaattttctagcactttggctgaaaaggaagataaaataaaggagaaaactgccaaaattgagcatgctgagcagtgtttaacaactttgaaattggaattgaaggctgctgagtcaaaaataagaaattatgagtcagagatatcaccactgaggcttgaaattaaaaagttgattgagagattgaaaactgaaaatgctagggctcagtcatatgagaaggatgtaatggtaattcaacaggagattaaccatctaaaggagaagtacaacaccgagtgcataaaatttgaggaagttcaggaaagatgtcaaattgctgaaaaagaagctgtaagggctactgaagtagctgacaaagctagggctgaagccaacttggctcagaaggagatgagcgagatgcagaggcttgcaattgagagactggcacatattgaaagggctgagaggaaaattgaaaatttggagagggagaaagataatttggaaggtgaattgcaaagagtgagggattcagagaaagatgcacttgttagggtttcaacactagaggaaaaggtgggacagagagagaaggatatagattcactcttggagaaagatggaacacagaggcgaaatagcacacagattcttgatcaacttttggaaacagaacgtgaagcatgtgctcaggcaaatagcagggccgactctctctctctccagttacagtctgcacaagcaaaaattgattctctccatcaagaactgactaagtttcaactgaatgaaacaatattggacagtgagctaaaaactgcctctcgtggaaagcgtttgagggtagatgatataggtgtagaatctggtcaggacatggattcgagccctagaatcttaaggggaactaagagatctaagagtacatctagtccacttaagttttcacatctggaagatgttagttccattggaggtgatgaagacaactatagtcagcaaactaacgaggatgattataaaaagtttaccatccagaagctcaagcaagagctgacaaaacacaactacggtgatcagctgcttgagttgaagaatcccaacaagaaagctatccttgctctgtacgagaaatgtgttcttcagaagtcatag

SEQ ID NO: 22 - DNA, 960 bp, Glycine max
  source 1..960: mol_type = other DNA
atggcttatgaaccaccattcctcgagacctacaagacccttgtgcaacaacacttaggggattcaaggaatgagttcatagtagagagatgtgatatacctttgatcgaccttggccggttaagtcttgagagagaagaatgcatgagggaaattgcagaagctgcaagagaatggggtttctttcaagttgttaatcatggaatttcacatgagcttctgaagagcctgcaaattgagcaaaagaaagtgttctatcaaccctttctgaacaagtcatcaacacaaggcaaagcttacaggtgggggaatccatttgctaccaatctgagacagctgtcatggtcagaagcctttcatttctatctgacagacatctcaaggatggaccagcatgaaactctgagatcaagccttgaagtttttgcaataacaatgttttcccttgcccaaagcttggcggaaatactagtttgcaaattgaatacgaaatccaactatttccgagagcattgcttgccaaaaagttctttcattcgactgaatagataccctcaatgccccatatcatcaaaggtgcatggcttgttgcctcacagtgacactagttttcttaccattgtacatcaagaccaggttgggggattgcaattgctgaaagatggaaaatgggttggtgtcaagcctaatccacatgccctggtggttaatattggtgacttatttcaggcattgagtaatggtgtttataaaagcataaaacacagagtggttgctgctgagaaagtagagaggttttctatggcatttttctattccccctctgaggaggcaattatacaaagccaaatcaagccacctatctacagaaaattcactttgagggagtatagacaacaaacagagaaagatgtcaagcaaaccggggataaagtggggctctctagatttcttctgtag

SEQ ID NO: 23 - DNA, 525 bp, Glycine max
  source 1..525: mol_type = other DNA
atgttaggatttgacgatggtgattctgattccgacaacgagcaggcactgtttgggaagtacccggagctacagatgtttaagtgggagaacagtctcattcaccttccttccaatttatcatctttgttattgccgcagacaccactgtttcactgtgagcgtgaagaaaccctcagggttcaccctcctccctttcttccgttgcaagaaaccctcgatcatctgtcagcgaacactgcccctactcaggacatcacccatcttcaacaggcacgcccagcgaacccaccccccacttcatcaccatctcccaatctctgcaagagatggaccaatgaagaacacaggctgtttcttgaagggcttgcctactttggcaagggtgattggaagaacatctcaaaacatgctgttaagacaagaaccaagactcaagttgctactcatgcccaaaaatattttcttcacataaaggaaaagggaaagggaaaaagaaagagcctctttgacatggccttgtaa

SEQ ID NO: 24 - DNA, 4827 bp, Glycine max
  source 1..4827: mol_type = other DNA
atggaacctcctgtcgtgagatccaggggccgcccacgcaaacggaggagagaagacgaagcaaccgtcactggtgacgcgaaaacgcttcccgaggcaaagaagacgacgccggttgcattgattggacgatacgtgctaaaagagtttcgaagaaacacggttcttctaggcaaagtcgcgcgttatgttagtggactgtacagagtcgtatatgagagtggcggtttcgaggatttagatagcaacgagattcgtaggatacttctccttgatagttatttcgacgacgatttgattcggaggaaggttgaattggaggaatcggtgttgccgaagatcacagcagaggaaccggagaaaggttcgagcgaattgcaaggtgaattatcggttgacaatgaagaggaacgagctgagacggatgatgatgaggccagggattcgagttccggtgcggaaatgccggaaaaggcgataccgtcgccgctgatgctgccgccgtcttccggaacaattggcgtgccggaaccgtgtgttttgaatctcttttcggtttacggatttttaagatcgtttagcattcgcctgtttcttagtccgtttactttggacgagtttgttggtgctttgaactgtaaagtttcgaatgcgttgcttgatgcaattcacgtttcgctgatgcgtgttttgaaacgtcaccttgaaaacatttcaccagatggctcgcggcctgcaacaaaatgccttaggtgcagtgattggagtttggttgatgctttgacttggcctgtttttgtgtttcagtatttagcaatttttggatacacaaaaggacctgagtggaaaggattttatgacgaaatcttttatggcgagtattatttattgcctgcaagtaggaaattgacgatattgcaaattctctgtgatgaagttttggcgtctgaggagctaaaagctgaaatgaacatgcgtgaagaatcagaggttgggattgatcatgataatgaagattgccttcctgctgaaaatgggcctagaagagtccatccaagatattccaaaactactgcctgcaaggatgctgaaactaagaagtatgtttcagaattgaatgctgaggaggatgatgttgatggcaatggagatgaatgtcgcttgtgtggcatggatgggaccttgctttgttgtgatggttgtcctgctgtttatcattcaaggtgtattggtgtgatgaagatgcatataccagaaggggcatggtattgtccggagtgcaaaatagatatgattgggcctactattgcacggggaacatcacttaaaggggctgaagtattcggaaaggatttgtatgggcaagttttcatgagtacttgcaaccacttactggtgctcaatgtcaacagtgatggcttttgtcttaaatactacaatcagaatgacatacctagagttctccaagtcctttatgcatctgagcaacatagacctatatacaatggtatttgcatggcgatgttagaatattggaatatttcagaaaagttcttacccatttgtgtcagtagactaactcctatggtcgaagaagagcataaagctgttagttcggtgaaggaagaatacagtttaatgtttgggaatgggatttgtggtgataatttggtgccatctcttgatgcttcattggttacaactcgaagtcctgctcctggaagcagtggcaatgcaagaactactgtgaatctgaagcttaatgaggaaactgcaatggattcgactgtttcaactgttaaccatcatcactctgacccaaagtgccaaaattctgttaataggtcagctgcagtaagccctgtcaagtgctccttggtaagcagacaatttaataattatgggcacgcaaatgacgtagggttgcctatgaatttgtctttgcaaactaaaggcgaccaatctggttttggaaagtgcaaaggcagtttaactaatgattttgtgtacatgggttgctcctataaaccacagtcatatattaattactatatgcatggtgatgttgctgcgtctgctgctgctaacctggccgttctttcttcagaggattctagatcagaaggtcatgtgtctggcaacttggggaaagcaacatctggaaacacttatttgctagcaaaagcattctcccaaacagcttcacgcttcttttggccaagttctgaaaagaagcttgtggaggttccaagagagaggtgtggctggtgcatttcttgcaaagcctctgtttcaagcaagaaaggatgcatgttaaatcatgctgctataagtgccaccaaaagtgcaatgaaaatcctttctggtcttgctcctgtaaggagtggagaaggaatcatccctagcattgcaacatatgttatgtatatggaggaaagtttacgtggcttaatagttggaccctttctgagtgaatgttatagaaagcattggcgtaaacaagttgaacgagccaaatcatttagtgatataaagccccttttactcaaacttgaggagaacattcgaacaattgctttttgtggagactgggtgaagttgatggatgattggttggctgaattttctacaatgcaaagtgctacatgtactcttggaacaacacaaaaacgtgcaacttgtgggaagcgtaagaaacagttatctatcaataaagttacagttggtggttgtcaggaaaattttgcatggtggcatggtgggaaatttaccaaatctgtatttcagaaagctgttttgccaaaatccatggtaaaaaaaggagcccgccaaggtggtttgaggaaaatttcgggtatattctatgctgatggctctgagattcctaaaagaagcaggcagctagtttggagagctgcagttcaaatgagtaggaatgcatctcagctggcacttcaggtccgatatctagattttcatatcagatggagtgatctgattcgtcctgagcataacctcctggatgtgaaaggtcaagatactgaagcttctgcttttaggaatgcaaatattcgtgataaaaaatttgcagagggaaaatttttgtacagagtagcctttggaatccagaaacatcttccttctcgggtaatgaaaaatgctgaaatagagcaaggtcccgaaggaatggaaaaatattggttttctgaaacacgcattcctttatatttggtaaaagagtatgaagtacgtaatggaaaagtgctatctgagaaagagtacatgcatattacatctcatatgcacaaaaggcggttgacagctacctacaaggacatatttttttaccttacctgcaagagagacaagttggacatgttatcatgctctgtatgtcagctgggtgttttaattgggaatgctctcaagtgcagtgcttgtgaaggttattgtcacatggggtgttccgtgagttcgacagtctctacatgtgaagaagttgagttcttggccacatgcaaacaatgtcatcatgccaaattacttactcaaaaacagtcctgttatgaatctccaactagtcccttacttttacaaggacaagaacgtagcacttcggcagttctgaaggggccaaggcctaatggtgatggtcaaggattgatgtctgctaagacaaagaatagtcgacttgacatgaaacgagttgcttctgatttccctttggaaacaaaaggccgtagcagaagttgctcttggggtattatatggaaaaagaagaataatgaagatacaggctttgatttcaggctcaagaacattcttctaaaggaaggttcaggcctgcctcaactggatcctgtttgtcgtttgtgccataaaccatatagatctgatctaatgtatatttgctgtgaaacatgcaaacattggtatcatgctgaagctgttgaacttgaagagtccaaactttttgacgtgctgggcttcaaatgttgcaagtgccgcaggataaagtcgcctgtgtgtccttactctgatttgtacatgatgcaagggggcaagaagttgctcacaagggcttcaaagaaagagcactttggggcatattctgattccggtacacctatcgacatgagaacatgtgagcctgcaactcttatctatcctgcaggggatgtctctagacaagacaatgatcctctgtttttctccctttcaagtgttgagctgattacagaactccagttagatgcagatgatgcaggtaatactgtctctggtccagggcttccgaaattacctaaatgggagggggagaataatggttcttttataggtaatcttcatgctgagttttcaacaagcaatgcaatggtgtccaaatctgtaaaagatttatcacctgtagaatatggctctgcggactgcaatcttctgaataactctgaaattgtaaattttgatgaattggtggattttgaacctaatacctacttctctctgactgaactgctccactcagatgataatagtcaatttgaggaagccaatgcatctggggacttctcagggtatttgaaaaattcttgtacattaggtgttcctgaagaatgtgggactgttaatttagcatccaactgtggatctacaaattcgttacaaggaaatgttaataaatgtcggcaatgttcacaaaaggaaccggcccctgatctctcttgtcagatttgtgggatttggattcacagccattgttcaccttgggttgaatcaccatctcggcttggtagttggagatgtggtgattgccgggaatggcgatag

SEQ ID NO: 25 - DNA, 2625 bp, Glycine max
  source 1..2625: mol_type = other DNA
atgaacagaaaaattaatgacaggttgaaaccggagaaagcttctttgtcatacgctgattttcatcatgaaattaccaaaaatgtggaagacaactctttaaaaccttaccgaaataagcaaaagcaagcaacttatcgaagggcaagtgaggaagatgagctggttaagtatatgtcgaatctaccaggttacttagagaagggagaaaaaattcccgataaagctttgaatgttggggtgcttgattgggccactctacaacaatggcagtacagccataaacatttgcccttaagtagccggagctcgacatctactggtaatacatcatcttctgtttcagcagaggaattatctggtaattctagcaaaggtcctgtttgttctccttcacgtcaaagaatatttcgtccgtcgctgaaatctcacttcatggcatctccaatgcaagactactctgcatctgtcaaatcctctggagggagttttggaaattgtcagaatcttagaggtgggtgcagtaatattgacacacacggcaaatatgctcaagttggtgatcatctttcccaaaaccatcctgctagtataccaaaaggatgtgacaggagacaactgaatccacatattagtaaagaaagtgatatcttgccaaatgggggaatgtatgaggcagcatcacatactacgactgaaatgagctctcaagatgatagaccagaaaagaaagtggagaatttcagacaaccaaacattgatgctgatgagcaagttatgcttgggaaaagcaaacctattgttcttattttgcctagagacattccccagaataatcattgtgaagttcctgatatgcaaacatccttgggtcaaaagctaggaagtcccactggaacaagattttcagaaaaacctaaggaacctccctgtagatatccaaattccaatatttccaaaacatgtcctctgccagatgaaattagaggaatccgttatcagccggaaaggtcaggatctagttccacagatccagaaggtattaaaattcctgcttctactttatcagcacctgtaccagtcagaacagggataataagtccatgcagatctagaaaggctgaggaaaagaaacacaacattggtgcatcttcatctgcaaatgggtctctcaagggattagatcagaaagtaacaactgagaaaccaagaagttcttcaccttttcggcgatttagcttcagcattggctttacaggtaaaggttctggctgtaaagaggttgcacatgtgccacatcagagctccatagcagcacttaaatctagttcagaaaatgtgagaggttatgctagctcaaaaatttcaggcaatgataaacctggtaatgctgtcaaaagcaggtctagtagccctttaaggagactactggatcccctgctgaaaccgaagacatcagaccatcgctcggtggagtcatctcaaaaagattcagtggtaataaagaagaattgtaggtcagctaatgggggatttgctatggagaaagaactggacagggaccagagggttggttgtacaaatacagcaattaatacagttgatttatcaaagaacaagaagtatgtgccgtcaacgtttcaagctcttctaagaattgctgtgaagaatggtcagccccttttcacatttgctgttgacaataatagcaacattcttgtggccacagtgaagaacttggctgtctcaaaggaagacgaatgcaatcgtatctatacatttttcacctttagggagggaaaaaagaagaacggaagttggatgaatcaagcaagcaaaacccaaggtcccgattatattcatcatgctgttgcccaaatgaaggtttctgattcacaccattatgattcaactagccagaattgtgtggactcctctacatcaaaagagtttgttttgttttctgtaaagttaaagcagggagatgctcaggtcactgactacaaaccaaatgatgagcttgctgctattgttgtcaaatcagctaaggctgtcaattttatcaattatgcacatcagagcagtcgccagaatgacagtcaagatctacatgtaacagttgtgctcccaactggggttcacagttttccaagtaacggaggaccttcatcactgattgagcgctggagaacaggtggatcatgcgactgtggtggttgggatatggcttgtaaacttaagattcttgcaaatgaaagtcaagcatgtagaaaatcaagaatatcaaaagcttgtttcccacatccatttgagcttttcctccaggtgaatgaccaagacctagagaaccagcctgctttcagtttttccccctttaagcctggggtatattcggtagcttttgattcctcattctcacttttgcaagcattctccatctgcatagcgttagttgatggcttgatctcatatgaactttctggatcaagaaactacatcgaaggaaaaaattcgagagaaactctgttggtgcaaacagatgaactaaaggcttttggcaaattagaggacattcctgcaagttatgttgcttatcctcccctctctccagttggtagggtctaa

SEQ ID NO: 26 - DNA, 1983 bp, Glycine max
  source 1..1983: mol_type = other DNA
atggatccctcacgccgtgccccacgcgccgtgatcgaccctataccgaagatccgccaagtggggttctttgccccgcccgagcgctctcagtcgggtcctcccgacccgacccactcctctcccccgatctccagctccctctccccggtcatgatcccgcccccgcgccacctctccgacaacctccttctccacgccccccgccccgcctcctcccccctctccgccgccgctgccgccggaggaagcggaaactcctttgactacggcgcggtcttcttccccgccccgctctctcccgcgataccgtcgtcttcgtactccagcagaatcatcgccaccaccggcgagggagggttcttcaaggggaacggcgccggcggcggaaaggtggcggcgtcatcgttccctcgcggaggattcgacttgacggcgatgaagaaggcggcggcggcgagtgttgtggtgccggcgagcgagttgactacggtttccgtggtaaatgattcgctcgggattcctgaaaaagaaaaaggaaataaagcaggagggtcagcagtggaagtgaaagaccaacctaatagttcaaagcagcagaaacccaaactctcaaaagctgaaagacgtgcactccaagaatctcaacgggctgcaaaggctgctgcaaaaggtgaaggaagtaaggcatctggaactgtggctacaatgaatgcaaaaccagctaaagctgtaaagcctgcacagaaagttgataatgcagcagttgcagcgtctgaaaagaagggaggtgagattccaccagaaaaggatagaaagaaagatgctcctcaaccacgcatgcagtatgacgacaagagtcgagtggagaaagctaaacgtcgtgcagtggtgaaacaaactgaagctaggaatagagttgagttgttcaggcatttgccacaatatgagcatgggagtcagcttccagatctcgaggcaaagtttttccatctttctccagtgcacccttctgtttataaggtgggtttgcaatatctaactggagatatagctggtggcaatgctcgttgtattgcaatgcttcaagcattccaagaggccatcaaagactacaaggttccacatgagaagactcttgtgagagacttgacagcaaaaattagtagttatgtatcatttcttattgaatgtcggcctctgtcaatcagcatgggaaatgcaattcggtttcttaaaagtcatatagccaagctacctttgaccttgtctgaatcagaagcaaaagcttctctccagtcagatattgagcgttttataagtgaaaagattatacttgccaacaaggtgatagtgaagcatgctgtcaccaaaataagagatggtgatgttcttctaacatatgggtcctcatcagctgttgaaatgatactattacatgcacatgagttggggaagcagtttcgtgttgtcgtagttgactctcgtccaaagcttagagggcaactcttacttcgcaggcttgtggagaaaggtcttagctgtacatatactcatataaatgctgtttcctacataatgcatgaagttactcgggtttttctgggtgcttcagcagttttgtctaatggaacagtatattcaagagtagggactgcatgtgttgcaatggttgctcatgcattccgtgtacctgtcatagtatgttgtgaggcctataaatttcatgaacgggtacagttggattcaatatgctcaaatgaacttggcaatccagatgtcatttcaaatgtactgggtagagaggatgtaaaacacttggatggctgggccaatattgaaaatctgcaacttttaaatctggtttatgatgcaacaccttcagattatgtttcaatgattgtcacagattatggcatggttccccccacaagtgtgcctgtgattgtaagagaatatgggagagaacaggtctggatataa

SEQ ID NO: 27 - DNA, 4596 bp, Glycine max
  source 1..4596: mol_type = other DNA
atggatcacaggccgaaatccaaggactcactctcttactccactctcttcaatctcgagcctttgatgaactttcaacttccaaaacaagatgatgattttgattattacgggaatagtagtcaggatgagagcagagacagcgaaggtggggggattacaaatcacggtaatgggaatgtgcatgaaaaggaagtgaatttgttcaagaagaggagatggtctctaaacagtgacaacgaggagaagaccagtttttacggggcacacatgacggaggagcgatatcgatcgatgctgggagagcatattcagaaatacaagaggaggtttaagggtaccttgagtagtcccgcccaaaatcaggctgctgctccacttgtgaaaagcaacacgggactaaaagctcgcaagtcagggaatgagcacaggggaggaggattacatgtggcggagagtacatcggaatggatgaatgattccagttctcagaaacctggaaactaccgcgatgcagatttctcaccacaatatggcactgataggattatgtatgaacctgcatctttggatattggggatggaatcatttacaagattcctccggtctatgataagttggctggagctctgaacctcccaagcttctcggatatccatgttgaggatttttacttaaagggtactttggatttgggctccttagctgaaatgatggctgctgataaaagatttgggaacagaaaccgagcaggcatgggagaagcaataccacaatttgaatcactgcaggcacgactgaaggtcatgtcagcttctaattcagctcataaattcagtctgaaaatgtctgatgttgatttgaattcctccattccggagggggcagcgggaagtataaggcgatctattttgtctgagggtggagtattgcaggtatattatgtgaaggttttggagaaaggtgacacctatgagatcattgaaagaagcctacctaagaagcaaaaggtgaagaaagaccctgctttgatcgagaaggaggaaatggaaagatgtggaaaaatttgggcaaatattgtaagaagagacataccaaagcatcataggaacttcactatttttcatcgaaagcagctcattgatgccaagagggtctcagagacttgtcaaagagaggtcagaatgaaagttagtagatcccttaaatggacaaggactgtcggtatgcgcacccggaaactagctagagacatgttactgttctggaaacgaatagacaaggagatgacagaagtgaggaagagggaggaaaaagaagctgctgaagctttgaggcgtgaacaggagcttcgagaggcaaagaggcagcagcaaaggcttaattttcttatacaacaaactgagctgtacagccactttatgcagaacaagtcgaacttactatcttcagaaactttacccaaggaagatgaagatgcagatgatcaagatgcactggttgactcttcagatgtgatgcccgatgaggaggtagatcctgaagaggctgaattgaagaaggaagctttgaaggctgcacaagaagcagtttctaagcagagaatgttgacaagtgcttttgacactgaatgcttgaggctgcgccaagctggtgaaactgattcacttccaccagatgttgctggagcgagtaatattgatttgcaaaccccctccaccatgccagtggcatccactgttcggacacctgaattatttaaaggggttcttaaagaatatcagctaaaaggtcttcagtggcttgttaattgttatgagcagggtttaaatggtattctagctgatgagatggggcttggaaagaccattcaggccatggcttttttggcccatttagctgaggaaaaaaatatatggggaccttttctggttgttgcacctgcttctgtattaaataattggaatgaggaacttgagcgcttctgccctgaactgaaaagacttccatattggggtgggctttcagagaggacagtacttaggaaaagtattaacccaaaggatctttatcgtagggaagctaaatttcacattctcatcacaagctaccagctattagtatctgatgagaagtattttcgtcgcgtgaaatggcagtatatggttttagatgaagcccaggctatcaaaagtgcaaccagtataagatggaagacactcctcagttttaattgtcggaatcgcctactgctgactggtacacccattcagaataatatggctgagttatgggctcttcttcacttcatcatgccaactttatttgacagccatgagcagtttaacgagtggttttctaaaggaattgagaaccatgcagaacatgggggtactttaaatgagcaccaacttaatcgattgcattcaattctaaagcctttcatgctgcggcgtgttaaaaaggatgtgatttctgagcttactacaaaaactgaggttactgtgcattgtaagctgagttctcggcagcaggctttctatcaagcaattaagaacaagatatctcttgctgagttgtttgatagtaatcgtgggcagctcaatgagaagagaattctgaatttaatgaacattgttattcaactaaggaaggtttgcaaccatccagagttgttcgaaaggagtgagggaagcacatacctttactttggagagattccaaattcccttccacctcctccatttggggagatggaggatgtatattattctggtggtcacaaccccatatcatatgagataccaaaacttgtctatcaagaaattatacaaagttctgaaactctcagctcagctgttggtcctgttgtctccagagaatcttttcataaacattttaatatttttagacccgaaaatgtttatcggtctgtcttctcagaagatatgtacagtaaaagtggaaattttggttttacccatatgatggatttgtctccacaagaggtgacatttctggctactggttcttttatggagcgactactattttctatgatgagatgggaacaaaaattcattgatgaagctgtagactttctaacggagaccatagatgatgatccggaatgtagttaccttgagaaagaaaaagtgagagcagttacacgaatgttattggtgccatcgagatctgagaccctggttcttcaaaaaaaattgcaaactggacccagccatgctccttttgaggccttggtcgtcccccatcaagatagggtgttatcaaatgccaggcttcttcactctgcttacacatatatcccacaaagtagagctcccccgattggtgctcactgctcagatagaaacttctgctataaaatgattgaagaattacatgatccctggattaagaggttgcttgtggggtttgcacgtacatctgataataatgggcctagaaagccagatagtcctcatcatttaattcaagagatagattctgaactacctgtttctcagcctgctcttgagttaacacacagtatttttgggtcttctccacctatgcggaattttgaccctgcgaaattgctcactgactcgggaaagctccaaacacttgatatattattgaaacgcttacgagcagaaaatcatcgtgttctcttgtttgctcagatgaccaagatgctgaatattttggaggactacatgaactatagaaaatataggtattttagacttgatggatcatctactattcaggaccgcagagacatggtcagagactttcagcataggagtgatatttttgtgttcttactgagtacaagagctggtggattgggtatcaacttgacagctgctgacacagtcatattttatgagagcgattggaatccaacattggatctacaggcaatggacagagctcatcgtttgggtcagacaaaagatgttactgtttaccgacttatatgtaaagagacagttgaagagaagattcttcttagagctagtcagaaaagtactgtgcagaaccttgtcatgactggtggttctgtcggtggagatcttttagctcctgaggatgttgtatcattgcttctagatgatgttcaattggaacagaaattaaaggaaatccctcttcaggtaaaggataagcaaaagaaaaaacaacctatgaggggtattcgggtaaatgaagacggtgatgcatcgatggaagatctaacaagctctgtagcccagggtacatcagataatgatctttccatggatccagagggttcaaagtctagtaataaaaagagaaaagctgcctcagataagccaacttcaaggccaaagaactctcaaaagatgagtgaatttagtactatgcctatggacggtgaactggatgatcttgatccagtgggtcagaaacccaagagacccaagaggataaagaagaatgtgaatgaaaagtttgaagatgcttttacttggactgctagcttggtcccagagcagtcccagtttccacctccacgagattttagtgtcgggggttctaaagcagaatcaggccaagacaactga

SEQ ID NO: 28 - DNA, 3483 bp, Glycine max
  source 1..3483: mol_type = other DNA
atgaaggttgaaattgaaggcagccacagtgattatgctgaatctgtttccagacaaccagaaaatagaggaagattggaatccaagaagaaactgaagaaagtgaggtcaataaggcttgtaagattgccaagcaagagatcatccaccagaggagggagacaccgccatgattatctcttcattgacacagaaagtttagaggggccaacaccaattgaaatggtagatgcatcaccaaattacatgaaggctaccagcagttctcatgcaaaagatagtattcagaatacacaaagaatactattcacaaagaagactttgacaagaatgtctactactttgaaactgaagagaagtctgaccagaaagttatctggaaggactgaaccaaagagggaactgaagtcctcaagatcaatcaagtttgcagctgtcaagggccaaaaatcaacaagaaagttttatgaatctaattatggaagtgatgaccagaattggaaaagtgcaagtgatgctggtaacaaactccagagagtcataaccagaagattaagcttgaagcccgtgaggatcttagcaaagatgcctactttcaaatcaaagaattcttccatggagagaggcaatcagatatctcaatccccatacacaagcttgcttagagccacttgtacttctgctctcaaggattcccattttcctgagaaaattgacctccctcaagagggaagtgactctcaaggagtatcagctgtgaaggtttgtccatattcttactgctcacttcacggtcaacgccacaccaatctgcctccactgaagcgctttgtgtcaatgaggaggcgcacgttgaagagccaaaagccaacaaaaatggattgtcaacctgacactagatcaaagcagattggtaatgcaaagaaagccgctcagaaaactcaaactgtccacaaagaagatggaagatctcattttcagaacaagaaaaagctggctaggggcttatggattaggcctcatggcactccagcatccacagtttctgaaggtgtggaatcaacatcaacagatagagtcgagttttctgctcctgatactgagatattggagagagaagtaactaacactggcaacacaagcaaaaatatgaagctggattgtgaagtactgaagatgagttctttacagaaagagtctacacatgctagcactactgatatggcaaggggaatgcaggaaagagataaaaaatttgtcaaaatgtggcagttgatgtataagcatgctgtattgagtaacacaggtgaaaataagcagcaatttgatgggaaggacaaggaaggaagagagcaagatttctttgcaactaatgaagtgaacaactcttgtcgtgatgattgtgatacagatcaggatatggatgaggaaaataaggatgcaattgagctggtgcagaaagcatttgatgaaatccttcttccagagcctgaagacctcttttctgatgatcaattcaaatctgaaggcattgattcagatgtggtacacttagaaaagagtgaagttgagagaaaaaggaatacttcaacatccacagaatctccaacagcacagaggatggggaccaaacctgaccaaagagcaccaagaagctggagcaatctgaagaagttaatcctcttgaaaaggtttgtgaatgcattagagaaggtaagaaacatcaaccccaaaagaccaagacgttttccttctgatgctaacttagaaatagaaaaagtttttctcaagcaccaaacagcggaagagaagaagaatgctgaggaatggatgcttgactatgcacttcaaaaggttgtttctaaacttgcacctgctcaaagacaaaaagtgacacttctagtaaaagcttttgaaacaattctgccatttcaagttgctgaaaatagtccacggttttctccaacaatggaacctcaagcaaatccagttcaaccccttgacaattcctcaaatcacagcgaggaagaaacatcattttctcatgatagcagtatggaactgacagaaaatactagtgatgatccaatgccagaactgcacaatcctactacgctcaaagaaagatgtcttgagtctcttgattttcctggaaccgagacagtcaagaatatgcctgcctttggagctactgaagaagacttgagtggaaagcaaagtttagctggcagttatgataatgaggagaagatatcaagtgacagtgataatatttatcttgtagagatcaaagatactacctcaagttccttaaatgagccagttgaaattataagaagcagtcatgaggaggctccaaccaatgaaacagtcaatgatgttccagaagatttgctatccagtgtgaatacagaaaatccagatatcaaatctgagtcacctggaagagatgttgaaaccaagaatctgaatggtgataatggggaaaaaatttccatgtccaaaagtttagtcctagaaggtttagttagatcactcagatctaatttgattggttcaggagcacctgtaaacgaaccaactgccaacaacagaaaagaaggaattgaaaatgttaaacaggaaactgagactcttgaagagttccctacaaaggaacaatctgaagctcatattagtgctgttgtagaacctgagacccctgtagagaaacagaataacacaggattgtggtacttggtgtataagcacatggtatcaaatatggatgaaaacaattccgagtcactaattgatggggcagatgaaaaagagtcagggtttgatgggagcagaacaagaggagcttccttctctcatgaaagtacacccgtgactgatgaagagatgaaatttaaggaccatgtcgtagctgatccagaagttgcacgccaacagaatgaagccatcaagatggtagaagaagcaattgattcaattcttccagatgatcaggatgacttatcagacaaggaatcactcactgacagcacaatttcagacaactccaaacaatccaacagaactgaaagagtatacagtgaaggcctgaaccaaaaagaagaacagatggaatccgggaacggaatgatccaaaaacaagaagaatcagcaccaaaggagcaaaacaaaacaaaccagaaaatgtctacaagctggagcaatcttaaaaaagtgatcctgcttaggagattcatcaagtcattggaaaaagtaaggaaattcaacccaagagggcctagatatctacctctagagcctgattcagaagctgaaaaagttaacttaaggcatcaagacatggaagaacgaaaaggtacggaagaatggatgcttgattatgcacttcgacaggttgtttccaaattaacccctgctaggaaaagaaaagtggagctgctggtggaagcttttgaaacagtcatgccaactataaaaacttga

SEQ ID NO: 29 - DNA, 219 bp, Glycine max
  source 1..219: mol_type = other DNA
atgccaattgctctaagtagaagggatctattgggttgtgctgaaactggtagcggaaagactgctgctttcacaattcctatgatacagcattgcttggcccaacatcccattcggcgcaatgatggtcccctggcattaattttggctcctagaagagaacttgctcaacaaatagaaaaagaggtgacatgtaataattttagaactatatcctaa

SEQ ID NO: 30 - DNA, 825 bp, Glycine max
  source 1..825: mol_type = other DNA
atggaaatcgttccgattcccgccgattcctacaatctcggcttcatcggcgccggaaaaatggccgagtccattgccagaggcgccgtccgctccggcgtcttgccgccttctcgcattcgcaccgccgtccactccaaccccgcccgccgcgacacattcgaatccttcggcgtcaccgttctcccctcaaacgacgacgtcgttcgcgaaagcaacgtcgtcgttttctcggtcaaacctcaactagtgaaagacgtggtgttcaaattgacgccgcttctgacgaagaacaagcttttggtttcggtcgctgctggtaccaaattgaaagatcttcaggaatgggctgggaacgacagatttataagagtgatgcctaatacacctgctgctgtaggcgaggcagcatcagttatgagcttggggggatctgcaacagaagaagatggaaatattatagccaaattgttagggtcaattggcaaaatatggaaagctgaggaaaagtattttgatgctataactggcctgagtggcagtggtcctgcttatgtttatttagcaatagaggctttggctgatggaggagtagcagctggtttaccacgtgatctttcattaagtctagcttctcaaactgtattaggagcagcatcaatggtcatccagactgggaaacacccaggacagctcaaagatgacgttacttctccgggtgggacaacaattacgggcattcatgagttagaaaatggcgggttccgtgggacactgatgaatgctgttgttgctgctactaagcgcagcagagagctttcctga

SEQ ID NO: 31 - DNA, 237 bp, Glycine max
  source 1..237: mol_type = other DNA
atgtttggtgggcatggaggtttattgaaggagtttgtgtatgtgggaattgggttactggactataaagaagaccgaattttggcaaagacaatatggtataagattcaatttggactatttcgaagctattgcaagataaagttagcgttgggctcttctagcatacttcgagtcatgaaaaggcgtgtaggggtattcattttacttgaagaaagcttcaaaataatgatttga

SEQ ID NO: 32 - DNA, 414 bp, Glycine max
  source 1..414: mol_type = other DNA
atgcatactatagagtttcaaaagcgaggactccctcacgtgcatttatttctatttctacatctagataataaatatccatcttcaactgacattgacgagataatatcagtagaaataccttcccatgaagatgacccagaactctacagattggtggaaaatcatatgatacatggcccatgcggaattttacaacccaactctccatgtatgaaagaaggcaaatgcagtcatttttatcctaaacaatttcagcctcagactcttttggattcaaacggttatccagactatcatagaagaaacaatggtcattcaatttcaaagaatggtgttatcattgataacagatatgtagtaccttacaatccaaaattactaaaaaaaatatcaggcacatataaatattga

SEQ ID NO: 33 - DNA, 1473 bp, Glycine max
  source 1..1473: mol_type = other DNA
atgagggagccatcgtcggtgggattcgaaggcaacggcgccgtttcttcgccgccgcaagctctcctcgagagactgaaagactacggtcaagaagacgctttcgccctctggtacgagctctcttacgaggaacgcgagtttctcatcaaggacattgagagtttagatctttcgagaattgaccggattattcggtgctcgctgcgatctcaaggactgccggcggcggccattgagcctgtgccggagagcagtgtgtcgacggtggaggagagaagccaagaggatcgagagagatggtggaaaatgggattgaaggccatttctgatggcaaattagctgtgttgcttttatctggtggccaggggactcgacttggaagttcagatccaaaaggatgtttcaatattggacttccatctggaaaatcactttttcaacttcaagctgagaggattttgtgtgcacagagactagctgctcaagctacaaatgagaattctgcttcttcagtgcaaatacactggtacatcatgaccagtccattcactgatgaggcaacacgcaaattctttgaaagtcataaattttttggtctcgaggcagaacaggttacctttttccagcaaggaacaatcccttgtgtttccaaggatggtagatttattatggagactccatacagggtagcaaaggctcctgatgggaatggtggagtgtattcagctctgaaatctaccaaattattggaggatatggcctcaaaaggaattaagtatattgattgctatggagttgacaatgcactagttcgagttgccgatccaacttttttaggttacttcattgataaaggtgttgctgctgctgcaaaagttgtccgcaaggcatatccacaagaaaaggttggtgtgtttgtaagacgaggtaaaggtggtcctcttaccgtagtggaatacagtgagttggaccagtcactagcttctgcagtcaatcaagcaactggccgtcttcgtttttgttggagtaatgtctgcttacacatgttcactctggattttctgaatcaagtggcaaatggccttgaaaaagacagcatttatcatcttgcagagaagaaaataccttccatacatggatatacaatgggattaaaacttgaacagtttatatttgatgcatttccatatgctcctacaactgcactttttgaggtgttacgggaggaggaatttgcaccagtgaaaaatgcaaatggatccaatgttgacactccagacagtgcgaaactacttgttcttcgactccatactcgttgggtagttgctgcaggtggcttcttaacacattcagtgcccttatatgcaacaggtgttgaagtgtcaccactctgttcgtatgctggtgaaaacctggaacctatatgtcgaggaagaacatttcatgcaccttgtgagatctcattttag

SEQ ID NO: 34 - DNA, 1302 bp, Glycine max
  source 1..1302: mol_type = other DNA
atggcgaataaactgaatataaggctaggagaaccaactctggtgccaccagcagaagaaacggaaaagggtctttactacttcctttcaaaccttgaccagaacatagcacatccagttcgaacggtttacttttataataagtctgcatgtagagggaacgaggaagcagcacaagtaataaaggatgctttgtcgaaggttcttgttcactattaccctatggctggaagattggccataagttcagaagggaaactgatcatagagtgcacaggtgagggtgttgtgtttgttgaggctgaagaagcaaactgtgtgataaaggacttgggagatttgacaaaacaacctgaccttgaaactcttggaaaactggtttatgatatccccggtgcaaccaacatgcttcagatacctcctttgctaattcaggtgacaaagttcaaatgtggtggatttgttttgggcgtaaacgtgaaccattgcatggttgatggtataagtgctatgcaattcgtgaacgcatggggtgagacagccagaggcatggacttgagcatttcaccagttctggaccgaactatactaagaacacgaaatcctccaaagatagagtatccacaccacgaattcgacgaaattgaagatgtatcaaatgtcacaaaagtctatgaggaagagatcctctacgagtccttttgtttcgacccagataagcttgagttgcttaagaaaatggccacatcagaagatggtgttgtaaagaagtgctctacttttgaagcacttacagcttttgtgtggagagctagaagtgaagccttagggatgcacatggaccctaatcaacaaaccaagctactttttgctgttgatggaaggtctaaattcgtgcctccaataccaaaggggtactttggcaatgctattgtgttttcaaatgctctatgcaaggtagaggagttagtgaataatccactgtcatttagtgtgggattggttggcaaagcaattgacatggtgacagacagttacatgagatctgctattgactattttgaagtgaaaagatcaaggccttccttaactgcaacacttttgatcacaacatggactaggatacctttccgaagtgcagatttcggatgggggaagcccttcttttttgggcctgtaactttgccagggaaggaagtcattttgttcttgtctcataacgaagaaagtaaaagcataaacgtgctcctaggtttgcctgcttctgccatgaaaaggtttgaaaggctgatggagatatga

SEQ ID NO: 35 - DNA, 828 bp, Glycine max
  source 1..828: mol_type = other DNA
atgttttggagtgacctcgtgagtctgattgtcttactcgccactgcttcggatcctgcacctctacaatttctggccctatattctgggtgtgccatgggggaatatttccatgataatggaatgcacgcattaataatctatgatgatcttagtaaacaggccgtggcatatcgccaaatttcattattgttacgccaaccaccgggccgtgaggctttcccaagcgatgttttctatttacattctcatctcttagaaagagccgctaaacgatcggaccagacaggtgcaggtagcttgatcgccttacccgtcattgaaacacaagcgggagacgtatcggcctatattccaacgaatgtgatctccattactgatggacaactatgtttggaaacagagctcttttattgcggaattagacctgctattaacgtcggcttatctgtcagtcgcgtcgggtctgctgctcagttgaaagctatgaaacaagcacaatatcgcaaagtcgccgcctttgctcaatttgggtcataccttgatgttgcgactcaggcattactcaatagaggtgcaaggcttacagcagtactgaaacaacctcaatattcaccacttccaattgaaaaacaaattctagtcatttatgcagctgtcaatggattttgtgatcgaatgccattagataaaattcctcaatatgaaagagacattctaacgactattaaaccagaattactacaatcactaaaaggtggactaactagcaaaagaaaaatagaactagaaaaattctttaaaaaaaaggtggaacttactacataa

SEQ ID NO: 36 - DNA, 1632 bp, Glycine max
  source 1..1632: mol_type = other DNA
atggtccaaaccttttctcttccggctccaatctctcaatggcctcaaggacaaggttttgcttctggccttgtaaaccttggtgaaatacaagtttccaaagtcacaaggtttgagtttatttcgagcagcagtgttatgctagacacaaaaaaagctgttacattctttaggcctgtgggagtaccagaaagtttccatatccttggtcactattgtcaacccagtggcaagcctttgcatggttttgtacttgttgccaaaatttgctctcctcaaaatgctgatacgatacctcctctgaagaatccccttgattttaagctagtttggagtcataatgcagcaagtatggaaattccaggtgtatacttttggctacctgaacctcctgaaggttacaaggcccttggctatttggttactaacaaacatgacaagcctttgttggatgaaatgtgttgtgttcgtgctgacctcaccgataaatgtgaaccttaccgccaaatactcgctgctggttctagaattccagagttttcatttcaggtatggagtttaagaccttgtgaccgtggcatgttagggaaaggtgtttctgttggtacttttttctgcagcaatggctggaccatgggagaagagctactacctgttgcatgcttgaagaacctgaatcctgtgctaccagcaatgccagatttgcaacaaatacatgcacttattaagcattatggccctactgttttttttcatcctcaggaaaaatacttgccatcttctgtggattggtttttcaacaatggagccatgctgcacaaaaagggtgtgtctaaaggagagggaattgatgcaagtggctcaaacttaccaagtggaggaacaaatgatggccagttttggatagacttgccaagtgatcatgatagaaagaattttgtcaaacgtggagacttgaagagttcaaggctttatgttcatgtgaaaccagctcttggtggaacttttactgacattgcaatgtgggtgttttgtcctttcaatggaccagccactctgaaaattggaattaagagcattcctctgagcaaggttggagagcatgttggtgactgggagcattttacacttcgtatatgcaacttcagtggagaactctatagtatatacttttctcagcacagtggtggtgaatgggttgatgcctatgacttggattatattaaaggttacaataaagctactgtttactcatcaaaatgtggacatgcaagttaccctcatccagggacctatatgcaaggttcttcaaaacttgggattggcattaggaatgatgctgctcgcagtaatttgtatgtggattctagtgttcactatgagcttgttgcagctgagtatcttgaaaatgatgtcactgagcctcaatggttgcagtttatgagagagtgggggcccaaaattgtttatgatacaaaaactgagttggataaggtaatcaatgctcttcctcgcatgcttagatattcggtgaggaacttatttaacaagtttccagtggaactttatggtgaggaaggtcccactgggccaaaagagaaaaataattggatacaggatgaaagatggtaa

SEQ ID NO: 37 - DNA, 591 bp, Glycine max
  source 1..591: mol_type = other DNA
atgacgactaggaacgggaaaataaaaaatttcactttgaatttcggacctcaacatcctactgctcatggtgtttcacgaccagtattcgaaatgaatggagaagttgtggaacatgcaaaaccacatattggatcactctacactgcctacacacgaattagctttcgagggactgaggaattagtagagtgcaaaacttatcttcaagctttaccttattttgatcatttagaggaacatgctcattcttcagccgtagagagacttttgaattgcgaggtaccattatgggctcaatatatacgagtgttattccgtgaaataactcgaatttcaaatcatttgcctactttaactactcatgctatggatgtgggagcatcaactctgtccctatgggcttttgaggagtgggagaaattgttggaattctatgaaagagtctcgggagctaggatgcatgccagtttcatacgaccaggtagagtggcacaagatctgcctcttggcttatgtcaagatattgattgcttcacacaccaatttgcgtcttgtatcgacgaattagaagaggtgtcaactggcaac

SEQ ID NO: 38 - DNA, 1413 bp, Glycine max
  source 1..1413: mol_type = other DNA
atggactggaggcgctggactaagaataagaagaaccaccaccaccctccgccgcagctgcaaccgcgcttggagcgcctcaacgccaaaaaaggcatcaattaccaatcaccaccttctcctccttccccttcttcttcttcttctcacttggatcgccaaaccagcttccgcctcttcggcttcgacggcgaattcgaccgcatcttccagaccctgggcctctccggcccagaagacttctccatccccaccgctgactgggaagcccacaaggcccgcttatctgggcccaccaccactcctcctcctcacacgcttcctcccacttcccagattcaagacgatgacgtttcggttcccgctgcggtttcttcttcctccccttggtcttccggggagcactacgtgggcaggggcagtgtcggtagattgaatttgagacacggcgaaaggagcgtgcttttcaccgattccgattccttcactacttcacacgacgatgacagtgatgttggtggagagagggagagagcgggtctcgcttctaattctgcagccgctgatgagttggttattccgtttaactcatccaacgagtggtttagacagactttcgcttcgtggcagaaaggtgacgtcttgggaaatgggtccttcggaactgtctatgaaggcttcaatgatgatggattcttttttgcggtaaaggaggtgtctttgctggatgaaggtggccagggaaaacagagcttttttcaacttcagcaggaaatatctcttttgagtaagtttgagcataaaaacatagttcgatattatggctcaaacaaggacaaaagtaaattatatatattccttgagcttatgtcaaaagggtcattggcaagtctctatcaaaagtatcggttaaacgattctcaagtttctgcatacacaaggcagattttatgtggcttgaagtatcttcatgatcataatgtggtccacagggacatcaagtgtgctaatatactggttaatgtaagggggcaagtcaagcttgcagattttgggttggcaaaggcaacaaaatttaatgatattaaatcaagcaaaggctctccatactggatggccccagaggttgttaacttaaagaatcaaggtggttatgggctagcagctgatatatggagcttagggtgtacagttttggagatgttgacaagacagcctccctattctgatttggaaggaatgcaagcattatttcggattggccggggtgaacctccacctattcctgaatatttatctaaggatgcccgggatttcatccttgaatgcttacaagttaacccaaacgatcgtccaactgcagctcagctattttatcattcttttctaaggagaacagttctatctccctga

SEQ ID NO: 39 - DNA, 2397 bp, Glycine max
  source 1..2397: mol_type = other DNA
atgccaatggcgtgcttctccattctctctcctcctcatcccactctcactctcactctcctccctccctcccactctcaatgcggcggcttcaccgttagagttagtgttgctctcaccgaaaggctctcccagcaccacaagaccaattccattattccttcaactcgcgctgctgcgaatggtggcggtttcgagacagaaactgcggtggtggaaaagcctcaattgaagactctcttccaagcttccagaggctacccttccccctttggtgccactgttcgagacggtggggtcaatttcgccatttcctctctcaacgcactttccgccactctctgtttctttactctctctgatttccagaataatcgagtgacggagtatgttcctcttgatccgttgattaataggactggaggtgtctggcatgttttccttaaaggagattttagagatatgctttatgggtacaaatttgatggcaagttttctcctctagagggacattactttgactcttctcatatactactggacccgtatgccaaagcagttataagcagaggggagtttggggctttaggacctgatggtaactgctggccccagatggctggcacggtaccttctgaggatgatgagtttgactgggaaggagatttgcctctcaagtatccacaaaaggatcttgttatatatgaaatgcatgtgcgtgggttcacaaagcacgagtcgagcaataccaagttccctggtacataccttggtgtggtggagaagcttgaccacttaaaggaacttggagtaaattgtctagaattaatgccatgtcatgaattcaatgaactggaatactatggtcacaattctgcacaaggagactacagggtcaatttttgggggtattcaaccatcaattacttctccccaatgatcagatactcatctgctggcatacgaaactgtggccaggatgggattaatgaaattaaattcctgatcaaagaggcgcacaaacgaggaatagaggtcatcatggatgttgttttcaatcatacagctgaggggaatgagaatggtcccattatttctttcagaggtgtcgacaacagtatgtattacatgttagcacccaagggggagttctataactattcaggatgtgggaacacgttcaattgcaaccatccagttgtgcgacaatttatagttgactgcttaagatattgggtaacagaaatgcacgtggatggttttcgctttgatcttgcttctattatgaccaggagtagcagtctctgggatggagctaatgtatttggtgctccaatagaaggtgacttgttgacaacaggaacccctctaagcagcccaccattaattgacttgatcagtaacgatcctatactttgtggagtgaagcttatagctgaagcctgggatgctggtggcctctatcaagttggcactttccctcactggggtatttggtcagaatggaatgggaagtatagagacacggtgcgcctgtttatcaagggtacagatggctttgctggagcttttgctgaatgcctttgtgggagtcctaatttatatcagggaggaggaagaaaaccgtggcatagtattaactttgtatgcgctcatgatgggttcactctagctgatttggtgacctataacaacaagaataatttgtcaaatggagaagacaataatgatggagaaaatcataataatagctggaactgcggacaggagggggagtttgtcagtacctcggtgaagaaattgaggaaacgacaaatgcggaatttttttctttctctcatggtttcccagggagttccaatgatatatatgggcgatgaatatggacacacaaaaggaggaaataacaatacctattgtcacgataattatcataattacttccaatgggacaaaaaggaagaatcctcatcagacttcttcagattttgtcgccttatgactaagttccgccaggaatgtgaatcgctaggcttagctgacttcccaacctctgagaggctgcagtggcatggtcattttcctggaaagccagactggtctgaaaccagccgttttgtggcttgtaccatggtagattcagtgaagggagaaatatacattgctttcaatatgagtcatttacctttcacagttaccttgccggagcgtcctggatacaaatgggaacctcttgtagacaccagcaagcctacaccatatgatttcctcactcctgaccttcctggaagagatattgccatacaacagtatgctcagtttctggacgccaatatgtatcccatgcttagttattcttccattatcctcttgcgcattccagatgaaaatctgtag

SEQ ID NO: 40 - DNA, 3144 bp, Glycine max
  source 1..3144: mol_type = other DNA
atgtcgaaaactccgaaggagaaggaaacggaggagcctctgccagaccacctccgctgcgggcgcacggacgggcggcaatggcgttgccggcggcgagtgaaggagaatctgaagctctgcgagattcactacctccaaggtcgccaccgccagtacaaggagaaggttcccgagtcgctgaagcttcagcgcaagcgcaaatctaacaacaacaacaacaacaacaacgaggaagaagaagaagaagaagaagaagagaaaccagagcctgataagaagaatgttctcgacgacaatgtcgaatctagggcacggagaacctcgagaatcgtgaagaagaagcgcatgctttctgaggattccgatgcttcagcttcttctcctccggcgaggaagaaggcgctgaagcagggcgatatgcagttggagctcttgaggatggtgctgaagagagaggctgagaagaacaaaaacaagagcaagagcaagaacaagaagaacaataacaaaaagaagaataagaagaaggaaaagaggaggaaggaggaaaaagaggaattgtgttacactaaagaagagttgagaagagaattgccaaatggagtaatggaaatctctccggcttcaccgacgcgtgattacaacaatgtaggttcgcattgtgatgtcaaagttggtgttgatagtaaaactgttactccgcgttactttaggtccaagaatgttgatagagtccccgcggggaagttgcagattgtgccgtatggatcaaacttgaaaaagggtaagaggaagaagtgtcattggtgccagagaagtgagtctgggaatctcattcaatgttcgagctgtcagagggagttcttctgcatggattgcgttaaagagcggtattttgacgctgaaaatgaaattaagaaggcatgtccagtttgtcgtggaacttgcccttgtaagtattgctctgcaagtcaatgtaaagacagtgaaagtaaggaatgtttgactggtaagagcagagttgatagaatattgcattttcattatttgatctgcatgctccttccagtactaaagcaaataagtgaagatcagaatattgagctagaaacagaagtaaaaattaaagggaaaaatatttctgatattcaaatcaagcaggttgaatttggatgcagtgagaaaaactattgcaatcactgcaaaacacccattttggatctccatagaagctgtcctagttgttcatatagcctatgctcaagttgttgtcaggaattaagtcaaggaaaagcttctggagcaatgaactcatccgtgttcaagcgacctgataaaatgaaaccttgcagtgctagtgagaaccatactttggaggagagagccacatctattggcaatttaactgatacttcagtattgcctgagtggacaaatggtaatggcattgatagtctatcatgccctcctacagagcttggtggttgtggtaaaagccaccttgagttgagatctgttttcccttccagttggatcaaagagatggaagcgaaggcagaagaaattgtttgcagctatgactttcctgaaacttcagataaaagttcaagttgctcattgtgttttgacactgatcatggtactaacagatataagcagttgcaagaagcagctctaagagaagattccaatgataattacttgttttgtcccacagtgatggacatcagtggtgataactttgagcactttcagaaacactggggaaaaggccatcctatagttgtccaagatgcgctccgaagtacctcaaacctcagttgggatccactgaccatgttctgtacttatcttgagcagagcattacaagatatgagaacaataaaaacttgcttgaatcctgtttggattggtgggaggtggaaattaacattaagcagtactttactgggtctgtcaagcgtcgtcctcagagaaacacttgggatgagatgctgaaactaaaagggtggctttcttcccaaatatttaaagagcagtttccagctcattttgctgaggtaattgatgctctaccagttcaagaatacatgcatcccctctgcggtcttctgaatttagctgcaaatttgccacatgggagtgcaaaacatgacattgggccatatgtctacatttcttatggctctgctgacaaagaaactgattcagtgacaaagctctgctatgactcatatgatgtggttaatattatgacacataccacagatgcccccctctctacagaacaacttacaaaaataagaaaactgctaaaaaagcacaaaactctgtgtcaaatggagactattgctactgaggagccacgggaacagaaattgaatggaatggcattattgcatggtccagaaacagagcgaaaaggctcatggagtatggtcgaagaaggaatgaactttttcaggagagtcaatagaacatcttgcatctcaactgaagctaaaaaagtttctagtcagagcatggacagcaatggagaatgtgatttcatttctgattctgattctggatccactttacttctccttgggactgttcaaactgctgaattgtcaaagcacaataatcctagaaatccctttgaaagctcaaaaagacataaaaaaaagtttactgagcatttgggtgcccagtgggatgtctttcgcagacaagatgttccaaagctcatcgaataccttaaaagacattatgctgaattttcttatacccatgactatgacaagaagatggttcatccaattctagatcagagcatttttcttgacagtactcacaaaaagagactaaaggaggaatttaagattgaaccatggacttttcagcaacatgttggacaagctgtcatcattcctgctggatgtccataccagatgaggaattctaagtctagcgttcatgcggtattggaatttgtgtcccctgaaaatgttactgagggtatccagttgattgatgaggtccgactattgcctgaagaccataaagcaaaagcagacctgctggaggtaaagaaaatggcgcttcatagcatgaatacagcaattaaagaagtacgtcagcttacaagcaaaacatga

SEQ ID NO: 41 - DNA, 2322 bp, Glycine max
  source 1..2322: mol_type = other DNA
atgagcacctttgaatcatcaacaacaagatcctttgtttctgtagaatcaacaacaccaattgaaggaacggaagctgacaatgtgaatagggactcattgttccgagggtgtgtattggaagggagatgggacttcattcttacggcatataaaaacgatagtcactatcacaaaattaaaataaacgaaagcagaggcacagcactacacgtggcagtgaatgatggcaaagtggaacttgttaacactctcgttggtgcaatcttaaaccatgaagggatggatgtgctgagggatgatagtgcattgaaaacaaccaatgagagaggggacactcctttgcaccttgcagcatcaagagggttcaatgctatgtgcaagtgcatcataggggagtctgaggaaaggaaggatttgattagggttaggaacaataagggtgaaacacctctcttccgggctgtgctcacatgccatacaaagacctttgtgtacttccatcatgtttccaaagatattccacttaggaactatgatggggataccatccttcaccatgccatttggagagaattcttggatttggcaattataataactcattgctatcctgaacttgttgacatgcgaaacaaagatggagccactcctctcaaagttcttgcctctaagccttcagccttcaagagtggaagcaatctcccatggtggaagcaaattctatattatggtatactcgtagaacaactagacgcagaaaaggcaataaaatcctatatggataaagttgacaaatttgaggccgacattgaactcaaagtgaatatacattcagaaagtagtgaagccaacaaagcacaaaaatttgtggaaaaacagtatgctacttctgttcggtttgtaaagagtgctgttcgattagcattcaaagtccttagcctctcgggattgggtgtcactgcacaggacttgaaagcaataaagaagataaggcagaagcacagatggagtcgtcaactcttgaatatttttatggaaagaccttatgagtcgtacattggaattactgggggtcgaccatttttgagagaagacagagacttagggcaacccgttattacccaacaacagttacaaatggtgggtggtgcagctagctcagggcaacaagaaaacaatagagtggaatccgcagcaaaaaatgaggaaaaggagacatttgtggcagtggcaaaagccggcatagttgaacttgtgaatgagcttcacaacaaagtaccaagtaccttccatgacactaactctcctgagaaggaaaacttactgattgtggcaatgaagaacatcaaatacaaaataggagaacaccatgttgacaaaaaggagactgcatttttggcagcggctaaatacggcattgtggagattgtatttgcacttcaatccaaaataccaagtgccgtgcatgaaaccaactccaacaatgaaaacgtgttgcttgtggctgtgaagaatagacaaaccaaagttgttgaggtgttaaggaaacatatggataaggaactctttgatagcttgattttagaagtggataacagggagaacactgtgttgcacttggcagctggaactggaactacaagcaacagtgaaaggacctggcagattgctggtgctgccatgcaaatgatgtgggatatcaagtggtatcagtacattagagccctagtgccggagcacttcgttttcagaaccaacaaagacgataaaaccgcaggcgaaatcttcaaacaaaaacacaaagacctggtgaaagaaagctccgagtggctaaaggaaacctccaactcctgctccgtcgtcgccgccctaatcgcgggcgtctccttcgccacatcgagctccgtccccggtggcaccgagaagggcaagcccgaacttgaaggccagccagccttcgacgtcttcgccatcgcatcgctcatcggcctctgcttctccgtcaccgccctcatcatgttccttgccattctcacttcgcgaaaacaggccccagacttccgcaagagcttgcccttgaagcttctctttggcttaagctctctcttcgtgtccattggctcaatgcttgtttcgttttgcgctgcgcatttctttgtgctcaaggacaagtacaagaacattttgttccccgtttatattgctacttgtttgcctgtgaccttctatgcggtggtgcagtttccgttgtatgctgatcttcttaaagccattttcaagaaggtgccacaacccagtattaccagcagccaattttag

SEQ ID NO: 42 - DNA, 2055 bp, Glycine max
  source 1..2055: mol_type = other DNA
atgaagagaacgaaaagtgatcctcccaactcgaggaggtttagattgtctcattttttgtttggcattggggtgttgtacttgttgtttgtgtcatgtaagtttccacagtttttgagaattgtatcaacgttaagtggggatgggagtgaagataggttggagggggcagctgttggggactctgaagattcagatttgagtaaatcttttgtgagttctgtttataaagatgcatttcacaggagattggaagataatagggaccaggagggtgcccccttgagaccaaacacggagccaaagaaggaggaagagcgtcttccggaatctccgaagcagattccactgcgatatggtaggataactgggaagataatgcgggaatataagaggaccaatgatttgtctgtgcttgagaggatggcggatgaggcgtggatattgggattgaaggcttggaaggaagtagataaggttgatgagaagggttcgatgaaaaactcggtccttgatggaaagcccgagtcatgcccttcttgggtgtcaatgaatggggatgagttgattaaaggagatagtttgatgtttcttccctgtgggcttgcagctggttcttcaatcactgtggtggggacaccccatcatgctcataaggagtatgttccccagcttgccaagatgaagagaggtggagggttggttttagtttcacaattcatggttgaattgcaagggctgaagtcagtggatggggaggatcctccgaagattcttcacttgaatcctcgaataaggggggattggagcagacagccagtaattgagcataatacctgttacagaatgcactggggaacctctcaaagatgtgatggtctaccgtctggggatgaagaagaaatgcttgttgatggatacagacggtgtgagaaatggttgcggaatgatattatagattctaaagagtccaagacgacatcctggtttaagcggtttatagggcgtgagcaaaagccagaaatgacctggccatttcctttagttgagggcagaatgtttgtccttacattgcgtgctggtgttgatggataccatattaatattgggggccgccatgtaacttcatttccatatcggactgggtttacacttgaggatgctacaggattggcaattaaaggggatgtggatgttcattcaatttatgctacttctcttcctacttcccatccaagtttctcacctcaaagagtactggaaatgtcagagacctggaaagcctcacctttacctaaacatcctattaaactttttattggagttctttcagcttcaaatcactttgcagaacgtatggcagttagaaagacatggatgcaatcagctgcaatcaagtcttcagatgtagtagcacggttctttgttgcactgaatccaagggcggaagtaaatgcagtgctgaagaaggaggctgcttactttggtgatattgtcattttgccctttatggaccgctatgagcttgttgtgcttaaaactgtgggcattagtgagtttgggattcagaatgtgactgctgcatatgttatgaaatgtgatgatgatacatttattagggttgatactgtcttggaagaaattgagaaggtaccccagggaaaatccctttatatgggcaatctcaatcttcggcatcggcctctgaggaatggcaaatgggcagtcacttatgaggaatggccagaagaagtatatcctccttatgcaaatgggcctgcatatgtaatttctagtgatattgttactttcatccgatctcaacacaaggataggaaactaaggctgtttaaaatggaggatgtaagcatgggaatgtgggttgagcgatataacaataccattgcagcagtccagtactcccacaattggaagttttgtcagtatggatgcatggagggctacttcactgcacattaccaatcaccaaggcagatgatctgtctttgggacaaattgtcaagaggtcgagctcgttgctgcaacttcagatga

SEQ ID NO: 43 - DNA, 1284 bp, Glycine max
  source 1..1284: mol_type = other DNA
atgggaacctcggtggcggatctagctccgggtctgtcacggaagctgaagaaggttcaagaatctcggatcgacacgccctatctcttgtcttcgctcaacacgctctcttccttctacgacgacaacactcctcaggcgcgtcgcaacctccgatccaccatcgagaagcgcgccctctccatcaactgcgaattcctcgacgcttcccacgccgcgcagctgataaatgcacttagagaagaagagctgaatgaaaactttttcaaggcattgtctcatgtccaagagattcatgccaattgcaaagtgttgcttaggacacatcatcagcgtgctggtttagagctgatggatatgatggctgtgtatcaagaaggagcttatgagcgcctatgcagggaaagatctgtccttttcaaatattgtgcagaagaggtagccaatatgagacacaacgcactatttagaagatttataagtgctcttacacgtggaggacctggtggattgcctcgaccaattgaagtgcatgctcatgacccactacgatatgtcggtgatatgctaggctggttgcatcaggccttggcatctgaacgtgaacttgtagctgtattacttgatccagatacaattaccgattctggtccaaagcaattctccaataactctgaggatggctctgggaagacagaatctgacttgatgtttgttcttgatagaatttttgaaggatctcaaccaagtcttatagtatcctacaaactcagtagtacacttgaattttactgctacactatctcagatttacttgggcgagaaactgcactttgtaatacactatgggcactgaaagatgctgctcagaatacattttttgatattttgaaaggccgaggggaaaagcttttgcggtatcccccacttgttgctgttgatctttccccgccaccagcagtgacggaaggggtatctgtacttcttgaaattatcgacaactacaacagcatgatggttcctgcttctggccaaaaacctgcttttggtccagttatatctgctatactggatccaatagttcagatgtgtgaacaagcagcggaggcacacaagtcaaagggagctggccactcatcaagaaggagtgggatgagttttgactgtggtcaacttactaaatcatcagttgatgcaattttgtcaaatagtagctctgtgtcttcctcactggtctgttcttactccttctctcttaagttttacagatatttgttgccttaa

SEQ ID NO: 44 - DNA, 3102 bp, Glycine max
  source 1..3102: mol_type = other DNA
atgttgtctgaattgggaagaagacctatgcttgggagtaacgagggttcttttggtgatgaattggagaaggagatagggatgttacttcgtgagcaacgcagacaagaggctgatgatcgtgagcgagagcttaatatttttaggagtggatcggcgcctccaactgtggacggttctttgagtgccgtaggagggttgtttgctggtggtggtggtggtggtgctcctgctgccttttcagagtttcgaggaacgaaggatgtgaatgggattgcttctgaggaagagcttaggtctgatccagcgtatctttcatactactactccaatgtgaatttgaatcctaggctgccacctcctttgctgtcaaaggaggattggagatttcaacaaagacttaaaggtggagcttctgctctaggtggaattggagataggaggaaagtaaacaggactgatgataatgctggaagatcgctttttgctaccccaccaggttttaacatgaggaaacaagagagtgaggtggataatgaaaaaacaagaggttcggccgagtggggtggtgatggactgattggtttgcctggactagggctgagcaaacagaagagctttgcagaaatttttcaggatgatttggggcataatacctctatcggatgccttccttcccgtccagccagtcgtaatacatttgatgacactgatatcattagttctgctgaagcagagttggctcatgttcaaggatcatctgcagcccaaaatgttggcttaccagcttcatattcttatgctgttgcagtaggatcttccttgtcaagaagcactactcctgatccacaacttgttgctagggctcccagtccctgcatcacgcctattggtggcggcagagccattgcttctgacaagagagctattgccaatccagatgcatttaatggtgtttcatctggcatcaatgagtcagcagatcttgtggctgcattgtcagtgatgaacttgtcagcagattatgtgttagatggtgcaaaccatttgccatcacaggttgaatcagatgtagatagtcataagagatatctttttggtaggctaggtggtcaagatcatgggaagcaacaggcttatttaaagaaatctgaatcagcacacttgcaaaattcaagtaagagcagcaggagtgggtcaggtctcaacaatccatccttggataggcaggttgagctacaaaagtctaccgttccttccaataactcatatttcaaaggatcgcctacctcccattttagtagaggaggaagtatgccacttcagtaccagcctttagatggtacaaattcatcatttactaactacggtatgagtggctatgctggaaatccagcactggcatccttgatgactaaccaacttggcactggtaatctgccacccttgtttgaaaatgttgctgcagcatcagcaatggcagcccctcgaatggactcgagaattcttggaggtggtttggcttctggagctgctgctccatctgatgtgcataatcttggtaggatgggaaatcaaattcaaggcagtgctcttcaggctccttttgttgatcccatgtatcttcagtacctgaggacacctgagtttgctgcagcacaacttgctgctctcaatgacccctctgtggacaggaactacttgggcaattcatacatgaatttgcttgagctccagaaagcttatcttgggtctgtcctctcacctcagaaatcccaatacaatgtaccgccgggtggtaaatcaggcagcttcactcctcatggttattatggaaatcctgcatatggtgctgggttgtcttacccaggaagtcctatggcaaactctgttgtgtccacttctccagttggatctggaagtcctgttaggcacaatgaactgaatatgcattttgcttctggaatgaggaatttagctggggtcatgggaccttggcatgtagataatgagaacattgatgaaagttttgcttcttctctgttggaagagtttaaaagcaataaaacgaagtgttttgagctgtctgaaattgctggtcatgttgttgaattcagtgcggatcaatatgggagtcgatttattcaacaaaagctagaaacagctactacagaagaaaaaaacatggtttatcaggaaatcatgccacatgcccttgctttgatgactgatgtctttggtaattatgtggttcaaaagttttttgagcatggacttgcatcccagaaaagagaattagccaacaaacttcttggccatgttctggcacttagccttcaaatgtatggttgccgtgtcatccagaaggccattgaagttgttgatctggatcagaagatagagatggttcaagagcttgatggtaatgttatgcgctgtgtacgtgatcagaatggtaatcatgtcattcagaagtgtattgaatgtgttcctgaagatgcaatccactttattgtgtcaacattttttgatcaagttgtgacactctcaacccatccatatggttgccgggtgatacagagagtgctggaacactgcaaagaccctacgacacaacagaaagttatggatgaaatattaggagcagttagcatgttagctcaggatcagtatggcaactacgttgttcagcatgtgctggaacatgggaagcctcatgaacgttcttctatcataaaggaattagcaggcaagatagttcaaatgagtcaacagaagtttgcctccaatgttgtggagaaatgtttgacctttggtggtccttctgagcgccaattactagtgtgtgagatgcttggcaccacagatgaaaatgagcctcttcaggcgatgatgaaagatcaatttgcaaattacgttgtacaaaaggtgctggaaacttgtgatgatcaacaacgcgagctgattctttaccgaattaaggttcatttgaatgcattgaaaaagtacacctatggaaagcacattgtcgcccgtgtggagaaacttgttgctgctggagaaaggagaattgctgctcaggctcctcctcaacctgcttag

SEQ ID NO: 45 - DNA, 747 bp, Glycine max
  source 1..747: mol_type = other DNA
atgaccaacccaatctctgagactgaaatccccgacgaaacccaaagccccaacgccatccccgaacaatgcccacaagaaccaccactacaccccgacccccaattctccgaaaccctaaccctccccgatcctaattcccccaatcctaaccaagaccaagattctaccatgcaaggcccgatccccaccaccctcaccgtcgtcgacgacgacgaccccgagcccgacgcctccaccggcgctgcgactggaggcggcggcggcaccaccaccagacgcacgacgaagcggaagaaaggcccgaagagaaccgcgctagagagaaggtctcgcgagaagctccaggtaatcgtcgcaaccctaaagcccattcctttcactccagcaaaaaccctcgatttcgagaggcaccagagcctcttgcagcggctggggctctgggacttcgtccacgtcgagctcgattcggggctccgcggcgatctcctcgcccagctcatcgcgagctacgtccccaccagtcggtgcagctacgtcaacggggttaggatcaatgtgaaccgcgccgatctaggccgcgccttgaagcttccggtgaagaagactggttgcggtggtggtgccaccgcagattcgatagactccgcggaatctatagcgtttgttgaggaggtggtgtatagttggatgcttctgcatgatgatgaagcttacatcatgcctaatgatgaaacaaaattttaa

SEQ ID NO: 46 - DNA, 675 bp, Glycine max
  source 1..675: mol_type = other DNA
atgactgcagatgacggtaaaatattatttgatgaagaagagctcaaaatgatcctagattttaagagaggtgaggactatattgaggttctttgtggcgctacaaacaaaaaatatggagattatgttgggaggctaaaaattaataacgaaggtcaatatttcatcacttgtgagtgttgccttgagtgtcccttggtgaatgtcactctagaagcatttgagaaacatgctttaagggaagggagcggaaggtggaagagaaacatctgggttcattgtgaagacgaagacaaagttccactctggaagacacctctgataaagtattatacacaccaggcaaatgtggctaaccggaaagactctgcaatgaggaaacaaaactttcacagggatgagttcctacgttgcacgagatgcgggaaggagcgcaggttccatctcaagagcagaccagacattaagaactaccatgatgcttcaaacaacaaatgctggaattgttctctttggccttatcaaaaatcaagtctgaaagcaagcaggggctgttctcgttcttcaacttgcctaggctgctcaacttgttactgtgaagggtgcatcaagtgccgctttgaggattgcaattgccaagaatgcagagactttatgctatatgctaaaccttaa

SEQ ID NO: 47 - DNA, 513 bp, Glycine max
  source 1..513: mol_type = other DNA
atggttgatggttctattcttgtaactaaaagatgtttgagagaggacttatgctcaaagaaaaggagtggaagctattctggagagaccataaaaaggcaaaggggttttgacctagaaaggcctgctgagagagacattttttatggcggctatgatgaacatgaggcaggacttacctcctacactacactagagagatgcaaaatatgcagcaacaatgatgatggttatgatgaagacatggaagtggatttgactctaagcataggaggaggaagccaagttaataataacaagaatagtagtagtaagaaaccttatctgcttccattagggtgctcagactcacccaataggaaaactaaggacctaaattcttctgtctctttccaatcctacagggtgggagatttcagtgaccccaccacccctatgagcagctcaagtgtgacatttgatcaagagagaaaggggccacattggctttctcaaggtttaaagcttaaatag

SEQ ID NO: 48 - DNA, 1338 bp, Glycine max
  source 1..1338: mol_type = other DNA
atgaagcagctcggcgagaacgtggggagtttagacgcggccattgagcaattgctgaatgcggagaagcatgcgaggctcgccggcgacgtcgccgccaccagaaacgccgtcaccgaaattttacggttgtgcttcgaagcgcgtgcctggaaaactctcaatgaccagattgctctcttgtccaaacgacgtggccagcttaagcaggctgtgacagcgatggtccaacaggctatgcaatatactgatgagacaccggatcttgaaactcgtatagaactcatcaaaaccttgaacagtgtatctgcaggaaagatatacgtcgagattgagagagctcggttgatcaagaaacttgcaaaaattaaggaagaacaaggacttatagctgaagctgctgatttgatgcaagaaattgcggtggaaacttttggcgccatggctaaaactgagaaaatcgcattcattcttgaacaagttcgtttgtgtctagactgccaggattatgttcgtgcacagatactctcaagaaagattagtacgagagtgtttgacgctgatgttacaaaagaaaagaaaaagcctaaagaaggtgataatgttgttgaagaagctcctgcagatattccatctctaccagagttgaagcggatctactatgaactaatgattcggtattattcccataaaaatgattatcttgaaatttgccgttgctacaaggcaatatatgagattccatctgtcaaagaaaatcttgccgagtggattccaatcctgaggaaaatatgttggtatttggttctatccccgcatgatccaatgcagtcaagtcttctcaattctaccttggaggataagaatctgtctgagattccaaactttaagttactgttgaaacagttggtcactatggaggttatccagtggacaactctttgggattcatacaaggatgagtttgagaatcagagtaacttaggaaagaatttgggtgaaaaagcagctgaagatctgagggaaagagtaattgaacataatattattgttatatcaaaatactatggaaggactactttgaagagacttgcagagcttttgtgtctcagtgttcaggaagctgagaagcatctttcagatatggttgtgtctaaggcattggtggcgaagattgatagacccatgggaatagtttgtttccaaagagccaaggatagcaatgacgttcttatctcatgggcagcaaacttggagaggctgcttgatcttgtggagaagagctgccatcaaatacacaaggaaaccatggtgcataaagctgccttgaaggtttaa

SEQ ID NO: 49 - DNA, 948 bp, Glycine max
  source 1..948: mol_type = other DNA
atgagtttgtcgcaattgggggaaatcttgtctcgtcttcccatgaaacctttgatccaattcaagtgtgtgtgcaaggaatggaactccttgatttcagaaccctatttcatcaaattgcaccttagcaaatctgctgcaaaggacgatttggaacaccttcaattgataaaaaatgtctgcctcggatccatccctgaaatccacatggaatcgcgtgatgtaagtttgatattccattccctacaaattgaaacgttcttgttcaatttcgcaaacatgccaggttaccatctggtcggttcatgtaatgggttgcattgtggggttagcgaaataccagaaggatactgtgtttgtttctggaacaaggcgacaagaacaatgtttgggtttggctatgatccgtcaagtgacaaatacaaggttgtagcaattgcgttgactatgctctcacttgacgtatctgaaaagactgagatgaaagtttatggcgagggtgacagtagttggagaaaccttaaaggttttcctgttctttggactttacctaaagttggtggagtgtatctgagtggaacccttaattgggagacttgcatatcactgtttcttcccgatgatttttgcttttttgatacaaatattggagtttttagagactcgctgtgcgtttggcaagatagcaacacccatattggcttgtggcagatgaggaagtttggagatgacaagtcttggattcaattaataaattttagttatttacatcttaatattcgtccttatgaagaaaaatccatgattttaccattgtgcatgcctaacaacggagacttcttcatgctgaaattcactagaaatgctgatgatgaataccaaacaattctgtataaccagggggatggtaagtctcaagtttctgtcgttccctcagatagtttcaga

SEQ ID NO: 50 - DNA, 231 bp, Glycine max
  source 1..231: mol_type = other DNA
atggccatttatcgaatcgttgcaatcgatgctcgatctcaaagagagggaagagatcttcaaaaattaggtttttatgatccaataaagaatcaaacctatttaaatattcctgttattctatatttccttgaacgaggtgctcaacctacaggaattgttcaggatatttcaaaaagggctggtgtttttaaggaacttagctcaaatcatcaaacgaaatttcattaa

SEQ ID NO: 51 - DNA, 1566 bp, Glycine max
  source 1..1566: mol_type = other DNA
atgaagaaatttacgatacttctttaccctttgagttgttctgccggatcggtcgctcaagacctttggtctctacccggacctgatgaaaaaaatgggatcacatcttatggactcgttgagaataattctgatctagttcatggcctattagaagtagaaggcgctctggtgggatcctcacatatagaaaaagattgcagtcagtttgataaggatcgagtgacattgcttcttcggtccgaaccaaggaatctcttaaatatgattcaaaatggatcttattctatcgttgatcaaagatttctctatgaaaaatatgaatcggagtttgaagaagggggggggggagtcctcgacccacaacagatagaggaggattttttcaatcacatcgtttgggctcttagaatatggagcccttggggctttctattttattgtattgaaaggcccaatgaattgggatttccctattgggccaggtcatttcgggacaagcggatcatttatgatgaagaggatgagcttcaagagaatgattcagagttcttgcagggtggaaccatgcagtaccagacacgagatagatcttccaaagaacaaggcttttttcgaataagccaattcatttgggaccccgcggatccactctttttcctattcaaagatcacccttttgtctctgtgttttcacatcgacaattcttcacagatgaagagatgtcaagggaacttcttactacttcccaaacagatcttcctacatctatatataaacactggtttatcaagaatacgcaagaaaatcattttgaattgttgattcattgtcaaagatggcttagaatcaatagttcattatctaatggatttttccgttctaatactctatctgagagttatcaatatttatcaaatttgttcctatctaacgaagcgctattggatcaaatgacaaagacattgttgagaaaaagatggcttttcccagatgagatggttgttgctatctgctccaataacgaatcattgatcccctatatcgataatacacattccagtttgggaacgtccggtgccaaagtcattgaatggttatttgatgcaattagaactgatttgaatgaaaaaacaatagtaaatggaaatagaacaaagtgtattagaataaacaaaatccgtaaaaaagttcctcgatggtcatacaaatttattgacgaattggaacaacgggagggaaaaaatgaagcagaaaattatcaaattcgttctagaaaagccaaacgtgtagtcattttgactaataaatctaatttttgtaagaagtacgatacttataatcctacaagagatactgataacgctgaaaaaaaaaatgaattggctttaatacgttattcccaacaattggatttttggcgagacataatcaaaggatctatacgtgctcaaaggcgtaaaacagttacttggaaattttttcaaaaaagggtgcattccctcctttttttggataaaattgaaaaacctttattcttttcttttgatagtttcaaatcatga

SEQ ID NO: 52 - DNA, 612 bp, Glycine max
  source 1..612: mol_type = other DNA
atggcctctctttttcttagacaggctctgctcctccgcgtttgcttccacaactaccaacgcttcaccgccggaggcgtcgccgcacgtcaccggctgtggtgcagcgcagccgccaacctaccgtcagacgaagcggagaagaaaacaaactccggcggcatcagcaacagctcctttgcacggaaagctcagtatcctccgtcaagtgaagcgcggtgggaggacgaccccgattaccgcaaatggaaggacaaagaaaaggagattctcagcgacattgaacctatcgttgttctcgccaaggatattctccactcacgaaggtacatggatggagcgcgattgaatgaggaggacgagaaggctatagttgagaagctccttgcataccatccacattctgaagataagattgggtgtggccttgaatccattatggttgaccgtcatccccaatatcgacaatcaaggtgtctttttgttgtaagaactgatggtggctggattgatttctcctatcagaagtgccttcgggaatacattagagataagtaccctactcatgcggaaaggtttattagagaacattttaagcgtgggagtggttga

SEQ ID NO: 53 - DNA, 2691 bp, Glycine max
  source 1..2691: mol_type = other DNA
atgtattattttcagcacaatcctggattacctgctactcgtcatcgtaggatagcaagctctttcagggataactctctttttcagatattccagatatcattaacttctctcttccagttaaaatctgatgctgggagcaagttgcaggagctgtctcttatgctttcccttagttgtttatcatttgattttatggggacatcgtatgatgaaagttcagatgaaattggtactgttcaggttccatctggttggaagcctaccctagaggattcctcaacactgcaaatcttttttgattattatgcaatgaatcaaatgttttcgaaagaggcattggagtgcttgttgcgtcttgcttctactagacgatcattgttttccaatgatactgctcgaatgaagtttctgtcacacttgatgctgggcaccaaagaaattttgcaaactggaataggtcttgctgatcatgataactatcatgccttttgtcgtcttctcggccgctttaaagtaaattatcagttatccgaacttgtgaatgctgaaggctatagtgaatggatacgcttggtagcagagtttacattaaaatctttgcattcttggaagtgggctggcagcagtgtttactaccttctaaatttatggtctagatcagtgacgtctgtgcggtacctcaagagtgacaaacctaacctgttagatgaatatgtgcctaaagttattgaagggtttgtatcatcaagatttgattcattgcagtctgaactatcagatgaacttggtgaaaatcctcttgacaatgttgaagtacttcaagatcagcttgaattttttcctttcctctgcagattccagtatgaaagttgcagttcgtatttgatgaagatagtggagcctatcatgaaaagctatatgaacgaaatccatgtggatagttatgaactctctgtaactgaatccaaacttgcttggttcacacacatagttgctgcaattctcagaacaaaacagatttctggttctagtggagaatcgcatgagatacttgatgcggagatttcagcatgtgttctgcagttgattaatatttgtgacagtgggtttcacagtaagaggtatggagatgtgagcaagcaaagacttgatcgtgctatactaactttcttacagcatcttagaaggtgttatattggtgatcaggctgtgtattcttccaagcagttatataccaggctatctgaacttcttggactgcatgatcatctcctactactaaatgttattgtgggaaagatgactactaacctaaagtactacactaagtgcaaggaggttattgatcataccttaaatctctttctggaaatgacatctggatacatgtcaggaaaactgcttcttaaattggatactgttaaacacatactatcgaatcagaatagggaccaattccctttcatagaaaattgggaatgtttccgcagcagaacaaccctgtactatactattggcatgctgatattcttggaagatagcctactgaaatttaaatctgcaatggaacctttcttacaggtttttgttagattggaatcaacacctgaagcattgttccaatcagatgctgtaaaatatgcattcgttgggttgatgcgggaccttcgaggcatagcaatggctactaatagccgtagaacgtatgggttcctttttgattggctttatcctgcacacatgccccttcttttgaaaggaattacacattatgctgatattccagaggtaacaactcctttactgaaattcatggctgaactggtgctgaacaaatcccagcgcttgaattttgattcttcttctcctaatgggatactacttttccgagaagttagtaaattgattgttgcttatggatcaagaattttgcctcttcctaataaagcagacttgtacacatccaaatacaagggaatatcaatttgtttgataattcttacaagagcactatccgggaactttgtcaactttggcatttttgaactttatggtgatagagcacttgttgacgctcttgatatcactgtaaagatcatattatcaattcctttggctgatatttttgcatttcggaaggtcgcagcagcttactttgcattcttggagtctctcttcagttgtcatttgtcatttgtcttgagtttggataaaactacatttatgcttgttgttggatctctcgaatctggccttaaagatagcgagaaaatatcatcacagtgtgcctctgctattgacaacttggctacattttatttcacccatgttactgttggagagtcagttacctcacctgctgctcttaatttagctggactcctttctgattgtgctgagcttttctctagaatattgagaaccctatttgaagttgttatatttgagaaccgtggcaatcattggactcttagccgagccattttgagcatgatactaatcagtgaagagatgttcacaaatgtcaaggctcaaattttggtttcgtatccaccagacctacaccagcggttttctttgtgctttaccaagcttatgacagatgtgatgctcagcttggatttaaagaacagggaaaaattctctcagaatctgatcagattcaagtctgagttttgtgccaaataa

SEQ ID NO: 54 - DNA, 1170 bp, Glycine max
  source 1..1170: mol_type = other DNA
atgggttatattgggactcacggtgttgcagctctgcacagatataaatatagtggagtagatcattcctatgtggccaaatatgtactacaacccttttggagtcgctttgttaattttttccccctatggatgcctccaaacatgataactcttatgggatttatgttcttactactgtctgcattgcttggttatatatactccccacaattggacacagctcctccaagatgggttcattttgctcatggactacttctgtttttataccagacatttgatgctgttgatgggaagcaagctagacgaacaaattcctcaagcccattgggggagctctttgatcatgggtgtgatgcacttgcttgcacatttgaagcattggcttttgggagcacagccatgtgtggaagaactactttctggtggtggttaatatctgcaattacattttatggtgcaacctgggagcattatttcaccaatacacttatactgcctgttataaatgggcctacagagggtcttatgataatatacatctgtcactttttcactgctatcgtgggtgctgagtggtgggttcagcaatttggaaagtctttgccattcttaaattggcttccttatcttgggggaatcccgacattcaaagctatattgtgtttaatgatagcttttggtgttacaccaacagtcacatgcaatgttagtaatgtttacaaggttgtgaagggaaagaatggaagcatgccacttgcattggcaatgctttacccatttgttgtacttgtgggaggagtgcttgtgtgggattatttgtcaccatcagacatcatggggaaatatccacatttagttgttataggaacaggacttactttcggatatcttgtggggaggatgattttggcacacttatgtgatgaacctaagggtctgaaaactgggatgtgcatgtccctcatgtttctcccattggccattgccaatgtacttgcatccaggctaaatgatggggttcctttagtagatgagagactagttcttcttggttactgtgcattttcagtgacactatacttgcattttgccacatcagtcattcatgaaattaccaatgccctgggaatatattgtttcaggataactaggaaggaagcttga

SEQ ID NO: 55 - DNA, 2745 bp, Glycine max
  source 1..2745: mol_type = other DNA
atggagttcagacatcgaaattacagtgccgagcttgaatctcacgcgctccctcgcctacgcgccggagctcatcctctctctgctccacctccacctccacctctctcccaggttgatgccatagattgtgggaacactgatttctatgatccactgagaggaactaataatgatgcaaatgccgcccctcctgatcatgacaacttgaatgaagctgctgatcatatgccaacaaaggagtggacctctttcaggagattactaacgcagaggtttcctgtctccaaaatggtttccgttgcttcaatgcccgatgttttaacgagaagtggaaaattacttgagaaatcttcaacaaatatgcacttggaggaattggaggatccacaaaaattcgcagacgagggtgtcaagacgattacgtggcaggagtatgtttctcgactacatgaactcaaagatgaaattactcgttcctggcttgctgaagatcgagtgacatccttaaagttatccataaaggttgctaagcttctgatggatacgtcagtatttgagttttatcctacactttttgttcttgtcacagatatcatggatatgcttgggaacctggtttggcaacgcataaagcggaaagctgagttttccgaagatggagctttacgctgcaacttagcagaaaactttcaagcaagggatatttgtgctgatgccaaagaaacttgctataactggttcaacaaaattggtgctgtgcaagagcttcttccacgcatttacttggagctggcaatattgccttgctggcggtttttgcttgagcaacctttagacagcctccgacgcttggtaatgatgataagaggattaggagatccagtagcatctgcatattgccgtctttatatggctcactgtgctcagaagctgccttcacatgatataggctatcttgttacatgtgtcaatgacatcagagttgttttgatgcaaatcttgtcagccaatgaaagaactcataaaaatgttaaactcaacaaaaaattgcaagtcagtctgatggaaccaaccattgagtatattatgaagtgtatatttactgggttgtctcagagacaagtcaatgaagttttatcagagtttggattgatgaagaatcaacaggatttggggagtgtttcatgtgtttcaatcattcttcatcatttactgaaggaactccctatagaagtagttagttccaatgttgtgcagatccttcatctcattgaatttagcaaggataattcctttgatcagcatatgaattacagattgcttggattcaggctgtacgaaagaaaatcccctgttgatattgttgatgctgtgttagataaagttattcaggttattgctctgtatgatagccttgacgaatacctgaaggttgtagatgcttatactgatcttattcttcagaatcagatggataaccatttgaaaatcattttggaaggcatttcaaagcgtacttggaataaaggagttacagaggatgaaatgccgagcttgcaatctcttgtggtgaagcttctgtctcattttaagcatctggaagatgtgttttctctggatcagtttcctgaaatcttggatgtaatgtatggcaagtcacaggatgttgtctttttgcatatccttaatatggcgacaaggaatggtcgcattagtgatccgacgagcatacagttgctttttgaaatttctctggctctacataataatatagagtttatgaacatgaaagatgatgatggccaagtggcatgttcaatagctcgctttgtgcacatggtagattatggaacagagatggaacaccatttagcatttctggttgattgccgaggagcttttggtagattaaatgagcttaaggaaactcttgttcactctagcaattctttagcaattcaagctttgaaatgtgctaagaaacatctgaattttgtcaaatcctgtgttacatttagtgaagtcacaataccttctatttctgctcacaggcagtttgatctttttctagaaactgcagaggttgcattcttagggggcttggtttctcattcagatggattgattgattcagcaatcagctgtttgcataccttagacataattgatggcttccgaactccaactgatgttgaaggactagtttcatctattagaaagctatgtggcttcttaattatggttccaggttgtactcttagtttaccagtcacctatttcccaaatagcttatttacattgatcagctcccggtcttggtttgaaccaaaaatgagggcacaaattttttctgctatcatattattattgacaactttatcacaaaagagactgccatatcatgcaaattcacagattccaggcaatgacatgttatactacggggattcatcgtacaaccaagagcttgtttctttgtctaaacttgttcttgagaacctgcttagtgccgttcaacaagaaccttcccaggctgctcgaggaattatggcacttgaagcttgcaattgcattgcttcttctttcatgctaagcaatgagttattgtcttcttgtcttacactggtcgaaactgccaagtcatgtttgagtgccaaagacagatacctccagtcaaccattcaacttttaaacaagcaatcgccaacttctgtagggactatggtgtctacttttgtatga

SEQ ID NO: 56 - DNA, 555 bp, Glycine max
  source 1..555: mol_type = other DNA
atgtcatctccaagcaagagaagagaaatggatgtaatgaaactgatgatgagtgattatgcagtggagactataaatgatggactcaatgagttcaatgtggagtttcatggtccaaaagaaagcctttatgaaggtggagtctggaaaattcgtgttgagcttcctgatgcttacccgtacaaatccccttctattggctttgtgaacaaaatattccacccaaatgttgatgagctatctggctctgtatgcttggatgtcattaaccaatcttggagtccaatgtttgatcttctaaatgtttttgaagtttttcttccccaactcttgctttatccaaatgcttcagatcctctcaatggtgatgcagcatcgttaatgatgaaggataaaaagctatatgaccagaaagttaaagagtattgtgagcggtatgctaagaaggaaaacattagcaactctacagctgaagagagtggagatgaggaagacatcagtgaagaagaaagtggatctagtgatgatgaaattcctggtcgcgctgatccttaa

SEQ ID NO: 57 - DNA, 2355 bp, Glycine max
  source 1..2355: mol_type = other DNA
atgctttcaggggtgaaattcatttcccgggaccaggtgcatgatgaggacttggattctgtctcgaaagaaaggaaaaaatcagataggaggagggaaaagaataagaggaaaaggaggagctctagggacagctctgatgatgatgatgatgggcttgagaagataaagaaaggatctagaaagaagaagtggtattcgtcggatgaagactctttgatttacacaactgaaagtgagagtgagaaagatgaaaagaagaggaggaggagagcgaaaaagaaaagggatgatggttcgtcacgtgattctggcgaaaggtcaaaaggaagatcacgatccaggagtggtaaaaaagaatatacatctgaggatgaggaagactcttattcttctgatggtagtgatagtttttcgggtcttaaaggtaagcaccaaaagtcagatagaaaagatgggagtaagaggaacaagatcgaaggtggcacggtaaattctacaagtgagatggaaattgcaagaaaagaaatgggattggattggatgcttaggtctgaaagtaagaagcctgtagttacggaatcagaggaaaaactgtcagaggaggttcctgttgaggagtcaaagaaggcaaatcctaaggaactgaatccatatttgaaggataatggaagtggatacccggaagaaagtggagctaaagttggtgccgaccaacttctatcttcttcccttgttggggatggaggagcaagttggagacttaaagccttaaagcgtgcacaagagcaagcagctcgagatggacgaagattcaatgaggttgtggaagaaaggtggagttctcttggtgagttgactgcagctgttgcatctcatgcagctgccccagctcgtgctcatctgcgtgctataaaaaatagacaaagagggataactgaagaaaattcacaagattctgataagcatggtcgaagggattctaaaaggcaggactacttaaaggatgtttctgttcggcaccatgaaatgaaagcacctaaagttcgagattctttgtcttggggaaagcgaaagagccaacaagtggtagctgagggtgccggggtcatctctgctgcagtatctagcctaaataagtttgccaatgatggaagctttatgcatgattttggtagcaagatgagtaataattctgacggctctgttttggatagtagtgaattagaaaaggtttcgttggaagctaatagacctgaagaaagtagtgcagtagtcaagaatgagatgagtgaaaaccagttggcagctaaggttatgcaacttcgtttgaagggaaagcatgaagaagctgacaaactgatgcaagaagcaaaagttatgaacacaaagcaaggaaatcaagaccattcaattagatcaagaaccgagggaagttctagcaggtatgctatgcagaaaatatctgctgagcagaagaaaggagaggatgatgctgatatgcatcttgctcgcaagatcatgcataacaagcagtttagggcttctactcaggctgatgatgaatatgactttgaggatggtccaagcagaaagagtagaaagaagcaaggaggtgatgatcacaagagtatccaaaagaagacaaatcgattcttgactcagcaagagcgctgcctattttgtttagaaaatccaaatcggcctatgcatcttgttgtttcaatcgcaaatttcacatatcttatgttgccaaagtggcagcctgtggtgcctggtcattgctgcattttaccaattcagcatgaatcagctacaagaactgtggacgataatgtctggacagagattcgaaacttcaagaagtgcctaattatgatgtttgctaagcaagagaaggaggtagtgtttcttgagactgtgatgggattggcacagcaacgacggcattgtatggttgagtgcattcctttaccccaagatattgccaaagaggctcctttgtactttaaaaaggctattgatgaagctgaagatgagtggagccagcacaatgcaaagaaacttattgatacaagtcaaaagggattgcgcaattcaattcctaagcactttccatattttcacgttgaatttggtctaaacaagggttttgtccatgttattgatgatgaaaagcagtttaacatcagccttggcttgaatgtcataagaggcatgctacatttggcagaggaggacatgtataggcgacgacgctacgaggctgtggaggtacaaaagcaagcagttgaaagcttttccaaagagtggaaacattttgactggacaaaacagcttcatgaaacttcgtaa

SEQ ID NO: 58 - DNA, 264 bp, Glycine max
  source 1..264: mol_type = other DNA
atgagaaccactcaacgtgctttggtgggtctatttgtcttaattctcacgcacttcatcattttctctatagtttcaatgactactgagactagaagtaatctgataggtaggtgcccaccacggaagctgctagatcgtgtctcctacttctctgcaagtttagataaattaaaaatagtttatgaggctcctcagagatcagtaaatacaagtttaaggaagaatcctccaagcaactcaaacccaacccataacaagtga

SEQ ID NO: 59 - DNA, 2592 bp, Glycine max
  source 1..2592: mol_type = other DNA
atgggttcattctctggcacttgtgaaattgtcgaagcaagggaagacctaaatacagagaaagatacggggatatatcagtccaattctggatatagcctggatgagaaatttcagaagcatcctgtgcagaaactgggatataagggtaatctagatgatgatattaataagctgtttgagtcaattactcttaaatcttcctcaagggatttgggctttatacatggtacaagccctaagatgaaaagtgcattgaaaaaaccaattgcagtgggtgcttctcggtccccacgagttggaccttctgagcctgtgactttgaagcaggcactaagagacctatgtatatctaaggcatcagaaatggcttccatgaaaagattatcaaagtcaactgcttctccacgaatatctgaggttgggaagatacatacattgtacaattcagttgtagttgaaggtagacgatctgagccttctaatgttgagagtaaaggtagtacaagtgaaatatctctagtgccagaagagagcaactcactttctttggataatgcttaccagtctcgttcaactgttaaaagcacttcgctgagcccaaatgttcaatcttccaaaattgctgttgcaattgcaattacacaaaatgatactggggcttcattgatgccaagtgacctggctagttcatcaagtaaagttggggttctatcaccgtcatcagagcctgctcaaatagagaaacaaacatctgaatcttcttcatctagttgtaacaccaatggaagtaaattagagttgcccgaaaatgcttcttcccctaaaaagataggaaataaagcctctgcatcaaagaatgggcggaaaggtaggttacaaacagtctcttcctcatctacttcggtaaatggcaatagagtgtgcaaactgtcacgcaatgcccctcgtacagtcaaatcaattatcaagaacaaaaattttggaaagaaaaaagtaaagcaggattcagtttctgctttatttgatcctacatgcagtgaagtaaatgacaagtcagtttctggtacaactcaacttgtttgtgagagatgttggtgtgctattgaaaataacaaaggtattacatcattggactctatcagtcctggagagggaataaactctgttaataactctggcgctgcatcagctggctgtaatagcagcagagaagttacaaaagtgaaaaagaacaccgtattgaaagagcaacttgaattttctcaaagttcaaagagtagccaaggtgactacagtagtagtacaagtaccagtgatgagagtaatctcagtgggtctagttgtggcaataggcctcacatgtcaaaggatgttagatggaaagccatacgccatgctcaaattcagaatggagtcttgggcttgagacacttcaatcttttgaagaagctaggttgtggagacattgggactgtatatcttgctgaactaattggcaaaagttgtttgtttgctattaaggtgatggacaatgagtttttggcaagaaggaagaagatgcctagggctcaaactgaacgagaaatattgaggatgctggatcatccttttcttccaacactgtatgctcagtttacatcagataatttgtcatgtctggtcatggagtattgtccaggtggtgatcttcatgttctacggcagaagcagcttggcagaagtttttcagagccagcagcaaggttttatgttgccgaagtccttcttgctttggagtacttgcacatgcttggagtggtttaccgtgatttgaaacctgaaaacattcttgttcgagaagacggccacattatgctcacagattttgatctgtcgctaaggtgtgatgttagcccaacacttctgaagtcatcttatgtggatcctgctaagatttctggtccttgtgcacaatcgagttgcattgagccattctgtattgaaccagcctgtcaagttccgtgcttcagcccaagactcctacctcctgccgcaaaagcaaggaaattaaaaaatgaccttggtgctcagctcagatcattgccacagctcgtggctgagcccacagatgcaagatcaaattcatttgtcggtactcatgaatacttggcgcctgagatcatcaaaggagaaggacatggagctgcagttgactggtggacatttggtgtttttctatatgagcttttatatggtagaacaccctttaaaggttctaataatgaagaaacattagccaacgtggtgttgcaaggtctcagattccctgacaccccatttgttagtatccaagcgagggatctcattagagggttgttggttaaagagcctgaaaaccgtttgggttcagagaaaggggctgctgagataaaacagcaccccttcttcgaaggccttaattgggcgttaatacgttgcgctatcccaccagaactgccagacttgtgtgaatttggagtttcagagatgaccccacagtcccagggaaagggtgttaagtatgtagagtgcaatgcagcaggagagctagttgaatttgagttgttttga

SEQ ID NO: 60 - DNA, 2184 bp, Glycine max
  source 1..2184: mol_type = other DNA
atggagggaatgtgggagaacattcttggaaagcatcagcagtcgctgaaatccctattcctccgaagcaagccttcttcccccaacgccgacgccgccgatgactatgccaattcccccaaacccatccctcagctctctcctctcgccaactccgtcgtctctcgctgttccaagatccttggaatgtcgacacaggaattgcaacactgcttcgattcggaactccccatgggcgtcaaagagcttctcacctacgccagacacctcctcgagttctgttcctacaaagcgcttcacaaactcattcaaatctccgatttcttaaacgacaaggattttcatcgtttaaccttcgacatgatgctcgcttgggaggctcccagcgtccacaccctcccagatacccctacttcaagttcaagtaaagaggaaaccgcaggggacgaggatgaggcctccttgttctattcgagttccaccaatatggctcttcaggtcgatgacaagaagacggttggtttagaggctttctcgcggattgctcctgtgtgtattcccattgccgatgttgtaactgtccacaatatcttccatgcactcactagtacctccgcgcaccgccttcattttcttgtttatgacaaatacctaagattccttgacaaggttatcaagaattctaagaatgtaatggccacatctgctggaaatcttcagcttgcagaaggagagatcatccttgatgttgacgggacaattccaactcaaccagttcttcaacatattggaatcactgcatggcctgggcggttgaccctcaccaattatgctctgtactttgagtcactgggagttggtgtatatgagaaagctgttcgatatgatctgggcacagacatgaaacaggtcataaagcctgatctaactggacccctcggtgctcgcctttttgataaagctgtgatgtataagtcaacttctgtagcagagcctgtttattttgaattccctgaatttaaagcaaacttgcgtcgtgactattggttggacattagtctggagatcttgcgtgcacacaagtttatcaggaaatactaccttaaggaggtacaaaaaacagaagtgcttgccagggctattctgggcattttccgctaccgtgcagtcagagaagctttccagtttttttcatcccactataaaactttactttcgtttaacctagctgaaactcttccgcggggagacataatcttgcaaaccatgtcaaatagcttaacaaatttgactgttgtttctggtaaacatgatattcctgcgactgtagatacaaaaaggcaaccggcagtgtctccggtagcagttatggcacttttctatcttggatacaaatcaaaaaaggtgactgatatttgtgaggaagcaacttttgtcagtgatatccgagttggtgagatacatcccttggaagtggcagtgaaaaagtcccttctggacactggaaaagcggaagctgcgcaggcaaccgtggaccaagtgaaggttgaaggaattgatacgaatgttgcagtaatgaaggaactactattcccagtcattgtatctgctaaccaactacagcttttggcctcatggaaagacttttacaaatcagcagcctttttgctactctcctgttatatgattataagggggtggatccagtactttctgccatccatttttatgtttatggcaattctcatgctctggcgaaggcatttcagaaagggaagaccattagaagcctttatagtaactcctcccccaaatcgaaatgctgtagaacaactgctgacattacaagaggccattacacagtttgaatcgcttattcaagctgcaaatattattctcctaaaattgagagctcttctacttgctatattaccacaggctacagagaaggttgcactattacttgtattcctagcagcagtgtttgcttttgttcctccaaaatacatatttttggtggtattcgttgagttttacacaagggagatgccatataggaaagagagcagtgatagatggataaggaggattagagaatggtgggacagaataccagctgctcctgtccagctcgttaagcctgttcatgaatccaagaaaaatgagtccaagaaaaaaaaatga

SEQ ID NO: 61 - DNA, 2097 bp, Glycine max
  source 1..2097: mol_type = other DNA
atgccgtcggacaccgtcggcaacgtggctctggtccccttcaccggagactcgccgccagcggtataccctctccaccacggcctcgctcctccgattagtcgtctctccatctcctgggcccgcggcagctccctccgcctctcccttttcgccggcgccgccgccaaggtcgttgaagtgaagctcgccggcgaagattcggagatccctgacgctcactggcgccgaatcgcgtacggctccgttgcgccgtttgcgctgcttcagagtcggaggagctccctttctgctctcttgaagactccttctccctatcgttcggattggtgggagcatgtgcttcagtatagcaaagagataggttcacttcttggggggccaaagttgcctgctagtcctataattgaggacccaaatgtaattgctaagagaggtgaagagcctacatctttgaaagctgcgtgggagttaattgaaatattttatgtggacaagcaatctcaagcatggctacctgaaaggcttgttgattggttagctgattatgccagcctctttactagtacacatgaaacaattcatggcaagcttgtggattttcagaaagaactagttaacatacaggtgattgaggaggatcccagatattgggacttgttgtcatctgcactgtcagttggttggcttgatattgtggtgaaaatgctgcggttgcatggatcttaccaattagatcagctcagtaatcgggagttggaaaatggacttgtagaggctgttgctgttcttatttccaaaatgccccgcatgcgtcctgaatctgttggaaagttaggtgaatgctataaatccaaacctgacttcatcaaggcctgggaaaaatggaggtcacaaattacaaaactggattgcagtcgattctggattcaatgtgataatcaacaaactcgtgagggcttgagaaacttgctacaaattatgctgggtaacactgagagtctctgcatggctacatgttattggattgaattatatatttctcatttcctgtacataaggccatttacaatgggaatagaaagcatgtataatttggcccagaaatgcatccagttgaaaccacgatctagtacccataggttgtcagtacttatgattggaattcttgaagaaaataccgaggttgttttagctgagtgttccagagaatttggcccttggttggtggcacatgccatagaactgttgactgcagggagtgagcaagcagagattcttctgcatgaggagcgttataacttggggggaatcagcatagtggaactacatcggcttgtatatgctcaaattctatcgtcacatgcattgacctggcaaattgctcctatatatttaacatcatgcatgaagcaaggaatgggcttgttagagaatttattgtacaggcaatctgctcaacataatgatgtgttgcttaagaacattgagatatgccgtttgtatgagcttgatcatattagttcaaatatcatgaagattgctggagtacatcactggaagcatggccataagggtgctggagtattctggcttcaacaagcccaagacgcaagttgtcttgataagattgcccaacagttatttgattctgttggaaagtctatatctgatgaaagcttcaagcaatgggaaggcatgattgaactattgggttctgaatctaagcctgctgggggacttgaatttttgcacaagtatagggatttcaagaaatcccttcagaaggtatctagtggaaaatcaactgatgcagcaaggcaagctgtaggctccctcatactgcttatgaaaaatccatctactcctcagcgcttttggttgcctcttctgtatgactcgttgaagttacttaattggcaggattgccctcttctaagtgtctctgagaccaatcttctgctgaacaaacttcatgagttatctctggcgaagctgcgaccgcaccatactgagcctagcttacctcctgatgcactaagctctattaggttggctctagctacaaatcttggtcaagccatacttgatgaatag

SEQ ID NO: 62 - DNA, 1221 bp, Glycine max
  source 1..1221: mol_type = other DNA
atgggaagtgcacgaactctacaatataagttgagcttctacaactgttcttcagttgagcatcaatacctgagaaactctgaacagtcactgtcagactcacaagatatgatctcctatccaatttatgtttctgatcttgacgacagtgtgctcagattggacctaacatcttgtaccaaaatgtttgacatcgttacaccagttttggcttacgggatgcagcgaaattcgttggatttgagatggttcgaagcaaattctagtaagtgcaaagcaaaaggaaagaaatgtaaatggaagaacaacaaaggtgacattgaatgtttcgactgcaaggacaaacaaaaaactattcgtgttcccaaatcttttatttactctactacatgttcgattcttttggggttcatagttattgccatttttaagatcatataccattttagacagaaagaagaggaccaagcaagggggaagaagttcttagaggaatacagggcagaaaagcctgcaagatttacttatgctgatgtgaaaagaatcacaggtggttttaaagacaagttaggggaaggagctcatggggttgtattaagaggaaaactttcaattgagattctgagggaaaaagggaaagagttcatcaatgaattggaaattatgggcaaaatccaccacatcaatgtggttcgtttgcttggctattgtgctaaaggaatccatcgtgctctggtctacaatttctttccaaatggttcgctacaaagcatcatatttccaccagatgataagcaggatttccttggctgggagaagctgcagaacattgctcttggtatagctaaagggattgagtatcttcaccaaggttgtaaccatcccattattcactttgacatcaatcctcacaatgtgttacttgatgacaacttcactcttaaaatttctgattttggcttagcaaaattgtgttccaagaatcctagtttggtgtccatgacagcagctagaggaaccttcggatacattgcacctgaagttttctctagaaactttgggaatgtgtcttataagtctgatatttatagttacaaaatattgttagacatgtcttctccacaagatttccatgtgttgtatgcagattggatgcatgacctagttcatggagatgtgcatatccatgttgaggatgacggtgatgttaaaattgcatag

SEQ ID NO: 63 - DNA, 1914 bp, Glycine max
  source 1..1914: mol_type = other DNA
atgatcactggtaaggatatttatgatgttttcgcggctattgtgcccctctacgttgctatgatattagcatacggctcagttcggtggtggaaaattttcacacctgatcaatgttctggcataaaccgcttcgttgctgtgttcgcagttccacttctttctttccacttcatctcctccaatgacccttatgctatgaactaccacttcatagcagctgattgtcttcaaaaagttgtcattttgggtgctctctttctatggaacaccttcacaaaacatggtagcctagactggacaatcaccctcttctcactttcaacccttccaaacacacttgtcatggggatccctctattgaaggccatgtatggagacttctcagggagcctcatggtccaaattgtggtgttgcaaagtgtgatatggtataccctcatgctgttcatgtttgaatatagaggtgcaaaactcctcatcacagaacagttccctgagactgcaggctccataacttccttcagggttgactcagatgttgtctcactcaatggtagagagccacttcaaacagatgctgagataggagaagatggaaaacttcatgtggttgttaaaagatcagcagcttcttccatgatatcttcattcaacaagtctcatttaacttccatgacaccaagagcatctaacctcactggggttgagatctattctgttcagtcatcaagagaaccaaccccaagaggttcgagtttcaaccaaacggatttctatgccatgttcgcaagcaaggcaccgagtccaaaacatggctacacaaacagtttccagagtaataatggtggtattggtgacgtttactcgttgcagtcttcaaaaggggcaacgccaaggacttctaattttgaagaggagatgttgaagatgcacaagaagagaggagggaggagcatgagtggcgagttgtttaatgggggtttggtttcttctaattacccgccaccgaatccaatgttttcagggtctacgagtgctgctggtggccccaagaagaaagatagcagtggtggcggtggtgctgtagcacctaacaaggagttacacatgtttgtttggagttcaagtgcatcacctgtttctgaggggaatttgaggcatgcagttaatagagctgcctctactgactttggaactgtcgatccttctaaggctgttccacacgaaactgttgcctcaaaagctgttcacgaattgattgagaacatgagccctggtcgtagagggagtggagagagggagcctgaaatggatgaaggagccaaaattcccgcaagtggatctccatacacttgccagaagaaggtggacatggaagatggcaatgcaaacaaaaaccaacagatgccacctgcaagtgtcatgacaagacttattctcatcatggtttggaggaaactcataagaaatcctaatacttactccagtcttttgggactcacatggtctctcatatcatttaggtggcacattgaaatgccaactattgtaaaaggttccatctcaatactgtctgatgctggtctaggaatggccatgttcagtctaggtctattcatggcattacaaccgaagatcattgcctgtggaaaatctgtggcagcattttcaatggctgttaggttcttgacaggtccagctgtgattgctgcaacctcaataggcatcggactccgtggagttcttttgcatgttgcaattgtccaggctgctcttccccaaggtatcgttccctttgtgtttgccaaagaatacaatctccatgcagatatacttagcactgcggttatatttgggatgctaattgcattgcccataaccatactctactacgtgctgcttggagtttaa

SEQ ID NO: 64 - DNA, 2451 bp, Glycine max
  source 1..2451: mol_type = other DNA
atggacgctgccaaagaagaatttctcagagactttggtgaacactacggctaccctaacggccccaagtccgttgatcaaattcgagctactgaattcaagagattacaattacaagatcttgtatacttggatcatgctggggcaactctatactctgacctgcaaatggaatctgttttcaatgatctcactactaacctatacgcaaatcctcacagccaaagtgactccagttctgcaactcttgacattgtcaagaacgcccgccagcaggtccttgactactgcaatgcatctcccaaagaatacaaatgtatatttacttctggggcaacagcagcgctgaagctggtaggagaggcttttccatggagttgtaacagtagttttatgtatacaatggagaatcataatagcgttcttggaataagagaatatgctcttggtcaaggagcagcagccattgcagtagatattgaaggagaattacatcctgaaatatcaggagaaacaattactacaaagatatcaccacaccaggttcagaggagaaaagtagctggatcactgaaagaagagccaacaggtgatgtgtataatttgtttgcctttccatcagagtgcaatttctcagggttgagatttgacttggacttggtgaagattattaaggaagactcaagcaagatcttgggaatttcttcagtttgccaaagtggacagtggatggtcttaattgatgctgcaaagggatgtgctaccatgccacctgatttgtctaagtatcctgcagattttgttgctatctcattttacaagctgtttggctatccaactgggcttggagctctcattgttcgaaatgatgctgccaagttactgaagaagacttattttagtggaggaacggttagtgcatcgattgctgatattgatttcattaaaagaagggaaggtattgaggaactgtttgaggatgggactgtttcatttttgagcatagtatctatccgccatggcttcaaaatcctgaattctttaactgtatcagcaatatcaagacatatagcatctcttgccttgtatacgaggaaaatgctcttggccatgaggcatggcaatggatctagtgtctgcatcctctatggacaccataattcaatgaaactgtgtcatgaaatgggtccaataatttcattcaacttgaaacggcctgatggctcttggtatggataccgtgaagtggaaaagctggcatcactttcaggaattcagctaaggacaggatgcttctgcaatccaggtgcatgtgcaaaataccttggcttgtctcatttggatcttatttcaaatactgaggctggtcacgtttgttgggatgatcttgatataatcaatggtaaacctgttggagctgtaagaatatcctttggctacatgtcaacatatgaggatgtcaagaaatttgttgattttgttgcaagttccttcatgtcacctcaaattcacattgaccatgggaatcaaatgaaaggccttgataagggttttgtggatactggttattatctcaaatcaattacaatatatccaataaaatcctgtggaggtttcagtgcaagcagttggcctcttagcaacaatggcttgacacatgatcgcgagtggattctcaaaagcctaactggtgaaatacttacacagaaaaaggttcctgaaatgggctttataagcacctttatagacctcagtcagggaatgttgtttgtagaatctccacgttgtgaagaaagattgcaaattcggcttgagtcagatgtttatggtgttatagaggagattgaactatatggacagaggtacgaggtatatagttatgacaatgagaccaattcatggtttagtgaagccatcggtaaaacttgctctttgttacggtattctagtttcgatcaggattttatgttaaataagatcaaaggtgcagccacatgtagagatccgaagaacaaacttaattttgccaatgaagcacagttcttacttgtatctgaggaaagtgtttctgacctaaatagaagattaagctcagatgtacagaagggcatatatggaaaagtgatgcaagtcagtgcaagtaggtttcgtcccaatcttgtggtatctggaggtaggccttatgctgaagatggatggagatacattaggattggaaacaagtatttcagttcgcttgggggatgtaatcgatgccagataatcaaccttacaataaatgcgggacaagtgcaaaagtcaaatgaacctttggcaactttagcatcctacaggagggtaaagcagggaaaaattttgtttggtatactgctgaaacatgtttctattgacggagagcagcaaaaaggtgatttctggcttcatgtgggccaagatgtgcatccggattaa

SEQ ID NO: 65 - DNA, 576 bp, Glycine max
  source 1..576: mol_type = other DNA
atggccagtgaggcagaattcaggaaggaggaggtgacccttcaagttgatggttccactggccgtataattgtgaaaggcgaaaggcaaacaaatgaacagaaacgagttcagtttgagctggcctttccattgcctccagattcagatgtggataacatatccggaaactatgacagtgaaatccttcacgtgcatgtccccaagcaagcttcacatcaagagcacaacaaagaaagtagtgatatagagaaagcttcaaattatggcagtgttgaaatgccacaagaaattgaaagtgaggaacacaatgttgttgatgagggaaggaatcatggtcatgaacaacataaggaagatagagagaagaagaaagaaagaagaaatgaaaatgcacaacacatggatgattattattctaaaaaattgacaagtaagttggagcaaaaacgcgacatgtcaagagctctagaggaggttttgatgagaaacaagggagttgttacaactgcagttgtggccttttcttttgggttgtatgtatctaataagttccattcatggaaagaaccataa

SEQ ID NO: 66 - DNA, 447 bp, Glycine max
  source 1..447: mol_type = other DNA
atggcatcgaagcggatcttgaaggaactcaaggatctccaaaaggatcctcctacctcatgcagcgccggtcctgttgctgaagacatgtttcactggcaagcaacaattatgggtcctccagacagtccttatgctggaggtgttttcctagtcactattcatttccctccagattatccctttaagccacccaaggttgcattcaggacgaaggtatttcacccaaatataaatagcaatggaagcatttgccttgacatattgaaggagcagtggagccctgcgctaactatttcaaaggtgttgctctcaatttgttccctgttgacggaccctaatcctgacgatcctttggtccctgaaattgcccacatgtacaagacagacaggaacaagtacgagtcaactgccagaagctggacccagaaatatgccatgggttaa

SEQ ID NO: 67 - DNA, 432 bp, Glycine max
  source 1..432: mol_type = other DNA
atgcatactttcttctttctgccttctttattggacatgttcattgatttttcaaagccagaaacgaaaagggcactgctaaacaaaagttctgaggataaaatacaaaagtatggtaggtccaaagctggatctcgtgtggttccatatcaagaagatgagagccaagactcagttcctgtcagtatcgtctctaaagatctccaaaggaatgacaaagagagtgaagatctccaaaggaatgacaaagagagtgaagatttatggctgatccgtgatcagttacatcaaattgagaaccagcagtcaagtctacttgatcttctgcagcatgaaggtgcttctcaagcctttgaagatgctcaaaagaagatgaaaagcatatcagaaacagtgataatttgcagcatgaaggtgcttctcaattcttga

SEQ ID NO: 68 - DNA, 1842 bp, Glycine max
  source 1..1842: mol_type = other DNA
atgccagagaagaagaacaagaaagtttctttcaccgaagaagatgctgttacgctcatgcagaggtacgacgcaactacggtgttcactttacttcaggaagtggcgcattaccctcaccccaagattgattggtgcgagttggtgaagaaaagcgcgactgagatttctaacgccagagaatatcaaatgctatggcggcacttggcgtatcgccattctttgcctgaaattttcgaagatggggatgaacctttgcctcatttccaggatgatgatagtgacctagagtgtgagctagaagcatttccccctgtaagtgtggaatgtgcatcagaggctgctgcatgtgtgaaggtaatgattgcttctcgtacactgagtgaatctgcccctagtagctcaacaatagaggctccattgactataaatgttccagtttgccattcatctagaactcctattgaaaattcacagccctctaatttgatgcaagggacaagcattatttttccagttaccgttcagagacagacactgccaactatatcatcgacagatggtatagaaaccaagggaatagttggtggtaacatggcttccaaaagaaaaagaaaggcatggtcagaagaagaggacatgcagctacgagctgctgttcaaagatggggtgaagggaattgggcaaccatggcaaaaggagacgactttcctattaagagaagtgctacacagttggctcagaggtggagcattttacgaaagaaagatggttgtacaaatacaggaaccataaccagcacacagtatactactgctgaacagctggcaactcgacactctttatctttagcccttgatatgccattcaaaaagttaactgctcctggaatgactgatgttaagccatccacatcagttaaaaatcaagcgcagattagaaatactacagagaaggtatccagtagtttagtaccacctcaacagccatctcaacaagctttgttgggatcctctgatttgcatgcaaaatccaaattagcagatgaaaaaccagtcttgaagggtaatctaatttcagatcatgtggtaaaatctgccacagcaactttggggacacgagttgatcccctttcaaatactatatcacaaattaaagttgctcaagtaaaaaatgctacagataccaagccagcagttagctctttgacaaaaccatccatttctaccaatttgccttctgacccaaagaacaaacatgttacctctttggctgataaaggtgctcaagtaaaacatgccgtggatactaagcctgctgttagctctttgataaagccatccatttctaccaatttgccttctgacccaaagaacaaacatgttacccctttggctgataaagttccattgaagcaagttgttaatcccacaaaagagttaaaagtttctgatcccagcaccacaccaaaagagaaggtacaagaaaatgagcctcctaaagttaccactggaagccaggttgatagcagcctagagaaggggagatttgaaaaaggtctagagacgagcacacctcttgttaaaattagttgtggtgaggaagtttcaaaagataaagcaaatccagtagtagtgtgtgaagagcaaggaagtgttaagaaggccactgagaataacaacattgacaaggggagtcaaaatttagatcatgacaagaagatagattctattaatcaaagttcaaatgatcaaaacgcgaatgataaacatgtaaatttgccagtacaagatgaactcagcctaagtgccaaggtagtaaaaagtgatggggaatgttga

SEQ ID NO: 69 - DNA, 1197 bp, Glycine max
  source 1..1197: mol_type = other DNA
atggctgaagcagaggatgccgtaagacgccgtaatgcggtcgccgagtatcggaagaaacttctccagcacaaggaattggaatccagagtccgatccgtcagggagaatttgcgagcttcaaagaaagagttcaataaaacagaagatgatttgaaatctcttcaaagtgttgggcagattattggcgaagttctcaggcctcttgacaatgaacgcctgattgttaaagcaagcagtggtcccaggtatgtggttggttgccgcagtaaagtggataaggaaaaattgacttcagggacaagggtggttcttgatatgacaacactcaccataatgcgagctcttcctcgagaagttgatccagtagtttacaatatgttgcatgaagatcctggtaatatcagctactcagctgttggtggcttatctgatcagatcagggaattgagggagtcaattgaactgcctctaatgaatcctgagctcttccttcgagttggaattaaacctccaaagggtgttctcctttacgggcctcctggtactggtaaaacattgttagctagggcaattgcaagtaatatagatgcaaatttcctaaaggttgtttcaagtgccataattgataagtacattggagaaagtgccaggttaatcagagagatgtttggttatgcacgtgatcaccagccttgcatcatttttatggatgagatcgatgccattggaggtcgccgtttcagtgagggaacaagtgcagatcgtgagattcagagaacgctaatggagctgcttaatcaacttgatggatttgatcaacttggaaaggttaaaatgataatggcaacaaaccgacctgatgtacttgaccctgctctcctgcgtcctgggagattggatagaaaaatagaaatccctctgcctaatgaacaatcaaggatggaaattcttaagattcatgctgctggtatcgctaagcatggtgaaatagactatgaagctgttgtgaagcttgcagagggatttaatggggctgatcttcgaaatgtctgcacagaagctggaatggcagcaattcgtgcagagcgtgattatgtgatccatgaagattttatgaaggctgttaggaaactgaacgaggcaaagaaacttgaatccagtgcgcactacagtgctgattttggtaaagactag

SEQ ID NO: 70 - DNA, 1134 bp, Glycine max
  source 1..1134: mol_type = other DNA
atggagtccgaactcaaagacctcaattcgaagccgccgaacggcaacggcaacagcgttcgcgatgaccgtcctctgctgaagccggagcctccggtctccgccgacagcatcgccgatatggagaagaagttcgccgcttacgtccgccgcgacgtgtacggcaccatgggacgcggcgagttgccgaccaaggagaagctcttgctcggtttcgcgttggtcactcttctccccattcgagtcgttctcgccgtcaccatattgctcttttattacttaatttgtagggtttgcactctcttctctgcgcccactggcgaagaggaacaggaagattacgctcacatgagtgggtggaggagaaccattattgtttcgtgtggacgcgccctctccagactcatgcttttcattttcggcttttattggatccccgaatcgaactctgcctctcaggaagacaagagtcggcagcccgaagagttgaggagacctggcgtaataatttctaatcatgtgtcgtacttggatattttgtatcacatgtcttcctcattccctagttttgttgctaagagatcagtggctaaacttccgctagtcggtctcatcagcaagtgccttggttgtgtctatgttcagcgggaatcaaggtcatcagacttcaagggtgtttcagctgttgtcactgacagaattcgagaagctcatcagaatgagtctgctccattaatgatgttatttccagaaggtacaaccacaaatggagagttcctccttccattcaagactggtggttttttggcaaaggcaccggtacttcctgtgatattacaatatcattaccagagatttagccctgcctgggattccatatctggagtgcgccatgtgatatttctcctgtgtcagtttgtgaattatatggaggtgatccgattacctgtttaccatccttcacagcaggagatggatgatcccaaactatacgctaataatgttagaaggttgatggctactgagggtaatttgatactttctgatattgggctagctgaaaaacgaatatatcacgctgctcttaatggtaataatagcctgcctagtgttttgcatcagaaagacgaatga

SEQ ID NO: 71 - DNA, 174 bp, Glycine max
  source 1..174: mol_type = other DNA
atgacttcgatctcgccaactccggcagatttgcgattgtatttacagtatgcaactttggctttgccatggtttatgattgttgttgatctctatataatgtgcatttcattgattgtttttgtatacgaagagggagtagaagtgaagggagaagaagatatgtatatttga

SEQ ID NO: 72 - DNA, 6126 bp, Glycine max
  source 1..6126: mol_type = other DNA
atggccgataacatggaaatcgatacaccctccgattcccaaccgctcaagcctcgcgatcgagtcgttcggaggcttgcccagtttggtgttcctgaggaacagctcgaccaacctggtttagttgcttttgttaaggataagagggcgttgatacctgagctggtgtctgtgatattgcctactgatgcggaagtggcggatgcatggcaagctaaattgtcttctaagaagacggcggtgggtgtgatcatgaagaagaggtttaacgagagcatggcgtggttgcagtggttgatatttgagggcgacccaggtggcgcactgagaaggctttccaagatgtctgatgggcagaggggtgtctgtgggtctgtttggggcaacagtgatattgcttacaggtgtaggacatgtgagcacgacccaacgtgcgcaatctgtgttccttgttttgagaatgggaatcacaagggccatgattactttgttatatacacgggcggcggttgctgtgattgtggagatgtaacagcatggaagcgtgaggggttctgctcgatgcataagggtgcagagcagatgcaaccacttccggaggaattcgcaaactctgttgctcctgtgcttggttcccttttcaattcttggaaagtcaagctaacattggcaagtgagtcggttaatgaaaaaaatcatgcagcaaatgagctaacttatgccgtggttgatatgcttttagaattttgcaagcatagtgagagtttgctcagttttgttgctaggttgttgttctcttctaatggtttaattaacatgctggtgagagctgagaggttcttaactgaggttgtagtgaagaaactccatgagctactcctgaaattgttaggagaaccaaattttaagtataattttgctaaagattttctcacttactatccaactgtcataaatgaggccacaaaagacagcagtgactctcctctaaagaagtacccactactatccacgttctctgtacagatacttactgtgcccactcttaccccacgccttgtgaaggaaatcaacctactaaccatgctgttgggatgctttgaaaatattttcatttcttgttctgaagatggacgcttacaggtttccatgtgggtaggtttatatgagacaactatacgtgttattgaagatattcggtttgttatgagccatgtggtagtgcccaaacatgtaaccaatgaccagcaagatatctcaagaacatggatgagacttctttcctttgtacaagggatgaaccctcaaaagagagaaacaggtcaacatatagaagatgaaaatgagcatgtccatttgccttttattttagggcattctattgccaatattcacactctattggtggatgggtcattttctgatgctagcaagggggaaatggatgcagaaattgtttggagctcttgtaagaatgattcagatgatggagataatctaaggcatgcaaaggtaggacggagatctgaggaaagctctgcatgtaatgtgaccagtgggaatagtgccttagcttctagaaagtttcgtgagataaaagctgatgactcttctcagctgcctcttccacgttctgtcactttgttgatatatgagtgtttaagggctattgagaattggttgagagtcgagaatacccctggtgtaattcctaatgcacaatctccgaacagtggtgctgtctgtgatgacaatttttcagcatttaagagaacaatatctaagtttggaaggggtagatatacatttggtaggcttacaagttcaattgaagatcatggtaagcagtgttctgaaaataatgcaattgattcagaaaatacctatattcgtcccacttttgatgataatgccatggaagaagatttccccttggaatcagatggtccacgttttttatcattgcctgattggccacaaatagtctatgatgttagttcccaagatatatctgtacacattccattacatagattgctttccatgctgttacagaaggcaatgaaaagatatttttgtgaatctgaagggtcagatgtgactcatgtttcttcagctaattcattgctgactagctacaatgacttcttcgaacaagctttacgaggaagtcatccatatggtttctctgcctatgtaatggagcatccattgcggattagggtcttttgtgctgaagtccatgccggaatgtggcgtaaaaatggagatgctgctttattatcttgtgaattgtatagatcagtacgctggtctgaaaaatgtctggagcttgatctatttcttcttcagtgttgtgctgcactagctccagaggatctatttgtcagtagacttctggagcgctttgggttgtcaaactacttatgtctaaatcttgaacggtctagcgaatatgaacctgttttggtccaggaaatgcttactcttattattcagatagtaaaagaacgacgctttagtgggctaactactgctgaatgtttgaaaagagaactaatttataagttatccattggtgatgccactcatagtcatttggttaagtctcttcctcgtgatctctccaaatttgaacaacttcaagacatcttggatacagttgctgtttattccaatccatctggatttaatcagggtatgttttctttgcgatggtcattttggaaagaattggatttgtatcatccacgttggaattcaaaggatttacaagttgctgaagaaagatacttgcgcttctgcagtgtctctgcactaaccacccaattgccccagtggaccaaaattcatcctcctctaagggggatagccagagtagccacttgcaaagttgttctacatattatccgtgctgtgctattttatgctgttttcacttttaaatcatctgaatctcgtgctcctgacagcgttcttctacctgctctacatttactgtcattatcattagatatctgttttcaacagaaagaatctagtgagaatacatgccatgatgtgtcacatcttcccataattgccctctctggagaaataattgagagttcttttggtgaacaaagcttgttgtcacttcttgttttattgatggaaatgcacaggaaggaaaatgtcgacaactttgtggaagcaggtggttgtagtctatattctctgattgaaagcttactgaagaaatttgctgagattgacaacagatgcatgaccaagttgcaaaaacttgcccctgaagtagtcagccatatttctgaatgtgttccaactagagactcaagtgtctcatcgtcggcttctgatagtgagaaacgcaaggcgaaagctcgagagaggcaggctgcaataatggaaaaaatgagagcccaacagtctaaattcttggccagcattgattccactgttgatgatggttcacaacttggtcatgaaggtgacttagacactgagcaagatgtagaagaatccgattctaaacaagttgtttgctccctttgtcatgaccacaattcaaaacatcctatctcttttttgattcttcttcagaaatctaggctcgtgagttccgttgacaggggtcccccatcatgggcccaactttgccgatcagataaagatcggacacctatcatcaacaccaacgagatggatacattgcctattaactgtaattcagttagtttgggatcaacttcatcttcccacttaagtcaatttgttcagaatgctgcaaaagaattagcttcttgtgggaaacctggggaagttcttacttttctgcaatatgtcaagaataagttcccagctttgtcaaattttcagctcccagacacatattatcatgataaagaaaatacaccatatacttttgagactttagaacagggtatgtacttctccgtgcgtgatgaaatgcatgatcttctgctatcttcgaatttgttgaatgaggatgagaaagtttcaactgtgggagggaattcaaattttataatagacactggatctgtattgcttgggaaatatacagccgatcttgtgcaagagatgtcagaagtttcttctgtgtctgaaaatgcttctaatgagactgcttctgtagaatcaacatcacagcatccagcatatgatggatttggccctacagattgcgatggagttcatctttcttcctgtgggcatgcagtacatcaaggatgtcttgatcgatatttatcttcattaaaggaaagatctgtcagaagaattgtttttgaaggaggacatattgttgacccagatcagggagagtttctctgcccagtgtgccgcagacttgcaaattgtgtcttgccaactttacctggagaattacagaagcctttcaagcagtctaccatcttgagtactagttcaataaatactgcaccccccttagctgaattgagtgaattgacttattcacttcgtctccatctaggcttaaagcttctgcaatctgctgccaatgcagttgggaaggataaattcctaaatgctattcctttgcatcacattgatagaactagaacaaatcttgaaaagttcatatgggggctttctaaaatgtattccccttgcaaagaagaaaagctatcaagattttccagactaaaccactcaatgctgatgtgggacactctcaagtactctttgacatcaatggagattgctgcacggtgtggaaagacttcttttacaccaaactttgctcttagtgcattgtatgaagaactgaaatcttcaagtggctttatattatccctgatgctgaaacttgttcaaaagacacgaagtaataattctcttcatgttctacaaagatttagaggtgttcaactcttggcagaatcaatttgttctggggtttccctgaattacgcaaataatgacgagtctgggagaggtgatatgttaagtatcttaaagcaaattgaaatggacttgtcaaacaccaacattagcttttggagtcaggcttcagatcctgtactcctccatgaccctttctcaacattgatgtgggttcttttttgtctaccacacccatttttgtcatgtgaagaatccttgttatccctagtgcatgttttctatatagtagccgtaacccaggctataattttgtattatgagaaatctaaagataaaccatcaagggaatcagctctttcagattgcctgattaccgacatatataacgttatggatgagtctggatatgcccaacagtactttgtgtcaaattattttgaccccaatgttgatattaaaaacgctattcggaggtttacttttccttacttgagaagatgtgcgttgttgtggaagatactctattcttctatcccagcaccattctgtgatgaggaaaatatattggatagatcatggaatgccccaaaggacataatggactgggccaatattgagatatttgaggtcgctaaaattcaagagctcgagaaaatgttcaaaattccctctcttgatatggttctcaaggatgaactttcaagatctacagtctctatttggtgccatcatttttgcaaggaattcgacttgcgcagaattcaacaaaatatgcatgtaacacctgctgttccatttgagttaatgagattgcccaatgtttatcaggatcttttgcagaggtgtatcaaacagcgctgccctgaatgcaaatcagttcttgatgaccctgctttgtgcctactgtgtggtagattgtgctctccaagttggaaatcatgctgcagggaaagtggatgccaaactcatgctgtaacctgtggagctggtactggagtgtttctgttgattaagagaacaacaatcctactacagagatctgcacgtcaggccccctggccctctccttacttagatgcttttggtgaggaggattttgaaatgcatcgaggcaagccactttatctgaatgaggaacgctatgcagctttgacttatatggtcgcttctcatggtctggaccggagttccagggtccttggtcagaccactattggttccttcttcctggtttag

SEQ ID NO: 73 - DNA, 1341 bp, Glycine max
  source 1..1341: mol_type = other DNA
atgtatggcggtgatgaagtatcagctatagtaattgacttgggttcacacacttgtaaagctggttatgccggtgaagatgctcccaaggctgtgtttccctctgttgttggagcaattgatcaaatggatgttgatgaagcagataatggtgaaaagaaccctggctctgctccggaatcaaataataataatgttagaaatgctgattctgacaaggccaagggaaagaggaaattgtatgtaggatcccagtccttggggtaccgcagagaccatatggaggtgctgtctccatttaaggatggagttgttgctgactgggatattgttgacagcatatgggatcatgccttcagggagtgcctattgattgatcctaaagagcatcctatgctccttgcagagccctcttctaactctcaacaacagagagaaaggacagtggagcatatgtttgagaagtacaaagcacctgcattatttttggcaaaaaatgctgttctgacttcttttgcatcagggcgtgctacgtctgtagttgttgatggtggtggaggctcaactacagttgcaccggtacatgatggttatgttcttcaaaaggctgtggcaacctctccaattggaggagaatttcttacagactgcttgatgaaaagtttggaaagcaagggtattatgataaaacctcggtattccttcaggagaaaggaaatacgccctggagagtttcagacagttgatcttgaatttcccaatactactgaaagctacaaactcttttcccagagggtgattgctagtgatatcaaggaatgtgtgtgccgtgctccagatactccatatgatgagagtgcgtattcaaacattccaatgacctcatatgagcttcctgatggccagacagttgaaattggagctgacagatttaagataccagatgttcttttcaatccatccctggttcagtcaattcctggcatggagagctttgcagaaattgctccttcagttcgtggcctgccccaaatggtgattgaaagcattaataagtgtgatgtagacattcgaagagagttgtttaatagtatactgcttgctggtggtacagcttcaatgcaacaactaaaggaacgccttgagaaagacttgttagaggaatcccctcaagctgccagagtaaaagtattggccagtgggaatgctactgaaagaaggtttagtgtttggatcgggggtagtatattggcatctcttggctccttccagcaaatgtggttctccaaatccgagtatgaagagcatggagcttcatatatccaaaggaagtgtccatga

SEQ ID NO: 74 - DNA, 1899 bp, Glycine max
  source 1..1899: mol_type = other DNA
atggctagtagttcagatgagaggtatgtttggccttggacaggcattgttgccaacatatttgggaagccaaagcatgaaccagtggagtgtgatagcatgtattggctgagaaagtttgaacaatacaaacccgaagaggcttatgttttgcattgtgcagaagatccaacagggtacgttgtgctggaatttggtacagaatggactgggttctcgcaaatgatgaagttagatacagattttcttgttgatcatcatggaaaaaaggattattatgagtcaaggaaaatgggttattcgtcaggcttatttggttggtgtgcacaggcagaagattacaactcagaagggcttgttggaaattttctccgtcagaaagctgagctgaaaacaacctctatggttgcacaagagtcattgaatgaaaaaactgaaactctggatcatttgtatggggaaataggctctgtaaacaaaaagattagtgagatggaatcgaagtacattgaggactacatgtcattggataaaatgatgaaagagattgagaagaagagagacttgcttcaccagactcgtgctgaagatgatcatgtaaattgtgctccagtgttgaagtcgatcgtaatgcgtggacgtgaaattacttataaagccatggaaaaaaataaaaaattgcaacaagagatagatacaatgaatgatgaactcgatcgctggtgtcaacaactgattgaacaagaaaagtcaactatacaacaaaggagaaaatttgaggaagaaaagaaaagccaaatggaatcactaattttagcaacagagaagcagatgaaggccagaagtgatgttctcagtttgcttgaaaagcatcagatggagaaaaaagctgtcagtgatgcactgttgaagcttgaaaaagagatgggaaatgaacaaaagttgaatttagaaattgctgaactagaggaacaactcaaggttttgaagtgtgtgaacttggaggaagctgatcatgagaataaaagaaagatagaaatagaagagatagaagaaaaattggaggacatgatttttgatatgtccgtaaaagatgatgaaaatcaagctttgaagaagaaggtacaagaagctaaaatcgagctagaagatgctaggcaacaaattattaagctattggctgttggtactgttttatcacaattaatgctttccgtgcaaaagctaattgaatgcatttgcttgacaattcaggaattaccacagttcttgaaaggggttactaagattcaaataaaaaaaattggggaggtcagtgctaggtcatttaaaaaagtgtgcatgaataggtataaaaataacaaaaaagcatcatcggagtctgtcaaactgtgcgcaaagtggcaaaaagaaattctggattcaacatggcacccttttaagattgttgatgtcgaagggaaggaaatacaggaagaaattgatgaaaatgatcctaaattattatctttaacaaatgatttgggagaggaggcatacgttgctgtggtgacagctcttaaggaattgcctgagtatcatcattctgatgatgctgagaacacccataattcaagtgagaaacaagtgatacctgagatatgggactcccaaaatggacgtagagccaccgtaactgaagctttgaatgttggaaatggagtcggaatcgcctgcatgaaggaagaaacaaaaagaataaaaggaaaagagaagaagattcagatgagaaaaatggagaaagagaagattgagggagaaaacacagagggagaaaacacaacaacagataaaactcaggagagagaagaaagagtaatggataagatacacacaattatgagtattaagcggaattaa

SEQ ID NO: 75 - DNA, 273 bp, Glycine max
  source 1..273: mol_type = other DNA
atgatttcttcaatgaaagaagtagccgagtcattgaaagaatttgttgaagtgactaagaagaagatggagaacaaaaaaaagatggagataaaagaagctcaagaagtggtacatgaagtggtgagtgagctagataatatacctaattttaatggtgcacttagacatagagcaattgattggttgacagaaaatcccattaagtttgcgattataaaagctcttccattggatgagaaagaggattacatcttatcttttatgccttga

SEQ ID NO: 76 - DNA, 996 bp, Glycine max
  source 1..996: mol_type = other DNA
atgctgctagctcaacccattttcctctctcgcaactttcccactctcaacaaccgaaactcgagaacttctcttcaacttcacaaccctcctgcactcataagagtttcaccgcaaagaacgccaataatagcctcttctggttgttttggttgcttcgtaaaaccgcttgatgtcagaaccaaaacctcgctgggtgcgttttttaagaaaagaaagggttgtgcacatatttgccatagtttgaaggaagagagcgaggttggtgggaatggcaataatggaagagactggacgacctccattttgctctttctactgtgggctgcgctaatgtactatgttttctttctcaccccaaaccagaccccgtcaagggacacgtatttcttgaaaaagctcctgaatctgaaaggagatgatggtttcagaatgaatgaagtgctagtatcattgtggtatttaatgggcttgtggccattggtatatagcatgctcttgcttccaaccggaagaagctcaaaaaacaatattcctgtatggccattcctgatactttcatgttttggtggtgcatatgttcttcttccatatttcgtactttggaaaccgccagctcctcctgtcgaagaaactcagcttaaaacatggcctttgaatttcctggaatcaaaagtaactgcaatgatatcacttgcggcaggggtagccatcataacttatgctggtctagctggacaagatgtgtggaaagaattttaccagtacttcagggaaagcaaatttatccatattacgtccattgattttattatactttccacatttgcaccgttttgggtttacaacgatatgactgctcgaaaacggtttgacaaaggttcttggctccttcccatatcattgataccatttttagggcctggtttataccttctcctgcggccatcattatcaacagtggccatttcacagactcctgttgagccagagtaa

SEQ ID NO: 77 - DNA, 2337 bp, Glycine max
  source 1..2337: mol_type = other DNA
atgaaaaatgagaaaagtgggattgaatgttcaggtggggttttgtttgattatggaggtgtggaacctgatggaggtttggttattgaatttaatgatggttttcggagtgagctggggtgtgagaatgctagggatgttgctgaaaatggtttgctgagctttgcgttgaataatgtgactcaagggggtgataatgatgcggttgagtgtacgcaggaggttaagaatgatggtttggtgaatttaagcgcggttgtggagaatgatggcaagagaaatggtgctgatagtttggttactaaagatggcatggaagattcacgtgccatagaagttgcaacctaccaattacaagatgggattccgtgtatggaagctgtggatgtcaacgctgaagaatctttgcatgtgaaagacttgtcaaggaattgcctggagctaaaaccttcatgtgagccagcctttcaggtagatgcaaatgtagatgtgatgcagaatcaaaccgtggtcgttgatgtttctgaaggactatttgagaatattcaaaatgagtgtcatggttttgatcttgttgtggatcttaattcttacaaaagcatgcataaggttggcacgtactggggatcagtgtcttcggaaatgaatttctgtgtgtctgatttagtatggggaaaggtgactggccatccttggtggccaggtcagatctttgatgcctcagctgcatcagagaaggcaaagaagcacctcaaggaaggctgtcacctgatagcgtattttggggatggaacatttgcttggaatgatgtgtcaatgcttaaaccatttcagacacatttttcacaaatggagaagctgagcaatttggaaaatttccaccatgctgttgattgtgctttagatgaggtctcgagacgggttgagttcagcctatcctgccattgcatgcctgaagatgtgctttctaagattaaaactcaagtaattagtaatgctggaatcaacaatcagtcgtgtagaagaaatggaggggacagaattatgaatgcaatgtcttttgaacccatgaaacttgtcaattttgttaaatcattagcacagtcgccacttgttgaatctgataggctggattttgtaattgcacgttcccaattgtcagccttctattgctcaaagggttattctcaactacctgagttcccagtgcttggtgtgttgttcgagaatgatatggaaactctactcatgagggagaaagagcaatgtgattatcaaacccatgtcagctttttgcaacaggatcacaagcacatctctggggatagcaagtggcacgggaagaaacttaaacttctgtcagatttgatgtctgagaagggcttctgtatttcaaatggtgaaggcacatcagaacaggaggctaagtcagttccacagcgtcgaggcaggaaaaggaagacagctttcaatacttcagaagattatttccataattcccaaaatggaaggctcacccaattacagtatgcttctacaaatgacatgaggtctcaactctgcttggctgccaaggatccaactggagaaagttgctctagtgacatggtacatttctttgcagaatttagaaaattcattagccatgattattatgcttccctggatcaggaaatgtctttggaacgaatgaactatgatgagaccggagtaacctctacagcagcattggcttctatgacacctgcaatggagccttgcagtgattcttattggactgatagaataatccaaagcattcccaaggagctatcattgacaaaataccagaatgagagagtggtttttttacctgagactctaactgaggctaatcctctttctttcaagttgcaaccgtctgctgaaactaccacagatttatgttataagcagcaagatactgatagaaatcttggatcagaatcttctaagcttgtggaacatttggatgggagttctaaagagaacttctgtcccactgctctgacactgaaatttacaaacttcgattctgttccttcaacaaccgatctcaacaacatttttggccgctttgggccactgattgaatcaaagactgaactgctagagaggacaaaccgtgccagagtggttttccaaagacgttcggatgctgaaactgcctttagtagtgctggaaaatacagcatatttggaccttcacttgttagttaccgcctcaagattttgcctcgtaagccaccacaaggtacaggaaagcgaggcagaaaaagaagaaaagaaacaagttctgtggatggcacagcagtttga

SEQ ID NO: 78 - DNA, 276 bp, Glycine max
  source 1..276: mol_type = other DNA
atgctcacgactcatcttcactctcgacccttctcttcatcttcacaatcgcgctcttcccttctcttcatcttcacgcaaccgcaccgtaagtcgttgtgccacccaccacaagctcattgctccactagaccaggtctacgacttgccagctgtcgctgcacctgggtcccattttcgaggattagggtaatttggaagccattggatgggtttgaacccggcatacgaagtggaggtgaagcgtgtgtcgcagatattagtggcgttcgttaa

SEQ ID NO: 79 - DNA, 3867 bp, Glycine max
  source 1..3867: mol_type = other DNA
atggttctctcaaccgagggcgcaacaaactccgtcgaggtggtggcgttcagtttcctaacggacgacgagatacggcggagcagccgcgtgaagataacgaacccgattctgatttttaatttaagttgtacttattttacatgtcttgaattgagttgtaagtcttgtggtcaaacaactaaacattgcccagatcattttggtcacattgagcttgtttcccctgtctacaaccctttgacgttcaatattcctagcaatattctacaaagaacttgtttctcttgccatcattttcagtcccttagaaaagaggtggatattcgtgcattccaattggagcttataatgaaaggggatattatcatggccaaaaatttggaatctattatcacagaaaaaagcaattctgttctaaggaaattcctgacgaaaaaacataagtgtcaagaatgtggagctgagaatcctagaatcagtaaaccaacctttggatggttttatatggttttacaatgcaatatatccttgggagatgcccatctaaataagtcagatccctcaaaggttctaagctggtggatggatcttcagcaatctgtaaacatgctatttgacaataaaactgcttctggcaagagagatgtggctactggaatttgtcgattgctggagaagaaggaaggcatctttcggcagaaaatgatgggaaagatggtttattttgcatgtcgctctgtcatatcaccagatccttatttagcagtcaatgaaattgggattcccccatattttgctttaagattatcagtgactccttggaatgtagtgaagttaaggaatgctatccttaatggtgctgaatcccatcccagtgcttctcattatgctgataaagtatcaatagtcaagcttccaccaaaagggaaggagatatctgaccatgagtttgaagggaaagttgtttatcgccacttgaaggatggcgatgttgtgcttgccaatcgacagccaacactccataaacctagtataatggcacatatagttcgcgttctaaagggggagaaaactgtacgcatgcactatgcaaactgtagggttactacagttaataacttctttgttttattttttccccttcgtgatctctgtacttataatgctgattttgatggtgatgagattaatgttcattttccacaagatgaaatttctcgggctgaagcttacaatattgtaaatgctaacaatcaatatgtgaaacctacaagtggggatccaatcagagctttgattcaggatcatattgtaagtgctgctcttcttacaaaaaaggatacttttctaagctatgaagagttcaatcaactactgtacagttctggtgtatccatggctgggctaggctcattctatggcaaacatgggcagaaagttttcatgtcaaattctgagagtgaaatgtttcttgcattgctatactatatcaccagaggttcaccatcatttactgttgagaagaatgcaaaaatcccatctaattttttcaagactcggatcaggaaggggaaaagatacactcaagatacatcaaaaaagaaggaaaagcctgatgaggataaattattgatatataaaaatgacttggtgctgcaagaactttatggatcaaatgttgctggcaacctactttccgcattaagtcggttattcactacctttttacagatgcatgggttcacatgtggagtagatgatcttatgttaacagaaggaaaggatgttgaaagaatgaatcagcttaatagttgtgagataattggtaacagtgttcatcgtgaattcattagagtgaagaatagtgacaatatagattcagtcacattgcaattgaatattgagaaaaaaatacgcagtaatggagaagcaaccttaacatcagataggaagatgacaaacaagcttaactcaacaacaagctcaggtatattaaaggagctactatctgaaggaatattgcaaccttcaggaaaaaattgtatttctcttatgactacatctggagctaagggtagtatggtaaatttccagcagatatcttctcatctaggccagcaagagttggaaggaaaacgagtcccacgcatggtttctggaaagaccttaccctgctttcctccctgggattgttcccctagagcaggagggttcattattgatcggtttcttactgcccttcaccctcaggaatactacttttttgatactgcagtcaaaacatctcggagtggttacctgcaaagatgcctaatgaaaaaccttgagtgtcttaaagtttgttatgaccatactgtccgtgatgctgatggttcaattattcagtttcactatggagaagatggtgttgataataaagaattggtttatagcaactattgccgtcagctagacagatccagtccatatattaacaaattacctgatgctcttgaaggaaaagcagaaaaattctcaaaacagagaaatcttggttctatggagcatgcagatttcgtaagattgatagagcataagtatgtctcatgtctcactcaaccaggtgaaccagttggtgtattagcttctcaatctgttggagaaccggcaacgcagatgacagagagaggggaggaatcaagttactccaagagtaactctaaaatgagttttcttttaattgttttaacgaaaatcctgatggctgctgcacgtgatatcaaaactccatttatgacatgccccttgagacatgagaaatctataaatttgaagaagatcactgtagctgacataattaagagtatgaaagtttctgttgtaccagtaactgttcttggtggtcagatttgcagtatatataaacttgtaatgaagctatataaatctaaacagtatccagaatatactgatattacccttgaagactgggaggaaactctaagggttaactttgtgagagactctgaagatgctcacgacaatggatcagaatctgagaaaaaaggtcaaagtaatgatgaagatgatgatggtgttgttgttgaagacactgaagggaatgaagatcttggatcagatgcacaaaagagaaaactacaaggtactgatgaggttgattatgaggatggtcctgaagaggaaacacatgatgaaaagaaaaaatctgaaccaacaaaaagatatgactgggcaatttttgtggaagctaaagggatgcattttgagatccactttagatttactggtgaacctcacattttgttaactcagattgttcagagaacagccaagaagttttgcctccaaaattttggaaaggttggtgaatgtaaagctattacatgtaaagaaagtggagtaatctactatggtaaagatgacagaaaaggggatgagatttcagcctttgagaaagaagaaataccagcacttcagacatcaggggtacattttaaaacattttgggagttggaagatgatctggatgtgaggtacatttattcaaacaacgtgcttgctatgctaaatgcttatggggtggaagcagccagggagaccatcatcagggaagtacagaatctcaatgccgactttatgacacactctggtggctactgcctgatgaacagaacggggagcatagcagactcaacatcccttttcatcaaaatgtgctttgagacagctgccaaatttattgttgaagcagcttatcatgggcaagtggacaatttggagacaccatctgctagaatttgtattggtttgccagtgaagatgggaactgggtgccatgacttgatacagaagctggagatatga

SEQ ID NO: 80 - DNA, 2175 bp, Glycine max
  source 1..2175: mol_type = other DNA
atggcacatgttcttctgcttcgccctcctctgcttcgcgtcatgtcctttcgcttcatcacctccttttccttccactcgcgcctttcttcattcccttcttctactctctccaaaaccaggacgagttttcctctgagaaaattttgggctccctgttcggttccaaatgcggtcactccaagggcgtttatgtcttctatctcgaccaccgaagcttttcacaatagtgaaagtcccacagcttatagttctgatcaaattcaggtgcttgaaggcttggaccctgtgaggaaaaggcccgggatgtatattggaagcaccggcccacgagggttgcaccatttggtgtatgaaattttggataatgctgttgatgaagctcaagctgggtttgcttccaagattgatgttgttttacttgcggatgattctgtgagcatcacagacaatggtcgcgggattcctactgacttgcatccagttacaaataaatctgccttggagacagtgttgacggtcttgcatgctggtggaaaattcggtggtgccaacagtggctactctgtttcaggaggtttacatggtgttggtttgtctgttgttaatgctttgtctgagttgctagagataacagtttggcgtgatggaatggaatacatgcaaacatattctcgtggaaagccagtgtcaattctgaaatgccttgtgcttccagatgaaaggaaagatcttcgagggacacgtgttagattttggcctgacaaagaagtattcaccactgctattcagtttgatcacaacacaattgccggacgcattagggaattagctttcctcaacccaaagcttacaatcacgtttcggaaagaggacaatgatccagaaaaagttcaatataatgaatatttctatgctgggggattggttgaatatgtgaaatggcttaacaccgataagaaagctcttcatgatgttctgagttttagaaaagaaacagatggcatcacagttgatatagcttttcaatggtctgaagatgcatattcagatacaatactgggatatgctaatagtatacgcactattgatggtggcactcatattgatgctatgaaggcttctataacaagaactcttaatagtcttggaaagaagtccaaagttattaaggagaaggatattactctaagtggcgagcatgtgagagagggtctaacatgtgttgtctcagtcaaggtcccaaatcctgagtttgaaggacaaacaaagacaaggttaggaaatccagaggtgcgaaaagtggttgatcaatctattcaggaatatcttactgagtatttagaattgcatccggatgttcttgattcagtactttctaaagctcttaatgcttttaaggcagctctggcagcaaaaagagcaagggaattggtgagacaaaagagtgttttgagatcttcatcccttccaggaaaactagctgattgttcatctacagatcctgaagaatgtgaaatctttatagttgaaggtgattcagctggaggaagtgctaagcaagggcgtgacaggcgcttccaggccattctccctctgaggggtaagattctgaatattgaaaggagggatgaagcagcaatgtacaaaaatgaagagatccaaaatttaattcttggtcttggtctcggggtgaagggagaggattttaagaaagatgcactccgatatcataagattattattttgactgatgctgatgtggatggtgcccacatccgaacactgctactgacatttttcttcagatatcagagggctttgtttgatgaaggttgcatatatgttggtgttccaccattgtacaaggttgtaaggggaaaacaagtgcactattgctatgatgatgctgaccttaaaaagcttcaaagatcattccctcctaatgcatcatacaacatgcaaaggttcaaagggttgggagagatgatgcctttacaactatgggaaacaaccatggatccagaacgcaggttattgaagcaattaaaagttgaggatgcagcagaagcaaatattgttttttcctctcttatgggtactcgggtggattttcggaaggaacttatacgaaactctgcaagcatgattgatcttgatcagctagatatttga

SEQ ID NO: 81 - DNA, 543 bp, Glycine max
  source 1..543: mol_type = other DNA
atgataattggggtcagggaagagtgccgcattcgagagcaggacttgtgcaatttccaggtcatcaacgaaactcgccaacgcttcggaagattcttctttcgccgacgtttccttgaatctctgtggagggacgttgacatgaacaggcttaaccatcatccttctgatgatctggatttgataattgtgtcacatgggctggcatctcgtgttttccttatgaagtggttcaagtggacagttgaacagtttgagcttctgaacaattttggaaatggtgagttccgggtgatacagttgggagtggtggagattggagaatacagcttggcagttcatcacacggacgaagaactgcttgaatgggggctctctcctgatatggtagctgatcagaaacggcgagccagtgccggcaagggtgactggaatgatcaatattcctggtaccttgatgcttttttttatcaccttcctgactctgatgatgagaatgtggacaaacatgacgagacagattctttgagtgaatgttcatag

SEQ ID NO: 82 - DNA, 1776 bp, Glycine max
  source 1..1776: mol_type = other DNA
atgaacctgttactcccactccctcatcaccataaccatctctcattctcccaaagcccatttctccacaaccccatcaaacccctccatttcctccccaaccctcccgcacgtgcgagcacaataatccgcatgggcgggggtccccgcacgtaccccggaggcgtgtcgaagtgggtgtggaagcgcatgcaggcgaagaaggccaagcagcttatgaaggcgcgcctctgccgggagcggcagatatacgagatgcggaagcgggcggagctcaaggcggcggtgtcggagctggagcggccgtgggaggttgtcgagagagaggcgtcggcgtcggcgccgaacttgttctcaattggggcggatgagcaggtgaaggtgcttgctgacaggttccagaggccaggcgggtttgacttgtggagtgagagggatgggccggtgctgtttgagagccctgatgagctgccctctgctaggtttttccccaaaggggttgtgcatagtgtgaagccttataggagggttgatgggtatggattggtgaaggatgctgatggagatggggagatgggtttgttgaagggaaatgattttggtggaggtttgagggaattgggtgatgggggtttgagtggattggaggatgttgataggcttgaattggaagatgatgatgatggagaatgttcatcttctgatgtgagttatgggagtgatggtggattgagaaggaatgagaatggggggaggtttttgtctgatgatgttggtaggtctagcaatcgagggcgagggcaagggcaatcttctcgaaattatgggaggaatggagcggatggtgatggcagaatgaggaagaatgggaatgggagaaggtttttgtccaataatgttgatcggtctagcggtggagaacagtcttctcctttgaattatggcggggatggaatggatggtgatggtagattgaggaggaatgggaatgggaggagggttttgtctgatgaggatagtagcgatggaaagcggtcttctcatttgagttatgggaggaatggagtggatggtgatggtgatggtggattgagaaggaatgggagagggagaaggttaatgtcatctggtgatggagggcaatcttctcgtttgaattatgggaggaatggagcggatggcaatggtagattgagaaggaatgggaatgagaatggaaggaggattttgtctgatgatgatggtaggtctagtgatggagggcgatcttctcgtgtgcattatgggaggaggaatggattgaattttgatggaagaatgagaaggaatgagaatggaagtaagtatgttgtgcagggtgttgacggggctgatggtggagaaagtttgccccattcttcaagctctggaaggtatggagcaagttttgatggcagattgaggaatagtgggagtggaaggagggttttatccaaggatgttgatgggtctggtgatggcggattgaggagtaaggagaatggaaggaggtttatgtcaaagaatgttgatgggtctaatgggttgtattcagggggagctagttctgtcaggaaacagagagggggtaattccattggaggcagaagtcgaggaaagtatactaataggacttcagagtatgcctcaccaagaggtagagatgcaaattctgaagtttatgacatggatttgcaacaagatgggagttatgggttcaggaagaagcgtgagcaacctgattctacaagttggtag

SEQ ID NO: 83 - DNA, 3621 bp, Glycine max
  source 1..3621: mol_type = other DNA
atggaggatagtgttagtagcagcaacatgaatgccaccaatgtggcgcaagctatacacaccgcccttgattgggcctctactcccaacgcgagacaaaacgccgtcgcttttctcgattccataaaaaacggcgatgtacgggtattagcaaacacatcattccttctggtgaaaaagagctggtcatctgaaatccggttacatgcctttaaaatgctgcagcatttggtgcgcttacgttgggaggaattaagacctgcagagcacaagaactttgcaaatctttctgttgatttgatgtctgagattgctgatccttgtgaaaattgggctctcaaaagccagacagctgcccttgttgctgaggtagttagaagagagggtcttaatttatggcaagaaatgcttccatctttggtttctctatccagcaagggtccaatagaagcggagttggttgcaatgatgctgcggtggcttcctgaagatattacagttcacaatgaagacttagaaggtgaccggcgaaggctacttttgcgtgggctaactcaatcattgcctgaaattttgcccctattatacactttactagaaaggcactttacagctgcaatgaatgaagctggtaggaaacagatggatattgccaaacagcatgctgccactgtaacagctactctaaatgctgtgaatgcatatgctgagtgggctccattgtcagattttgctaagtctggcatcattcatggttgtggtgtactactatctgcaccggattttcgtcttcatgcttcggagtttttcaaacttgtctctccaaggaagaggcctattgatgcatctgcttctgaatttgaccaagctatgagcagcatctttcaaatattgatgaatgtatctagagaatttttgtatagatctggttcaggtcctgggtccatggatgagggcgaatatgaatttgctgaatttatatgtgaaagtatggtgtcgctgggttcttataacttgcaaagtattgctggtgatagcaccatacttccactttaccttgaacagatgctgggatttttccaacatttcaagtttggtattcattttcaatccatgcacttctggctggtgctaatgagggatttgatgtcaaagccaaaaaattccacacattcagctgctgacagttcagctgtgagtagcacaggttctggagaagttgaaaatgctaagaaaaagactctaagctttgtgagtgatgatttttgtggtgcaattttagatacatctttccctcgcatgctcaagagagagaaaattctgcatgagactgccatttctttaggggcattagagttgtggagtgacgattttgagggcaagggaactttcagccagtatcgttctagactgttggagttgattaggtttgtttccttttacaaacccctgatagctgctactaaagtttctgaaaaaattgatacaatcatcaaaggtctcttgctttcatcagcaccaactcaggacttagctgtgatggaaagcatgcagttggctttagagaatgttgtaaatgcagcttttgatggatcaaatgactttaccaaggcaaatgctgaagttcagttagcattatgtagaacgtttgaaggtttacttcaacaatttatttccttaaaatggacggagcctgcacttgttgaagtacttgttcactatttggatgctatgggcccctttttgaagtatttcccagatgcagttggcagtgttatcaataaattatttgaacttttaacatctattccccttgtaatcaaggatatgtcaatgcataatgcacggcatgcaaggctgcagacctgtacatcatttattcggatagcaaaaactgctgacaaaagcatcttgcctcatatgaagggtattgctgatacaatgggatgtttgcaaagggagggtcgtttgcttcagggagagcataatcttttaggtgaagcttttcttgttatggcttcttctgctgggatccaacagcaacaagatgttttaaggtggttactagaacctttgagcattcagtggacacaatcggagtggcaggataaatatttgtcaggtccacacggtttggttcagttgtgctcagatgcgcctgttatgtggtcaatttttcatacattgacattctttgagagggcacttaagaggagtggattgaagaaagccaattggaattcagaaaacagctcaacaccaaactcaactcctttaaatccaatggcctctcacatatcatggatggtgactcctctcttgaaactactgcgctgtatacattccctttggtctccatctgtaagtcaagctttacctggtgaggtcagagctgcaatggtcatgggtgatgttgagaggttcagtcttcttggagaagggaactctaaattaccaaagggtgtcacagatggatctaaggttgacatgaataaagaaggatatgctgaacccaatgaatcagatatacgaaactggtttaaaggcatcagagacagtggatataatgtattgggtttgtcaacaaccgttggggattcttttttcaaatatttggatgtgcactctgttgctgttgcactaatggagaatatacagtcaatggagttcaggcatataaggcagcttgttcattccactttgattcctttggttaaaaattgtcctttggatatgtgggagatttggctggaaaagcttctacacccattgtttgtccatgctcagcaggcacttagctgttcatggtctagtcttctacaagatggtagagcaaaggttccagatgttcatgacattcttagtggatcagacttgaaagtggaagtgatggaggaaacaattctgagggatctcacacgtgagatgtgttcactcctctctgtcattgcttctcctccccttaatactggaatcccttctttggaacaatctgggcatgttagtcgattagatatgtcttctctaaaaaacttggatacagttgcatcatgctccatggttggtttccttctaaagcatgaaggtctggcccttccaacactacgaatgtgtttggaagcttttacatggacagacggtgaagctgtgactaaaatttcttcatattgctctgcattggtagttcttgcgatagtaacaaatcacgcagaactcattgaatatgtttctagagatctttttacatccattattaaaggtttagcccttgaatcaaatgcaataatcagtgctgatctagttggcatttgtcgtgaaatatttgtttacctttgtgatagacacccagctcccaggcaggttttgatgtctctccccaatattaccactcatgatttagttgcctttgaggaatctttaacgaaaacattcagtccaaaggaacaaaagcagcttacgagaagcttgtttcaattagcaactggtaacaaattgaaagcacttgcagcacagaaaaccgtgaatatcattacaaatgtttcaacaagaccgcgcccagccaatgctcctgagtccaaagttgatgatggtgatgttgtggggttggcagccatcatgtga

SEQ ID NO: 84 - DNA, 786 bp, Glycine max
  source 1..786: mol_type = other DNA
atggtgaaaacaaagcagaagcgcacaggaaaagttcctggtaaaaaaagtggtacttcttcacattgccttgttaattctggaaacggtgacaatcagcatgaggatagtgattggattatagtgaaaaagcagagagttaccattctggtgcctgctgcacctcttaatgagagatcattaacagcaaaccaaggaccaaatcacatgcatcttatgcctcctgaaatagcaagtaaccatgtgcagcttcccatggaaacatctacgatgcatccttcaggtaatgaacatgagaaggccactttggctctccgaaaggagactcgtgccgagagaagagctccccctaccttacctaaaccaactgtggtcaatccaccgtctttagatgaaataatagaatcagaaaatccacatcaaacgaacagtttgaagtcacataaactacttggaatttctgatacatcaaaagttattaagcagcccagaacactacttgcaccaagaaggtcctccaacttggaaactctgaataaaagcttaagagcatcaaaccttgagaggaagctcgaaagagccggtggacttagcaaatggctgacatcgcttggactggcgcaatttgtgagaatttttcaggggaatagtctcagtaagtatcagctggtaaatctaaccatgaaaaaactcaaggatatgggcgccagtgcagtgggacctcgcaggaaactgatccacgccatggactgtgtttgtcagccatattgctttgaggcattgtaa

SEQ ID NO: 85 - DNA, 2382 bp, Glycine max
  source 1..2382: mol_type = other DNA
atgttcaaattcttgaaagaggtggtgagtggatctggcacgggcctcaaagatctcccatacaatatcggtgaaccttatgcttctgcttggggctcttggcttcattttcgcggcacctccaaggacgatggttctccggtgtccatattttctctgtctgggagcaattctcaggatggacatttagctgcggggcgtaatggcgttaagcgtcttcgcactgttaggcatccgaatattttgtcgtttcttcacagcaccgagattgaaaccgtcgacgctggttctcccaaggttacaatttatatggtgactgagccagtgatgccgctgtccgacaagattaaggagttgggtcttgaaggtacacaaagggatgaatattatgcttggggcctgcatcagatagcaaaagctgtgagcttcttaaataatgattgtaaacttgttcatggtaatgtttgcttggctagtgttgttgtcacacaaactttggactggaaactccatgcttttgatgttctatcagagtttgaagggagtaatgaagcttcctctggacaaatgctgcaatatgcttggcttgttggatcacaatacaaaccaatggaactggcaaaatctgactgggctgcaattaagaagtctcctccatgggccattgattcttggggcatgggttctctaatctatgagctattctctggtatgaagttgggcaaaacagaggagttacgcaacactgtctccattcccaagtctctgcttccagattaccagcggctactgagttctgtgccctctcgtagattaaatacatcaaagcttatagaaaacagtgagtattttcaaaataagttggtagacacaatacatttcatggaaattcttagtttgaaagatagtgttgagaaagacaccttcttccgcaagcttccaaatttggcagaacagcttcctcagcagattgtgttgaaaaagttacttcctttattagcttctgcgcttgaatttggttcagcttctgcccctgctttgactgcattgctgaaaatgggttccagtctttctgctgaggagttccgtgtcaaggtgcttccaacaattgttaaactttttgcatccaatgaccgagctattcgagttggccttcttcaacatattgatcagtttggggagtcattatcagcacaagttgttgatgagcaggtttaccctcatgttgcaactgggttctctgatacatctgcttttctgagggaacttactctcaagtccatgctaattctggctccaaagctgtctcaaaggaccttttcaggatcattattaaagcatatgtcaaagttacaggttgatgaagaaccagctatacgaacaaatactaccatcttattgggaaatattgcaagccacttaaatgaagggacaagaaaaagagtgttgataaatgcatttacagtccgtgctttacgtgatacctttccccctgctagaggagcaggcattatggctttatgtgcaaccagttcctactatgacatcacagaaattgctactcggattcttcctaatgttgttgtactcacaattgatcttgatagtgatgtgcgatctaaggcatttcaagctgttgatcaatttttgcagatggcgaagcaacattatgaaaagacaaatacagcagaggctactgagggtacagccattggaatctcttcacttccaggaaatgcaggtttacttggatgggctatgagctccttgactcttaagggtaaaccttctgatcatgctccagttgcttctgtcagttctagtgcacgtactccaacatcttccaatgccagcccagctgtagatgctccttcaacagcacctgttcgtgttagctccacaccagattttgctgaacaccttgtccctacatccccaacgtcaacagatggctggggggaactagagaatggactaggtgaaaatgacaaggatgggtgggatgatctggaaccacttgaagaaataaagccaactccagctcttgtaaacattcaagcagctcaaaggaggccagtttctcaacctgtttcacagataaaacaagcctcaagtttgctatccaaaagtacgccaaagttgagcaaggatgaagatggtgatttgtggggttccatagcagctcctgctccaaaatcatcaaaacctttaagtttgaagtcaactgtaactgacgatgatgatccttgggcttccattgctgctcctgcacccactactaaggccaagcccttatcagctggtagaggaaggggtgctaaacttgctgctccaaagttaggtgctcagcggataaaccggacaacatcatccgggatgtaa

SEQ ID NO: 86 - DNA, 969 bp, Glycine max
  source 1..969: mol_type = other DNA
atggagagcatagagcacagaacggtggaagtgaacggcataaagatgcatgttgcagagaaaggagaaggagcagtggtgttgttcttgcacggcttcccagaactctggtactcatggcgccaccagattctagctctgagctccctcggttaccgtgcggtggcacctgatctccgtggctacggtgacacagaggccccttctacggtgaacagctacacgtgttttcatctggtgggtgatatcattgctctcatcgactctctgggtgtggacaaagtgttcctggtggctcatgattggggtgccatcatcggttggtacctctgcttgttccgaccagacagaatcaaggcttatgtgtgcctcagtgtccccttccgccccttcctcggaagaaaccccaaacagaaaaccgttgactttttccattccttgtatggagatgactactacatctgcagattccaggaaccagggaagatggaagctgagatggctggcgttgacactgcgtatctgatgaagaacatacttacaacgcgcaaaaccggtccacccacttttccaaaaggggagtatggaactggatttaatccagttactccagacaccttgccctcttggatctctcaagaagatcttgattattatgttactaaattcaacaagactggcttctctggaggcttgaactactacagaaacctcaatttaaattgggaactgacagcaccatggactggagcaggcatcgtagatgtaccggttaagttcatcacaggtggcgttgacttggtgtacacttctccggggatgaaggagtatatccacaacggtggtttcaagaaagatgtgccaactttagaggaagtggtggtgcaggaaggggttgcacacttcaacaaccaagaagcagcagaggacgtgagcaatcacatctacgacttcattaagaagttctga

SEQ ID NO: 87 - DNA, 2184 bp, Glycine max
  source 1..2184: mol_type = other DNA
atgtccaaacctcaaaatgatccccttgtgcaatcggaaacaacttgtggaacacttctctatgaacttcagataatatgggatgaagtaggggagtctgagtctgacagagatagaatgctgtttgagcttgaacaagagtgtctagaagtatacagaaggaaggtagatctggcaaatcggtctagagctcaattaaggcaggcaattgctgattgtgaggcagaacttgcagccatttgttcatcaatgggagagagaccagtgcatattcgacagactgatcaaaatgctggaagcctgaaggaagaacatgcaagaattcttccacagttggaggagatgcaaaaaaggaaaattgagcgtagaaatcaatttatagaaattcaagagcagattcaaagcatttcaattgaaatatatggtcctagagagtatattcctgctgtcgaagatgaaactgatttatccttgagaaaacttgaagaattgcacaggcagctacatgcacttcagattgagaagagtagtcgcttaaagcaggtccaggagcacctgtgtacattaaattctctttgtttagtgcttggttttgactttaagcagacaattaatggaattcatcccagtttagtggactcaaaaggatctaagagtgtaagtaatgataccattcagcaattagctgttgctatacaagaactgcgggaagttaaattacagagaatgcagaagctccaagatcttgcgactacaatgttggagctctggaacttgatggatacccctattgaagagcaacagatgtttcagaatgttacatgtaatatagctgcttcagaacatgaagtaactgaaccaaacaccttgtccgtggacttcatcaatttggttgaggtggaagtagctaggttagaagcgttaaaatcaagcaagatgaaagagcttgtattaaagaaaagaacagaactggaggagatttgtcgaaagactcatttgatcccagaaatagataatgcagtggaatctgctgttgaggccatagaatctggatctgtggaccctgctttcgtgcttgaacaaattgaacttcagatttcccaagtcaaagaggaagctttgggcagaaaagagatacttgaaaaggttgagaaatggttggcagcgtgcgatgaagagtcttggcttgaggagtacaacagggatgataatcgctacaatgctgggagaggtgctcatcttactctcaagcgagctgagaaagctcgtgccttggttaacaaaattccagcaatggtagatggtctgacttcaaaaactatttcatgggagaaggaaaaaggcattgagttcacatatgatggtattcgtctactttctatggttgaagaatacaatatattacggcaagagaaagaacaagaacgtcgtaggcagcgggatctgaagaaactccagggacaaatgatagctgaacaggaggcactatatgggtcaaaaccaagcccttcaaagccccaaagtgttaaaaagggacctaggatgtcgaccggaggaggtgcagctagtagaagagtctctctgggaggagcaatgcttcaaactcctaaaccggattcaaaatctactcactcacgtgccatgagaaaggttgataaagtgcaccaaattgagcatctaaattacctggatgatggcatttcaggtttatcagcagctagaagagggctggatattgctggcgctcctgttaaaaagcactcatttggtgctgggactcaaatcatagaatctcctctgatacgacaacctttttctcccatctcttctaacagtgtatcttcaaaagctaatgtggcaaatgctacagatgaactgagtaaacagaatgagaagttgcagagaacagtgtcgcttaacaatgggccatttactactccctccaagacagttcctactgtggtagatgaagagaataggactccaaatattcctgccccagctaccccttcgacagtatcagttccaatgaatatggccatgactccagttccttcttcaattttgaagaacgtaagcttgaattcaactcccatatcagttccttatggaaataatgacttggttcaggaggttgaatattcattcgaggaaaaaaggctcagttactatatgttagcgtga

SEQ ID NO: 88 - DNA, 222 bp, Glycine max
  source 1..222: mol_type = other DNA
atgccttctgatcctgtgtggaactatcggggaaacttgttaaaaatgctggaattggtagtcttccacaggtggtccaaacctctctgttctttgttgttctcagttgaaacttacatcgatcccttactgaaaacctcatgccaaaacaaagtcactaaactggaagggaacttccatcgcggagcaaaacgaaggaaacttacatcaatcctgttctga

SEQ ID NO: 89 - DNA, 966 bp, Glycine max
  source 1..966: mol_type = other DNA
atgtcgaaggtggtttgtgtcaccggagccagcggcgccatcggatcatgggtggtcctactcctcctccagcgtggttacaccgtccatgccaccgtccaagatatcaaggatgaaaatgagacgaaacatttggaggaaatggaaggagcaaagagtcatctccatttttttgaaatggatcttcttgacatcgactccattgccgctgccataaagggttgttccggcgtaatccaccttgcatgtcctaacatcattggtcaagtcgaagatcccgagaaacagatattggaaccggcgataaaaggaacggttaatgtgttgaaggcggcgaaggaagcaggggtggagcgcgtggtggcgacttcatcgatttcgtcgattatgccaagtcctaattggccagctgataagattaagggagaagagtgttggacggaccttgaatactgcaagcaaaaggggttgtattatcccattgcaaagactctagcagagaaagcgggttgggactttgcgaaggagacaggttttgatgttgtcatgataaaccctggtacagcattgggccctctgcttcctccaagaatcaattcaagcatggctgtgcttgttagtgttctcaaagggggcaaagagacctacgaggacttctttatgggaacggctcacttcaaagacatagcattggcacatattttggcacttgagaacaagaaagcagcaggaaggcacttgtgtgtggaatctattcgtcactttagtgatcttgtggacaaggttgctgagttataccctgaatacgatgtggctaagttgccaaaggatacccaaccagggttgttgagagcaagtggaaaggatgcatcaaagaagctgattgatttaggtctagaattcactcctgttgagcaaatcatcaaggatgcagtggagagtttgaagagcaggggctatgtctaa

SEQ ID NO: 90 - DNA, 963 bp, Glycine max
  source 1..963: mol_type = other DNA
atggataccctttcaaggattagattcctccccacttgttacagtcttcagttcccaaaaatctctgcattatctcgtttcccaaatgctttttccacagccactcccctggtttcagtgctaagagtaagagctggtcctccttcagtgagtgatacagatgaggatgtcttgcaaatattttttaaggagagagaattaaatggggattttatatcaagagcttccgatttattatggagaagagatttcagaagttctggtgattatgatattagcgagctcaccgacaacacttctcaacaaatagagcagatcatagagactgacagtgatggtggttttttgaaacttacaagaacccaagagtggctaacaggtgacaattctccaccaataaacaagaaggtgactgctaaggcattacaggacagcagtgcaagacgcatgaaactgaacatgctcaaatatgaatctctcaagagggaattactgcttctatctgtgggtattggactggcttgtagtggatattgcttggttattttttccgtacaggctgctataagttatgcgattggagtccttttcagttgcttgtaccttcaactcttgtatcaacatgcagacaacctatccagtgaagatgttcctcaaatattcaagaaaaagaagtcgaagaaaattggtataagaagtgaagaccttgaagatttcttggagaggacaatcaagggtagcggtatatctctttcatctcccaggctagttattccagcaacaatatatgggctttggattctgtttcatcagtattttaccaatgacatttttgatttccagcttgtgccagccatgttcggaatgtttgtctacaaggctgctgttctggtgcaagcttatagagacaatgaaggtttacggtttgtatttcccgaaaatgaagatggatcaagctattga

SEQ ID NO: 91 - DNA, 555 bp, Glycine max
  source 1..555: mol_type = other DNA
atggctaagtactatgatattgatgatattatcattgaggaagagactgtgtcagttatatttcagagggctgcatctggagtgggaatagatcccagttcttcaatccttattgaaacaggttcaaaagttgaactgccgttttggcttgctcatgagttacagttgagacaagcagtatcagtcaatgttcctccttgtttcaatcaaaaaacaaggcaggagctccatgctgattctgcaggtgtggatctgaggtccagatgtccatttttctatgaatttggttgcaagatagcacccatagttggcgaccgaaccattgggtttctgttgttgactgcatttaagagtaggtacaaggaaattctcaccaaggcacatactgtagcatttgcacctggttccaaattttggactattctaacaaaagaagagatctacttgtatgagacagctcaatctgcaatggcatctttcaagaagtggcgaatggggggacccagatttcagatagcttctgttctgggaagaaagagaaaatctaaggaatag

SEQ ID NO: 92 - DNA, 1392 bp, Glycine max
  source 1..1392: mol_type = other DNA
atgggaatcgatcaacttcggtggcggtggtggaacgccgtcgttttgatcggaatggtcggcgttacagtagtaaccgttaccggttccgacagtcacgagaagcaggaacaagtggtgtctcgaattgccttcggatcatgctccaaccagagcgctcctcagcccatctgggatgcagtggtggacttccatccccaaatttttatatggctgggtgataacatttatggagacttcaaacgccctttcaaaatatttggacaggaaaggactgttggaccgtggaagaatgttccacgatttgttccttcctctgagcaggaaatgaaggctggatatgaaaaagctaagtctaatcctggctatgctcgactccaacagaatgctaaggttattggtacatgggatgatcacgattatggattaaatgatgcagggaaagaatttcatggaaaaataaccaaccaaaagttgcttcttgatttcttggatgaacctcaagatagcccacggcggaaacaggctggtgtatatgcctcatatacgtatggtcctgtaggtagagatatcaagattgtcctcttagataccagatatcacagagaccctgtaggaagtgatggaaccattttggggaattcacaatggttgtggttggagacagagctcaagggtccaccaacagctcttaccataattggatcttctattcaggttatatcaaatctttcagctaccattcatccattgttcgcaatggaatcatggggtcgttttccaaaagaaagagatcgcctttttaaattaatcgctgatagtaagagggctggagtattttttattagtggagatgttcactttggggaaatcacgagatatgattgtgctttggactatccactatatgatttaacctcaagtggggttactcaatcagttgaggaggttgtcccacctttcttgcgttcttttgtgagatttgtggcatggttgaccccatctactatgagagttaaggacgaaaattgcagatacaaatcttgtatttatggtcagccaaactttggaactattgagatagattgggactctcacccagtgactctgaaattcaaagtcagggacaaggatagtgtcacagttacaggggttgatgtttcattaacggaattacaaccatcaaattcagagattttagacagggtaaaagcagagcataataattcaaagcattgcacccttgaagttagtctgccatggattgtaagatatcgcctggcaatcttgttcttttccaccttatttgtgatgtttgttgcattcctagtgctagtttacacttgcttcagactttgccgacttgaaagctgcaaaagaaagcacgattga

SEQ ID NO: 93 - DNA, 180 bp, Glycine max
  source 1..180: mol_type = other DNA
atgccactcaaacccagagctagctggttctccccgaaatgcattgaggtgcagcagttgactagacatctagaggtaaagcactatttcgatgcgagccacgagagtggtactaaatcgaggcaaactctgaatactagatatgacctcaaaataacaagggtcaagttcggccagtga

SEQ ID NO: 94 - DNA, 3864 bp, Glycine max
  source 1..3864: mol_type = other DNA
atggtccttatgaaatcgttctctaggctctaccaggtgaatccgaattatcgaatggcatccaataatccttacgagataatggacgatgagggatttgactgggaagcagctgctagagaaatcgacgtggtttgccagacgaccagtgacgtaaagggaaagggaagggtttttgagaagagaaagcagcaatccactctcgataagttcatatctattgccaatgccaatgccgaaaaggaaaccccaccaccgcagttccaagaagatgaccaaagccctgctttcatccaccacattgacaccgaggctgccaaaacttggatatatccagttaatgttcctctccgtgactatcaatttgatattacccagtccgcacttttctcaaacacattggtggccttgccaacgggacttggcaaaaccctcattgccgctgttgttatgtataactacttcagatggtttcctcaaggcaaaatagtctttgctgctccttctcggccacttgtcatgcagcagatagaagcatgccacaatattgtgggaatacctcaagaatggacagttgatatgactggtcagctaagtcctcccaaaagagctcaattttggaaaacaaagcgagttttctttgtcactccacaagtgttggaaaaggatattcattctggtacatgtttggtgaagtacttggtatgtttggtgattgatgaagctcacagagcaatgggaaattacgcttattgtgaagctgttcgcgagttaatgtctgtaccagtacagctgagaatattagcgttaactgcaacgccaggatcaaagcaacagacagtccaagctgtcattgataatttgcatatctccagacttgaatatcgcagtgaaactgaccatgatgttatctcatatgttcacagtaggaagatagaattgatacagagatctttgttgcaatattaccaggttgcaatgggcccagaagcggtagaaataaacaataagtttatggaagtaatacgccctatagtagccaggcttacaaatattggggtcattcagaacagagattaccgcactttgagcccttgtcaattacttgaaataagggaaaagtttcgccaacggcctaggcaagatctttctcatgtcaactatgaagatgttgaagggtattttggcgttctcattactctttattacatccataagttgctctcaagtcacggaataaggccagcacatgagatgcttgagagaaaattgaagcaaggatattttgcaaaatttatgagtaaaaatgaagttattctgaaagcaaggcagctaatgcagcaaagtttgtctcacggagcatctagtccaaaattgtccaaaatgctagatgtattactggagcatttcaaaactaatgatccgcaaaactccagggtaattatcttctccaattacagagaaagtgtcagggatataatgaatgcacttggagatattggggaattagtcaaagctactgagtttattggtcaaagttcagggaaagcaatgaaaggtcagtcccaaaaagttcaacaggctgtgctgaagaaatttcggtctggcgcctataatgttattgttgctacatcaattggtgaagaaggtcttgatatcatggaagttgatcttgtcatatcttttgatgctaatatttcaccactcagaatgatccagcgaatggggagaacaggaagaaagcatgatggacgagtcgtagtttttgcttgtgaagggacagagttgaagggctacttgcaaaaacaggcaaagagcaagactataagtaaacacatgcgaaatgggggcataaatagctttacctttcatcctagtccaaggatgattcctcatgtccttaaaccagaagttaaatgcgttgagctgtccattgagaaatttattcctcgtccaaagaatgtgaaagatgatgagctccacatttctccatcaaaggacaaactaactgttgcggaaattgatttacttgaaacatattttcaccctactgtggaaaacaatagtagaatgtctcttattgcctttccccactttcagacctttccttctagagtgcataaagtgaagcattcctctggaacattgatgcttatagacatgatgcagcgcttgcaaggactagtatcatttccagaagatgacaaaacttcttcacttcaggaagatctgtgtttgggacatggtaagccagtcaccactaccgaacttgatgaagctaaaaaggacaatgaatcgtgctctaggcataaaatgagaaggaatttagtctctgttaattgtctggagatggattcgtgtcatttggggattcaaagcaaagacttagttgacctaactcgacaagatggtactttctgtgacttgggtaagaatcaagaggaagcatgtgagggtgatgagacaattcctgaaactccaattgctaagagaagtttgtcaaatgaaggagataatgatggtcaaatggtaaatcttgtggagattgagacttcttcattggctgcaaatgcatgcattaatggcatgagagatgaagaacttagtccacgtttaactaatttgattagaagtggtgttgttccagagtctccggttgacgaaagagggaaatcaagatacaattctatcatacgtgattttgtgttacctgttcatctccacaaagaacaggatgttagttctttaagctctagtgaaactaaagaggtcattattgataagggcaccaacaagaatgtctgtacttcccccattaatgaaactcaaagccctttacttgacctgaaaaattgtgcaattagaagaggacgagtcttcctttctcaaattgaggaaggccatgcacataacactgatccaagcttcagcgaagaagcatatccagcagattgtggtgaaatgtccgaaagcattaaacctgcacgcaaatttaaaaggttgcggaaagctgaagatactgaaagaaatatgaatcagaaaaacaataaactttttgcttcaacagcaaactttctcaaaccatcttctgcctccaatcctgcacaatataagcatggccaaggtaaaaggaaatcaacacacaatgtgagagacttcattgaggaggaagccgaagtatcttcagatggctatgtatctaatgatgaagatgatgaggaaggcagttcatttgacagtttcatagatgacaggaccaaccctacagcagccagtcagcctgaagctagtagaatggacatgatggcaatttacaggcgttctttgctcagtcaaacaccaagtaatggagggcttgatctttgtgccacctttactcctgaccgtgtgactatggcagccagtattagtgaaagtgaggattctacagggaagacagtggatcacttccacgcagagccaaccaaacagtcagcaaatcggactttggaatctgttagtatcaaccagataacctcagaagcagtggcttcaacttgttatcctatgggaactgggacagagacaagaagtcacaaacgaagattggcattttaccattctggacacttcccaaatatgaaccttcaacgagaatttgaacttcaatcaaagaaagacgtagtgcacactgatgcaactacagatgttctatgtgatgatcaattttataatgatcttgatcttgacgagttggaggcacaagcaacattgcttctaaaacagaaattagatttgtctaatcagaaacaagatacggtccctcaatctcacacatccaatcttgatatttttcagtctccatcatttgaccttgggatatga

SEQ ID NO: 95 - DNA, 2949 bp, Glycine max
  source 1..2949: mol_type = other DNA
atggaactcaccatcccacgcaaccccaaccctcttctcgtttcctcacccccaccactctctcgaaaccccaacgtcttcaccttaaccgtgccgcgccgccgcagaaggattcggtttcgggtctcggccgccgcggagcccgatgggccgtcgtggtcgcaatcgctgctgcgcgggtcacggcggttttggggaaagtttggggaaatggtaaagaaggagacgggtctcgacttcgaaaacaggagtgtgaagaaagttggcgagtttgtgaacggagatgaattgcgaaggttggggactgattgggtgtttcggtttgtcgattggaatcgttgggaacggtggaagaatatcaaggattgggaacctaagcgaattggtgcattggttctttacatttttgttgtgacatttgcttgtagaggagtatatgtaacaattcaagcacctttcctaagtcgccagaaaaaagagttgacagaagcttacatggaagcgttaattcctgaaccatcccctaccaacattaaaagatttaaaaagggtatgtggaagaagacaatgccgaaaggcctgaaaatgaaaaagcttattgaaagacctgatggaacacttgttcatgatacttcttatgttggagaggatgcatgggaggatgatcgagaggctcctgaggagcgtgtaaaacaaattatagaggatgatgaaaggttaaacaaagaagagaagaaagagttaacaaaaggcttgggcatttcaggtgaagttcaaactgatggcacatggcgtgatagacttaataaatggagagagatccttagtaaagaacgtttttctgagcaagtggattccttaaatgccaaatatgtagttgaattcgacatgaaagaggttgaaaatagtcttcgcaaggatgtggcagaaaaggtgacacccactcagggtacaagggctttgtggatagctaagagatggtggcgctatcgccctaaacttccttacacttatttccttgacaaactcgatagttctgaggtagcagccgttgtctttactgaagacttgaaaagattgtatgtgacgatgaaagaaggttttccattggaatttgttgttgatatccctcttgatccgtatatgtttgagattattacaagttccggagttgaagtagatctccttcagaagcgacagatccattatttcatgaaggttgtgattgcattagtacctggaatactgattctctggcttataagggagtctgtaatgcttctacacattactaacaagaggtttctttacaagaagtataatcaactctatgatatggctcatgctgaaaatttcatcatgccagttggagatgttggtgaaacaaaatcaatgtacaaggaagttgtgttggggggtgatgtctgggatcttcttgatgagttaatgatctatatgggaaatcctatgcaattctatgaaagggatgtgcagtttgttcggggtgttctactctctggacctccaggaactggaaaaacactttttgccaggacgcttgcaaaagaaagtggattaccttttgtatttgcttcaggtgcagaattcactgatagtgaaaaaagtggtgcagctaggatcaatgagatgttttccattgctaggagaaatgcaccttgttttgtatttgtggatgaaatagacgctattgctggaagacatgctaggaaggatccacggagaagggcaacttttgaggctcttattgcacagcttgatggggagaaggaaaaaactggtgtagaccgtgtatccctcagacaagctatcatatttatctgtgccacaaataggccagatgaattagatcttgagtttgttcgtgctggacgtattgatcgtcgtctgtacattggtttgcctgatgcaaagcagagagttcaaatttttggtgtgcacagttccggaaagcaacttgcagaggatgtggattttgacgagcttgtcttccgtactgttggattctctggagcagatataagaaaccttgtcaacgaatcagcaataatgtcggtgagaaaagggcattccaaaattttccagcaagatattattgatgtactagataaacaactgcttgagggtatgggtgtccttctgacagaggaagagcaacaaaaatgtgaacaaaggttatcctttgaaaagaagagactgcttgctgttcatgaagctggtcatgtagtgctagctcacttatttcctcggtttgactggcatgcattttcacagctcctgcctggtggtaaggaaactgcaatatctgtattttatcctcgagaagatatggtagaccaaggctatacaacatttggttacatgatgatgcaaatggtagtagctcatggtggtcgatgtgctgaacgtattatatttggtgatgatataactgatggtggaagtgatgatcttgaaaagataacgaagattgctagggagatggttatcagtccccaaaataaaaagttggggttaattgctttaacaaaaagggttggcttaaatgatcgaccagacagcccagatggagagttgataagatatagatgggacgatccccaagtaattcctgctaatatgacactagaggtgtctgagctttttacaagggagttgacaaggtacattgaagaaacggaagaacttgcaatgaatgctttaaggaataacaggcacatcctggacttgattgtgagggaactattggagaggtcaagaataaccggattggaggttgaagaaaaattaaaggaaatgtctccagtaatgttcgaggactttgtgaagccattccagataaatccagatgagaagggaccattaccgcataatgatcggctacgatatcagttacccgacttgtatcctgctcctctccatagatgttga

SEQ ID NO: 96 - DNA, 1158 bp, Glycine max
  source 1..1158: mol_type = other DNA
atgaatacgataacgacgacgacgatgacgatgagtgtattaaacatctatggctatggctatggtgttgtagctgttgttgtgttccttatattattgctttggcagcgtcacagatttttcttcgcttcatctccgtcgtctccatccacttcattactgctcttcgattctatcaattcaaactcaaactccagaagcagaacctcaaattttgtgacagatgcagatttgaaatttttaatggagattttggatgagaagctcggcagtgataaatgggaggatgttctagataaaagaaaccatcatctaagctacagtgtgaaatgctgcaggcctaagaatggtcccctgaaatatttgagcaagactgtgttcaatgacatttcatcagagatgctaagaaacttctacatggacaatgattacagaaagcagtgggataaaacactggttgagcataaacaactgcaggtagacaaatctgatggtactgaagttggtcatacaattaaaaagttccctcttttaaagcctagagaatatgtgctagcttggaagttgtgggaggggagtgataaaacattttactgttttatgaaggaatgtgagcatcctttggcaccacgacagagaaaatatgtacgagttgagttttttagatctggctggcaaataagagaagtacctggtagcaatgcctgtgagatcacaatgtttcatcaagaagatgctggtttaaatacggagatggctaagctggcctttcgcaagggcatatggaactatgtatgtaagatggataacgcgcttagaagatactctgttataggctatcatttatcaagttcagttaccacttcaatcgatttaatgcagaaggtacctgcttgcttggacactatttcgagtaacatctctccagcaaatcctaccgtcttccatgatcaagttactgatgaatctcaaattaggatgatccaaagaaggccatcaagaaagttgatagccaatggtttgttgcttctagggggtgctactgctatatgcctgtctcgtggtcactctagcctaggtgctaaagttgccatggcatacatcataaacaaacttagtaagcgtggtgctagatcaaaccaaagcaaacaaagttga

SEQ ID NO: 97 - DNA, 834 bp, Glycine max
  source 1..834: mol_type = other DNA
atggacgccgccggcttagattccgatggccgtgaattcaaaacccccgaggacatgtggagggaacaggcgggtgaccctagcaagaagacccagtggtatcgcgacggcgtctcctactgggagggtgttaaggcaaatatggatggggtattgggagggtttgcaaatgtgaacgagcctgacataacttgcagtgaggatttcttgaacattctcttctctgaacgttttcatgctgctgatgccagacaccaaccacttgttgttcttgattgtggttctggcattggcagagtcaccaaaaatctgttaataagatacttcaatgaggttgatcttctggagccagtatcacatttcttagagactgcacgtgaaactttggctctaggatgtcagacaaattcagatatgcacaaagcagttaatttttactgtgttcctcttcaggattttacaccagatactgcaagatatgatgtaatatggattcaatggtgcattggtcatcttacagatgaagattttgtttcatttttcaagagagccaaggttggcctcaaaccaggtggattttttgtcctgaaggagaatattgccagatctggatttgtgcttgataatgaagaccgaagtgttacaagatctgatttgtacttcaaagagctattttctcgatgtggattgcatgtttacaaatcaaaggatcagaaaggattccctgaagaattatttgctgtgaagatgtatgcattaactacagaccctccaaagaaggctccccgagcaaaatccaaaacatcaactaatagacctagaactattatgtga

SEQ ID NO: 98 - DNA, 1200 bp, Glycine max
  source 1..1200: mol_type = other DNA
atgcgaagctgcatcatcaacctctggatcgtggcggcactcgtcgtccccgtgctcctctctctcagaagccctcgaggcgaaacccaaacccaaaccacttcttctttctccgattcttctaccttttctttgaagcttgcaaatggatttggcttctatgctgtctctttccctttcaaccaaaggatacgtgatgggtccaatctcgaatacgatttctatagggacacgtgtccccaagcggagggtgttgttcgttcggcgctcactcgcatatacttcgaccacagggacgttgcacctgctctgcttcgtctcttcttccacgattgcttcatcgaggggtgtgatgcttctttgctgttggatgagaacaatggtgatagaaaccgttcggtggagaagcaggctgtgccaaatcagacattgagaggttttgacaaaattgagctgataaaagaagaggtggaacaagcatgtcccgggattgtgtcttgtgctgacatacttgctcttgcagcaagagattccattcttctggctggtggacctttctatccagttttaacaggaagaagggatagccatcaatcattttttgaggaagcaactgatcagattcccagacctgatgataacgttacacgcacactaaatctcttcaatctcagaggattcaatgcaagagaaacagtcagccttcttggaggacacaacattggcaaaatcggctgcgacttcattcagcaaaggctgtacaactttcaaggcacaggacaaccagaccccagcatccccctcgatttccttcgccagatgaggctaaactgcccagacagcaagaacagcagcaccagcatagacgagtttacgatctcaaagccagtgagcagtgatttccactccaaaatggggatgtcatacatgcaggcactgtcatcttcagtgtcatcaggagcatcatttgacactcactactaccagagcctgctaagaggaagagggcttcttttcgcggatcagcagctgatggccgagcagaaaaccgccagattggtctctgcttatgcttcagatgatggatcaacctttcgaatggactttgctagggtcatgttgaaaatgtctaaccttgatgttctaactggacttcaaggtcaggttcgagtcaattgctcactacctgtgtcttcttaa

SEQ ID NO: 99 - DNA, 3282 bp, Glycine max
  source 1..3282: mol_type = other DNA
atgtggtataatgaaagaacatcaaagcataaaaatagtagaagctcgagattcagcttatgttacaaatcaattaaccgttcaagaggacctcctacaattagaattaagggtcaaccatgtcaccgcatagatagtttattaccaatgtctggaaaagaatccaaatttgcacaattatatatctttgacacagataatgaagttaaaaatataattaatgccatcagccagcatgatggaattcaaccgcaaattgtttcaagtttagctgaaatgctggataaacacaatgtgcatgctaaaagttttaggatggctagagatagattggcaaatagtgatgtcgataatgtcacattaagattgatatctactcgtgaaaaggatggtcggacatacaatgttccaattgtttcagaagttgctgcacttattgtaggtgattttaatcccaactcaagaagagatattattcttgagactcaaaatggacactatctaggcctacaattccctctgcttttcccttatggtaaagatggatataggcctgacatacttcaccgtgcaacaccaggcaacaaaaaaagagagagaaatcctcaaactttatttcattctagaagactatttcaaaaattcattgttgaagcatataccatggttgaatcggaaagactcaactacatcaggaacaaccaaaagaaacttagagttgacaagtattgttgcttacaaaattcactggatgctgggacaagccaaggtttaaataaacgcaagagagtaattttgcctcctacctttgttggcagcccccgctacatggatcagctatactttgatggtatggcgatatgcagccatgttggtttcccaaatctttttatcactttaacatgtaatccaaattggccagaaatccatagattacttgcgccattgaatttgaaaccagcagatagaccagacattatctcacgagttttcaagttgaaatatgaagaaatgctttcagacctaacaaagaatcacctacttggaaaagcagtggcatgtaaattggtgcaaaatcatatggctcatggcccatgtggaattatacgctctgaatctccatgtatgaaagaaggaaagtgtagcagattttatcctaaaacgttccaacctcgcacccttttagatccaaatggttatccaatatatcataaaagaaatgatggtcgtacaatttcaaagaatggtgttatcattgataatagacacatcgtaccttataatccaaagttactgaaaaaataccaagcgcatgtcaatattgaatggtgcaatcaaagtacttcaatcaaatatttattcaaatacataaacaaagggtacatttctccctgtgaagctgcttggagaatttttagtttttcaatacatgctaggaagcctgttgttgaaagattatatttccatcttccaggccaacacagtgttctttatgaggaccatgatgatattgatgatgtcctatccaagccaagcatttcagatttcaaatttttagcttggatgaacgccaacaaatgtttttcagaaggaagaaatctgacttattcagaattcgtttcaaagtttgtatataatcaaaagactagatcatggcaacttagaaagaaaggatgcactattggaaggcttatatgggtcccaccaatgacaagagaattgttttatttgaggatgatgcttactgcttgcaaaggagcaatttcatttgaggatattagaactgttacaaatattcaataccctacatatagagaagcatgcttttccatggggtttttacaagatgatagagaattcgttgaagtaatcaaagaggcaaaggattggggcacaactaattatttgaggaaattatttgtgttgatgcttttaacagaattacacatcgatgataatcatctgagtaatttggcattgctagagattgaacaattgttgcacgtaaatcaaaaatcacttaaagactatccttcaatgccatatcctcaggatgctgattgcacgtcctatctagccaatagcctgatattagcagaactaaattacaataaggatgataccagatcagaatttcagcaacttttttcatccatgatagatgaaaaaacatcaatttataaacaaatcattgaagccgtcaacaaaaatcaaggtggcatgtttttccaatatggatttggaggtacaggaaaaaccttcatatggagaacattggcaacttcattgagagcaaacaatcaaattgttattatagttgcttccagtggcatagcttctctattgttaccaggcggcaggactgcgcattcaaaattcaaaatacctgtacccatttttgaagactcaacatgcaacattcatcaaggaagtgaattagctgaattggtaaatcaaacaagcctaattatttgggatgaagcaccaatggctcacaaattttgttttgaagcgcttaaccaaagtttaagagatataattaaaactaaacccaactcaaataaaatatttagaggtaaagtcatcaacaatgaacaatcaccaaatgaacaagaaattgccgcatttgctaagtggatccttgacattggagatggaattattggacatgaaaatgatggctattccacaattgaaatccctgaggacctgttgataactcaatatgatgattcaatccatgccatagttgactcaacattcccagatttaacacagcatcacaacgatggtcagtacttcaacagtagggcaatattagcttcaaccaacgaaacagtcgaacaggtcaatgattatatactatcattaatcccagaaattatcataggtgagtacatggaatacctaagttctgactctatagacaaatcaaaaactagtgaaagctgtcattttcaaacaataactattgaattcctcaattcattgatgatatctggtctgccaaatcagtttatcaagcttaaaataagaacttttataatgcttttaaggaacttagatcagactcaaggcctgtgtaatggcactagattaataactacaagattggcaaagcatgtaattacggctgaaatcatttctggcaaaaattcaggtcatatggtttacaacccaagaatgttcatgtccccttcacaatcaccatggccttttaaacttttaaggaggcaatttccaatcatgttgtcttatgcaatgacaattaacaaatcttag

SEQ ID NO: 100 - DNA, 2112 bp, Glycine max
  source 1..2112: mol_type = other DNA
atggattcgtggctcaaagccgctgaaggtttatttgaagttgtagatcgacgagcgaaggctgttgccagtgacttatcagaggagcagggtgatcttaagtctccagcatcaaatgggcaaggatctcagggcaagaagacaaaatcaaaaccaaaggctcaaaagggactatccgactcatcaactacaattagtgatactactcaggagaaaagtggatctccatcagcacctgcggacatagcaacttcaatagacaaagttgatcctgaaataattgatgggagcgcttcaacatctacaaaccaaccaaaggagccacgacctagcgatgcaacatcacccttgttaggctcttcattatcaaaaatgctaggcgatgatgttggtaaacatgacccagatgatgtggaaacattagtaaatgatgctgatattggagttgccactattgctgctaatggtgatactgtccaagagagtgcttcagatgtttgtgagatggatcctcctccagctcccaaagaaattgagggccccagtgatgaacccactagtactgggcaaattatcaaatctagagatttagatgccagtaaaaatgtggatatagagaaatctgaatctgtggcttctgacacagctcctaataatgatactatactcaaagattctgatgtaaaacttgaatctgttgtggatgaaaaaagccaagaggatcacaaaactgatatctcccccaaaaaagtacaggatcaacttgatgaggctcaaggattacttaaaacaacaaaatctactggtcagtctaaagaggcaaggttagctcgggtctgtgctggattgtcatcccgtcttcaagaatacaaatctgaaaatgcacagttggaggaacttctcacttcagagagagagctgagtaaatcatatgaagctagcataaaacagctacagaaggatttgtctgaaagtaaaagggaagtgaccagagttgaatcaaatatggttgaggctttggctgcaaaaaatgctgaaattgaggcacttttaagttcgatggatgctgttaagaggcaggctgcattgtcagaaggaaatctagcttccctgcaggcaagcatggaatcaatgatgagaaatcgagaactaagtgaaacaaggatgatgcaggctctaagagaggagctagcatctgctgagcgaagagcagaagaggagcgtgctgcacataatgctaccaaaatggctgctatggaaagagaagtggaattggagcacagagcagttgagtcatccactgcacttgcaaggatacagagagtagcggacgagaggacagcaaaagccacagaacttgagcagaaagtggcacttctcgaggttgaatgcgcatctttaaatcaagaactacaagatatggaagcccgtgtgcgccgggaacaaaaaaaggcaccagaagaggcaaatcaagtaattcagatgcaggcatggcaggaagaattggagcgtgcacgtcaaggtcagagggaagctgaaaacaagctatcatccttggaggctgaaatgcaaaaaatgagagttgaaatggctgccatgaagagggatgctgagcattactcacgtcaggagcatatggagttagagaaacgctatcgggaactgactgaccttttgtactacaaacaaactcaattagaaaccatggtcagtgaaaaagctgctgcagagtttcaattggaaaaggaaattaagcgtcttcaggaagcaaaggcagaggcagaaagaagtagagtttctcgtcgagcatcatcatcttgggaagatgagactgaaataaaatcccttgagccgcttcctttgcatcaccgtcatttggtcggtgcaagtattcagttgcaaaaggcagtgaaactattagattctggggctgtcagggccacaagatttctctggcagtatccaacagctcgagttattttatttttctacttggtgtttgtacatctcttcttgatgtatctcttgcatcgccttcaggtacaagctgacacattggccgctagagaagttgcagaatctatgggactttctaaccagaatttaccgtga

SEQ ID NO: 101 - DNA, 3273 bp, Glycine max
  source 1..3273: mol_type = other DNA
atgtttcgttctgcaagatggagaagcgagaagaacagggtcaaagctgttttcaagctgcacttccatgccacccaggtgtttcaatctgggatggatgcattggtgctgtctatagttccaggggacattggaaagccaactacgaaattagagaaagctacagttcgagatagaacttgtagatgggaaaatccggtctatgaaactgttaagtttattcaagaacccaagactgggaaaatcaatgacaaaatatatcattttttggtttcaacggggttaccaaaagctagttccattggggaggtttctatgaattttgctgattatgtggaagccactaagccctcctctgtcgctcttcccatcaggatttctcactgtgatgctgttttgcatgtatcaattcagaggctgcaagaaaacggtgatcgaagagaggaagaagaatgtgaagatgtgaaactaaagtccgatgataggagctcaaggaatcaattaagcaacggcaatacggatgaaacaagtagaagttgttcttctgaagatgtgtctgccaaagcaatcatcaatagagctgaattgagttctaattataggacatctagtggctctgatatgacattgtcaagttctgatgacagctctggatttgatactccccgggaaattggacctagaaaaaagaacatccacctcaacacaaaaaggtttcttccagacccagttttacaccatgcttcagaacctcaaaaccttgctctcaatgcctcaacatcaatgcatgttgtacatcagagatcacactgggactggtcagcgggttcggagcataggctaagtacagatgattcaacatatggttctcatcataattctctcccgaaggaaagcaaccaacaaccttctcctttagagattgaaagtctcaaggctgaacttgctgctttggccaggcaggtgaatgtgtcagacttggaattacagaccctcaggaagcaaattgtgaaggaatgcaaaagagggcaggatcttgcgaaagaagtcatcgtcttgaaagaggaaaaggaagcactcaggacagaatgtgacaatctcaggtcttttcagaaacgcatggatgaggccaaagtgaggaacagatcacaattggaaggtggagatcttcatgctcttgttgaagaaataagacaagaattggattatgagaaggacctaaacgcaaatctccgattacagttaaagaagatgcaagaatctaatgtcgaactggttcttgcagtgcaagatctggatgaaatgttggagcagaaaaatagggacatatctaaccattcctatataaatgagcaggataaaaattcccaagagaaaaggaaaaacctctctaaatgtgaaacggatgatgacgaagaacagaaagcattggaggagcttgttaaggaacacactgaagccagtgagacacacttactggagaagaaaatagtagacctctatggtgaaatagagatgtataggagagacaaagatgagttagagatgcagatggaacagcttgcactggactatgagatactgaagcaggaaaaccatggccttgcatataagctggagcaaagcgatctgcaagaacagttgaaaatgcagtatgaatgttcatctcctcctgctacaatgaatgacattgaaaaccacatcaagaatctggaagatcaactcaaggaacaatcagaagacttctcaaattctctggctaccattaaggcacttgaaagccatatcagaggattagaggaggaaatggagaaacaagcacaaggatttgaagctgatctagaagcagtgatgcatgacaaagttgaacaagagaaaagagccatccaagctgaggaagctttgcgaaagaccagactgaaaaatgctaaaactgctgggaggcttcaagaggaattccaaaggctctcctcgcaaatgaccaccacatttgatgtgaatgagaaggctaccatgaaagcattgacagaagcaagtgaagtgcgtgcacagaaaagattactggaagaaaagctgcataatgtcaaagaagaactggagtcatctaaagctgattatgaggtaaaactgaaccaactttcgaaccaaatagatacgatgaaagttcagatacaacagatgttgttggaaattgaggacaagtccaagcagcttcaaaatcagaagaagcacgaggaacgagttattagggatttctctaatgagatcgtgttactaaaatctgagaatggaaagcttaatgaggatatttcatgcttacatgatcaagtagaaggaaaagaaattttaagaactgacttggaagctatgaaaaaatcaattgaggaatctgaggcactcgtacagaaaggaactgtggaaagaaatgaactggtgggtacaattgcattgttgaagaaggaggcagaacagtcacttaatgagctaaacagaatgagacatcttaaggataaaaaagaaaaagagattagagtcttgcaatcagagttggaggctgttagagctcagtacagtgacctgaaactctctctttctgaggacgaaattgagaaagaaaaactacaaaagcaagttttgcagctaaagggtgaattgaagaagaaagatgatgctttaatcagcaccgagaagaggttcagggaaagtaatggacgtgcacaacttactgatggaactaaaaatattccaaagaacaaaaaaactgcttcagttcctcagaattcaaaggaaatagccagtctgagggagaaaataaaaacacttgagggaatgatacagtcaaaggaaactgccttggaaacttcaacaacttcattcttgaagaaggaaaaggaactccagaccaaaattgaggagctggaggacaaattggaggaattcaaccaaagtattgctttgcagaaggtggttcaggatagaagtacagttgagcatctcaatgctgctgcatcttcctctggggttgcattgttattcaagagtaacgtaaacttgccggagaaagaagcaggaacctccataatggatacttcagatagcattcttgctgacttattaaccgagctaacatcattgaaggagagaaacaaatcaatggaaagcgaacttaaagagatgcaagagagatacttagaaatgagcctcaattttgccgaggtagaaggtgaaagacaaaagctcgttatgactgtacggaatctccagaaaggctaa

SEQ ID NO: 102 - DNA, 339 bp, Glycine max
  source 1..339: mol_type = other DNA
atgaaattttttgttgttcctcctgcttggtcaaattcaaattttttgcctttcatttctattgcatgttcatcattttgtgtgcttcgcttttgtgcttttccaagacctaatttttttggtttctggggtttttcctgtgacggtcgaatggttcacataagtttctcatatgttcttccatgtgttcttcgattgtctgcttttttttgtgtgttcctcacccttcttgactgtggtccaacattttgggtttcttggggttttgccggtcagattcgaatgtttcccatatgtttcccctatttttgttgcatgttcttctattctttgctttaa

SEQ ID NO: 103 - DNA, 651 bp, Glycine max
  source 1..651: mol_type = other DNA
atggcgaaaacgaggccaggcagaaaggacgtggactcatacaccataagaggaaccaacaagatcgtccgagctggagactgtgttctgatgcggccctcggacacgtcgaagcccccttacgtggcgcgcgtggagatgatcgagcaggataataggaacaacgtgaaggtgcgtgtgaggtggtactacaggccggaagagtcgattggtggtcggagacagttccatggtgcgaaagaactgttcctctccgaccactacgatgtgcagagcgcgcacaccattgagggcaagtgcgtggtgcactctttcaagaactacactaagcttgagaatgtgggtgctgaggattactattgtagattcgagtacaaggccgcttctggggcttttacccctgaccgtgttgctgtgtattgcaaatgtgagatgccttataacccggatgatctcatggtacaatgtgaagggtgcaaggattggtaccatcctgcttgcatgggcatgactattgaagaagctaagaaactagatcattttgtatgttccgaatgttcatctgatgatgatatgaagaaaccccaagctacattttctgcatcactgggagctgatggcaaggtggagccgaagcggcggaagagatga

SEQ ID NO: 104 - DNA, 1491 bp, Glycine max
  source 1..1491: mol_type = other DNA
atgagtagtacacaggtgccaaagaaaagagtggcatttgtgctgattgatgggttggctgatgtgtcactgccaaggcttggatacaagactcctcttcaggctgcaaaactgcccaacttggatgccatagcatctgctggagttaatggactaatggaccctgttgaggttggcttagcttgtggaagtgacactgctcatctttccttgttgggttatgatcctagagtttattatcgtggccgaggagcatttgaatccatgggggcaggattggccatgtcacctggtgatattgcttttaagtcaaacttcgcaactcttgatgagaaaactggaatagtcaccagtaggagagccgacaggcactttgaagaagaaggtcccctactttgtgctgccctggatggaatgaagctgccatctttccctcaatatgaagtcagagtcaggtatgcaacagaacatagatgtggagtggttgttaaaggaccaaatctgagtggaaatatatcaggaacagacccattaaaggacaaccgcttacttttgaaagcagaagctttagatgattctcatgaagcaaggaacactgctgctgttgttaacgagctgtccaaggaaataacaaaaattttggtttctcatcctgtgaatgctaaacgtgctgcagaagggaagaacatagcaaatgtagtccttttaagaggctgtggcattcgaattgaggttacaccatttataaatagacatggtttgcggccatgcatggtagctccaacgaaaataattgctggcctgggcttatcacttggtattgatattctagatgctcctggagcaactggggactatcgaactttactaacatcaaaagcatcagcaatagctaaggcactctcagctcctttacagtcctgtcccagagtttttgttcctggagaggatgagcttaaagcaggccggtcagatggctatgactttggatttcttcatattaaggcaatagatgatgctggccatgacaaggcgagcatcctcaaggtcaaagcattagaagctgtagataaagccttggggcagttggcaaggcttctctgggaggcagaatcaacaggaaagtttcagtttttcctttgtgtcacaggagatcactctactccagtagaatatggagatcatagctttgaatcggtcccgttcacaatttgccggttgaaagactttgttggtgcaattggggaatccaccatttccaaaacttctcttgacccatttcctattccgagtgttaagtcgggtgaagacttgcttgatgatttggaaacagaagagagaagagacaaatgttgtcaagcttatagtggtgattcggtttatgagctaaatgaagtggcagctgcaaagggatgtctggggcgtttccctgggggagaaatgatgggaattgtaaagaaattccttaacttagatgcgggaactgcttag

SEQ ID NO: 105 - DNA, 1434 bp, Glycine max
  source 1..1434: mol_type = other DNA
atggatactaagtcggctgtagaattaatggagtcttccgcagaggttcatttttctggatttcacatggatggttttgagcaaagaaaggctagcatagaacaaccaacaacctcagaaactgacatgtataaacaaccttttgtcataggtgttgctgggggtgcagcatctggcaagacaacagtttgtgatatgattattcagcagcttcatgatcaacgagttgtacttgttaatcaggattctttttacaataacttgacggaggaggaacttacaagagtacaagattacaactttgaccatcctgatgcttttgatactgagcaattgctacgcgttatggacaagttgaagcatggcgaagctgtagatattccaaagtatgattttaagagttacaagagtgatgacatgttaagaagggtgaacccttcagatgttataattttagaaggcatccttgttttccatgatccacgtgttcgggagttgatgaatatgaagatatttgttgacacagatgctgatgttcgtttggcaagaaggattaggcgtgacactactgagaagggtcgggatattggagcagttcttgatcagtattcaaaatttgtgaagccagcttttgatgactttattcttccaacaaagaagtatgctgatataattataccccgtggaggagataatcatgtggccattgatttgattgtacagcatattcgcacaaagcttggtcagcatgacctgtgtaaaatatatcctaatttgtatgtcattcagtcaacttttcagatacggggtatgcataccctgatacgtgatgccaagacaacaaagcatgattttgtattttactctgatcgattgattcgattggttgtggaacatggtttggggcatctgccatttacagaaaagcaagtaatcactcctactggttctgtatacactggtgttgatttttgtaaaaggttgtgtggtgtctctgttatcagaagtggggagagtatggagaatgctttgagagcatgctgtaaaggtatcaagattgggaaaattttaattcacagagaaggtgacaatggtcagcagctaatatatgaaaagttgccaaatgatatctctgataggcatgtgttactgttggaccctatacttggcacagggaattcggctgttcaagcaatttctttactcataaaaaagggtgtacctgagtccaacattatatttcttaatctcatatctgcacctaaaggtgtgcatgtggtatgcaaaagttttccaagaataaagatagtaacatctgagattgagattggtttgaatgaagatttccgtgtcatacctggcatgggcgagtttggagatcggtactttggaacagatgatgatgatgagcaagtggtagtttcttcccagtag

SEQ ID NO: 106 - DNA, 465 bp, Glycine max
  source 1..465: mol_type = other DNA
atgagtttccgtcattccatcatgaacgctcttcccattctcctcttcttcctcttcgtttctgcattagtttctgcgaagaaaactggtgatgtgacggagctgcaaattggcgtgaagcataaaccagtatcatgtgaagttcaggcacacaaaggtgacagagtcaaagtacactataggggaaaactcactgatggaactgttttcgattctagctttgaaagaaataatccaattgaatttgagcttggtactggtcaagtgatcaaaggttgggaccagggattattgggaatgtgtcttggtgagaagcgtaagctgaaaataccatcaaaacttggctatggagagcagggttccccacccactattccaggtggtgctacactcatatttgacgctgagcttgtgggagtgaacgacaaaagtttaggcgaaggaaaaggaaacaacgaactgtag

SEQ ID NO: 107 - DNA, 1698 bp, Glycine max
  source 1..1698: mol_type = other DNA
atgtcagactccttggtccgtgtttcaagacgggccgaatggggagcccacaggccgacgccaggagcgcgcaggtgccgaggcacgccaagggcgcgcgctgccatccacaatcgcgatgatgacgtctccacgagcatatcaacagcccgggcttgggccaccatcgcaatccgcgtcggtcaatgtcccgagtcgatcggcggaccggctctcaccctttcggtgataattcatcaattgtctgggtgtgccagattctcgctgcaagaattgaacataggaggaaatcaaatcaatggtacgctttctgacctttcaatattctctgccttgaaaacattggatctttcagaaaatcaattaaacggcaaaattccagagagtaccaaattgccatatctattagagtctttgtcaattggatcaaactctttagaaggtggaattccaaaatcatttggggatgcatgtgctttgcgctcgttggacatgtccaataatagcttgagtgaagagttttcaatgataatccatcacttgtctggatgtgctagatattcattggaacaattatctctaagcatgaatcaaatcaacggcacactacccgacctctcaatattctcatctttaaaaaaattatatctttatggaaacaagctaaatggagagattcctaaagatattaaatttccacctcaactggagcaactagatctgcaatcaaattccttaaagggtgtgctcactgactatcatttcgctaatatgtctaagttatacttcttggagttatctgacaactctttattggccttggcatttagccaaaattgggtcccaccatttcagttgcgcagcataggattgcgatcttgcaagctaggtccagtatttcccaaatggttggagacacaaaatcaatttcaggttgaaactttgtacgaattagacctttcaaataatcatttctctggaaaaattcctgactgttggagccatttcaagtcattaacttatttggacttaagtcacaataatttttcaggaaggatacccacgtccatgggatctcttcttcatcttcaagcattgctattgagaaacaacaacttaacagatgagatacctttctccttgaggagttcagaacaaatgttcaaaaataatgtgttactacttttaaaaagcattgatctctcaagcaatcacttttctggagaaattccactggaaatagaggatttatttggattggttttattgaatttatcaagaaaccatttgaccggaaagattccttcaaatattggaaagctaacatcacttgaatatcttgatttgtcaagaaaccagtttgttggttcaattcctccgagtcttactcaaatttattggctcagcgtgttagatttgtcacataaccatctaactggaaaaattccaaccagcacacagttacagagtttcaatgcctcgagttatgaagataatcttgatctttgtggaccgccacttgagaaattttgtattgatgagagaccaacacaaaaacccaatgttgaagttcaagaggatgaatattcacttttgagtcgtgaattttacatgagtatgacatttggatttgttataagcttttgggtggtgtttggctcaatcttattcaagcgttcttggagacatgcctatttc

SEQ ID NO: 108 - DNA, 1293 bp, Glycine max
  source 1..1293: mol_type = other DNA
atggcaccgaagcgcgtttatgaagcatggaaaggaagtaataaattcctatttggaggaaggttgatatttggaccggatgctaggtcactgcttgtcaccttgttgctgatcattgttccagttattgtcttttgtgtatttgtagcaaggcatcttcggcatgaattttcttcatataatgcaggatatgctattttggtggtggcagttctctttaatatttatgtgttgattcttctctttcttacttcctctcgtgatccgggcgttatcccaaggaatttacatccaccagaggaagagttccgttatgactcttcagtttctgttgaaattgggggacgccaaactccgagccttcagttccctcgaacaaaagaagtaatggtaaatggccattctgtgaaggtgaaatattgtgacacttgtatgctctatcgtcctcctcgttgttcacattgctccatttgcaacaattgtgttgaacgttttgatcaccattgcccttgggtgggccaatgtattgggctgaggaactaccgttacttctttctgtttgtttcttcggcaactattctatgtatctatgtgttttccatctcagctttttacatcaaggttctgatggatcattataaggggacagtttggaaggcaatgaaagaatcccctgcatctgttatattaatggcatattgtttcatctctttgtggtttgtcggtggactaactggctttcatttgtaccttattggcacaaatcagactacctatgagaacttccgctacagagccgataacaggatcaatgtttacaatctgggttgttttaacaatttcctagaagtattttgtacaaaagtgaagccctcaaggaacaatttccgagcttttgttcaagaggaggtaccgaggccaccccctccggtcatttcccgggaacccgaaccagatctgggtggtggagatccacgttctaaagttgaagacgatctagatatcggtgaagacctgttaaagatatcacagcgtcggaatattgaagaaattgacgaggacatccggagcagaggaagcaatggacctccccataatacttctgaagtagattcagttttgggttcagatcgccgggccccaacaattcgatctgaagcaaggcactcaagtgagggaagaagtgaaagctgggaaatcgggtctgaggtccttgccaattcaactgtaactgaaagcagaagctatgttgtatcaaaggaggtgcgccaaaaacttgggggttctttctga

SEQ ID NO: 109 - DNA, 336 bp, Glycine max
  source 1..336: mol_type = other DNA
atgtggtcattggatagggacccttcacctcttcctcccttagacaatcagtcatttgtggaactaatattgaagtcaacaaaagttgacttaaagacaccctgtagttttgagtttcggcttcgcatctctctcaatgctgacaacctaattttgattccgcaagtcagaaacactgctaacaaagcattttctttttcctttacactatgcaactatttatcggtatcagatatcaggaaaaaaaacaacatccctgctgcacaaattaatatccaacactctcaagctttccagtttcaaactgtgggcgctgaatttgctttgtctttctga

SEQ ID NO: 110 - DNA, 456 bp, Glycine max
  source 1..456: mol_type = other DNA
atgagtgacatgttaacaacatttacgaagaagaaagaggtggataccatcatcagagacacctttgataaagtcgtggtcctccgctttggtcgtgcctctgaccttgtctgtctccagcaagatcacattctttctaaagcagcaagggatgtgtccaagtttgccactgtggcactggttgatgttgactctgaggagattcaagtctatctcaagtattttgacatcactttgataccatctacagtgttctttttcaatgctcatcacatgaaaatggattatgggactgctgatcatactaaatggattggtgctttttacgcaaagcaagacttcgtagatgttgtagaggcaatatttagaggagcgatgaagggaaagcttattgtgaattgtcctctcccgccagaaagtataccaaaatttcagatactatacaaggatgtctga

SEQ ID NO: 111 - DNA, 429 bp, Glycine max
  source 1..429: mol_type = other DNA
atgctttgtcctctagtcccgcttattctaagtctatctaagtctatggtagagctagagcccgggaaggttctccatgacagacaagaaaggttttttggtcccttagtagtaatagtagttactcccagcccctttcctttacagaataacaaaaaagtttatttgttgcatccgaggttcaaagctctgctcatagaagggatggagacaactacttttagaatgagagaggcatggaccaatggtgtcaaaaaaaaaaaggcatttattttgtccaatccaatcacagcttattccgcctacaccttctttgccgaaagcccttgtttgtggctatcttctacattctctgccatcttccgtcatccccttgatcgactatccctaaagcaaagagccaatgccagcgaatcttctaagccataa

SEQ ID NO: 112 - DNA, 6546 bp, Glycine max
  source 1..6546: mol_type = other DNA
atggccactaagtctcaagcaaactccaggagaatgtattcatggtggtgggacagccacattagccccaaaaattcaaaatggctccaggaaaatctcacagatatggatgccaaggtgaagcaaatgattaagcttattgaagaagatgcagattcctttgccaggagagcagaaatgtattataaaaaacgcccagagcttatgaaaatggttgaagagttttaccgggcatatcgggcattggccgagagatatgatcatgcaactggagtgatccgccatgcccataggaccatgtccgaggcatttcctaatcaagttcccatgatgctgacagatgatttacctgcagtttctcctatggagaccgagccacatactccagaaatgcgccaccctgaaagtgcctttcttgatccagatgaacctcaaaaggatgcttctgctcctttccatgctatcaaaagaaatggtggttatgctggtgaaccctattctcctttgaacaaaaccggtttgaagcagctcaataatctatacatccctggagaacatgaaaatcttccaaagtttgctagaagggggctcaattttttcgagacgcaggaggagagtaatgagaaaaacagtggaaacaacaacaatttgtcccagtctgaacgtgtgatgaaagctgagacagaaattttggccttgaagaaagccattgccaaattagaagatgaaaaggaagctggcttgcttcagtatcagcagagtttggagaaattgtctaatctcgagttagaagtgtctactgcacaagagaattctcaaagacttgatgagcgagcaagtaaagctgaagctgaagttcaagccttgaaggaagcccaaatcaaattacaggctgaaagtgaagctagtcttcttcagtaccacgaatgcttggagaaaatatctaatctggagaaaaatatttcttttgcaaaaaagcaatcaggagaacttaatgaacgagctactagagctgaaactgaaactgagtccctaaagcaggaccttgctagagtagaagctgaaaaggaagctacccttgttcaatataaccagtgcttagagacaacatctaaactggaggagagaataaaagaagctgaggagaatgcaagaaggattaaggagcatgcagatatagcagaaaaagaaatcaaggctttgaaattagaagtcactaaacttaatgaagaaaaggaagatgccactctccgttatcagcaatgcttggagataatttccagtttggagtataagctctcttgtgctgaagaggaggtgcgtagtctaaattccaagatagttgatggggttgaaaagttacagagttctgagcagaagtgtcttctcttggagacatcaaatcacatgctgcagtctgaattacaatctttggcacaaaaaatggggtctcaaagtgaagaacttaacgagaagcagcaggaattgggtagactttggggttgcatacaagacgagagactgcgattcatggaggctgaaactgctttccaaactcttcagcagttgcattctcagagtcaggaagagcttagatctcttgcttctgaacttaccagtaaagtggaaatactggggaatgtggaatcacgtaagcaggctttagaggatgaagtactcagggttagcgaggaaaaaaaaattctaaatgaggtcaaaatttcttcatctttgtccatacaaaatttgcaggatgagattttgaatttgagggaaacaatagagaaagttgaacaggaggtggagcttcgaattgatgaaagaaatgctcttcagcaagaaatttactgtcttaaagaggagcttaatgatgtgaataaaaaacatgaagccatgatagaggaggtcaggtcaactgacatagacccacagtgctttggatcatctgtgaaaaaattgcaagatgagaatttaaggttgaaggaaacttgtgcggctgacaaaggagagaaagaagctcttttggtaaaattggaaaacatggagaaacttctggagaaaaatactgttttagagaattccctttcagacttgaatgccgagctggatagtgtgagaggaaaagtaaatgtgttggaagaaacatgccaatctcttctagaggagaaatcaaatcttgctgctgagaaggccaccttgttttctcagttacagtccacaactgaaaagctggagaagctatcagaaaagagcaacctattggaaaattcactgtttgatgtgaatgctgaacttgaaggattaagggtgaagtccaaggtattagaagacacatgccaatcacttgaccatgagaagtccagtatctttcaagagaaagaaactttagtttcacagttgaacataactcatcaaacactgaaagatcttgaggaactgcacagtttattggaattgaagcatttggaactaaaaggagaaagggagtctgcacttcaaaaggtagaagagctactagtttccttatattctgagagggaagagaattccagagttttgaaattgaatgaagatgaattggctgagaaggaattacaaattcatattctacaagaagatgcaaattgtaagaaaaaggaatatgaggaggaactggacagagctatacatgctcatcttgaaattttcatcttgcagaaatgtgttgatgatttggagaaaaagaacttctcccttctagttgagtgtcagagacttttggaggcttccagaatgtcttacaaaatgatatctaaattggagactgaaaatgttcaaaagcaggttcatgtgaattctctgtctgagaaaattaaaatactaaggattgggttgattcaagtgttgaagactcttgacaataacggtgggcatttcagtgaagatatgtttgaagaagaccaaatgctcctgaaccacatatatggaaaacttcaggagaggcagaaatcttttgacacagttttcaatgaaagccaacaaatggccattgagaattcaattctgattacattcctagagcagttgaaactaaaggtagaaaatcttgtgacacaaagagattcccttgatgaggatttcagtatccagtctaagcagttcttggcattgcaaatagaggtccaaaaggtgttggaaaataatcaggagttgaagttgacaataagcaaaggagcggagagaatggaagtaatgacaactgagatagacaatctacggaagcagctgtcagacttggaaaagagtcacaacaatttacaggaggatagttgcaagatattggaagagaagaagtccttgacgagaagttttctatatctgggtgaagagaaaagtaacttggaagaagaaatatgtgtcatgatccatgagacaatagctcaatctaatatttccctgatttacgagaatgttatctttgaaaagctcctggaacttaaagagctaggtgaagatcttgataaacattgttcagcaaataatgaccttgatgagagattaaaagtaatggtgtgcaaattagaaaatgcagaaatggaaaattcacatcttaaagagtcattcataaagtcaaatgttgaactacatttagttgaatctatcaatgatcaattgagctgtcagattagtgatgagagggagatgttgcatcaaaaggaaaatgagctgttggaagcagctgagatgtttcgtgttttacatactgagaaaacagaattgcaaagaatggtggaagatgtcaagattaaatatgatgaagccagggcgatgcttgaagaacaggctaatcaaattttgaaattgtccacagacaaggatcatcaaaatgaagagctcacatgcctgtgtgaagtgaaccagaagttggagtctgaaatggggtacctacgtcaagagcttggagaaactaaactgagggaaaagaagctaggtgatacagtacttaagggaacaaatgagattgaacaatgggaaactcaggcttcaacactctttgctgaacttcagatttccgctgtcaatgaaacattattagtgggaaaggtctctgagctagctgagatgtttcgtgttttacatactgagaaaacagaattgcaaagaatgatggaaaatctgaagattaaatacgatgaagcctgggtgatgcttgaagaacaggctaatcaaattttgaaattgtcctcagacaaggatcatcaaaatgaagagctcatatgcctgtgtgaagtgaatcagaagttggagtctgaaatggggtacctacgtcaagagctcggagaaactaaactgagggaaaggaagctaggcgacgaagtacttaagggaacaaatgagattgaacaatgggaaactcaggcttcaacactctttgctgaacttcagatttcttctgtcaatgaaacattattagagggaaatgtctgtgagctagctgagatgtttcgtgttttacatactgagaaaacagaattgcaaagaatggtggaaaatctgaagattaaatatgatgaagccgaagtgatgcttgaagaacaggctaatcaaattttgaaattgtccacagacaaggatcatcaaaatgaagagctcatatgcctgtgtgaagtgaatcagaagttggagtctgaaatggggtacctacgtcaagagctcggagaaactaaactgagggaaaggaagctaggtgatgaagtacttaagggaacaaatgagattgaacaatgggaaactcaggcttcaatactctttgctgaacttcagatttctgctgtcaatgaaacattattagagggaaatgtctgtgaactagctgagatgtttcgtgctttacatactgagaaaacagaactgcaaagaatggtggaagatctgaagattaaatatgatgaagccagggcgatgcttgaagaacaggctaatcaaattttgaaattgtcctcagacaaggatcatcaaaatgaagagctcatatgcctgtgtgaagtgaatcagaagttggagtctgaaatggggtacctacgtcaagagcttggagatactaaactgagggaaaagaagctaggtgatgaagtacttaagagaacaaatgagattgaacaatgggaaactcaggcttcaacactctttgctgaacttcagatttttgctgtcaatgaaacattatttgaaggaaaggtctgtgagcttgccgatgcatgtgataatcttgaacacagaaactactccaaagacatggaaactgaacatctaaaagaaagagttagcaagctggaagttgaaaatggaagactgtgcgaacaattagctgcttatgttcctgctgccagtgctttgaatgattgtataacatctctggagatgcagagtcttgcacatgaaaagcctcacgactatgaagaatcaaagcattttcaggttaaaagtttggtgaataatgaatgcactgaaaatggtcgacaaacagatgaagatcagactgttatggcaccagatgcactctcttatttccaagacatgcagagaaggatcaatgcaattgcaaggacagttaagcagttaaatgaaagtcttaaaccgaagaatgaagagaacattcaagcaagcaagcatgttactcaagcagatcaagcaaggccatctatcccagttaccgagattgaagttctgccgaaagacatcatgcttgaccaaatatccgaatgttcgtcgtacgggataagtaggaggagagaaattcttgaggctgatgatcagatgcttgagttgtgggaaacagcagataaggatgccacgattggcaagcaagctgaaaagacacagaagatggctgctggcaatcatcaaagaggaacaaccaaggaacccaaaaataggtatccttcaacagattccttggtggaaaaagagttgagtgtggacaagttagaggtctcaagaagattgacactaccacgcgaagaagggaaccaaagcaagattttagaaagacttgattctgatgcacagaagttgacaaacctccaaatcaccatacaagatttgatgaagaaagtagagataaatgagaagagcacaaaaggaaaaagtgttgagtttggtgaggtgaaagggcagcttgaagcagctcaggagaacatcacaaagttgtttgacgccaaccgcaagttgatgaagaatgtggaagaaggtaccgtgtcttctgttgggaaggatgcagcagagttaggtgaaattggaagtgtcagcagaaggagagtttcagaacaggcacggagagaatcagaaaaaataggacaattgcatttggaggtgcaaagactgcagtttttactgctgaaactaggtgaaggaaaagaaaacaaagagaaaacaaaaacggctgatcgaagtccacgagtccttttgcgagattatctctatggcgggacgagaaccaacaaccaaaagaagaagaaaaagctaccgttttgttcatgtgtgcgacctcccaccaagggagattga

SEQ ID NO: 113 - DNA, 993 bp, Glycine max
  source 1..993: mol_type = other DNA
atggcaagaagagatcttgaaagtggcggtggagtaaccaagaacaacagaagtgcagaggaaaactactctgcgcccgaatcttcccatgtttatgattctgagacccattggacatcatggctcgtgcccatgtttgtggttgctaatattgctgtctttgttattaccatgtacatcaacaattgtcccaggaacaacattcgtttccaaggtcgctgcgtggcaaggtttcttggaaggttctcttttcagcctatgcaggagaatcccttgcttggtccttcttcttcaacattgaccaagatgggagctcttcgatgggataatgttgtgaatagacatcagggatggagacttgtcacttgcatttggttacatgctggggttattcatttgctggctaatatgttgagtctggtcttcattggcattcgccttgaacaacaattcgggtttattaagattggaatcatatacttggtgtctgggtttggtggaagtgtactgtcgtctctatttatcagagaccacatctctgtgggtgcttctggtgccctttttggacttcttggcgcaatgctgtctgaacttatcacaaactggactatatattccaataaagcaatggctttaataacccttctagtgatcattgtaatcaaccttggcattggcattttgccacacgtagataattttgctcacatcggaggattcctggtgggattactccttggctttattttgctgccacgtcctcagttcggttggttagagcagcgacgtcttcctgccggtgttcagatgaagtcaaagtacaagactcaccaatatgttctaggggttgtgtctcttattctgttgattgcagggttatcgactgcattggtgatgctatttcggggtgagaaagggtatgaccactgccattggtgtcgctacttaacatgtgtccctacttctaaatgggaatgcagcaatgacagttaa

SEQ ID NO: 114 - DNA, 1383 bp, Glycine max
  source 1..1383: mol_type = other DNA
atgaaccccaacgatggcaacaactctcccagtcccaagcccttggagatggtcaacccggttcctccgtttctttctaagaccttcgacctcgtcgacgccccgactctcgacccgatcatctcgtggaactccacgggcctcagcttcgtcgtgtgggaccctctcgagttcgcgagaattgtccttcctcgacatttcaagcacaacaacttttccagcttcgttcgccagctcaatacttatggattccgcaagattgacacagacaagtgggagtttttcaacgaagctttccagcgagggaagaagcacttgctgaagaacatccaaaggcgcaggtcatctcaatcccagccggttggtagctatattggaatcggatcttctaccgaggcaggggggtcagaggttgagattgagatagaaaggcttaggaaagaaaagactatgctgatggaagaggtggttgatttgcagcaagagcaacgaagaacagctcaccacgcaggagaagtgaatctaaggctccaatctgcagaacaaaggcagaaacaaatggtttctttccttgccaagttgatccaaaaccccgcctttttagcccgccttagacataagaaggagcaaaaggaaatagattctccaagagtggtaaggaagtttgtcaaacagcaccatgaaactggaacaacaacaacagcagaaactctccaagaagggcagatagtgaggtatcaacctgattggagaaatatagccatgtcttctgaaactccaaaactaagttcagtttccattgaacaatctcctcactacctttcacagggtttggcaggagaaatgagtgtagtaggtgcagaagaccttactgctcaaattgacaatattgtatcagatgatttggctgcagtgcacgggattacgccgtctcaagaaatgatgattggagaaggatcatctacctttggagctgaagaccccctcttcaaagggaagagtgtcatgagtccaattctagaagttcctccagagtattttccttccttcccagagggtttgaccaaggagaagaaagactttcaagatttttctgctcttggaactgaaggcatgataaagctagaagatatatgggactctggcctcaatgttagtggtgctgctttaagcagtgggaatgagctgtggggcaatcatgtcaactatgaagagtttccacaatttggagtcacaagtggtatgtctgactcagatatctgggatattggcttaggaagttttggaattgatacgtggccaactgatgaaccttctcttggtgaaacagatggtcaatctggtcagccaaatgaagataggtctaagaattttgatccatag

SEQ ID NO: 115 - DNA, 1083 bp, Glycine max
  source 1..1083: mol_type = other DNA
atgaagatcttcattatctttagtatcaccttcacatgtggtatttttggaaatgttaattcaaatgtcaatcctctcccgtatgaagctattttcaattttggtgactctataagtgatacaggaaatgctgctgcctatcaccatgttcccaaggatggtaaaagcccttacggctcaacatacttcaaacatccatcgggacgtttgtcaaatggacgattgatcatagattttataaatctcaccaaaggtcaagacattaggcatggagtaaattttgcatttgctggtgctggtgcacttgatatgaactatttcacaaacaatagactcaaggcaccagcaacaaataattcattaagtgttcaacttgattggtttaagaagctcaaaccttccctgtgcaaaaataaaaaagagtgcaataactacttcaaaaaatcattgtttatagtaggagaaattggtggaaatgatattaatgcgcctatctcgtataacaatatttctaaacttcgtgaaatagttcccccaatgattgaagaaattaccaaggccaccattgcattaatagaagaaggagctgtagaggtagtggtgccaggaaactttccaattgggtgtaattctggtgtcttgacagtggtgaatagtggcaacaaagatgactatgatcaatttgggtgcttagcagcttacaatgttttcattaaatattataattggcggcttaatcaagccatagaggcactaagacaacaaaaaaaccatgttaagataatatattttgattattatggtgatgccagacgtttatttcaagcaccacaaaaatatggcttttcatctagtaagaatgagactttcagagcatgttgtggaacaggcgagccttacaatgttgatgaacatgcaccttgcggaagtttgacttcaacaatttgctctgatccttcaaaacatataaattgggatggagctcactttactgaagaagcttataaactaatagcaaaggggctagttgagggtccttttgcaagcccttctcttaaatctcctctttttaagatagtataa

SEQ ID NO: 116 - DNA, 3906 bp, Glycine max
  source 1..3906: mol_type = other DNA
atgcagaactatcaacatgcaaagagatctgattctgcaaaagccaggatcaacagaaaacgtattatgcagcagaagcgacatggtcatgctcagtcatcttcacaaccaatagttaactgcacttcagaatataatagatattctgatcttggtgatcaactcatccaatgtagatattgtaatgcacaaatgtggtatgatgaaagaatatcaaaaaataagaattgtcataatccaaggttcagtttatgttgtggagatggaaaagttgaattgtcattattacaaaatctaccccaatatctccaacaacttctatttcatgatgataccattgatagtaaaaattatcaacataacttacgagcatacaacatgatgtttgcctttacttctgctgaaattaagctagataaaacaattaataattcaaaaggacctcctacaatcagaatacaggggcaaccatgccatagaataggcagtctattaccaatgcctgggaaaaaacccaaatttgcacagctatatatctttgacacagaaaatgaagtgcaaaatagaattaatgtcatgagtcaatattccggaattcagagtcatattgtttcagctttaagtcacatgctcgatcaacacaattctcatgctaaaagttttagaatggccagagatagattggcaggtgatcaacccaataatatcaaattgcaactcatagctgctcggggaaaagatggtcgtgcatataatatgcctaatgttccggaaattgttgcacttattattggcgattttcatccaggctcaaaaagatatattattgttgaaactcaaaatggagaattacaaagaatccatgaacttcaccctagctatctaccactacagtatcctctactctttccttatggagaaacggatatagagctgacatacttcacccgaaaccgtctgacaatgagagagtggtttgcttatagatttcagtctaggtcaaatgaagcacagactttattgcattctcgaaaattatttcaacaattcattgttgaaggttatacaatggtggaatctgaaagacttagctatatcagaaacaaccaaaagaagcttagagttgataagtattctagcttacaaacttcattggatactagaatggctaaaggcttaaccaagggaaaaagagtcatcttaccctcaacgtttgttgggagctcacgttatatggatcaactttactttgatggtatggcaatatgcagtcatgttggttttccaaatcttttgattactctaacctgtaatccaaattggcctgaaattcgtagattactttcacctttgaatctcaaaccaacagacaggccatatattgtatcacgaattttcagattaaaatatgaacacatgctgtcagacttaacaaatggtcaattaataggaaaagtggttgcatatatgaaatacagaattatacttaccatcgtaactttatcttttgtggatcacaaatatgcatactatagacctcagactcttttggattcaaacggttatccagtctatcgtagaagaaacaatggtcattcaatttcaaagaatggtgttatcattgataacagatatgtagtaccttacaatccaaaattactgaaaaaatatcaggcacatataaatattgaatggtgtaatcaaagtacttcaatcaaatatttatttaaatacataaacaaaggctatgacagggtgactactgttatgctttctgatagcaataacgcaactcaaaatgtaaatattcaaaatgatgaacttaaagaatatctagattgcagatacatttctccttgtgaagctgcttggagaatatttgcttttccaatccacggcagaaagcctgttgttgaaagattgtattttcatcttccagatcaacacatcattctttatgaagatcatgatgatatagatgatgtactatcgaaaccaactatttcagattcaaaattcctagcttggatgaacactaataaagattttgatgaggccaggaatctaacttattcacaatttgtgtcaaagtttgtaccaccaaccacagaagaattgttttatttaagaatgatgcttggcagctgcaaaggacctacttcatttgaggatatcaggactgttgcgaatatccaatatccaacatatagagaagcatgctttgcaatgggctttctacaagatgacagagaatatgttgaggcaatcaaagaggctaaggactggggcacaactaattatctaaggaaattaagtaatgatgatactcttagaaatttgacattgattgaaattgaacaactattgcacataaaccaaagatcactaaaagactatcctacaatgccatatcctcaggatatcaatctcacatcctacctacaaaacaacttggtgttatcagaacttgactacaaccatgatgcaacgagatcagaatttgagcatctttttgcatccatgacagatgaacaaaaaacaatttatcacagaattatccaagttgtcaacaacaatgaaggtggaatgtttttcctatacgaatttggaggcataggaaaaacattcatatggagaacattagcaagttcattgagagcagagaatcaaattgtgattattgttgcttctagcggaataacctctctattagtaccaggaggcagaactgcacattctagatttaaaattcctataccaatttttgaagactcaacatgtaatatccatcaaggcaccgaattagcagaattactgaaccagacaagtctaataatttgggatgaagtagcaatggcccacaaattttgttttgaagcgcttgatcaaagtttaagagatattatcacaaataaatcaaactcaaatcaaatatttggaggcaaagttatagtatttggtggagatttttgtcaaatactaccagtcatcccaagaggaacccgctcagacattgtaaatgcagcaatcaattcatcctatctatgggattcatatgaaatattgaccttgacaaaaaacatgcggttacacagcaatctagaatcagttgatgaacaagaaactgccacatttgctaaatggattctagacattggagatggaattatagatgatgaaaatgatggttatgctacaattcaagttcctgctcatcaactcattactcaatatgatgatccaatcagtgctatagtgaaatcaacattcccagacttagatcagcaccacaataatcctgaattctttaaatccaaggcaatactagcttcaacaaatgaaacagtagaacaaatcaatcattacgtgctatccttcattccaggtgaccacatggaatatctaagctcttattcggttgataaatcagaaaccaatgaagatttgtatttccaatcaattactactgaattccttaattcattgaacacatccggtttgccaactcattctatcaaacttaaaattggaagtcctataatgctgttaaggaacctcgaccaaaatcaaggtctatgtaacggtactagattagtggtaacaaagatggcaaaacatgtaattgcagctgaaattatctcaggtaaaaacattggcctcgctgtttatattccaagaatgtcaatgtccccttcacaatcaccctggccgtttaaactattaagaagacaatttccgattatgctatctcatgcaatgacaattaacaagtcacagggacaatcactatccatggttggactttatttgccgaaaccggtattcactcatggccaattatatgttgcatattcaagggtcaactcagcaaaaggattaaaaattctgattcatgatgatgagtag

SEQ ID NO: 117 - DNA, 990 bp, Glycine max
  source 1..990: mol_type = other DNA
atggtgacggaccaagatatagcgaaaggggtggagtccctactgcgtcactctgacccaaactccataactacggtaaacggcgtcgttcagcagctggaggccaaactagggttagacctctctcacaaggccagtttcatcagagaccagatcgatcatcttctccggtcacagccacagacatttgctcctcaccctcctccacttcacaaagactattttgcccctcacacccaactgcacttcccaaccacccactttgctccccattttgccctccatgacgagatcaacttcctgcagcacccccaccctcctccaccgcgtaaagtcgagacctttcctcctcaaaatgttgcccctcctcaagtgcccaaagaaagtgtgcaaactggaagcaaaagaagaggtggtgctggtggtctaaacaaagtttgtggtgtttctcctgaacttcaggcggttgttggtgagccggcaatgccaagaactgaaattgtgaggcagctgtgggcatacataaagaaaaacaacctccaagatcctggtaacaaaagaaagataatttgtgatgatgccctgcgtttggtatttgagacagactgcaccgatatgttcaagatgaatcagttgctagctaaacacattatcccacttggtcctacaaaggagtcacaggctaaacgagtgaaggtggatactgaaattaagactgaaagtgctgaacctgctccatctaccgtggcaatatctgaagcgcttgccaaatttttgggcactgagggaagagagatgcaacagtctgaagccataagacttgtttgggagtacatcaagcttcaccatttggaggatcctttaaattcaatggtgatattatgcgatgcaaagcttcaagagctacttggatgtgaaagcatttctgctttaggaataccagagatgttagcacgtcatcatctatttaaacagtctgacacccgttag

SEQ ID NO: 118 - DNA, 780 bp, Glycine max
  source 1..780: mol_type = other DNA
atgtctagtaagaaggaagagaaatctcaggctgcggctgaaagaatcaaggctgcagccctgagtgctgcgaaaggccttagtcgtgcccaggctgaaagggctgcgactgcagctgcccgaaacgtcaatgcttatgggcagaaggaagaagggcccagcagatggcaggagaaaagggaagcaaagaggcagatgtatttgatgagtaccgaaaaagctgttaaattgggggaaagaaaagacctcaagcctgcaatgtcggcagccggtggattggctcagtgccaaaagtgtttccaaagtggccactggacatacgaatgcaagaatgaacgggtttacatttcaaggccatcccggacccagcaacttaaaaaccctaaattgagggtgaatgtgtctgtgacttatgatttggatgataatattcctgatgctaaggaggaaaaggcaaaaacaagttccaaaaaaaccaaaaggaagcatcggaaggactctgattctgctagtgatagtgaggattctgttttcgagactgatagtggcagtgggtcatcatctgttacgggatcagattattcttcagaaagtagttcaggttacagttcatcttctgattcagaggaggaacggagacggaggagaaagaagaagcagaagagagggagacgcaagaggtacacttcatcagaatcatctgattcagattctgcttcagactctgattccgatgataaaagcagccggagaaagaagaggaataatcgaagacgttga

SEQ ID NO: 119 - DNA, 2001 bp, Glycine max
  source 1..2001: mol_type = other DNA
atgtcaatgtcggattccgattcctcctcctcctcttatggcgctgaatacaagagtctcaaacaagttagccgcgaccgattattacatgaaatgcttaggtcagcgaaaacaggagattcaaaatctacatggaaggtactcataatggacaaacttactgtgaagataatgtctcattcttgcaagatggctgatatcactgatgaaggtgtttcattagttgaagacatatacaagcgaaggcagccattgcccaccttggatgctatatacttcatccagccaaccagagagaatattattatgtttttgtcagacatgtctggaaggaaacccttgtataggaaggcctttgttttctttagttcacctattgccagagaattggttatggaaattaaaaaagatgcacaggtgttacctcgtataggtgctttgagagagatgaacttggagtattttaccatagatagtcagggcttcattacaaacaatgagagagctttagtggagctatttggggatgaggagaataatcgtaaagctgttgcatgtttaaatgtgatggctactcggattgcaacactttttgcttcattaagagaatttccttttgtccgctttcgtgctgccaagtccctagatgcaaccacaatgactaccttccatgatcttattcctacaaaacttgctgctggtgtctgggactgtcttatgaaatataaaaaaactatacctaatttccctcagactgagacctgcgagttgctcatcattgatagaactattgatcagattgctcctgtgatacatgaatggacatatgatgccatgtgccgtgatttgttgaatatggagggaaataaatatgttcatgaggttcctagcaagactggtggtccacctgagcgaaaagaggttctcttggacgatcatgatcctatatggcttgaacttcgtcatgcacatattgcagatgctagtgaacggctgcacgagaagatgaccaacttcatttcaaagaataaagctgcacaaatccaacatggttcaagaggtagtggtgaaatgtcaacaagggacttgcagaagatggttcaagcacttccacaatacagcgaacaaattgacaagctctccctccatgtagagattgcaggaaaaattaacagaattattagggagtcaggacttcgggaacttgggcagctggagcaagatcttgtttttggagacgcagggatgaaagatgtgatcaaattttttactacaaacgaagatacaactcgtgaaaataagttgcgcttgttaatgattcttgcgtccatatatcctgagaaatttgaggctgaaaagggtctgaatttaatgaaggtagcgaagttgacagatgaggatgcaattgcaataaataatttgagaatgcttgggggagaacctgataccaaaacgacttcgacgagttcttttgctcttaaatttgatatgcacaagaaaaagcgtgcagcaaggaaagatcgatctggtgaagaggatacatggcagttatcacgtttttatcccataatagaggaactcatcgaaaaagttagcaaaaatgaattatcaaaactggactatccttgtctaaatgacccaagtccaactttccacggtacaacttatgctgtaccagtaactcacaatcctcctgctcattcaatgagatcaaggcgcacaccaacttgggctcgacctagaggctctgatgatggatattcaagcgactcggtgctaaaacatgcatccagtgatttcaagaaaatggggcaacgaatatttatattcattgttggtggagcaaccagatctgagcttaggatatgccacaagcttactggtaagctgaagagggaagttattctaggctcgtcaagtatcgatgatcctgcacaatatattacgaaattgaagatgctgacagcacaggaactttcattggatgatctccagatatga

SEQ ID NO: 120 - DNA, 1332 bp, Glycine max
  source 1..1332: mol_type = other DNA
atggcttcttcaaaatcaaaggttagaagggcatgttctttcaccaacctcctccttagctgtttgaatttctcactctttatcctctctgcttcctcatttgcacccactattctcctcaagatgcccccaacttcattcggcatggctctccttatggtctctggcatctcactcctctcatcttttgtgggcttctactccccgctcacacacttctgttacctcactcacatttcacttctactagcctcattaataggacaagtgctgaccatattggccttgttcacaaaagagaaagcaagcatgtcccttctgaagtcaccaagggaccccaaagaagccaaacttttggtgaggctggagtgtggggcattgatggcaatgttcatgctgcagtgtgtggtgctgatgctgggttgtgcagtgcatagtttctgggtgaaggattatgaggaactcgaagcagagaaagccgcctcggcgaggaagaggagtaggagaattgcggaggtgcaagaggaatccatggctaatgctagcaagatggcagaaatcaaagctaaagagttggatgagaaaatgaaaagcaagaccatcgacactgacttcttgaacacgaagaagaaacaagtttcgttccaactttcgagtttgatcacaccacctgtctccactgtgaggttctgcgatctcaactttggccgcttgcaaccttccgatgaagaattgggtcctcacaaaagatttgagtttggcaacttcgttgcacgagaagccctgcttgaggaagagtattggtctgcagcgtgcctaagggcagaagagtgggcaaatcgaacagataaactatatgttgttgatcggcagaggaattttgctgaacaggaatttaatgcgataaaaaagcggtgcaaggagctacaagacggtcacagtagcacatgcatcatcacggttaggaagccacaaaagaatgtaaaactcccaataatagaaagtgttgtaggaactcttgatttgaatatcatatatttgcggcgaggggagacctttcctgggatcgaccgaacagcatcaagcagatatggttatattgcaaacttgtgtgttgccaaatcactttatcgcaagggtgttgcaagcaaaatgttgtattttgctgtggagtctgcaaaatctactggtgtgtcacgtgtgtatgcgcatgtggacagaaacaataaacctgcccaaatattataccaaaacttagggtttgagataattgacactgcaaaccccctgttgttgaaaaatcaaacatccttgctttacttacagatgtag

SEQ ID NO: 121 - DNA, 1209 bp, Glycine max
  source 1..1209: mol_type = other DNA
atgtcttgttgggcatgcaaaaaatgcacctttgtgaatctcccttctcaaaaaggggaatgcgaaatctgcttttcccccgcgtccccattgtcaatgggtccttcttgttcttcgtcgcccccaaaatggtcgtgcaaggcctgcactttcttaaacccttacaacaacccttcttgcgaggtctgcgccactcgatgctccgttctctccctttccaatctcaccgacttgaatgacgccaccgatcacgattcttctgtcggttctgttttctttcctcttcgaacctgcaacaagagaaaggccattgatgatgacgattcttctgaggtcatcaatttcaaggtcaagccatccaatatcaccggtgatgagaatatcgacacaggaaaagcgtttaagatattgagttacaatgtttggttccgggaagacttggagttgcacaagaggatgaaggctattggcgaccttgttcagttgcattcacctgactttatctgtttccaggaggttactcccaatatatatgacattttcaagggatccgcttggtggagtgtgtattgttgttcagtttcttctgagatggcttattcaagaccctacttttgtatgctgttaagcaaactgcctgtgaaatcattcagcaataagcccttcagcaattctataatgggaagagaactttgcattgctgaggttgaagctgcaagtggcaagccattggttattgccactagccatcttgagagtccctgtccagctcctccaaaatgggatcagatgtacagcaaggaaagagtagtgcaggccaatgaggctataaaccttctcaagaaacagccaagtgttgtttttgggggagacatgaactggaatgaccaacaggatggtcaatatcctctacaagatggatgggttgatgcctggtctcagctaagaccaaatgaaagtggttggacttatgacaccaagtcaaaccagatgttgacaggcaaccgtactctccaaaagcgattagatcgctttatttgccaattcactgattttaagataaccagtgttgacatgattgggatggaagcaatacctggtgtttcatacaacaaagaaaagaaagtaagaaaggagataaaacaactggtactcccagttttgcctagtgatcattatggcctgcttttgacaatttctagtaagtaa

SEQ ID NO: 122 - DNA, 3000 bp, Glycine max
  source 1..3000: mol_type = other DNA
atggctgagaccacaaaatacattcccaactcacaattagagcttgaagagatattgaatgaagcagaacatcgatggcttcgacctgctgaaatatgtgagatacttcgcaatcataagaagtttaaattaacaccagatccacctgttatgcctccagctggatcactattcttgtttgatcgaaaagcacttcgttactttcgcaaggatggtcaccggtggaggaagaaaaaagatgggaagactgtaagagaagctcatgaaaagttgaaggctggtagtgtggatgtcttgcattgttactatgctcatggtgaggataatgaatacttccaacggagaagttattggatgcttgacgagcagttagagcacattgtccttgtgcattatcgtgaaattaaagagggctgcaagtctggcatctcacatttgcctgtagttccagtaaccctggttggtagttctcaaaatacttcagtgctttcctctaccaaaataaattcaccaatatctctagttcagacatctttcacatcaagtgcaaataaagtttatcagaatggacgagcctcagagcatgaggatgtcaattcaaagaatggtcctcaagcctcctctcatgctcagcctatcagcaattatgtacttcacagtgcaccttggcttacacatgaagctgcagggttttctgagttgttaaggaaccccttgatttcatcatggccatcttctttccctagctactctcctggtactggcttgtctccctggacctcaattcagaactctagcagaaacacaattaatatgcatgatggaaagcatcatgttgaagcctctgaagcagacttaactgttcgtaagctaagcaatgctgggttagattctgttcaccgaatgcaagatggtgtaattttcagagataggctcatcactgacatgtgcgtccagccagttatagatttgcctactgttaatcaggtgaaaaacgagcatggtctggattcatttcatgctcaagtccatgatcataatgatcatccagttgttgcaactactaaaatcctagttgaacaaaaacttcaagatggtggtttatacaatgatgaatcagaacaagttgaatatggagaaatgaagaaacttgatagttttggaaggtggatggataaggaaattggtggagactgtgataattccttgatggcttcagattctggtaattattggagcacacttgatgctcatagtgaggataaggaggtatctagtttacgccacatgcagttggatgtggattcattaggtccttctctttcccaagaacaactatttagtatccatgacttttctcctgattgggcatatactggagttagaacaaaggttttaatagttggcacatttctggggagtaaaaagccctctagtgagaccaaatggggatgtatgtttggtgaaattgaggtttcagctgaagttttggcggataatgtcataaggtgtcagactcctttgcattcacctggacgtgtaccattctatattacctgcagtaataggttagcctgcagtgaggtcagggaatttgaatttgatgaaaatccaaccaaatttttaggtcctgagggtattaaaatctcaccagaagaagaggtacgacttcaaatgcgacttcttaaactagtagacttgggacctgacaataagtggttgaagtgctccgtttcagaatgcgaaaaatgtaaactcaagggaacaatgtattccgtgagagatgatagtggagtatttgaagaaactttccagattgatggaattggtcatataaaccatagagatatattatttcagagattagtgagagacaagctttatgagtggttgatatataaggttcatgaaggaggaaaaggaccacatgtgttggatgatgaaggacaaggagtaatacatttggcagcggcacttggttatgtgtgggctatggccccattagttgcagctggtatcagtcctaacttcagagactctcgtggaagaacaggacttcactgggcatcatattttgggagagaagaaactgttattgtgctagttcagttaggtgcaaccccaggtgctgttgaggatccgacatcagcatttcctcgaggacaaacggcagctgatttaggttcaagtagaggacataaaggcattgctgggtatttggcagaggcagatctgacaaatcaattatctgtattgactgtcaaggaaaatgaaactggcaacattgccacaacaatagcagctaacagtgctcttcagtctgttgaagatgactcatctagtatgacaatggatgagcaacattatctcaaagagtcacttgctgtgtttcaaaaatcagctcatgcagctgcttcaatcctagcagcctttagagcaaggtcattctgtcagagacaattagcccaaagtagcagtgatatttctgaagtacttgacgtagttgctgattctttgagcaaggtacagaacaagggtcactttgaggattatttacattttgcagctttaaagattcaaaagagatatcggggatggaaaggaagaaaggattttttgaagatacgtgaccgcattgtaaaaatccaggctcatattagaggacaccaagttcgtaagcagtacaaaaaagttgtgtggtctgttagcattgtggaaaaagcaatcctgcgctggagacggaaaggggctggtctgcgaggattccgggttgggcagccagttggtgttgtggttaaagatgccgagaaaagtgacgagtatgaatttcttagcattgggaggagacaaaaatcagatgatgtaaagaaagctctagatagagtcaagtccatggttcgtaacccagaggcaagggatcagtatatgaggctcatcatgaaatatgaaaagtttaagattgatgatggaggaagcagtcaatcacagcatgtcggttag

SEQ ID NO: 123 - DNA, 429 bp, Glycine max
  source 1..429: mol_type = other DNA
atgaggaacagtatggatactaataggaaggaagtcacctcacagtttgcggagtttcgatcctatgaaggccaggttatgtatgttggggagaagattgttccgatcaagcttgatctttgtgttaatcatacgctcgtgaaggaccaatttttatgggacttggacaactttgagagtgatcatgaagagtttgccaaaatctctacaaggacacgagcattgaatatcccgaggttggagaaaattgcaattcaaagtgtggtttcagcaagagaaaccagaatgagcaacaagggtcgtcgaggggctgaatatactccagtcaggaaaagaaaggagtgggacgtatatgaacctattgtcgacctactatccaacgaggaagttgatgccattgaagcaaaggaagagagaaatttctggtga

SEQ ID NO: 124 - DNA, 561 bp, Glycine max
  source 1..561: mol_type = other DNA
atggcttcctctctacacaactatgttcttgctatcttacttttggccataacccaattcaaaaccacatctgcgggctcccatcaccacctccagcacctcaaatcccttcacttctctctgtttcaacacgagaccataaacaaaaccggatacatcatagtggatggtataaaaggaggagcaggggtgactcaaaccacaaccccttttggcaccttatttgcctttcaggaccctttgactgttgcagccaacaggtcctccaaactagttgggattgcagaagggactacagtcacatctagtctcgacgggcttcggagcatttcgatcgccaagctaaccctgcgtttgaagcaccacaagggctccctttccattgttggtgtcacaaacaatgtcaaaccctctgatcttccagtggtaggaggcactgaagatttcatgtttgtgcaaggctatattagtacttctccagttgatctcaagggtcttactgttgtctacaagattgagtttcatctttactggcccccatatgcaactcaagcctcatga

SEQ ID NO: 125 - DNA, 2034 bp, Glycine max
  source 1..2034: mol_type = other DNA
atgccacgatctagttccgccgtgcaccaccaccgtcagagctccgataacttcatgttcgacgctcacggaaggtggttacactcttcggcttacgcgcaggagctcgggactcgatcttcgagcttgaggagaaacgacgatgatcgcgttttaaccagtggcttgcttgatctgcattctttcgataccgagcttttacctgagggacaaagttttgatggctacgagtcgattctctctggcaacaaacttgtaccaaggtctcggggcttgcctgagagccatctccttaaaagtgtctcagcagataaagagagagcaaacaatgttgccaagatcaaagttgtggttcggaagagaccactaaataagaaggaaatagcaaagaaagaggaagacattatttacatagattcaaattttctcacagtgcatgaaagaaaactcaaggttgacttaacagaatatattgagaaacatgagtttgtttttgatgctgtgctgaatgaagatgtttcaaatgatgaagtgtatgctgagactgtggagccgattgttcctttgattttccaacgaacaaaagcaacctgctttgcatatggtcaaactgggagtgggaagacatataccatggaaccattgcctctcaaagcatcccacgatattttaagattaatgcaccacacataccggaaccaaggtttccaactgtttgttagtttcttcgaaatatatggggggaaactttttgatctcctcaatgaacgaaaaaaactttgcatgagggaggatgggaaacagcaggtttgcattgttggcttgcaagaatatagagtatctaaagttgagacaatcaaggaatttattgagagaggtaattccacaagaagtactggtacaactggagcaaatgaggaatcctctcgatcgcatgctatacttcagctttgtatcaagagatcagctgatgggactgaatcaaagcctacccgacttgtgggcaaactatcttttatagatctggctggaagtgaacgtggtgcagatacgacagataatgataagcagactaggattgaaggggcagaaataaataaaagtttacttgctctgaaagaatgcattagagccctagacaatgaccaagggcatatccctttcagaggaagtaaattgactgaagttctgcgagattcttttgttggtgattcacgcactgtaatgatatcatgcatttcacctagctcaggttcatgcgagcatactcttaacacattaagatatgctgacagagttaagagcctgtcaaaagggaacacctctagaagggatcctctttcttcctcaaaccttagagactctactgtgttgcctggctcttcagttttatctcatgatgataccttggaggatgaaacaacatatgtttccagtgacaagaatcgatttggttggcccaaacagctagaaagggaaccctctcctccgaataatgtggaccgtgttccaagtggtagaatggggggaaatttgataccatctgtgtattctgatccacaaaatggtcaaagaggcagtcaaaaggccagaactgcaaatgaatatgattacctaggaccaacatatgaacaggatagaacgagaaaaacaagtaagagggtggataacaaccaattatctgccgtgggggacaagaggaagatagaatctcgtgttaaacttgtggatgaattgcattttgaggctaatcattctgatcctgacgataatttaaatgccctcctgaaggaagaggaagatctcgtaactgctcaccggagacaggtggaggaaacaatagacattgttagggaggagatgaatttactcgttggagctgaccaaccaggaaatcaactggatgattatatttccaagttgaatactattttatcactaaaggctgcaggaatctttcaattgcagacacagttggctcaatttcagagacgcttaaatgagtataatgttgtcgtaacgtctggtaattga

SEQ ID NO: 126 - DNA, 990 bp, Glycine max
  source 1..990: mol_type = other DNA
atgagcaaaagagctaataagaggaaagcgaagcaagacgaacagcccaaacaacgcaagaagcgatccacaacaattaaacctgccgagcacgaagagtacttcgaggatcagcgcaacttggaagacttatggagggaaacattccctgtcggaacagagtgggatcaattggattctgtgtatcaatacaagtggaatttctctaacctggaaaatgcgtttgaagagggtggtgtacttcatgggaaaagggtttatctctttggttgcaccgagcctcaactggtctggttcaaagatgaaagcaaagttgtctgcatacctgttgttgtagctgttgtatcacctttcccaccgtctgataaaattgggattaactcagttcagagagagttcgaagagataattcccatgaaacaaatgaaaatggactggattccatatatcccccttgaggaccgagaaagccgagttgatagattaaagtctcaaatatttatcttaagttgcacccaaagaagggctgcattgaaacatctgaagttggatcgtgtaaagaaatacgagtattgcttgccttacttctaccatccgtttaaggaagatgaacttgaacagagcactgaagttcaaataatatttccagcagagccaaagccggttttctgtgaatttgattgggaattagatgagcttgaggagttcactgataagctcatagaggaggaggagttattggaagatcaaaaagatacctttaaggaatttgtcaaagaaaaagtacgggaagcaaagaaagctaatagagaggcaagggaagctcgaagaaaagccattcaagaaatgagtgaggaagctagagctgcatttgagaatatgagattttataagttctaccctgtgcaaagtccagatgcacctgatgtatctaatgttaagtctccattcataaacaggtattatggaaaggctcatgaggttctgtaa

SEQ ID NO: 127 - DNA, 2808 bp, Glycine max
  source 1..2808: mol_type = other DNA
atgctccacaacaagtccttcgtcaagaaaaccaaaggtggcaaagtcataaagcaagtgcgggagcactacctgcgcgacgatatatactgtggcgcaccgttttgcaccgtttgcgattcttccagtgctcgcctcagtgcttcgccttccatcattctcgtcgtcgacaccaatgtcgttctcaaccagattgatttgctggagaatccggcaattgacgatgttgtggtgttgtccattgtgttggaagaggttaagaacaagaacatgtctgtttacaaccgtataagagctatctgtagcaattccatgaggaagttctttgttttttccaatgaataccacagggatacatatattaaggaaatgagtggggaaactaaaaatgataggaatgatagagctattcgagtggctactcagtggtatcagaatcatcttggtggtgcagtaaaggtgttgcttttaactaatgacaaagagaacaaaaggaaggctagtgaagagggtattttggccgaaacagttgagtcatatgtgaagtctttggaccaacctgatttacttgatctgcttgtgcggcctgcatctgaagatgtggacatggaagaggttgaagatcttagaccatccaagaggaaagtcatctactccgagcataagccgatgtcagaaattacttctggtttgcatcgtggaatatatcatcaagggaaacttcgtgttaaccgttataatccatttgaagcatatgttgggagtgagagcattggtgatgaaataattatttatgggcgctcaaacatgaatcgagcatttgatggggatattgtggcagctgaacttctgcctcaggaccagtggcaaggagtctcatctctatctatagcccatgaagaagacgaggatgaggatgaagatgttcatctagctccaaatagtgctgatgatgcacctagaactatttctcagcagggttcttctgaacaagtaaatgctgtgcccagtcgtccttctggtcgtattgttggcattataaagagaaactggcattcgtattgtggatctttggagccaatgcctatgcctgcagggagtggtggtattgcccatgccttgtttgtctccaaagatcgcagaattcccaagattcgaatccaaacccgtcaacttgagaaccttttggataagaggattattgtggcagttgattcttgggatcgtcagtctcgatatccatcaggccattatgtgcgaactataggggagataggtgatagagatactgaaagcgaggtggttttgatagaaaatgatataaatacacggccattctcttcacaagtgctggcatgcttgccaccattgccatgggtagtctcttctgaagatttgtctaatccaatcaggaaggatttgcgtcatctgcgtgtcttcagtgtggaccctccaggctgcaaggatattgatgatgcactccactgtcacgtccttccaaatggaaactttgaagttggagttcatattgctgatgtgactaattttgtttatcctggcactccccttgatgatgaggctgcacaaaggggtacgtctgtctaccttgttgagcggcggatagacatgcttcccaaacctcttacggaggacatatgttctcttcgttctgatgtggaaaggcttgcattctctgtcatatgggaaatgacacccgaagcagatattattgccacctcatacacaaaaagtgtcataaaatcttctgcggcattgtcttatgttgaagcacaggcaaggatggacgatagtcgattgatggatccgattactgcagatttgaggaatatgaatagtttagctaagaaaatgagattaaggcgtattgagagaggagctttgactcttgcatctgctgaggtcaaatttcaaattgacacggagactcatgatccacttgatattggaatgtaccagatccgggaggccaaccaaatggtggaggagtttatgcttgcagctaatgtttccgttgcacaacaaattcttaaaagttttccattgtgctcattattaaggcgtcatccaacaccaacaagagagatgcttgaacccttactacggactgctgctgcagttggcttgcacttggatgtctcgtcatcaaaagcgttggctgattctcttgaccatgctgtgggtgatgatccatacttcaataagttaatccgtatactggcaactaggtgcatgactcaggcagtttatttctgcagtggggatcttagccctccagaatatcatcattatgggcttgcagctcttttgtatacccatttcacatcacctataagaagatatgcagtagatgtcattgtgcacaggctacttgctgcttctttagggatatccaagttaccacctgtatttcaagacagtctccagctcactagtattgcagacaatttaaattatcggcacaggaatgcccagtatgcagggcgggcatctgtagagctacatactctaatttatttcaggaagaggcctacagacacggagggtagaatagtgaaaataagatctaatggattttttgtgtttgttcccaaatatggcatagaaggacctgtatatttgacaaaagcagaaaagggaagtggagaatggtatgtagatgagcaacagcagaagattaaaaagatggatggtagcctttcatacaacgttttgcagacagtccaaattcacatggaggttgtggagcctcagcctaaccggccaaaacttcagcttaccctcatctag

SEQ ID NO: 128 - DNA, 999 bp, Glycine max
  source 1..999: mol_type = other DNA
atgagcatgttaggcctgagagacctagttctcatagctcaaaccccttcttcccttcaccaccaacaccaaggccaacccatttcagaaaaccagactcacactcacactccaaaccttcctttgccttcttcagcagcttctctcagtgttggctttggaattttccctctcctcactgccacaccatgtgtgccacagcctcatcaaaacaatgaggttcaagagtgtggtggtggtggtgccaatagtaacactaccaccaccaattattggaacctcaagatgtgccaagaagtgaataatcctccaaagaagggtgtgatcaacgtggctgatcatgatgatgagaagggtgtgatggagagtgaggaaaacggggtctatgggcctgactttagggtgtgccaagattgtggcaacagggctaagaaagattgcgtctttagaagatgcaggacttgctgcaagggacgtgggtatgattgtaacactcacgtgaagagcacgtggatcccctccgttcgccgccgcgagcgcgagatcacggtggcttccggtggcggtgttggcggcaatggtggttgtaagaggccaagagctgtggtggggtcatctcaaaatgccacttctcatagttccaactctaatgctacaactcctaagagcttagctactagctcttttcatcaagatgctagcttcaagcagtctttaccgggtcatgttcgtgcaccagcagtgttcaagtgccatagagtctctgccattgggaatggtgaagatgagtttgcatacttggcaacggtccagattagtgggcatgtgttcaaagggtttctctatgaccatggtgttgatgggaaaaccgcaaatgtagtcccttgtgtctcggaactgcagctgggaaacaattgcagtgggaagaacagagaatgttcctctgcaattgcaattggggttgcaaataacaatgcataccctgcttctgcgagttga

SEQ ID NO: 129 - DNA, 783 bp, Glycine max
  source 1..783: mol_type = other DNA
atgcgagcacagacgctgatgacacagtggtgcacaagagcagcaacgtgtggccaattgaggatgagcactaacaccaacacccccaaacacgatggtctcgacgccattttcaaacataagcgcttacttcgaacgcaggtcagaaaaaccctaaaatccatccaaccctccctccgatctcaccaagataacaccgttcaagacatagttttgggagctccgtggttcaaatccagtcgcaggctatgcgcgtacataagctgcgctgctcttcgcgaagtcgacactttaaaaatattgtcacaaattttgcaacataacaatgctactgatggaaagaaactctatgtgccgcgcgtggaggataagaatagtcacatgcgtatgctccacatatcgggaattgatgatctcattgcaaactcaatggacatcttagagcctgctccggttgatgttgatggaaatgcgcgtgaagatgttatgcaggcaaatgatccagttgatttgttccttttacctggactggcatttgacagatctggaagacgtttaggtcgtggcagaggttactatgatacattcctgaagaattaccaagaccttgcaaagacgcggaattggaagcagcccttgcttgttgcactgtcgtattccgaacaaatactggatgaaggactaataccaatgacttcatctgatcttccagttgatgctcttgtatctccggaaggtgtgattcccatcagtaaaactgccttaaacagcatggatctctga

SEQ ID NO: 130 - DNA, 216 bp, Glycine max
  source 1..216: mol_type = other DNA
atgatcatccccgttcgttgttttacctgtggaaaggtcattggaaacaaatgggacacctatttggaccttctccaggcagattacagtgaaggagatgcactggatgcattgggattggttcgatattgttgtaggcgcatgcttatgacccatgttgatctcattgagaagttgctcaattacaacactctggacaagtctgatcccaattaa

SEQ ID NO: 131 - DNA, 3051 bp, Glycine max
  source 1..3051: mol_type = other DNA
atggtaacaagaagtacatcaagattagtcaatgttattaatgaggatagtgatcaaaattctgataatacaactgaagcagaaaactcaaatacagttccagaagcaattataaacccaattaattctaaacaagggaaaaccccatcgaaattatactaccaacatccaactgcccctgtccttctactagaagaaagaggagaaaataatttcaagagtttttctgcaaataacatctatgagtggaatatagatgctcaaacagaatacaatatcatgaatacactccaacatatgacaatggtagccacaacttaccaaacctcccatgaattcaagactgatctcaatggtaaagtcattactaatgatgatgataaagagattcctaacgctgttaacaccttaatctttacaatagcacaacattttattggagacccatctttatggaaagataggtctgtagaattattatcaaatcttaagtgtagaaccttagcagattttagatggtacagggatactttcttgactagggtctataccagagaagatagtcaacaacctttctggaaagaaaaatttctagctggtcttccaagatctttaggagataaggttagagataaaatccgtagccaatacaatggagatattccatacgaaagtttaagttacggtcaattaatttcttatgtccaaaaggtagccttaaaaatttgtcaagatgaaaaaattcaaagacaattagctaaagagaaagcccaaacaaagagagatttaggatctttctgtgaacaacttggcttgccagcctgtccaaaacagaagaagaagacctctagaaaagaaacccatgaaaataaggtagttaccaaaaagagatttccaaggaggagatactcgcacaagccttcaactagtaaagaaatagaaaacccaaaacaaaaattaaaatctaaagtaacatgctataactgtggaaaacaaggtcacattagcaaatattgtaggttaaaaagaaaattaagaaatcttaatctagaaccagcagttgaagaacacattaataattttctcatggaatgttcggaggaagaagaaacagaaacttcattctcctctgatgaaaatctcaatctaattcgacaagatgatccattgtcatctacagaggatgaaagacaaatcaatactttaacaagagaacaagacctcctgtttgaagcaattaattctatacctgacccccaggaaaagaaaaattttctagaaaagctcaagaaaacattggaagtaaaacttagacaaaaagatattattatcaataacaaatttgatgtttgtaacattcttagaaggttagaaaatacttcagctaaaccaacaacaatccaagacctccaaacagaaatcaataatcttaagaaagaagtaaaagaacttcgtcagcaacaagaaattcaccagaatatcctttctcaactcgaagaagaaagtgattctgaaagcatcgctaacagtgacacaaaccacatagaaaatccagaagataatatgtttatgggggccgattcaaattgtatcttagaaggattaatcccaacaaaattcttcgaaaaaacatcagagaagttaggcacagcaaatggatcaaaattaaaaattaattttaaactgtcaaatgcaattatagaaaatcaaggccttaggatcaacacaagctttcttttggtaaaaaaccttaagaatgaaatatcaaaggaaggaataactacaaattatttaggaagaaaaattatatttgatttttccactaaaccaatctccagaaacataaatcacatagcaaataaaattaaccaaatcaattttttaaaagaagtatcttttagtaatattcaaaaccaactagaaaaaccccaaataaaagaaagaattcaatctttgttgaaacatattgaatcaagtgtttgttctgatttgccccatgccttttgggatagaaagaaacacattgtcgatctcccttatgagaaagacttcagggaaaaacaaatttccaccaaagctaggccaattcaaatgaatgaagaacttcttcaatattgtcaaaaggaaataaaggatttgcttgacaaaggtctaatccagaaaaatgtaaagaatcttgcatcaaaacaaatttttgctagatggcaagcaattttaagtgtctttgattttcaaatagaatacataaatggcacctcaaattctctacatgactacctcacacaatattgggacaaaaatcccttcaaagccactgctaagtcttttccctcgggattccattacagaccaacatacctcaacaagacaataaagttttatgaatttattttagttgatacaaaatcagtatccattaaacacttcaaagatccaaaagaccaaagcctaaatacccattctacaatccaaatcctaaaagtaatgcaaccaaggcattacggatctaatctcaaccaacccaaaaagttttttgcaccttttgacccagcaggatatacttattgggactatattaatgcctggactaatgtgttctggcatcagaacaataaatacaaacattcatggctaatctacttcaaaaataatacctacagatatggaagaatagaaaataataaacaatatccatctttgcaaagacatgcatttgtcaaatggtggactcagtttgatacttcaaaggtggctcctgaccaagtcaacaactggtttcagagtcaccctgaacttctcaaaccagctgacctagaaacatcgttgttttttaaccaaaaatctcaacttgtagtctttctagcaagctccaaatccaaggagagtcttgcaaagaatctcaatgaagttctccaaatgcttcaacaagaagaagaggaagaatcttcaaagaaagaagctgaatcttctgaagattttgatgaccaagatgatgatcccttctaccagaatgaggatgattgtttcggcatctcacttgaagaagattaa

SEQ ID NO: 132 - DNA, 378 bp, Glycine max
  source 1..378: mol_type = other DNA
atggcaaattatgaggtcaatcttataccaatgactactcctgtattcactcaagccaatgatgctgacctgtccaaactgtcaatccaaatagttcttccatttgacaaagaaacagaaagtttgccaaatcctaatcaagaaacagctcgcttgagaaaggttgaaggaggcattgttgcaataatgaagtttagcggaaaacccacagaggatattgttcgtgaaaaggagaaaaccctgcgtgccaatattattaaagatggtcttaaacctcagcctggttgtttgcttgcacggtacaatgatccaggtcgaacatggacctttataatggtatgctgcaaaacctgcttgagtgtgtcagttgatttctaa

SEQ ID NO: 133 - DNA, 726 bp, Glycine max
  source 1..726: mol_type = other DNA
atggaatcccaattaattgatggtaatgatgagatggaagtttctggggatgtgggtttgagtacgtgtgaagttcagatgcatcaagaaacatatgaagtggatccaaatgaggggtgtagagtactagagagttcttcttggggagagttgggtatctgtgaagatcatgctattcaggaaccatatgaaggcatggaatttgaatctgaagatgctgctaagctattctatgatgaatatgcccggcgattaggatttgtcatgcgtgtgatgtcttgtcgacgctcagaaagggatggtaggattcttgcccgcagacttggatgtaataaagagggttattgtgtcagcatccgaggtaaatttgcgtcagttcgtaaacctagggcaagcacaagggaaggttgtaaggcaatgattcatatcaaatatgataagtctggaaaatgggtgataacaaagtttgtgaaggatcataatcatccattagtagtctctccacgtgaagcacgccaaacaatggatgagaaggataagaaaattcaggaattaacagcagagcttcggcttaaaaaacggttgtgtgcaacatatcaagaacagttgacttcttttatgaaaattgttgaagagcacaatgagaagctatctgccaaaattcatcatgtagttaataatcttaaagaatttgaatccatagaagagcttttgcaccagacataa

SEQ ID NO: 134 - DNA, 2172 bp, Glycine max
  source 1..2172: mol_type = other DNA
atggcggaggagaagagggcgccgtcgtcgttcgtgaagaaaggcgaccggcaaatgttcacggtggagctctggccaggtgagaccaccatagtttcgtggaagaagctgttgaaggacgctaacaagcacaacggatccacttcggcgccacaacacgtcgcaatcgctccgggtcaacctgtggaggttgaagaaacagatccttctcagccaaaccgtttcagtgctgtaatagagaagattgagcgcctttacacgggtaaggacagtagtgatgatgaggatctgcttgatgttcctgatgatgatcagtatgatactgaagactcttttatagatgatgctgaactggatgaatattttgaggttgataattctgcaatcaaacatgatgggttctttgtaaatagggggaaattggaacgcataaatgaacctcctgtactacctaatcagcaaccaaagaaaaggcgcagaaaagatatattgaagaatgctggtgaaaacaatgatggtcatggatcaaataaaaatgtaaaagttggcaggccagcatctgccaaaacagcttcactacaggcaaagaatatgttaaatttatctgagaatttggttgcacctggtgaccatattgaagacttgaaacttccaaatcaatcggatgtctctggaattatttcgaaaaagaaaactgctgatactaaaccaatattgaacccttctgtctctttgaaaacatcaagtgatgatgctcctgctgtaacagatgcaaaagatgttgacaagcagaagataggagcttttcaatctaagaacattagtgataaatatatagatggaagtggatcgtttgatgcatctcatcataaatacaatgaaaaaagtgcatatgctcattccaaatcccaacctggaagaccctcaagtaatattgatgatatcaattggacaaaagaaaaaaatggtatgcgtgaactgccagatcttaacttgtctgagggaaagtctgctacccaagcaacaaagtctgaaaacatgcacaagaaagagggttctagtgttaggccaaaaacttcaatgcttgaaaaggctcttcgtgagttggaaaaaatggttgcagaatctaggccaccagcagtggataaccaagaggctgatgctacatcccaggcagtcaaaaggaggttgcctagagaaataaagctaaagcttgctaaagttgctagactagcggcaacgcacgggaaagtatcaaaagagttaattaaccgtcttatgagtattcttgggcatctgattcagctaagaacattaaagagaaacttaaaaataatgatcaatatgggtctgtcagcaaagcaggaggaggataataggtttcaacagataaagaaggaagttgttgatttgattaagatgcaggccccaactctggaatccaagcagcagctgaaaggtgaagcatctggtgattttcaagaatttggtactgatggaaaaccaataactaaaaggaagtttactatggatgctgcattggaggacaagatttgtgatctctacgatctttttgtagatgggttggatgaaaatgccggtccacagattagaaagttgtatgctgagcttgcacagttatggcccagtggttacatggacaaccatgggatcaaacgtggaatttgcagggcgaaagagaggcgcagagcactatacaacaaacataaggatcaggagaaaattaagaggaaaaagttgctggcacctaagcaacaggagaacgttcgatttgatactaatacaattacttcacagcagaacctacgagagagatcagctccagagtctagcagtcatgcttatacttcagggaacaagcaagtttctaatacaagcaccccaagtccaatgaatggtctaaaacaagaaaaagcaaagggaagttcaagcagttccgtggatgatgtcagggttgcagatggtgttttgacaaagaaggtaaagagaaaaccagaacttgagttggaaggagcacatttaggtcctgagaaagtagcttccttgcagggagaagaaagacccaggtccctaaagcagtctacagggccacttcccaccaaatcaaatcttcagccaacatctctgcctgatcttgaacagtcaagctaa

SEQ ID NO: 135 - DNA, 477 bp, Glycine max
  source 1..477: mol_type = other DNA
atgggtgttttcacattcgaggatgaaatcaactcccctgtggctcctgctactctttacaaggccctagttacagatgccgacaacgtcatcccaaaggctcttgattccttcaagagtgttgaaaacgttgagggaaatggtggcccaggaaccatcaagaagatcactttccttgaggatggagaaaccaagtttgtgttgcacaaaatagaagcgattgatgaggctaacttgggatatagctatagcgtagttgggggagatgggttgccagacacagtggagaagatcacattcgaatgcaaattggctgctggcgccaacggagggtctgctgggaagctaactgtcaaataccaaaccaaaggagatgctcagcccaaccaagacgacctcaaaattggcaaagccaagtctgatgctcttttcaaggccgttgaggcttaccttttggcccatcctgattacaactga

SEQ ID NO: 136 - DNA, 2370 bp, Glycine max
  source 1..2370: mol_type = other DNA
atgaggagatttttggttgatagagcaagtattgagaatgtgaatgttgtacaacaagaagccgaattagaaccgccacctaatgtggttaatgagtttaacccaaatgagattgtgcgtgatccaggtcataggaaacaaattaatgagtatgctccagatattcaagatcaagtgaggagggcatatatattgaagggtccaatgcaaccacatttgccaagctttcctcgtactccatttcgaagtgtttctagagcatttagtaaatcatggtataagaattacacatggttagaatacagtgagatcaaggatgcagcttattgtttttattgctttctctttaagcaacccgggagggccgaacactttggttttgaagtcttcactaaaagcggatatagagattggaagcatgcatctcaaggcttgaaagatcatgttggtagtcataatagtttgcacaactcatgtgtcaagcactacgatgattataataatcaaagacaaagtgtgacaagtaagtttgctaaagcaaccaaggaatcagaagaattgtataagattcgtttgacttgttctttagattgttcaagatatctcatagcacaaggcatggctttccgtggccatgatgaatcctctacttcactaaataagggcaattttagagagatggtagattgggtaaaatctcagaatgaacaagtgagggatgcttttgaccgtggtggaaaaaattgcacaatgacttgcggtgacattcaaaaggagcttgcaacgtgttgtgcacatgaagttaccaaggtgattatggaagagcttggtgatagacaattctccgtgcttattgacgagtcacgtgatatatccgtcaaagagcaaatggcggtgatgttgaggtttttgaatgacaaagggaatgttgtggaacgatttattgctctacatcatgtcacagatacttcatctaagtcattaaaggatgctctttatggtattcttgataagtacacattatctatttcaaggatacgagggcaaggatatgatggagcttcaaatatgagaggtgaatttaatggtttgcaaagaaaaattctagatgaaaatccttatgctttctatgtccattgttatgctcaccgtttgcaattggttgttgtgtctgttactagtagttgctcatctattcatgatttctttgagtacatcaccttgattgtgaatacaacaagtgcatcttgtaagaggagggatgctttgacagaggcacaacacaaagatattttaaataaacttgagagtggtgagatatctagaggaaggggcttacaccaatcatctagtctcactagacccggggatactagatggggttcacatcatactacattgcttcgtttggatcaaatgtggtcctccgtgttaaaggtgcttagtatggttgatgaagatggacgtggaccatctcaagcagcaggtttgatagaaaaaatggagagctttaaatttgcttttattttgaggttaatgttaaagttgtttggtatcacaaacgagctttcaaatatattgcaaagaaaagatcttaatattgtgaatgccatggaattagttgatgttgtcaaagctcggttgggcacaatgagagagagtggctggaataatttttttgccgatgtccaaggattttgtgttgctaaaagtattctggtaccaaatatggatgacgaaataccagttcggggtcgttcaagagcagaagggaggactatcactaatcttcatcattaccgtgcagagattttttatgttgctattgataaaatatgtgtggagatggatcaccgctttagtgaaggaagtaacattatacttgattgtttctcatgtcttgaccccaagaactctttctccaagtttgatgttgataagcttgctcgtcttgctgatatttatcatgcagacttttctgatgatgaccgaggaacaattagggatcaacttgaaacttatgtgcttcaagtgagaagaaatgcttctttttccacttgtgaagatgttcaaagtttggctatgaagatggttcaaattgagaaacatttggtatttccattggtttataaacttattgagctagctttgatattgccggtgtcgacagcatccgttgaaagagctttttcagcaatgaagattatcaagtctaaattgcgcaataagatcaacgatgtgtggttcaatgacttgatggtatgttacaccgagcgggagatattcaagtcacttgatgatattgatattattcgaacatttaccgcaaagaagtctcggaaaggacacttgcctcgtaattttatttaa

SEQ ID NO: 137 - DNA, 336 bp, Glycine max
  source 1..336: mol_type = other DNA
atgggtttttatctatcggaccaattcttacaaaatcctccattgacaaaaaagcctccaagaaacttgagaccatgcatgaagacagacaacaataaaatcaacatcaagaagtccaagaccataaaaattgaccgtgttgagacaagcctcaaagaatcgagtcgcgacttgtctctcttcaagttcaagtctgggggtgtggtggctctagttctctttgtggtctttgggttgctgaattcactctacgaaggcaaggtagttgccaaattgccctacaaaccttttgtcatctctggtccccctgaatttaaagccacattttgtatataa

SEQ ID NO: 138 - DNA, 864 bp, Glycine max
  source 1..864: mol_type = other DNA
atgggtggtctttttgaggctataactggttatggtcttggtgggtcttctatggctttgtttggaagagttggtggaggtatctatactaaggctgctgatgttggtgctgatcttgtcggaaaggttgaaagaaaaatccccgaagatgatccaagaaatccagctagaaggcagtatgacagtgctggatttgagctttttgacgttttcattcacattcttcatgatatagttctatttgaaatgcaacaatctttaagatgttctacctatacttatcaggcccttgatgcggatagcatggacatggaaattgatttatctaatcttgggactgtgaacacaatgtttacagctttattcagcaaaatgggtgtccctatcaagactaccatttcagctaatgttcttgaagaagctctgaatggaacagaagatagtgagaagactggcaaggtgacatctgcaggaatgtatttcttacattttcaagtgtatagaatggattcgacggtgaatgcgtgctatgtgcagttagcaatggccaaggatcctgaaggggccttctttaaaaggttggaaggtcttcagccttgtgaagtctcggaactaaagcctggcactcatatatttgctgtttatggagataacttctttaagtctgctagctatatgattgaggcagtatgtgcaaaatcatatgaagataccacccaaaaactgaaggacattgaagctcaaattttaagaaagaggaatgagctacgccaatttgaagcagaatatagaaaggttattattctcttaagtaatcagctctcaggatgcgtcatctgtgatcaagtatgcaaaaagccataa

SEQ ID NO: 139 - DNA, 1635 bp, Glycine max
  source 1..1635: mol_type = other DNA
atgcgtatagtcattgagctgaagcggggatcagatccattgattgttttgaataatctttatcgcttaacatctctgcaatctacatttagctataatatggtgggcattctaaatggacaacctaaacaaatggggctgaaggaattactgcaggcattcttagacttcagatgctctgttgttgagagacgtgcaaggtttaagttatcacaagcacaagagagaagacatattgttgaggtatgtgttcttattgggtttgataatctggatggggtgattcgcataatacgtgaagcttcaagtaactcggctgcagcagctggtttacgtaatgcattcagtctctctgagaaacaagctgaagctcttttggacattagcctaagaagactgtcccttcgtgagagtggaaactttatggctgaacgtaaatctttgatggaacaaatttccaagttagaggaactgttgtccagcaggaaaaatatacttgagcttatagaacaagaagcaattgaaatgaagaataagttttccaatccaagaccttcaatgttggaggacacagatgatggtcaacttgaggacattgatgttattccaaatgaagagatgatattggcacttagtgaaaaaggttatgtgaaacggatgaagccaaacacttttaacctacaaaatcgtggaacaattggaaaatctgtaggaaaattaaaagtgaatgattcgatgtctgattttctcgtttttcgtgcacatgatcatgtcctatatttcagtgataagggaacagtatactcagctcgtgcctacaaaattcctgaatgtagccgcactgctgctggtacaccacttgttcagatcttatctttatctgatggtgaaagaattacatctattattcctgtgagtgagtttgctgaggatcagtttttgctgatgcttactatgcaaggttatatcaaaagagtatccttgaatttgttttcatcaatcaggtcaatcggaatcatcgctattcaattggttcatggtgatgagctgaaatgggtccgtctttgctcaaatgatgactttgtggccatggcttctcacaatggaatggtcatgctaagccaatgcactcagattcgcacactaggtagaaatacgcggggggcacttgcaatgagactaaaaaaaggggataagatggcaagtgtcgacattataccatcagccatgtggaacaacttggaaacctcatcaaaatttcctgggagcaatgcaaagaaccaaaatgggccatggctattgtttgtatctgagaatggttatggaaagagagttcctttgtgcagtttccggatatcgtctttgaatcgagttggtttgataggatataagttttctgctgaggatcgcttggctgctgtttttgtgaatggtgaaagtgatgaacaagtggttcttataagccaaagtggcactgtcaacagaattaaggttcgggatatttcaatacaatctcgatttgcaagggaagttattttgatgcgacttgatcattctggaaagattcagtctgcttcattgatctcagcaacagattgtgagcctgaggaagttcttactattgcacaaggctag

SEQ ID NO: 140 - DNA, 1248 bp, Glycine max
  source 1..1248: mol_type = other DNA
atgctaaattttaataggaccagaagtcagcccaggtctacaagatctatgtctttgggaggcatggattatgtggatccaaaaaggaagggcaattttgttggaaaagtttttcttgctgcagcattaacggcattatgcattatcatgatcaagagatctccatctttgaatcccccaagtccgttttccatccgtgaaccaggtgtcactcatgttttagtgacaggaggtgcaggctatattggttcacatgctaccctacgacttctgagggaaaattatcgtgtcaccatagtggataacctgtcacgaggaaatttgggtgctgttagggttcttcaagagttatttccagaacctggaaggcttcaatttatatatgctgacttgggagataaagaatctgtaaataaaatatttttggagaataaatttgatgctgtgatgcactttgctgctgttgcatatgtgggagagagcacactggaccctcttaagtattatcacaatattacgtcgaataccttgttggtattggagtctatggctaaatatggtgtgaagacattaatatattctagtacatgtgcaacgtatggggaacctgaaaagatgcctattatagaaataacagaacagaaaccaattaatccatacggaaaagccaagaagatggcagaagatatcatctttgatttttccaaaaattctaagatggcagtaatgattctgagatacttcaatgtgattggatcagacccagagggcagactaggtgaggctccaagacctgaacttcgagaacatggtcgaatttctggtgcctgctttgatgcagctcgtggtattacaactggcttaaaggttagaggaactgactacaagacacccgatggaacatgcatacgagactacattgatgtaactgatttggttgatgctcatgtgaaggctcttgaaaaggcacaacctggtaaagtagggatctacaatgttggcactggaaagggtagatcagtgaaggagtttgtgaatgcttgtaaaaaggccacaggggtggacatcaaagtagactaccttccacgtcgacccggtgattatgccgaggtgtatagtgacccttctaagatcaaccgggaactgaattggactgcacaatacactgaccttgagaagagtttgcaggttgcatggaaatggcaaaaatctcatcgtaatggttatggcattttatctgcaatctga

SEQ ID NO: 141 - DNA, 993 bp, Glycine max
  source 1..993: mol_type = other DNA
atggctaacgaaatccctcacgatgaggtagtgtgcgttaccggcgccaacggtttcatcggttcgtggctggtccgaaccctcttggagaaggagaacccacgctacaccatccacgccaccatcttccccggctccgacgcctcccacctcttcaacctccacccctccgccgccagccgcctcaccctcttccccgccgacctcctcgacgccgccgccctctcccgcgccatcaccgcctgctccggcgtcttccacgtggcctccccctgcaccctcgaggaccccacagacccccaaagagaccttctcgaacccgcggttcagggcaccctgaacgtcctaaccgccgcaaggcgcgtgggcgtgcggcgcgtggtgctcacctcctcgatctccgcgatggtgccgaacccggggtggccggcggggcgggccgcggacgaggcgtcgtggacggacgtggagtactgtaagggaagggggaagtggtacccggtggcgaagacggaggcggagagggcggcgtgggcgttcgacggcgtggaagttgtggcggttctcccggcgacgtgtttagggccgcttctgcagccggatctcaacgcgagctccgccgtgctgcgggagctgatgatggggtcgagggagacgcaggagtatcactggttgggtgcggtgcatgtgaaggacgttgctaaagctaatgttttgttatatgaaacccccactgctgctggtagatacctctgcaccaatggtatctaccagttttctagttttgctgccatggtctctgaattgtaccccgaattccctattcacaggttcccagaagaaacgcaacctggcctgacagcatgtaaagatgcagcaaagagactaatggacttgggtcttgtcctcacaccaattcaagatgctgtgagagaagcagtggagagcctcatagctaaaggcttcctgcaatgcacaccctcacagagttag

SEQ ID NO: 142 - DNA, 2664 bp, Glycine max
  source 1..2664: mol_type = other DNA
atggcttcacattgcttccttcgttttcctccttccttaaaccctaaatacaaaagacttcccaaaccccgttattacccttccatttcctcgcgaattcaaaccccaaaacctgacaatgacgacgacaatgacaaaaccccaaacgacaaccgcttcgacttcctcaaactctccgtaaccctcactgtcatctctgcctccctccctcaacccgctgccgctgccgccgccgccaccaggaaggtaaagaaacggtcaccgaaaaaacaatcagccaaaaaagccgaaggtctttcgccggaggagctaaaaacatggacaagtggccttcccgtggtctccgatcgccttccctacagcgaaattatcgaactgaagaagagcgggaagctgaagcacataattaaacctaattccgcgaaattgaggcaacgtggcgaagcagttcttgtagttttggacgattcgagagtgttgaggacagtgttgccctcgcttgagagccatagcaagttttgggattcatgggatgagttgaagattgattcagtgtgtgtgaatgcgtacacgccacccatcaagagtccagagttgcctacctctctgttggcaaacatttgggtgcccccttttgtgcagaaattcattacttacgtttttgaagagaggcaaacaaagcccaagaaggagtccaagaaagcggcggagtatagggaaatgaggatgcagttgcagagggagaaggaagaggagctaaggaagtcgagggaagagagggagaccatggagaggaacacgagggctcggaagaaggaggaggagaggaggaagaagagggagatcaggaagaggaagtataaggaatccttgcgccaggcgagtgaccgaaacgagagaatggcgtatttctggtctgatttggcaaataatagcaatgtggccaatgcacttggggtgcttttcttctacattttttacaggactgtggtgcttagttataggaagcagaagaaggactacgaggacaggctcaagattgagagggccgaggctgaggagaggaggaagatgagggagttggagagggagatggagggaattgaaggtgatgatgaggagggtgagcaggggaaaggggaggagaatgcttatttgaaaatggccaagcagtttatgaaatcgggagcgcgtgtgaggagagcacagaacaagaggcttcctcagtatctagagagaggtgtggatgtgaagtttagtgatgttgctgggctgggtaaaattcggcttgaacttgaggagattgtcaagttctttactcacggagagatgtaccgaaggaggggagtgaaaataccaggtggcatacttctttgtggccctcctggagtgggaaagacattgctggcaaaggcagtggctggtgaggcaggggttaatttcttctctatttccgcttcacagtttgtggaaatatatgtcggtgtaggggcttctcgtgtccgagcactttaccaagaagccagggaaaatgccccatctgttgtcttcattgacgagctggatgctgttggaagggagcgtggcttgattaaaggttcaggtggacaggaacgtgatgctactcttaatcagctcctggtgtccttagatggatttgaaggcaggggagaggtgatcactattgcatccacaaaccgaccagacattctggatcctgcacttgtgagacctggcaggtttgatcggaaaatatatatccccaaacccggtcttattggccgcatagaaattctaaaggtccatgctcgtaaaaagccaatggctgaagatgtggattacatggctgttgctagtatgactgatggaatggtaggtgcagagctggcaaacataattgaggttgctgccatcaatatgatgcgtgattcaaggactgagattactactgatgacttattgcaagctgcacaaatggaagaaagaggaatgctagatagaaaggaaagaagcactgagacatggaaacaagtagctataaatgaagctgcaatggctgttgtggctgtgaactttcctgatcttaaaaatatagagtttgtcacaattgctcctagagctggtagggaattgggttatgttcgggtgaagatggattcagtcaaatttaatcaaggaatgctcacacgtcaatcccttcttgatcatattactgttcaactagctccccgtgcagctgatgaactttggtttgggagtggtcagttgagtacgatatgggctgaaactgcagacaatgctaggtctgcagcaaggacatttgttcttggtgggctttctgagaagtatcacggaatgtccaatttctgggtgtcagaccgaattaatgaaattgattcggaagcaatgcggattgtcaactcgtgttatgaacgtgcaaaagagatcctcgagcaaaatagaacgctgatggatgctttggtgaatgagcttgttgagaagaaaagcttaaccaaacaagagttcgtccgtctagtagagttgcacggcttcctaaaaccaatgcctctcagcatacttgacatacgagttgctaagtgtagagaattccaaaaattgatcgatagtggaaaggaaacaactagtttgagtagccacgcataa

SEQ ID NO: 143 - DNA, 1404 bp, Glycine max
  source 1..1404: mol_type = other DNA
atggcagagacaccaaccctcagaatccacgaagtctgccccatctcgccgccacaagaaaccccatcaaccactattcccttcaccttcttcgacgtcctatggctacgcctccccccagtggagcgtctgttcttctattccttcccaaacccaacaacaacctcttcattcttcgacaccaccattctcccaaatctcaaacactccctctccctcactctccaccacttccctcctctcgccggcaccatcacatggccacttcacacacccctccccctcatcacctacacccccggaaactcaatccccttcagaatcgccgaatccaacgcagacttcaacaccctctcttcaaacctctcagaagttaacaaccaccgccgaaacctaataccccacttacccacttcccacgaagaagcttcggtgttagcccttcaactcacccacttcccaaaccaaggctattcgataggaataacaagccaccacgcagcacttgatggaaagtcttcaactttgttcatgaaatcgtgggcgcatatttgttcttacctcaatacctcaccggaagaaccgttgctgttttcactaccgaagcatctaacaccttcgtttgatagatctgtcataagagaccctttggggatcggtgagatttacgcgaagtcgtggacgagcttcggtggagccaccaatgaccgaagcttgaacgtgtgggataccctcggtggaaatcaaaccgatttggttaaaggattgtttgagttgacaccgttggatatcaagaagctgaagaagttagcggagtccaagtttgttgtcggagacaacaagaagaaagttagggtgacatcatttacggtcacgtgcgcttacctgttgtcatgcgcggtgaaagcggagcaacccaactgcgaaagagtgccttttgtcttcaacgtggactgtagggcgcgtttggaccccccaattccggaaacgtacttcgggaactgcgtcgtggctttgttggcttcggccaagcgagaagagcttttgggggaagaagcgtttttcaaaagcgttatagggataagcgaggagttgaacgggttagagggtgacgtgttgaacggcgcggacaaatggattccgaagattcaatcggtggtatcggagactcctaggttgttctccgtcgccgggtccccgaggtttgaggtttacggcattgactttgggtggggaaggcctgagaaagtggatgtcacatccgttgataaaacgggtgcgttttcgctctcggagagtagggatcatagtggagggattcaaattgggttggcgttgaccaagaatcaaatggaggcgttttctagggtttttgctcaaggacttgagtccttggaatcatga

SEQ ID NO: 144 - DNA, 1068 bp, Glycine max
  source 1..1068: mol_type = other DNA
atgatgaattccacagaagaagagtatcgtggctgcgagattatggagaaagatgttcatgtcacgtttctcgagttaatgtactatttactcccatctccgtacgagtcccaagagatcaaccatctcactctcgcttactttgtcatctctggacttgacatcctcgactctctccacaaagttgcgaaggatgctgttgtcagttgggttttgtccttccaagctcaccccggtgccaagactgatctcaatgatgggcaattctatggctttcatggatccaaaacttcacagtttcctccagatgagaatggggttttgattcacaacaacagtcacttggcaagtacttattgtgccatttccatattgaaaattgttggttatgaattgtccaatcttgactctgaaacaattgtgacttctatgaggaaccttcaacagcctgatggaagtttcattccgattcatactggaggcgaaacagatcttaggtttgtgtattgtgcagctgccatctgtttcatgttggataactggagtggcatggacaaggagaaaaccaaggattacatattacgttgccagtcttatgatggtggctttggattagttcctggtgcagaatcgcatggaggtgcaacttattgtgctatggcatctctccgattaatgggattcattgaagataatattctctcaagttgtgcttcatcttctttgatagatgcgccattgctgctggactggatcttgcagaggcagggaactgatgggggttttcaaggtagaccaaataaatctagcgatacatgttatgcattttggattggagccgttttaaggattttggggggcttcaaatttgttgacaataaggctctacgtggatttttgctttcttgtcaatataagtatggtggtttcagcaaattccctggggagtatccagacctataccactcctactatggattcactgctttcagcctgttggaagaatctggcttgaaatcacttttttcggaactgggaatcactgaaaatgctgcactggcactctag

SEQ ID NO: 145 - DNA, 810 bp, Glycine max
  source 1..810: mol_type = other DNA
atggcgattattcattattcagcagcttcacgttgttgcatgccatcgctgcatcgcgcttcgccttctcgctcggttctcgcgccctcaatcttcttcttccgcgacgctgctccgaccctcaggctcgacgtctcggttcggtcccagcctccaggttcggacggcttcggcgaacccatcaataacaaacacgcttctcgtgatgcctccgttgggttatccgcttcttcttcttcgtcggtcatcgattttctcactctctgccaccgcctcaagatcacaaaaaggaaaggatgggttaatcatgggataaaaggtgctgaatcgattgctgaccatatgtaccgcatggctttaatggcattgattgctggcgatgttccaggattgaatcgagaaagatgtattaaaatagcactcgtgcatgatattgcagaagctattgtaggagatataacaccatctgatggtgtgcccaaggctgaaaagagcagaatggagctggaagctttgaacaaaatgtgtgaacttcttggtggagggatgagagctgaggagatcaaagaactttgggaagagtatgaaaacaattcttctgtagaggcaaatcttgttaaggattttgacaaggttgagatgattctgcaagctctggaatatgaaatagaacatggaaaagtgttggatgaattcttcctttcaactgcagggaagtttcaaactgaaataggaaaaagttgggctgctgagatcatttcaagaagaaaatctttatcagcaaaaagaccaagttcatatagtggctga

SEQ ID NO: 146 - DNA, 2373 bp, Glycine max
  source 1..2373: mol_type = other DNA
atgctatctttcgccataaccactcatgctttccccgttattgccattaacaaatggaagcagagacatatgcaaaggcaaatgcaaagggtgtcactttcagcttccattcaccatgacagcttgagagtgctcgaatgggacaagctttgcgacttggtcgcttccttcgccaccacttccttgggccgtcaagccctcaaggatcagctatggtctcttaatcaaacttttgaggaaagtcttaaacttctcgaggaaaccaatgctgccgttgaaatgaacaagcatggcactctcagattgcacttggggcaccttgatgctatgctggtcaaaaccgccattcaacatgcccggagaagtataccagtgagtggttatgaggcacgggccattgtggctcttctgcagtgtgctgagatcgtgcagggtgatctcaaggctgtaattaaggaagacaaagattggcataatcgtttcatgcctcttacagaagtgataatggagtttgtcatcaatagatctttaattaaagcgatagagcaagtggttgatgaagatggctctattaaggactctgcgagtccagcccttaagcaggcacggcaacaagttcaagtgattgagagaaaggtacaacagttaatagagagcataatcaggaatgaaaagagtgaaacatcaacccttgaagtgaataacattgatggcaggtggtgtgtaagagtagattctggacagaagaccagttttaagggtctattgttgtccagtggatccggagttggaagtactatagagccactttctgctgttcctttaaatgatgagttgcagcgagcaagaagtttggtggtaaaggctgaagcagatgtgcttttggcattaaccaaaaagctggaccttgatgatattgagaagacattgaacagtttggttgaactagatgtgatcaatgctcgtgctacttatggtctgtcatttggagggtcaagtcctcatatatttctgccagataggagtagctcttctactgcagaagcattcttaccaaggagcgaaaatctctatggaccattacccagcaaaagggaatggacgctatatcttctaaaagcttatcatcctttattgctccagagacataaggagaaattgcggaaggccaaaaagaatgtcaatcttgctacttcagatgctgcactggacaatgctccgccagtacctgttgattttttggtatctcagaaaactcgtgttatagttataactggccctaatactggtggtaaaaccatatgtttgaagaccgtaggattggctgctatgatggcaaaatcaggtctctatgttcttgcttctgaatctgcgcaaattccttggtttgattctgtttttgctgacattggtgatgagcagtccttatcacaatctttgtctacgttctctggccacttgaagcagataagtaatattaaatcacagtcaacaagccagtcgctagtgctactagatgaggttggtgcaggaacaaacccccttgaaggagcagcactaggaatggcattattggaatcttttgcacaagacagttgtttgttgactatggctacaactcatcatggtgaattgaaaacactaaaatacagtgatgaggcctttgaaaatgcatgtatggagtttgatgaagtgaatttgaagccaacatacaaggttctttggggtgtaccaggccgctcaaatgcaataaatatagccgagaggttgggactaccatctgttgttgtagatactgctcgtatgttatatggttctgctagtgcagagatagatgaggttataactgatatggaaaggttaaaacaagaataccaagaactattggatgaagcacgtcattatctgaggcactccagaggactttacaatagtctattgaacaccagaaggaagatcatagaatatagtactaatctaagatttaagaagatgagagatgtgtccgaggctgcagcgatggcaagatccatccttcacaagaaagtgagggaattggatgcatcagctaagcaaccttcacagaataataaaaccattagcagctctaatttatcagcaaccaacaaaagccaaactgttgcagagaacaaagaacccactattgctgataaaagtgcatcttctgttaaagtttttaatcgatcaagatcagataaatctgggcctcccaaggttggtgatatggtacatgtctcttccctcgggaaacaggtgactgttttaaaagtggattcatccaaaggagaaattgtagttcaagctggaaacatgaagttgaagctgaaactaactgacattcaaagatcatag

SEQ ID NO: 147 - DNA, 474 bp, Glycine max
  source 1..474: mol_type = other DNA
atggcttcttcttcttcttctctgtttcgttccattcccatttcttttccacacacccaatgcgctctcaaatccccgcgcctctcgcttcgtgcttcggccactccgagccagaaggctcggttcgtcgctcgccgcaaggagtccgtttcggttcggcaactccaacgccccctaattgagtacatgcggttaccagcgagtcagtattcagtgttggatgcagagaggattgagcgggtaaacgaaaacacgtttaggtgttacgtttataggttcaagttcttcaactttgaggtttgtcctgtgttgttggtgaaagtggaagagcaacctgatggatgttgcatcaagctcttgtcttgcaagcttgagggctcgccaatggttgctgcacagaatgacaagtttgatgcagaagctgaggcaattttcttgtactcttctctaggaaaggaaagtgaaaacatgtag

SEQ ID NO: 148 - DNA, 960 bp, Glycine max
  source 1..960: mol_type = other DNA
atggtttctcgccatttggcttcttcgctcttcctctgtttcctctgtttcgagatctctgtttcgtcccttcgattgcccggtttggaccaagacgccaaggccacgcatggatcggtgctaaggttgaacagaggtggctcttcagtgaagtttgatcccacacgggtcactcagctctcgtggagtcccagggcttttctgtacaaggggtttttatccgaggaagaatgtgatcatttgatagttctggccaaggacaagctggagaagtctatggtggcagataatgattccggtaaaagcataatgagtgatatcagaacgagttctgggatgtttctcaacaaggcacaggatgaaatagttgctggtattgaagcccgaattgccgcatggacattccttcctgtagagaatggtgagtcgatgcaaatattgcactatgagaatggtcagaagtatgaaccacattttgattacttccatgacaaagctaatcaagttatgggtggccatcggattgccactgtattgatgtatttgtctgatgttgagaagggtggggaaacaatttttcccaatgccgaggcaaagttgttgcagccaaaagatgagagctggtctgaatgtgctcacaaaggatatgcagtaaagcctcagaagggtgatgccttgttgttcttcagtctccatctggatgcaagtacagatactaagagcttgcatggaagctgtccagtcattgagggcgagaagtggtctgcaaccaagtggattcacgtgagtgactttgaaaaaccattcaagcaagtggataatggagagtgtgttgatgagaatgagaattgccctaggtgggctaaagtaggtgaatgtgacaagaatccactttatatggttggtggggaaggagtaagaggaagctgtatgaagagttgcaatgtctgcacttcttaa

SEQ ID NO: 149 - DNA, 282 bp, Glycine max
  source 1..282: mol_type = other DNA
atgttgttcttctcgtacttcaaggatttggtggggagggaagtaatcgtggagctgaagaacgatttggccatcaaaggaacattgcattctgttgatcagtacctcaacatcaagctcgaaaacactagcgtcgtcaatcaagaaaagtaccctcacatgctttctgtaagaaactgcttcatcagaggttcggtagtgagatatgtgcaattgcctccagaaggggtggacattgaattattgcacgatgccacaagaagagaagcccggggcggttag

SEQ ID NO: 150 - DNA, 744 bp, Glycine max
  source 1..744: mol_type = other DNA
atggtctatttccatagctcaatctctctctgcaagttcgttgatcaatcatctttcatggcgaattcaatttgttctgcggatttcggttcgaaatcgagacaaatcaatcaccttcagaagaatcggagaacccctagttcatcatcatcatcatcatcatcatcgaattcccttcagattccaccctgtgatcgatctcgatcggcaatggttgatgttgtgatgttcattgctgttgtttgtgcctgtggctttttgttcttcccttatgtagagtttttggtgacaaagtgttacgaggttattaagggtgttgtgtttttgattaaggaggaggtttctgtggcaccttggatctatatttccatagggttgagtgttgtgtttgctgcattggccacgtgggctgtggtggcttgcaccactaggaagtgtgggaatcccagttgcaagggtctgaggaaggctgctgagtttgacattcagttggagactgaggattgtgtgaagaatttggcttctgcgtcgtcgaatgtagcgaaagatggtggtggtggaacgaagaagggtctctttgaattgcctcgtgatcaccatagggagctggaggctgagctcaagaagatggcaccaccaaatggaagggctgtgctcgtgctccgggcgaggtgtggatgttctgttggtaggttagaggttccaggaccaaggaagcatcttcgaaagatcaacaagaagtag

SEQ ID NO: 151 - DNA, 1611 bp, Glycine max
  source 1..1611: mol_type = other DNA
atgtttaaccattctgcgctccattattattttctcctcttcttcacgtgcacgtgcttagcacgacagcaatgccagttcaagcaggagtgccaactcgataccatccatgcactgaaacctgacaacctcatcgaatcccaaggtggtgtcacagagacatggaacgctagccaccctgagctatgttgcgctggcgtcgcctttatcaagcgcaccatcaaccccaatggccttcacttgccatcctacgttaattaccccgaactccatttcgtactccaaggtgagggagtgttgggaattgtaattcctggttgtgacgaaacttttgaagagccacaacgggagagggaacatgatcgccaccagaaggttcgttacctgaagcagggtgacatattcgcagttccacctggaattccttactggacctacaactacgccaatgtttctcttgttgtaattaccctccttgacactgccaatttcgaaaaccagcttgatcgtgtccccagaagattctatcttgctgggaacccaaaagaagagcacccttgtggacgcaagcaagaagaaggtaacaatataaacatgttcggtggtttcgacccacggttcttagcagaagcatcgaacgtgaaggtggggataacaaagaagcttcagtcacacataggtgaccaaatcataaaagtggaaaaaggtcttagcattatcaggccacccttggaacacgaagttagagaagcagaagtagaagaaaaacccaagacccgagaacactgtgaatgccaaaaagaaagaaaacacaaagaaggagaaggagaagaagaggtggtacaagagaaagaaataagaaagcgcaagcaccacataggagaacacgagggatgtggtgaatgcgaagataaagaagaggaagagcaaagtagaagccgagaacgcggcgagtggcatgaacataaaggacaacaacatggaaaagaaaaaggaagagagagatataaggaaggtggtgaagggagagtgcgtagcaatgtgttggaagaaatcttgtgcactttgaagctgcacgagaacattgctgacccatcacacgccgacatattcaaccctagagctggtcgcgtacgcaccatcaatagcttgaccctccccgttctcaaattgctccgtctcagcgcccaatgggttaaactctacaagagtggtatttacgtgccacactggagcatgaacgcaaacagcgtggcctacgtgacgagtggaggagggtgggttcaggtggtgaactcccagggaaagtcggtgttcagcggcgctgttggaaggggtcgagtggtggtggtgccgcagaactttgcggtggcgatacaagccgggagggatgggatggagtacatagttttcagaacaaacgacagagccatgatggggacgctggtggggcccacttcggccatcactgccatcccaggagaagtgcttgcgaatgcttttgggctgagcccggaggaagtgagcgagctcaagaataatagaaaggaggctgttttgtctagccctgcttctcatcactctccaaatcccctcattgtcaccatgtaa

SEQ ID NO: 152 - DNA, 1455 bp, Glycine max
  source 1..1455: mol_type = other DNA
atgagtaactctacagagagaaagccacatgcactgttgacaccattaccacttcaaggccatatcaacccattgttgagattagcaaagctgcttcatctgagaggctttcacataacctttgtccacactgaatacaacattaaacgcttgctcaactcaaggggtcctaaagcccttgatggccttcaagactttcattttgagaccataccagatagtcttccccccacatatggtgatggtgatgtcactgaagacgcagtgtctcttgctaaatcagtgagagagaagatgctcgtaccctttcgcgatcttcttgctcgtcttcatgattcttccactgctggccttgtacctccagttacttgcttggtttctgattgttggatgttttttactatacaagctgctgaagaactttcacttcctattgctctattttcaccgatcagtgcttgttccttaatgtttgttttgcactaccgttctctttttgataagggtctcctaccactcaaagataagagttatttgacaaatgggtatttggacacaaaagtggactggataccaggaatgaaaaactttaaactgaaggacttgcctgagattatatggaccatagatccaaatgacttcatgttaaaatttctcattgaagtaggagataatatgcaaagaagctctgctattattttaaatacttttgctgaacttgagagtgatgtattgaatggtctcacctctatgttcccttctctttaccccattggccctctcccttcatttttaaaccaaagtccacagaaccacttggcttctctaggttccaatctttggaaggaagatactgagtatcttgaatggcttaaatccaaggaaccaaagtctgttgtttatgtgaactttggtagcatcacagttatgtctccagagcaacttttggagtttgcttggggtttggccaacagcaagagaccctttttgtggatcattaggcctgaccttgtcgtgggtggctcaatgattttatcatctgagtttgtcaatgaaactttagatagaggcctaatagcaagctggtgtccacaagaggaagtgctgaaccacccttcaattggtggattcttgactcattgtggatggaactcaacaattgaaggcatttgtgctggcgtgccaatgctgtgttggccattttttgctgatcagccaataaactgtagacacatttgcaaagaatggggcattgggattgaaattaacaccaatgcgaagagagaggaggtggagaaacaggtcaatgaattgatggagggagagataggaaagaagatgaggcaaaaggtcatggaattgaagaagaaggcagaggagggcaccaaactaggtggtctttcacacattaacttggagaaagtgatttgggaagtgctgcttaaaaaaaattag

SEQ ID NO: 153 - DNA, 1341 bp, Glycine max
  source 1..1341: mol_type = other DNA
atggcttctcgaaaccatgaggtatcaatggtaaaggagaatggtggtggtgggaacaacctgaaggggttgagcaaggaaatgagacatggcagaacggcacataacatgtcatcatcttctttgcgcaagaaatctgacctaactcttgtgtctaaggtttgctctggccatgtaaggaatgtgttggtgaatttgcaagaggttattcttggaaccaagctctctattcttttccctgccattcccctagccattgttgctgaaggctatggctttggaagatcttgggtttttgtgttgagtttacttgggctcacaccacttgcggaacgagtcagcttcttgacagaacaagttgctttttacactggtcctgcagtgggagcacttttgaatgcaacatgtgggaatgctacagagctcatcatagcaatttttgcccttagccataacaaaattgctttggtcaaatattctctcttgggttccataatttctaaccttcttctggttcttggaacctctctattcattggtggcctagcaaatcttagtcaggaacaaaaatacgacagaaaacaagcagatatgaacttgcttatgttgtttgtggcattgctttgccacttgctgccattgttgtttcattatgttggtgcctcagcagctgacactggagactcatctctgcagttgtcaagagctgctagcattgttatggtgattgcatattgtgcttaccttgtcttccaactgtggactcacaggcagctatttgaagcccagaatgaagatgatgaagagggtggcagtgattcagaagcagtgataggattctggagtgggtttacttggctggttgggatgactatgaccattgctttgttgtctgaatatgtggtgcaaacaattgaggatgcatctgattcatggggtttgtctgttagcttccttagcataatcttgcttccaatttttggcaatgcaactgaacatgcagcagcaatcatatttggtttcaagaacaaactggacatctctttgggtgtttctttgggttcttcaactcaaattagcatgtttgtggttcccctatgtgtgattgttgcttggattatgggtatcaaaatggacctcaacttcaacctcctagaaacagcttctctttctttggcaataacaatcacagccttcgcattacaggatggaacttcccactacatgaaaggccttgttctcatactctgctatattgttattggggcatgctttttcgtacaaagaacaccccctaaccaaactaatgtttctaacataacgcttaaatccgcaaactga

SEQ ID NO: 154 - DNA, 3231 bp, Glycine max
  source 1..3231: mol_type = other DNA
atggcagcagcactggtaggtggtgcctttctctctgctttccttgatgtggttttcgacaggctggcttcacgtgagtttgttcacttgatccttggaaataagcttagcaagaagttgcttcaaaagttggagaccactctcagagtggttggagctgtgcttcatgatgccgagaagaaacagatcacaaacaccaatgtcaaacactggctcaatgatctcaaagatgctgtctatgaggccgatgacttactcgaccatgttttcaccaaagctgccacccaaaagaaggtaagaaactttttttctcgcttttccgataggaagatcgttagtaagttggaagacatagttgtcacacttgagtctcatttaaaactcaaggagagtcttgatttgaaagagagtgcagtggagaacttgtcatggaaagctccatcaacatctgtggaagatggatctcatatatatggtaggcagaaagataaggaggccataatcaagttgttgttggaggataacagtgacggtagtgaagtgtctgtggttcctattgtgggcatgggtggggttggaaaaactactttggcccaactggtgtacaatgatgagaatctggtagagatatttgactttaaggcatgggtttgtatttctaaagaatttgatgttctgaagatcacaaaaactatgatagaggcgattactggagagccttgtaaattgaatgatctgaatctacttcatcttgaattgatggacaagctgaaagataaaaaattcttaattgtcttggatgatgtttggacagaggattatgttgattggagtcttattaagaaaccatttaaccgtgggattaggagaagtaagattcttctaacaacccgcagtgaaaaaacagcatctatagtccaaactgttcacacctatcatctaaaccaattgtcgaatgaagattgttggtcagtgtttgtgaaccatgcgtgtctttcctctgaatcgaacgagaacacaacaacactagaaaaaattggaaaggagattgttaaaaggtgcaacggactgcctttagcagcacagtcgcttggaggcatgttgagaaaaaagcatgacatcgtggattggaataatattctgaatagtgacatttgggaactttctgaaagtgagtgtaaagttattccagcactgagacttagttatcattatctccctccacatttaaaacggtgctttgtttattgttccttgtatccacaagattaccaatttgaaaaaaatgaattaatcttgttgtggatggccgaagatcttttgaagaaatcaagcaaaggtaggactttagaagaggttggtcatgagtattttgacgatttggtttcgagatcatttttccaacgttcaaacacaagtagaagtagttggccttatggcaaatgttttgtgatgcatgacctcatccatgatctagccacatcactcggtggagatttttactttagatcagaagaacttgggaaagaaacaaagatcaagaccaagactcgtcatttgtcatttacgaaattcaattcttcagtcttggacaactttgatgttgttggtagagcaaaatttctgagaacttttttgtccattatcaattttgaagctgctccattcaacaacgaggaggcacgatgtatcatagtgtcgaagcttatgtacttgagagttttatcattttgtgacttccgaagtttggattctttgcctgattcaataggtaaattgatccatctgcgctatttagatctttctgattcaagtgtagaaacactgccaaagtcattgtgtaatttatacaatctgcaaactttgaagttgcgtagttgcagaaagctgactaagttgcctagtgacatgtgcaatcttgttaatttgcgtcatcttgagatattttggactcctataaaagagatgccgagaggaatgagtaaattaaatcatttacaacatctggatttctttgctgtgggcaagcacgaagagaatggaatcaaagaattgggaggactttcaaatctttgtggtgaacttgaaattaggaagttggagaatgtttcccaaagtgaagaagcgttggaggcaaggatgatggataaaaaacacattaatagtttacagttggaatggtctagatttaacaacaacaggaccaacttccaacttgaaatagatgtgctttgcaagttacagcctcactttaacattgaatcgttgcaaataataggctatgaaggaaccagatttccagattggatgggaaattcttcctactgcaatatgattagtctaaaattgcgtgattgtgacaactgtagtatgcttccttcacttggacaactaccttctctcaaggtccttgaaatttcagtattgaataggctgaagactattgatgcaggtttctacaagaatgaagaatgtcattctgggacgtcctttccctcccttgaatctctggccttttatggcatgccttgctgggaggtgtggagttccttcgattcagaagcttttcctgtgcttaaaagtctttacatacgtgactgccctaagctagagggaaatttgccgaatcaccttcctgttctgaaaaaacttgcgattaagtattgcgagctgcttgtctcttctctcccaacggctcccgccattcaaagtttggaaataagtgaaagcaataaagtagcactgcatgcgttacctctattggtagagactatagaagtagaaggaagcccaatggtggagtccatgatggaggccatcactaacatccaaccaacttgtctccggtctttaacattaagggattgctcgtcagccgtgtcatttccaggtggtcgtttacctgaatcactgaagagactattttttggtcaacgtcaccttggttttttcggtcaatgcgacaaagcttgttaccaggaacaacttgaggcacttgcggactcgcttgtcaggtttttcatcattgacatgcaagctcagatggcaagagaccagaaggagatcctgcttaagaactgttggaatttgaatcagtgggaacttgcagcaagcaagcaacagaagcaagggggtgcaattagaaaacaagaccaagagagcaattacaagacccacttacctcaaggattaggtgtgaaacaagcaggtgcatcatatctggaagaatga

SEQ ID NO: 155 - DNA, 2451 bp, Glycine max
  source 1..2451: mol_type = other DNA
atgcctccgaagacggcgaagcgagggtccgcgtcgagcgcctcgaagaggggtggcagaggtggcaggggaaccccgaaggctgcacagaaccaacagcagcatgaaactgcggaggagacagtgaaggttgaagagaaacaacaacccgtggttgttgttgctgctgaggaggaacagcagaaggtggtggtggaaaaagagattcctgctgttgaagagaacgagtccgctgtggttattgaggataaggctattgacatgaatcaaatggctccggaggctgtggaagaaacgtcccatgcggctagtgggttgagcttggtgaaaaatgatgaagaggaggttaaggagtccatagatgaatatgagaaagatgaacgcttagatttggaggataatgatcctgagtatgaacctgaggagtatggtggagttgattatgatgagaaggaaattgaacaagatgagggtcatgaggtaggaaatgaagtagaggaagaagaggctgaagataatgtaggtgaagaagagggtgatacaggtgaggaagaagtcgaagatgttcatgacgaacttgagggtgaagaggagcatgagcatgctggtgaggagcgtgagcatccagactttgctgacgttgaggaagaggaacaccgagaagtcgtgaaggagaggcaaaagcggaaagaatttgaagtttttgttgggggcttggacaaggatgctactgagagtgatctgaggaaggcttttggtgaagttggggtggttacagaggtcaggttgatgatgaaccctcaaactaaaaagaacaagggatttgcattcttgcgttttgaaactgtggaacaagcaaaacgagctgtagcagagctcaaaaatccagtgattaatggcaaacaatgtggtgtgactcctagtcaggacagtgataccctctatttgggaaacatatgcaagacatggacaaaggaagctttaaaagagaagttgaagcattatggagtcaccaatgttgaggatttaactttggtagaagatactaatgatgaaggaaagaaccgtggatttgcgtttttggaatttccttctcgttctgaagctatggatgcctttaagcgactgcagaggagggatgttgtatttggagttgataagcctgcaaaagtttcttttgcagattcctttattgatccgggtgatgaaattatggcgcaggttaaaactgtgtttattgatgcattgcctccttcatgggatgaagattatgtccgggatcttctcaggaaatatggtgagattgaaaagattgagcttgccaggaacatgccagctgctcggaggaaggactacgggtttgtgacatttggctcacatgatgctgccgtaaaatgtgctgatagtattacaggcacagagttgggtgagggagacaagaaggcaaaagttagagcaaggttgtctagaccactccagagaggccgcggaaaacacattagtcgtggggactatcgttctagtcggggatctggaatgatgacgcggccttcatggagccgacctgcacctcgttcttttccttctcgtagtgtaagaggagttggaagtcgtgctccacctgtcagaccagttagtgtgagagatagacgccctatgatgtccatacctgcaagaagtaggccagtgcctcctccatctagatcttatgataggagaccagttgctcctgcatatccaaaaagtagcatgaagagagattatggtcggcgtgaggatataccacctccaagaagtagagttgctgtagattatggctcaagggttgcttctgaaagacgaacatcttacagggattatcctgctcgtggtcctggctacaccgagctccctagaagcacatctcgtgctgcaccaaggagaggctatgtggatgatgggtatggccagagatttgagagggctcctcctcctcctcctcctccccatctaagttaccgtgaaggacgtccccgagattatgatgctctgtctggctcaaaacgttcttatgctgctatagatgatgttcctccacgatatgctgatactggtgctcgccaatcaagagctcgactggactatgactatggcggtagtgcttcacagtatggagatgcttatggtgatagacttggaagatctagtatgggatatggtggtggcagcagaagttctatttccagtcaagattcacatgggttgtatagcagtcggcagggcatgagttatggaggaggttcttttggtagtggtgatgttggtggcatgtattcatcaagttatggtggtgattacatttctcgtggaagtgatgttggtggcagctcatattcatcaatgtattctggaaggggtgtgggtggtggtagcagttatatgggtggtggcggatcaggatcttattattga

SEQ ID NO: 156 - DNA, 624 bp, Glycine max
  source 1..624: mol_type = other DNA
atgtacgctgatcgcgtggagggtggagcaaggaggccagtgaaggaacgccttaatgatgtgaatggtgtcagtggctctactcgcccgcagcagcagcgtcaaatcactggcaagaggtcaaggcaagatgacaagtgggaacatgatctctttgacaatgacgaaccccggatcactaatcgcaaagttagtgctcaagatcttcgcttcaagcttcaaaggaagggtcttcaacctgcatctcagagtggaaagagttctgccccaaacgtgctggatctccgtgaaaggctatcagggacaatggcaccacaacctacaaattatgatctgcctaagacaaaagttattaaaccaagcagtaaaagtgttggtgttgaggctcctgcagtgcagatcaaaaggccagctgacccagctcctaaaaagtcatggaaggctggtccttcagttgatgaatttttgcggtccttgggtctcgaaaagtatctcataacttttcaggctgaagaagttgatatgacagctctcaatcatatgactgatgaagatctcaaggctatgggcataccaatgggaccaagaaagaagatacttttagcactggagtcgaaagtctaa

SEQ ID NO: 157 - DNA, 8598 bp, Glycine max
  source 1..8598: mol_type = other DNA
atgcatcacgtgtctgtcaatattagtgatgaaaatcaaagaggcagaagcggagatttacttcttgttgccaagttagataattggggtatttggtgggtttcaatggtgaagcttgatgaaagaataaacatagttcagtcagttgagtggatggatttccaattttctgataatcttcttgtctgtcttaattcttctggcttgattgttctctattctgcaatgtccggtgaatacatgacacatttaaatgttttacaggagacttgtggccttaacccccattttaatttgcaagggttagaaaagttatattcacacgataatatatatgctaaacaagagtgtagcattaaggataacatgtctgaccagcagagtgattcttttaggagatcattcaagaggttagttgttgcttcacacacctcccttttagctgtggttgatgaatgtggtgtgatctatgtgatctctttgagggaatatatacctgacaaaagttattcatctgagaagttgcttccacattgtcagcaatttgggctgggaatgttggttggctggggagttggtggttctgatatagatcgccaagcagtatactccaatttatctggacattttcaatcaaatgatttaaacataaaacatggaagtgttgcttccttagacaaagctgtggcaggtaatgcacttcagaaaactaatggctgcacattcaaagaaaaggggaatttggttggctcctattctagtggtttttctgctacttctaaagtaaataatggtcacaaatttcttggttatgatgtgcaatcacctgttatgagaaagatattacttcccaattttagagtctctgaggatgattctatttgtttttctcctttgggaattactattttctctaaaaagaagtgtgtaaagaatcagaagggttctcagcttatccattttaatctgcaagtgaagttagaagtccgtgatgacaacttcttagacagtgtatatgatgtataccactttgatggaaaagatgttattggagaagcaattggttgcacattccaaggttgtttttatattgtgagagatggtggtctatcagtatatattccctccatttcaattttatcaaattttcttcctgttgagtacattggttatcgtcaatcaagtaaggatatggggatttcagttctacttaaagacaatctaaaaataaaagaaccaaccaaaaggttttctccttggaaagttgaaattttggacagagttcttctgtatgaaggcactgaaatggcagatcaattgtgtttgaaaaatggatgggacatcaaagtttctcgtattcgtcaattacaaatagcattggactacttgaaattttatgaaatagaaagatctttggaaatgcttgtggatgttgatctagcagaagagggaattttgagattgctttttgctgctgtgtatcttatacttaataaaggtggcaatgatagtgaaacttctgctgcttctaggcttcttgcactggccacttgctttgcaacaaagatgctccataaatatgggttgctacaacataaaaaggatacatgcatagcagagggctttaacaagacgggattactatctcttccaccaattgaaccagttaaactaaaaactgaggtggattttgctcagaaactttgtgagatagctcatttcttggagatcatccgaaatctgcaatgtagacataggtcaatatttctaagggccagtcaaggattggtcgacagtggagaagagtcatctttaataagtacagacatgttgcaggaagaatcccaactttcaattcttccatcagatttagagtcattggatgtgttgaaccaacatgagctttcttttcctctaccaggcggcaacaataatgaaaatcttgtgctggtgcctgttgattctgagtctcatttggtctcagatgaatttggcagcatatctcatttaaccccattagaaggaattttaggaaagaaagttttgcctgtggaaaatccaagagagatgatggcacgttggaagctaaataatctggaccttaagactgtggttagggatgctttgctctctggccgtcttcctttggcagttctgcatcttcatcaaatgaatgatttcgttgctgataaagagcctcatgatacttttactgaagttcgtgacattggcagagctgttgcttatgaattatttttgaagggtgaaactgaacttgctgttgctacacttcaaagacttggagagaacattgaatcttatcttaaacaacttctgtttggcactgtaaggagatccttgcgaatccagattgctgaggaaatgaaaagatatggttatctaggaccatatgagtggaagatattggatgatatgtcactgattgagagtctctatcctagcagtagcttctggaaaacatataatcgccgactgaaagaaatcagcattgcaccggactctgttttaccagtggaaaataaattaaggctcttgcataaccattcatttcatagtcatgtcattgaatgtggtgagattgatggaattgtctttgatgcatggattgatattagtgaaagttcctctgctctggaagttgatgaagatgatgctcatgttggatattgggccgcagctgctgtctggtttgatgcctgggaccaaagaactgttgatcgtatgatattgaatcaatctgttcattcggacaatcctatattgtgggagtcacaacttgaatatcatgtttgccgtaaccactggaaggaagtctttagactgctggacttgatgcctgcatatgtcctatcggctggaagccttcaactcaatttggatcttttacagcctgcctcatctttaggatgcaatatgaatatgaagtcttctaattatggaaatttcttgtgctcttttgaagaattggattctgtatttatggaagttccagatgtccaaatgtataggttttcacctgatatatgttctgggtggatgaggatgcttgtggaggaaaagcttgcaaaaagatttatatttttgaaggaatattgggaggggacattggagatgataactctcttggctcgttcaggtttcatatctggcagagataagatctgcttggaggatgatcttactaagatgtcatctgtcagagatggagctgtacaagctttgcataaaatatttgtacatcactgtgcacaatataacttgccaaatcttttggacctttaccttgatcatcatcgtttggccctcgaaaatgattcactttatgcattacaggaaactgctgttgattgtgaatgggcaagatggctactcttatcgagagttaaggggtgtgaatatgaggcctcacttgccaatgctcgatcgataatgtcacgtaatttagttcctagaagtggcctcagtgttctggagttagatgaaataattcgaacagtcgatgacattgctgaaggagggggagaaatggcagccttggcaaccctgatgcatgctgctgttccgattcaaagctgcttaaatagtggtggtgtaaataggcatagttattcctctgcccagtgcacattggagaaccttaggccaactttgcaaaagttccccacattgtggcgcactctcgttggagcatgtcttggacaagacacaatggccttgctggtccctaaggcaaaaactgctttatccgactatcttaattggcgtgatgacatcttcttctctactggtcgtgatacttcacttctacaaatgcttccatgctggtttcctaagcctatacgcagattgatacagctttatgttcagggtcctcttggatgccaatcattttcagggttccctacaggcgaaactttgctgcacagagacattgatttattcataaatgctgatgtacatgctgagataaatgcaatctcctgggaggcaacgatccaaagacacattgaagaagaattatatggtcctttacttgaggaaaatggattggggcttgagcatcttttacatcggggacgggcattggctgcttttaaccaaattcttggccatagaattcaaaatctgaagtcagaaggggaatctagtacttcagctcatggacaaactaacattcaatcagatgttcagacacttctctcaccactaggacaaagtgaagagactctactttcatctgttttgccaattgctattatgcattttgaggattccatgcttgttgcttcgtgtgcttttcttatggagctatgtggtttatcagccaacaaactgcacgctgatattgctgtgctaaaacggatatctctgttctacaaatcaagtgaaaataatgaaaatctcaggcaattatcacctaagggatctgtgtttcatgcaatatcccatgaaggtgatgtaacagaatctcttgctcgagctttagctgatgaatatttgcacaaggattctcctgtaactggtactgagactgtaagtaaacaaccttcacgagctcttatgcttgtgttgcaccatttggaaaaggctagccttcctcggttggttgatggaaaaacatatgggtcatggctattaagtggaaatggtgatggaaatgagttaagatctcaacgaaaggctgctagtcagaactggactttggtaacaaatttttgtagattgcaccagcttccattaagtactaagtaccttgctgtattagctagagacaatgactggattgaatttttgtctgaagctcagattggaggatattcatttgatacagtagtccaagtggcatcaaaggagtttagtgatctgcgtctccgacttcatatgttaacagttttgagagcaatgcagtcaaagaaaaaggcaagcactgtattgttcttggattcactggagaaaggcagtgaaacaacctttcctgatgaaaacatgggtgttccagttgaactcttccaaatattagcagagtgtgaaaagcaaaaatgttctggagaggctctcttgaggaaagcaaaagagttgtcatggtcaatattggcaatggttgcttcatgtttccttgatgtctcttcattgtcatgcctgacagtttggttggaaattactgcagcaagagaaacttcatccattaaggtgaatgatatcgcttcccagattgcagacaatgttggagcagctgtaaatgctactaatgccttgcctgtcggtgacagagtacttacatttcattacaataggcagagtcctaaacgtcgacggctaataactcctgtttcactagactcctctgcttctgcaatatctgacatctctagctcttctataagtgaaaaaatatttgattcccaaggtaaaactatggagaatgatagaaaaatagaacattttggatgcattaatgttcctagtaattccgatgaagggcctgcttctctctcaaagatggttgcagtgctttgtgaacaacaattgttcttgcccttgctaagggcattcgagatgttccttccatcatgccccttgcttccattcatccgcgctcttcaagcattttcgcaaatgcgcctctcagaggcttctgcacatttgggttccttttctgcacgaattaaggaagaaccaatctacttacaggaaaatgtaggaagagaggcgcagattggggcatcatggattagttctactgcttcaacagctgctgatgctgtgctttcaacctgcccatctccttatgaaaaaagatgcttactgcaacttcttgctgctactgacttcggtgatggtggacatactgcagcatactatcgaaggatttattggaaaattaatttagcagagcctttacttcgtaaagataatgagttgcatttaggtgatgaaatttcagatgatgcttcactcttatctgcactagagaataataggcactgggagcaagcaaggaactgggccaagcagttggaggccaatggtgcaccctggaaatctgctacgcatcatgttactgaatctcaggctgaatctatggtagctgaatggaaggagtttctttgggatgtgccagaagagagggttgctttatggagccactgccacacactattcatcagatattccttcccttctcttcaagctgggttattttttcttaaacatgccgaagctgtggagaaagatcttcctgcgagggagcttcatgaacttttattgctttctctgcaatggctgagtgggatgataagtctttccaatcctgtctgtccattgcaacttctgcgtgaaattgaaaccaaagtatggctcctggccgttgagtctgagactcaggtgaagagtgaaggagatttcaattttaccttttccaccagggagagtggtatcaagaacgactccagtattattgatcgaactgctagtataatagcaaagatggacaaccatataaatacaatgaggagtagaattgtagaaaaatacgagtccagggaaaataaccaaatcccccacaagaatcaagtgatggatgctggtctttcaactacttttgctgggaatatgaagacaaaaagaagggccaaaggatacatggcatcaaggcgcccacctcttgaatccacagataaaaatgctgatactgatgatggctctagtactattggtttaaagaatgagttgcagctgcaagaggaaaacataaaagtggaaatgtctttttctaggtgggaagaaagggtcggaacagcagagctagaaagggctgtactttccttattggaatttggacaaattgtggccgccaagcaactacagtataaattctctcctgggcaaataccatctgagtttagacttgttgatgcagccttgaagcttgctgctatttcaacacctcctagcaatgtatcagtgccaatgcttgatgaggaagtgcgttcagttatgcagtcatacggtataatgaatgataagcactatgttgacccgctgcaggttttggagagtttggtgaccatttttattgaaggcagtgggcgtgggctgtgtaagagaataatagcagttataaaagctgcaaacaccttgggcctctcattttttgaggggttcaacaagcaaccaattgaactactacagctgctttcgcttaaagcacaagattcctttgaggaggccaactttctggttcagactcatccaatgccagcggcaagtattgctcaaatacttgcagaatcttttctaaagggtgtgttggctgcacatcgtggaggatatatggattcacagaaggaggaagggcctgctccattgctgtggagattttcggacttcttgaagtgggcagagctttgtccctctgaaccagaaattggacatgctttaatgcgtttggtgattactggacaagaaataccacatgcgtgtgaggttgagcttcttattctgtctcaccacttctacaaatcatcttcttgccttgatggagttgatgttcttgtggcccttgctgcaactagggttgatgcttatgtcttggagggtgattttccatgtttggctcgcttgataactggagttggaaatttttatgctctcaatttcattcttggcattcttatagaaaacggtcagttggatcttcttcttcagaagtattctgctgctgcagacacaaacactggcactgctgaggctgtcagaggatttcgaatggctgttcttacatccttgaagcatttcaaccccaatgaccttgatgcatttgccatggtctacaatcattttgatatgaaacatgagacggcagctcttttagagtcacgtgcagagcagtcatgtgagcagtggtttcaccgctacaacaaagaccagaatgaagacttgttggattccatgcgctacttcattgaagctgctgaagttcactcttcaatcgatgctggcaacaaaacacgtaaggattgtgctcaggcttcccttctgtcccttcaaatacgaatgccagacttccaatggctttatagatcagaaaccaatgctagacgagcgttagttgagcagtctcgatttcaagaagctctgattgtagctgaagcttataacctgaaccagccaagtgagtgggctttagtactctggaatcagatgcttaaacctgaagtaatggaggagtttgtggctgaatttgtggcagttttacctcttcagccttcaatgctgattgatttagctaggttctatagagctgaagttgccgccagaggggaccaatcccatttctctgtctggctcacagggggaggattgccagctgaatgggctaaatatttaggaagatcattcaggtgcttactgaagcgaacaagggatttgaagttgcggatgcagttggctacagtggcaactggatttggggatgtcattgatgcatgtacagaagaaatggataaggtcgctgacaatgcagcaccactagttctgaggaagggtcatggtggagcttacctccctttaatgtga

SEQ ID NO: 158 - DNA, 912 bp, Glycine max
  source 1..912: mol_type = other DNA
atgacgaagatcgttgataccctaggtcctaaatctcgatcggtcgaggttacttctgcttgtcttaaagctagaatgccaggtttttctaagtgcatgggaaatgtctatttatgtccctacaatttgtgtatgcatgcattgctgttggttaggtttcttgtacgtgataggaaagaagttgagtgttgcttactctattgttgttgcctttgccaggtcatgatggacacttttggtgcaaagatgcatgccgatggtcaggttgttctgactcctaattggggaaaagaagcgtcttcacaaatattgcctatcaatctcgatggactggcaatgtcaatgacgaagggagacaccatttttattggtcattacttgttcacaggaagtgaaactacttctgatgtcatttgtactatagagaatttgacaacattggctgggtcattgttcactttgcatgcctctcaaattcatgttgacttgcctaccctcagtgataaagacaatgagggagtaaaacacaaaattgattatctatcattgtcatatactaggcatgctgaagataatttggtcattgatcttcccccaaagaaggtgagcatgactacacgtgttgtggatagcatgactgacaacttaagaccaactcgtgcagaagccactaatgttgccaatgctattttagatggaagtgatgcaatacttctaggtgctgagaccttatgcgggttataccctgttgagactatttccacaattggcaaaatttgtgcagaagtcgagaaagttttcaatcaagatctttattttaagaaaactgtcaaatatgttggagaacccatgatccacttggaatctattgcatcctctgaagtacgagcagctattaaggtgaatgaataa

SEQ ID NO: 159 - DNA, 5865 bp, Glycine max
  source 1..5865: mol_type = other DNA
atgaatgatctgatccgagggaaaatggtgaacatggccttgttactgagttttctggtgacggcgacggtactggcgtcgtcgtctcacgctgcttctagtggtcctcacattgccgacgttaacatactcttgccgccgaaaatgaccttccctgtcgattataggctccaaggaagtgacggttgcttccaatggtcatgggatcatcatgatattctgtctgttgagccagagtataattcaagcagcaagtgctcgactagtgcaaggataagatccattgccccttatagtggtcggaaggaaactgctgtttatgctgcggatttgcagactggaattgtgattcgctgcaaagtcttcattgacaacatatcaagaatacagatatttcataattctattaaacttgatttagaggggcttgccacactacgtgtccgtgcctttgacagtgaagaaaatgtattttcctcattagttggcttacagtttatgtggagccttatgcctgaagccaacggattaccttgtcaccttgttaatgttccgctaaaggattccccccttagtgattgtggaggactgtgtggtaacttagatatacaaataaaacttgaagacaatggtgtattttcagatctgtttgtagtaaagggaattgagattggtcatgagattgtgtcagtgcatctgctcgagccacagttaaagaatttggctgatgagattgttcttactgttgctgaagctatgtcacttgatcctccatcaccagtctttgtccttgttggtgcagtcattccctacacccttaaagtcatccggggaaatgttcctcaagtggtcaccttaccctcagcacatcaccaatggtctgtctcaaatgcttcggttgctcaggttgactcaaaaacagggctagcatatgcatggaacttgggaatggcagctgttattgttgaagatactaggattgctggccatgtacaagtgtcttcactaaatgtggtcctaccagcttctttatgtttgtatatatcacctttatctagttctggtgatccagtggaaggaatcaaatctattgctctaacgacccgttggtatgttgtatctggccatcagtatcttatccagataaaggtttttgcacatgatcatgatgcgcaagagatttatattacagagaatgacgatgttaaggtgtatgataatgattctggtcactggaaaacattttgggtttcaaatgatattgctgtaaaacatggctggcggaattctaaaatcttgaaggcttattcaccaggacttgaaaaattgacagcttctttgagctatcctggtggggctgatgacaaaaaggagattatcaaagctgtgcaagaagtcatggtttgtgatcgagtaaagtacaccttgggcaatgaaagtggaatcatcttactgccctggtctcctggtgtttatcaggaggtggagttaaaggctataggaggttgtgcaaaaacagtgagtgactacaaatggctatcttcggacttgtctactgtatctgtgtcagcttttggtgttgtccaggcaaaaaaacctggtaaagctactatcaaagtgttatctgtatatgattcactaaattatgatgaggtccttgttgaagtctccattccttcttctatggttatgcttcacaattttcccgtagaaactgttgttggatcgcatcttcaagctgccgtgacaatgaaagcagcaaatggtgcctttttctatagatgtgatgcctttaattctttgataaagtggaaagctggaagtgagtcttttgtaattgtcaatgcaactcaggagttgttatacttggaaacagtgccaaatactcaattccagtcatcagttgatggctctccttgttcgtggacatatgtatatgcttctaatcctggtcaagctgtgatccacgccatattttctaaagaagatcaccactacagtcttggtcctggtgtattaaaagcatcttcacgtattgtggcatatctaccccttattgtgcgtcaggcaggtgatggaaaccaatttggtggttactggcttgatttggttcaagccgaaagtaacaagcagtctcatagcttagaggagttatatcttgtacctggtacaagtttagacatagtacttgttggtggacctgaatggtgggacaatggtgttgatttcattgaaactgtagaagttttggatgaagggaatgctctggctgaagatggagttctagtgcatcgggtctctagtaatttgtatggagttttatgtcaaaaactgggatcctttaaacttcttttcagacgtggaaatttggtcggggatgaccatcctctgccatcagtagctgaagtttggctgtcagtaacatgcaacattccttcttctattgtacttatagctgatgaaccagtgaatgaacgaagaattattaaagcagcagctcaagctgaacgtagttcaggtagacttcgtgatacccctgttattgtggcaaatgggcgctctattcgtgtatctgcagttggtatcagtgattcgggagaggcttatgcaaattcatcttctctcagtttgaggtgggaacttggcagttgcgagggactggcatattgggattatgcttttgatatagtgaagtctaacagttgggagagatttctggtcttgcaaaatgaatcaggattgtgcactgttcgtgctactgttactgactttgctgatagcttgggagatgatacatttcatcgatttactaaaactgaaaatgttctgacagatgcaattcggttacagcttgtttctaccctaagagttgatccagagttcaacttgatatatttcaatcctaatgcaaaggtaaatttgtcaattattggtgggagctgtttcttggaagctgttacaaatgattctcaagttgttgaagttattcaaccaccatcgggattagaatgcctacaattgattttgtcccctaaaggattagggacagctaatctgactatatatgacattgggctaacccctccacaaagagcttctgcgctcgttcaagttgcagatatagagtggatcaagatcatatcaggagcagagattagcctgatggaaggaagtttgcaaactattgacttattggctggtactaatggtggaaataattttcatgcttctcagtttgtttacatgaatcttcatgtacatgttgaagatagtataattgagctggtggatactgaagacttctcaagtcttgttggtggacatgttaatgcaccaagttttaaaataaaggggagacatcttggaattacaaccctttatgtcagtgctatacaacatttgggacatgtaatacaaagtcaagccattaaggtagaagtttatgcagccccaagaatacaccctcacgatattttcctactacctggtgcatcatatgtgcttaccatggaaggaggcccaactctgggtgttcatgttgagtatgaaattgacaatgataaaattgcgagtattgatagatattctggacggctcttagcaagttctattggtaacactacaattattgctagtgtctttgcaaatggtaatactgtgatttgtgaagcacggagttttttaagggtgggagttccatctactgtcacattgcatgtacagagtgaacaacttgggattggccgcaaattgccaatttatccgttatttcctgagggaactttgtcttctttctatgagctatgtaaaaattaccagtggagtattgaagatgaaaaggtattgagcttcaaggtggcagagaccttgcatgaggatagtatccagttgactgcttcagcaggaagtcaagttaatagttattttgatgacaatgaccttggttttattaatgtcttgtatggaagatctgcaggcaagaccaatgttgctgtctctttctcatgtgaactttcaacttctgggtcaaggacacagtcaaggttttacagttcatctttatcagttacagtgattcctgatcttcccctggctttgggagtccccataacctggattcttcctccctattatacaatgacaagccctttgccttcatcatcagaatcccattctcagaatgatagtcggaatcgcagaggaaccattagctattctttattgagaagtttggagaaaaatgaagctctgcaaaaagatgctatattcattgatgcggatagaattaaaacaaccaaaagtaacaatcttgcttgcattcaagctaaagatcgtacaactggaagaacagagattgcttcttgtgtcaaggttgctgaggtgactcaaattagaatagccagtaaggaggttctgctcaatataattaaccttgccgttggtgcagaacttgatcttccaacaagcttttatgatgctctaggaaatcctttccatgaagcatacaatgcagtacctttttatgctgaaactaactaccctgatgttctctgcgtaaacaaaacagctgatggaaaaggcaatgtccatatcaaggcaattcagcatggtaaagctcttgtgcgtgtagcaattagtgaagatctgcaaaaatcagattatgtgctgattagagtgggtgcccatatatatcctcaaaatccagttcttcacataggaagtcctcttaacctaagcataaaaggtttgagtgatacaatttctggacagtggtttaccacaaatggaagtgtaatatcagttgatacattatctgggatggccaaagctattggggaaggttcagcacaagtatctttccattacggaagattgagactacaaacaacaattacagtgttgaaaggaaattatatttttgtgaatgctccaaaagagacgttaactaatgtcccgtacccttcaaaaggatacaacttctccgtgaaattcagtgaatcccttggtgcacctggagaaaagaagagaattttattcaattgtagagtagatccactatttgtggggtatgtgaagccatggctggatcaggactctggcaactcatattgcttatttttcccttactcaccagagcatttggtgcactcagtacctaagttagaaggcatgaggccagatgtatcactctccatttctgcttcccttgaacacgaacatgtttctggatctgcttcagcactttttattggaggattttctataatggagatgagcaagaattcgatgcagctgaatttgacgccaggctctaacaaaacttgcattactgtcttgggaaatactgatgttgaaattcactggcatcaccgagacttgattatgatcagtctcatccataaagaagattttggcattcgagggtttgcacggtatgaggtcaaattgcttaaagccaagagattcaaagatagaatcatcatcactctcccagctaatggccagagtgtggagatagacatcaaccatgaacccgaggaaacagcatcatccagcgtaaccataaataaggccttctgggcaagcatcctggggtatttgctgctgttgattttatcaattgctatcatcacacgtttcttggacagaccagagagatctcagcaaacaagttcttcagttactactactccaagcattgcagcgcccacaactcctgacagaagcaccccatcatctgttgtaaatgactcctctcctcgaacacctcaaccatttgtagactatgtaaggaaaacgatcgatgaaactccatattataagcgagaagggagaagaaggattaatccacagaacacgttttag

SEQ ID NO: 160 - DNA, 627 bp, Glycine max
  source 1..627: mol_type = other DNA
atgaacttagtcgatacaaagctatatgatagcacttccatttttttgattcggcaatatgatctatgctatgatttatcattcatggacgttgataagatccttccatctagcaacaccttaggatggcacagccctaaagttaatgtcgaggttcaaatgaaaaaagacttatggtggatacctgggcaccccgagacgaggaagggcgtagtaagcaacgaaatgcttcagggagttgaaaataagggcaagagacaacttggtgaattgaaacatcttaatagccagaggaaaagaaagcaaaagcaattcctagaatgctgcaccctagatggcaagagtctagtaaccaaaagcatcactagcttatgctttgacccgagtagcatggggcacgtggaatcccatttgactggacatttagggataaagcactatttcggtgcaggccacgagagcggtaccaaatcgaggcaaactctgaatactagatgtgtcctcaaaataacatggatcagggggtgcatagacaaccaagaggtttgcctagaagcaaccacccttgaaagagtgcgtaatagctcactgatcgagcgctcttgcaccgaagatgaacaaggctaa

SEQ ID NO: 161 - DNA, 291 bp, Glycine max
  source 1..291: mol_type = other DNA
atgagaatcatggcttcaccaccagctctctttacttcgtttcctttgtcaattcttcctttatcctctcctctctcacacaaaacaacattgcctctgaaatctacatcattcaaggatccatcaacacaaaatgcagcttctatgccattctttccagcatatgccaagggtagcagtgtgggagaaaaacttgagactagaaaacagattcaccatcaaattcttgcccatttccctgcttcgctgttgctaaaatgcttctgtcaatattgcttgctccaactgtga

SEQ ID NO: 162 - DNA, 4287 bp, Glycine max
  source 1..4287: mol_type = other DNA
atgcagccaggcggtcgatctcctaagccagtgagcagtccaacatcaacatcacagttaaaatctggttcagatagtgtccaaaacagtgtttcttccttttcttctcatgttaagggtaggaaaagggagagggtagatcaagggcaagaatctgtaaagcgagaccgttcaaccaaaaatgatgatggggattctggtaattttaaggcggatagcattttgaagacagagattgccaaagtaacagaaaaggggggtcttattgatcctgaaggggttgagaaattagtgcagctgatggttcctgatggaaatgagaaaaagattgatttagctagccggtcaatgcttgcagctgtaattgcagcaaccgagaaatttgattgccttagtcggtttgtgcaactaaagggtttgcctgtatttgatgaatggctccaagaggcacataaagggaagcttggtgagggtattgggtctagggatggtgataaatctgttgatgactttctttttgtattactccgagcactagataagcttccagtaaaccttcaggctctacaagcttgtaacattggtaaatctgtgaatcatttgcgaactcataaaaacttggaaattcagaagaaagccaggggtttagttgacacatggaaaaaacgtgttgaagctgaaatgattataaatgatgctaggtcttgttcagttcagactgtcccctggccagccagacaacgactttctgaagttgctcaaggtgggaataaacattcaagtgggtcagctgatgttgccatgaagagctcagttacacagctttctgcatccaaaactgcctctccgaagattgccccaggagagaatactacaagatctacatctgcatctccagggtctacgaaatcagtgccttcacctgcacccgcaaccgcaaacttgaaagatggacagccccatgctgcagctgttagtggttcatctgatcttcctgtggcaaatgcaagggatgagaaaagtagcagttctagtccatctcataacagtcaatcttattcaagtgaccatgccaaagctgggggtttctcaggaaaggaggatgccaggagctctactgcaatgagtgtgaataaggtatcaggaggctcttcacggcaccgaagatctatgaatggatttccgggttcaactccatctagaaggcaaagggaaactggatcgagcagaaattcctcacacaagaatttaatttcagagaaaatatctcaatctggattaagggaaaaagcaaatgatggaactcttcttgaggggcatactccgaagctgattgttaagatcccaaaccgaggtcgaagtcctgcacaaagtgccacggcaggttcttctgatgatccttcaatcatgaacagtcgagcttcatctcctgctctttcagagaagcatgatcagtttgatcgctgctcaaaggaaaagagtgatttttatcgagctaatattggtgcagacattaatacagaatcgtggcagagtaacgatttcaaagatgtattgactggttctgatgagggtgatggatcaccagcagccattactgatgaacagtgtaggactggtgaggattgtaagaaagtatcagatgtatctaagacaacttcttcatcacctgggagtgagagtaaagccaggaatttgcaggatgcttcatacagctccatcaatgctcttattgaaggtgttaagtacactgaagctgatgatgttggaatgaatcttcttgctcgtgtggctgctggagagatctcaaaatctgaatcgggaatgccagctggatcccctaaaaaaaacaccactacaattgaacagtcatatgcaggtaatgctgcagtagtcaagtcctctgaagagtatcttgttcaggataagtgctactctaatgctgaacatatgaagcaggattctagatctggtgattttgggacagatgatgatattcgggcatttgaagggaaagctacaggagaacacaatccatctagtatggatttacaggttactgaaactagtttagaaagcaaaggaaaattaattgtaaaatcatctggtacatctgctggcattcctgaaagcaccttccaagaagtcagagatattgactctagcaaactggtaaaggaaaagaaggttgttgtgagagtggatgctgtcaataatgttgatgaggtcaatgttgttgcaagagaaggtgaaacagaggcaattgaaaaattgtcacacacctgtgaagaagttgatgtcaagtgtgataaccatgcttctgaaggactaagctgtgacaaagagacagctggcaagtctcctgccacttgtgtgccctctgactctgtaaaagcgacagatgagaatgcgctccaatcatctggttacattgttgataaggttcctgaatacctaaatgaaagggaatctgaaaaaaatgatgatatggctgctcaggatcatgctaagcagtctttgaagcaaaaaaatgagtctgaaaatgatgctattatggtgcccgagaacagaggtttatgttcaggtgcaactggtcttgatgctgaatatgtggaagagaattcaggtacaaaagaggtttgtgaccaagatgcaggagcaggacagatactgcacacagatttacctagctttccttcacgagaaatggatcagcattcaggtcaaagggattcaaaattggctgccatggaatcagaggaagcagaggaatgcacctctaccactggagatgcctcttctgcatctgttgctggtgtctcagaagtggacacaaaagttgaatttgatttaaatgaaaggctcaatgcagatgacgggaaatgtagtgagatacctgggtctacacctgctgcaagattggtcagtcctgttccattttctgcttcatccatgtcttttggtattctctcgatcacagtggctgctgctgctgcagcaaagggcccctttgtgccacatgaagatttattgaaaagtaaaaaggagcttggttggaagggatccgctgctactagtgcatttcggccggctgagcccaggaaagttatggaaattcctcttgatatgtctactacccctattcctaatgatgaagctagaaagcagagtcgggtaccattggattttgacttgaatgtttccgatgaaataatacttgatgatctttcttctcagaactgtgctcgtcagactgattgtgtaactcgttcagatgatgggcatgatccaaataaatcaatggcctcacatgttcgttgttcaggaggactaggtcttgacttgaacctagtggatggagcttctgacgtgggcaattgcaccttaagcagtagtcataaaatggatgtaccacttacgcagtttaaatcagccgccagtggtcctccaaatggaaagatgagtgtccttagggattttgatctgaatgatggacctatagttgatgaagtcaccactgaacatttgatgtccacacgatctgccaggaatagtgtaccatctcaacctcctatttctggacttaggatgagtaatgcagaagtaggcaacgtctcttcatggtttccttccactgggaacacctattcagccgtaacaatatcttcaattatgtctgatagaggtgataagccattttcaattgttgcccctaatgtgtcagaaagggtgttgggtcctgcaactggcagcaacccatttggacctgatatttataggggggcagttttgtcatcttctcccgcagtgccatatcaatctgcaccatttcagtatcctgtcttccctttcaattccagctttcctcttccatcagcatcattttctggtggatcaactccttatgtagatacaacttcaggagggaggctttgcttccctgtagtaaattcacagcttataggttctgttggtaatgtttcagcccattaccccaggccttacgttgttagtttcccagatggtagcaatagtagtggtgctgagaacagtaggaaaagggcaaggcagggtttagatcttaatgctggacctggttcagacttagaggggagagatgagagctcacctcttgtaccacggcaactttctgttgctagttcacaggcccaacttgaagagcaagcaaggatgtttcacttaagcagtgatgttcttaaaaggaaggaacccgatggggggtgggatggctacaagcaatcatcatggcagtaccataatttgtaa

SEQ ID NO: 163 - DNA, 567 bp, Glycine max
  source 1..567: mol_type = other DNA
atgaattgtctatcattgttcaccttcctctatgcaatttgtgcgcactccatcacctccacgacatatccttccattctcgacatgaaaaccggagagtgctccctccgcagcatcagcatcggcgatggtgttctggtccctccgtggcgagaagtgtacgaggagaggcgaatcttcgacatcacccacaggtatgtccccgagatgctggtgtgggactcgacagaggggctcgggcaccacttcctgtggctcgaaaagagcatgaagaatggctcacgcgctaacaactccaacatgaaggtcggtgttcacaccgacacccatgttgacgcgctcggtcacatttacaacaattactatgacgtcgacttcgtggttgactcacttgacctaacactcctcaatggccttacacttttggttgatgttccacgggataaaaacattactactgaggttacgaaatccttgaatatccctagaggtgtaagccacgtgcttttcagaactttaaacactgataggccactcatgtttaagaaagaatttgacacaagctag

SEQ ID NO: 164 - DNA, 1422 bp, Glycine max
  source 1..1422: mol_type = other DNA
atgttggacgagagcaagttcgaggtacacttcaaactgtgggcccttcgaatcccttgccaacattgcaagctcgccacgcgaatcctcaacggttacttgcttgacaagccccgagttaaacccatcaccgaggacccaacctgtgacaaaaaccgttaccttatactctccgacaatgttcacaatcaagaagaatatctttctttcagcggaaattcttattttttacttgatttgaattggtggttgagggcagatttatctgatattccaaaacaaaagctagatgagctcaagggcttgtgtgagattgaagttgtgccctattctttgactttagggtattcttactggagtgcagatcatgtgctgaagcagatactgcctactggagtggagcttctaactgcaatccaaacagccactgatggtcaaattgcccatttaaacctacatgatgagttacttccccacaaagatgttattgcaaaggttatttatgataaaaattatccaagaatcaaaactattgttaataaagttggaaccattacaaatgaatttcgtgtgccagagtttgaaattttagcaggagagcataatatgattacagaagtgaagcaatatggtgccacttttaggcttgattacagcttggtttattggaattcgagattggaacatgaacacaaaaggttagtctcaatgttccaagctggagagaccatttgtgacatgtttgctggtattggtccttttgccattcctgcagcacagaaaggatgcatagtctatgcaaatgatttaaatcctgatagcattcactatctgaggattaatgccaaaatcaataaggttggtgattgcatttatgcatacaacatggatgctagaaaattcatctcccagatgatggaagtgccaaatactgaggtcacattagaaaactcacatgaagtccctattttggatacacgagacaatgctgaatcaaattctaaaaatgagttgctaactgggaatggagaggctcatggaactgacatccttgaaggtggtaggagaaaaggaagcgcaaacaaaagaatgagaggctctgaaatctctgtcacaaaaacttgggaacatgttgatcatgtaataatgaacctacctgcatctgctgttcagtttctagatgcattcaggggattaatccagaagaaatattggaaaggatgtctaccatggattcactgctattgcttcattagggcaactgaaactcctgagactataatagctgtagcagaatctgctttagacgctcatatacaagactcaagatttcatagggttagggatgtggctccaaacaaggcaatgttttgtttaagcttcaggttgccagaagcatgccttaaagaagattgtccataa

SEQ ID NO: 165 - DNA, 951 bp, Glycine max
  source 1..951: mol_type = other DNA
atgaatccgcttccttttcaatctcctcctccatggtttccaatattaccctccgaaccaccaaattcaacttccttttgggacaacagaaatgtctgcgatcgcctcagagaattgcaaaacactctcaatctcgcaaaggcaatgcagaaggagctagagatgttgatgatgataaaagatggtaaagggtcttcggaagatttaaaacatgcttcaaacgaaccttgtctttctggttttgtaaaatgtttagaagacaaaagggttagtgtagaatcccaagaatcattggctgtggaagctgcaaattctttgacgtcaaagctaagagctcagctggagccattccggtatgtcgcggacgaggcgagtccgtgggagcatgccaaggaaattgctaatagcaaggtactgaagatgaaagcaattgcgaagtctaaagccaaagaagaggagaagaaagtagaggctggggttgcacttattaagtacctaaatcatttaatttctccctttatttttcaacattgtatgttcgttagctcctcaccatcaaattacttgctcttttgttgtctttgtcattttctcccagaggaggatgacaagtttcttgagaaagttcaagctgctgtggaagaagaggagcgtgaagctttggctgcacaaagagttcacagtgtgactgaagaggatctgtgcagtgatgagaagaaatctagtaaaataggacagatcagtgagagcgatgatcctttggcaaatttacccattgagttctaccattattatcatggaagcaacaatgatatgggtacacttattgaggttagaagaggatggaatgcctatatcagaccaggaggaagccgaataccagggcactgggttcagcctcttcctccagctaatgagatatgggcatcttacttggtgagacctaaatga

SEQ ID NO: 166 - DNA, 3285 bp, Glycine max
  source 1..3285: mol_type = other DNA
atgccgacgccggtcagtgtagtgaggcaatgcttaaccgacgaggccgcgcgtgcactcgacgacgcagtcgccgtcgcgcgccgacgcagccacgctcagacgacgtcgctccacgccatctccgcgctcctggctctcccctcctccgccctgcgcgacgcgtgcggccgcgcgcgctccggcgccggcgccggcaccagcgccgcccgattctccgccgcatactccccgcgcctccagttccgcgccctggagctctccgtcggcgtgtccctcgaccggctcccttcgtcgaagtccacctccgccggcgaggaggagccgccggtgtcgaactctctcatggcggcgataaaacgctctcaggcgaaccagcggcggcacccggagagcttccacatgttccagcaaagccaacagggaacagcatcgacgtcgtttttgaaagtcgaactaaagcacttcgttttgtcgatcctcgatgaccctatcgtgagtcgcgtcttcgctgaagcagggtttcggagctgcgatataaaactcgccttgctccaaccgcctctgcctccggttcagcaccggttcaactggtcacctccggttttcctctgcaacctcgacccggcccagcccgacgaaaatattcgcaggatcatggaagttctcgcgagaaaaaacaagagaaaccctttacttatgggcgtttatgcaaagagtgctctaaagggtttcgttgagatggttcggaatggaaggggaggttcggcattaggttcggagttgagggtggttcgtttggagagggagattggggagtttgtgaaaaagggtgggagtggggaagagaaatttggtgttaggttgaaggaattggaacagcaatgtgagggttctggttctggggttgttgttagtttcggggagattgaggtttttgtaggggaagatgttgatgttgatgttgtaaggttcgttgtttcgggtttaacgaggttgttggagattcgtggtgagaaggtttcgttgctgggcgtggcggaaacttctcatgcttattccaagttcttgggattgtttcccaatgtagaaaatgattgggatcttcatttgctcactgtcacttctgccactccttccatggaaggactctactcaaaatccagcttgatggggtcctttgttccatttggagggttcttttctacacctgaaatcagaagtccggtaagctgtgcaaacgggtcttttacacgttgtgacacatgcaataaaaaatgtgaacaagaagttgcagatcttctgaaggtagatccttcatctagttattcaacaagctcacactggttacaaaaggttgttaacatggacgcgcatagagggtcggacgtggcaaagactaatgaagaaaacacaagtttgaatgataaaatcttgggatttcaaaagaaatggagtgatatttgtcagcgtcttcatcacacaagttcactacctcaatttgatatttctcaaacaaggtcccaagctccaacagttgaggttttacggtttggtctggcttttaaggaaagcagcaataaagacccatcacacagtgaattccaatattctagtcaaatttcctgcatgcccaaagagttgcatagtatttttccttcgaagcagttatctgttccattaccttctgatacagtctgcattaatactggaaccgatcatgttccaaaagtttcagagacactgcaaattcacatgaacaccccttgggttgccccttcccttatggccaataagagtgcacttgaccacagatcatcttcattccgtactcctgtgaccacagatttaggattgggaacgttatatacatcaactgctcaggatccagataccccaaaactacaagatcaaagaaagcatcttcagcacctgtcagattctgtttcaactgattgtgatggtatgaatgaaaatacttctcaccgaattgctagattttcctgctctggttcaaatttggaaggtaaatttgatttagcagatttcaagtctcttgatcgacttctcactgaaaaggttggctggcaggatcaagccatatgtgctatcagtcaaactttgtccctttgtaaatctggtgcaggaaagcgtagaggctcaaatggtagagcagacatatggttggctttccttggacccgatagacttggaaaaaggaaaattgcttcagttcttgcagagactatatttggaaaccctgaaagcctaatctctgtggatcttggcttccaggacagcttttacccattgaactcggtttttgaataccaaaaatcacgttgttatgatgtgcttaggaggaagacaattttggattatattgctggggagttgagtaaaaagccacattctgttgtctttcttgaaaatgtagataaagccgatgttttggtgcagaatagtttgttgcaggcagtaagaacaggcaaattttcatattcgcatggaagggtgattagcatcaataacacaatctttcttgtaacctcaactgtctgtaaaggtaatggctcttttgttttggaagagtctaagatgttttctgaggaaagaatccttgaagccaaaagatgccaaatgcaattattacttggacatgcttctgaggatgccggaagaattggtagcacaaatgttaaggttgtacccggaaaagggttttccaaatcatcatctctgaacaaaagaaagcaggctgatattagtgactccaaggagggggcaacaagcaagatgcagaaacaagacagtgaggcatctcgatcctatcttgatctaaatatgcctgtagaggatggtgaagagggtgtcaatgatgaccacgaaagtgaatccataacagaaaacacagatgcctggttaagtgatttctttgatcaaattgatgagaaagtggtgtttaagtcattcaattttgatgagcttgctgaggaagtattaaaaagaattggcatgctatttcaaaggacatttggttcagagcttcagctggaaattgattatgaggtaataacacacatacttgctgctgcttggttatcagacaagaaaaatgcagttgaggattgggttgaacatgttcttggcaaaggctttgttgaagctcagcagaagtaccttcctgcagctcaatatgtagtaaaactggttaattgtgaatccatttttgtggaagagcaagctcctgatgtgtgccttccagctagaattaacacggactaa

SEQ ID NO: 167 - DNA, 2943 bp, Glycine max
  source 1..2943: mol_type = other DNA
atggcatttcgatttggagtccttattcttgctttgctaatttgtttgagtgtcaattctgtggaatctgatgatggagcaacgttgttagagataaagaagtcattcagggatgtggataatgttctctatgattggactgattcaccatcatcagattattgtgcctggagagggatagcatgtgataatgtcaccttcaatgttgttgcactcaatctttcagggttgaatcttgatggtgaaatttcacctgcaatagggaaacttcacagtttggtctctattgacctcagagaaaacaggttatcagggcagataccggatgagattggtgactgttcttctttaaagaacctggacttatcatttaatgaaattagaggggatataccattttctatttctaagttgaaacagatggagaatctgattttgaagaacaaccagttgattggaccaattccttcaactttgtctcaaattcctgatttgaagattctagacctggctcaaaataatcttagcggagaaataccaaggcttatatattggaacgaagtattgcagtatctaggcttgagagggaacaatttggttggttcactatcaccagacctgtgccaattaactgggctgtggtattttgatgtgagaaacaatagcctgacaggaagtattccagagaacataggcaattgtactgccttccaggtcttggatttatcctacaaccaattaactggagagataccattcaatattggattcttgcaagtagcaactttgtcgttgcaaggcaataaactctctggacatattccatcagtgatcggtctcatgcaagcacttgctgtcctagacttgagctgtaacatgttaagtggaccaattcctcctatcttgggcaatttaacttacacagaaaaattgtacttgcatggaaacaagctgacgggcttcatccccccagagcttggaaatatgtcaaagcttcactatttggaactgaatgataaccatttaagtggacatatcccacctgagcttggaaagcttactgatctgtttgacttaaatgttgctaacaacaatctcaaggggccaattcctagtaatcttagctcatgtaaaaatctcaacagcctcaatgtgcatggaaacaaattgaatggatcaattcccccttctttgcagagcttggagagtatgacctctttgaatctttcttccaacaatcttcagggtgcaattccaattgaactgtcacggattggtaatttggatacattggatatttcaaacaataaattagttggttccatcccttcttcccttggtgacttggaacatcttctgaagttgaacctgagcagaaacaatttaacaggagttattccagcagaatttggaaatcttagaagtgttatggaaattgatctttcagataatcagctctctggcttcattcctgaagaacttagtcagcttcagaacatgatatccttgagacttgaaaacaacaaattaactggcgatgtggcgtcactttcaagttgtcttagtctctctctacttaatgtgtcctataacaaactatttggtgttatccccacgagtaacaactttaccaggtttccccctgacagtttcattggaaaccctggtctttgtggtaattggctgaatttgccatgccatggtgctcgtccttcagagcgagttacattatctaaggctgccattcttggaattactttgggtgcccttgtgattcttcttatggtattggtggcagcttgccgaccacacagcccctctccttttcctgatggatcatttgacaaaccaattaatttctcccctccaaagctagtgattcttcatatgaatatggcactacatgtgtatgaagatatcatgaggatgactgaaaacctaagtgagaagtatatcattggatatggtgcatcaagtacagtttataaatgtgttcttaagaattgtaagccggtggctatcaagaggatctattctcactatccccaatgtattaaagaatttgaaactgaactcgagactgttggcagcatcaagcaccggaatttggtcagtctccaaggctactccttgtccccatatggccatctcctgttttatgactacatggaaaatggcagtctatgggatcttcttcatggacctaccaagaagaaaaagcttgactgggagctgcgtctaaaaatagcacttggagcagcacaaggacttgcttatctacaccatgattgctgtcctagaatcatccacagggatgtgaaatcatctaacattatattagatgcagactttgagcctcatctcactgattttggcattgccaaaagtctctgcccctcgaagtcccatacttctacttacataatgggcacaattggctacatagaccctgagtatgctagaacttcgcatctcactgagaagtctgatgtgtacagttatggtattgttttactcgagttgctaactggaaggaaagctgttgacaatgaatccaacctccaccatcttattttgtccaaggcagcaaccaatgctgtgatggaaacagttgatcccgacattactgccacatgcaaggacctaggagctgtaaaaaaggtttatcagcttgctctattatgcacaaagaggcagccagctgataggccaacaatgcacgaagtgacacgtgtactcggaagtctcgtgccatcaagcatcccaccaaagcaactagctgacctaccacctgcttcaaatccatctgccaaagtgccatgctacgtggatgagtatgcaaacctcaaaaccccacacttagtaaactgcccctcaatgagcacttcagatgctcaactcttcctcaagtttggagaagtaatctctcaaaacagtgagtga

SEQ ID NO: 168 - DNA, 1455 bp, Glycine max
  source 1..1455: mol_type = other DNA
atgggtttggtggggattctgtgttcttccgtatttctttttatcccaattgagtctactggcagagcagagaatatgtggcaccaaaacttaggtctggattccacgggaactgtgagttttacagtcttcagtgcaagtatatttgtgcttgttgctcttgttctctccatgtatttagtattcgagcatttagccgcctataatcaacctgaggaacagaagtttttgattggtctcattttaatggttcctgtttatgcattggaatcgtttttgtcagtattgaattctgatgcagcatttaacagtgaaattatccgagaatgctatgaagcttttgcattatactgctttgagagatatttgatagcttgcttaggtggtgaggagaaaacaattcaatttatggaaagcatgagccgaacagaatccattattcctcttctgaaagaagcatatgcatatggagttgtagagcatccatttcccttaaatctcttcttggaggattggaatctaggccctgagttctatcaatctgtgaaaattggcattgtacaatatatgatactgaagatgatttgtgcaatagtggcaataattcttgaatcttttggggtttatggagaggggaagtttgaatggaaatatgggtatccttacttggcactagttcttaactttagtcagacatgggccctatattgccttgtacagttctatgctgttattaaagataagttgaaaccaatcaaaccattggcaaagtttctaacttttaagtcaattgtcttcctgacatggtggcaaagtgtggctgttgcatttcttttttatatgggagcttttaggggatcattggctcaggagttgaaagcacgtattcaagactacatcatctgtatagagatggctgtggctgctgttgtgcacctttatgtcttcccagcagaaccttacaaaatgggagagagatgtattcgtaacgtagcagtgatggacgactatgcatcgctaggatcgcctctggaccctgaggaagttcaagacagtcagcgctcaacaagaacgtggctgggagcacataataatcagagagaaaaaaatcctatgaaatttacccagagtgttcgtgatgtggttgttggaagcggtgaaattattgttgatgacatgaagtttacagtttcacatgtggtggagcctgttgaaaggggcattgcaaagataaacaaaaccttccatcagatatcagaaaatgtgaaacgccacgagcagcggacgcgaaataccaaggatgattgttaccttgttcccttgcgaacgcaaatgtcagaattttctgacgttcacgatactatgggtgaaggaagtgtcagtgatagtggtatgtccagggtaaagcgacagcatttccaatgcaaagcagcagccaccagaagtagaagatag

SEQ ID NO: 169 - DNA, 3036 bp, Glycine max
  source 1..3036: mol_type = other DNA
atgaggattgagcaagagagtgatggagtctccgcttccacaaagcacctcgtctttgcgtattacgtcactggtcatggttttggccatgccactcgagtcactgaggtggtgcggcatttgattcttgcggggcatgatgtgcatgtggtcactggtgctcctgatttcgttttcacctccgaaatacagtctcctcgcctaatcattcgtaaagtgcttttggactgtggagctgttcaggctgatgctttgactgttgatcgtcttgcctctttggagaagtattctgaaacggcagtaaagccccgtgctcaaatcttggctcaagaaaccgagtggctgaactctgtcaaagctgacttagtgatctccgatgttgtcccagttgcttgtcgagcagccgcagatgctggcattcgctctgtttgtgtgaccaatttcagttgggacttcatctatgcagagtatgtcatggctgctggacttcatcatcgttcaatagtttggcagatagctgaggactattctcattgcgagttccttatccgtctcccgggatattgcccaatgcctgcttttcgtgatgttattgacgtccctctggtagtgaggaggttgcacaaatctgcaaaagaggttaagaaggaacttggggtaactgatgatgtgaagctagttattctcaactttggtgggcagccatcagaattgaagctaaaggaggaattcttaccacctggctggttgtgcctggtttgtggtgcttcagagactgcagaccttccgtctaactttgtaaaacttgctaaagatgcatatacgccggacattattgcggcatctgactgtatgcttggaaaaattggatatggcactgttagcgaggccttggcatacaagtgtccttttgtctttgtacgtagagactatttcaatgaagaaccttttttgagaaatatgcttgagttttatcaaggtggtgttgaaatgattagaagggatttactgactggtcactggagaccttatcttgaacgtgcaataagtttgaaaccctgctatgaagcaggcattaatggtggtgaggtggcagcccacatcctgcaggagacggcttttggaaaaagttatgcatcagataagcttagtggagcaagaagattgcgtgatgccatagttcttgggtatcaactccaaagggcccctggtcgagatattacaatcccagaatggtatgcaactgctgaaaaccaacttggccgctcaacacctggttcccctatggatgatggaagatctgcgtttagcttggacattgaaaactttgacattcttcatggagatattcaaggacttccagatactgtggcattcttacagaacctatctgaattacaggacaagcacactaggcgggagcgtaaggctgcggctaatctcttcaactgggaggaagaaatatttgtaacaagagctcctggaaggttggacgtcatgggtggtattgctgactattctggaagtcttgtcctgcagatgccaattaaagaagcctgtcatgttgctttacaaagaaaccatccaagtaaacataggctgtggaaacatgctgaggctcgacagaatgacaaagggggaaatccaactgctgtcctgcaaatcgtatcatatggctcagaattgagcaatcgtggcccaacatttgacatggatttatctgattttatggatgaagaagacaaaccaatctcatatgaaaaagcaaagaaatactttgctcaagatccttcccaaaagtgggcagcatatgttgcaggggcagttttggtcttaatgactgaaatgggtgttcaatttgaagacagtatcagcatgctggtttcatctgcagtcccagaaggaaaaggcgtatcatcttctgcatctgtggaggttgctagtatgtatgctattgcagctgctcatggattaaatatcagcccaagggatctggccatactctgccagaaggtggaaaaccacattgtaggcgctccatgcggtgtcatggaccagatggcttcttcatgtggtgaagccaacaaacttcttgctatgatttgccagcctgcagagattgttggccttgttgacattcccagccatatccgtttctggggaattgattctggaataagacacagtgttggaggtgcagactatggttctgttagaattggcgcctttatgggtatgaagatgataaaggccaaggcatctgaggaattgtctgagagttgggctgctaatgggttgaactatgatgaagtggaacaagatgacatagaactacttaaacaagaaacctccttagattacttatgtaatttgccacctcatagatttgtgactctttacgctaagacaattcctgagtccattgtcggtgagacatttttggagcaatatcaaaatcataatgatcctgtcactactattgatccgaagcgtacttacggagttagagctcccacaatgcatcccatatttgaaaatttccgagtcgtgaccttgaaagcacttttaacatctgcagcttcaacctatcagcttacagccttaggagaattgctatatcagtgccaccacagctacggtacttgtggacttggctctgatggaacagataggcttgtaaacttagtgcaagagttgcagcacagtgcagcatctaaagctgaaggtgggacattatatggagccaagataactggtggtggttctggtggaacagtttgtgtagttggcagaaactgtctcaagagcagtgaacacatttttgaggttcaacataggtacaagaaagctactggttatttgcccttcatttttgaaggttcatcacccggtgctggaaaatttgggtacctaaaaatacgtcgtcgagctaccccaaaaaaggctaacgccatcaaggatgatggtgcactcacctcagaaaaggctaacgctagcaaggatgatggtgcagtcacgatgaagaataatagttaa

SEQ ID NO: 170 - DNA, 813 bp, Glycine max
  source 1..813: mol_type = other DNA
atggaggagtcttcttacaccggtcttcccactagccatttgctcggttcagttcctgctgtcgtaactgaagaaaataatgcctcgaaacatgtggccaccagtgcgaatatgcaaatattccctcccaacaatggaggagacagaggaccgggatataatactcttggcagtcctactgaagcctttgaacaacagccagcaaacaactggaggggagtctttagcatctcatcttattcagagtatttcaatgtagacacggatgttgtcctaatcagattgataagttccttgaatccagttgctggagactttttcagcaagatagatgctaaccctgatttatatgggcttatatggatctcaacaacattggtttttgtgcttgcctcgcttggaaatctggccacgttccttatgcaaaaacatgtggataacagtacctcttggagctttgacgtcagctatgtgaacgtggctgcatgctctatctatggctatgcgattgtggttccattggcatactacttcttccttcagtatatgggttcaaatgctagccttataaggttttggtgcatgtgggggtattccctcaccattttcatcatgtcttccttcctgttgataattcctgttgagtttctacggtgggttataatcctccttacgggcgttgcctcagcaagctttgttgccttaaacctgaggtcttacatagaaggcaatgaactttcagtggcgattattgcagcatttttcttgcaaatggctttggcaatcttcatcaaggtttggttctttccgtag

SEQ ID NO: 171 - DNA, 456 bp, Glycine max
  source 1..456: mol_type = other DNA
atggggaaccaataccaccaagcaacagatgggcttctgagccttttcacaaaggccaatcacgatctctctatggttcaccatcgtctcgaaaaggagtttcagcaggtttaccccgacaacgctaaccctatgaagctggtttctagaatcaagaaggttcaggaagatatatcaatcttgaaggggcagtgccatgagcttctggctgctaagcaggatttgattgataaggctcagagagttctggtggagaacagaaatttggtgcagcgtatgcagccgtccttgggcatctccccctctggtgaagatgatgctgcattcactaacttcaaacaggtaattgaagaatggacagcacaagtaagatccaaaacagggaatgaaacacatgatgctgattctggatatatcaacaaactactattctcagctatagttcaaagtaattga

SEQ ID NO: 172 - DNA, 1119 bp, Glycine max
  source 1..1119: mol_type = other DNA
atggcagattcgatttgcagaacactgagggatggagcgttggagggagagcttgcgcctactctcacaatcaaggactccctcgcttcacctttcgcctttcatgtattctctcacattctacttcaactctcgtcacacgtcatagcacaaaaatctcagtcacagggtatagtaattgttgcactttctcggagtccatcgtcttacacttctttgttaaagatgaatgggatggatgttgcttcttctaataaatggattcacattttggattgttatactgatcctcttggttggaaggataaggcaaggaaatctgcgaatgttacggatccttctcagcaaatttcgcttgctactagttcttataaaaccgtgaaggacgtggataagttgtttttggtaattgctgaacttggcagaggattggttggagaaaataaagcccgcttctgtgttgccatagactcgctaagtgaacttttgaggcatgcatctatgcagtcagttgcaggccttctaagcaatctgcgtagccatgatcaaatttcaagtatgtttggattattgcattctgaccttcatgaggagagggctgtggcagttcttgagtacatgtcctccatggtggccagtgtagacccgtatcatcattctgcagatggtcagaaaaggtgtttagagagttccttatatgatcaaaactttaccaagggaaaatttaatgtcaggttcaaacgcagaaatgggcgagttagagtaacgtgtgaagaatttaaagttgagcctggaggaatcagcttctcacctgtttcatcagtagatggaaccgtcattgcaggcatagtgccaaaggtgcagttcaatctccagctgtcagagaaggagcgaattgataggtcaaatgttgtgcttccttttgaacaccagggaaatggtaaaccaatacaaatttatgatggtagaagatcccttgaagagagcaacagtgaggcaaatcccatttcaagtggtaaaaaggaggattctgccatgggtgaaattatatattttcgtgattcagatgatgagatgccagattctgatgaggacccggatgatgatttagatatatga

SEQ ID NO: 173 - DNA, 2598 bp, Glycine max
  source 1..2598: mol_type = other DNA
atggggggtataggtaagacaactgttgctaaggtcgtatatgccaaactatgttatgaatttgaaagttgctgcttcttggaaaatgtaagagaacaatcgcagaagcatggattaaattatttacatgataagcttctctttgagctgctgaaggatgaacctccccacaattgcacagctgaagttgtaggatccaaatttgttttgagaaggctcgcaaataaaaaagttttgattgtgcttaatgatgtgaatggttttgagcaattagaatatctggcaagagaatttgtctgcttgggaccaggtagtagagttattataactacaagagataagcatttgcttattcgaagagttgataaaatacatgaagtcaaggaattgaactttcaagattcccttaagcttttcagcttagttgcattcagagacagcaatcctcaaatggaatatatagagctatcagaaagggcagttgcatacaccaagggcaatccattagctctgaaagtgtttggcttcacttttccattccaaaatatagaagtatgggaaagtgcacttagcaaactcaagaaatatctgaacgtacaaatccaaagtgtgttacaattgagttatgatgaattagatgatgcagagaagaacatttttctagacattgcattcttttttgaaggggaaaataaagatcatgtcatgaggctactagatgcctgtggcttttatgcaactatcggaatagaaacccttcaagataaagctcttgtaactatttcaaaagacaataaaatacacatgcatcagctgattcaagaaatgggcataatgttagacatgtctcaaattagggaacttcacttgagtgctgacatcttcaaaaagatggctaaactaagattgctcaaattctattctcccttcaatgggagatcatgcaaaatgcaccttccgacaggtcttgagtctcttccgcacaaattaaggtatcttcattggaatgaataccctttaatgtccctaccatcaactttctctggcgagatgcttgttcagcttcgcatgccacgtagccatgtaaaaaaactttgggatggattacaggatttcgtcaacttaaagggaatagaccttactgcttccacgcagttgatggagctcccagatttatccaaggcaacaaagcttgaaatacaaaatatcgcccactgcgtaaatttgagtcatgttcatccctctattttatcacttgacacccttgttgattttgttctatatggttgcaagaaactcaagagtcttcatttaagatctgtcaagtacattgtgctcaatggttgcttcaatctacaggaattttcactgacctcaggagaaataaacgtgttgaacttgagagggacagcaatcgaaacattagcatttccatcggtcgtctcagcaaaattgaaaagctctctgtctgagttaccttctctgacatgtcttagtgaactgaatcttcataactgcagacagcttgacatgccaaatttgcataacctacttgatgctttgcgttctgtcagaaagctgattttggacgagtgttgtaacttttctcgagtcccttgcaatatcaagcatctttggtgccttgaatacctgtccctaagggattgcacggggcttcgatttatacctcagcttccaccatctgctgaacatttagatgccatcaactgcacatccctggagacagtgttgcctctaatgcctttgagacaaccgggacaaaatgacataagcatttcatttgaaaactgcttgaaattagatgaacattcaaaatatggtattacagaatatgccaacttcacaatgaagcatgttgcatatgcaaatgacagtggttcacatcatctgaataaaagaggtggtgctgtttgttttccaggaagcaaggtgccagagtggtttgaaaacaggacaacaacaccggcttgtgtaactgttcaacttcctccaccttcccacttgttgggttttgctttctgtgttgtgctttctcagtttcaatcaaatgctaagtatgagtatcatcagatagtatgtcgatggtgcttggaagatgaaagccaagccaatggtgatgagtgcatcactgaaaacctaaatgtgtcatttgaattctatgttgaaactcataggttttgtgagcctaaacacattggcttgattggtatcaaagagtgtggggtctgtccaatatatacttcaaaatattacagtttcacaaaccacagagaactggacatgatgttggaacatagagctagagagattacaatggcggtaggaaactctgatgagacagaaagcaatggcataaaaactcaggaggctaaaaagctggatgaaaaggttcgacgttttgttgaacaaaagataggtgttgagatgaaaaaggccaaattgaaggagaccctggaaagatgtgaaggtcaaaaaatgaaactgaaacaagaggctaagaacaaggtaactgaaagtaaagagataatgttggcgattgagaattctaaagccttgtatatgattctgccctgttga

SEQ ID NO: 174 - DNA, 1533 bp, Glycine max
  source 1..1533: mol_type = other DNA
atggttgtcttttcgcgaagtgcgacgacgacgacgacgacgacgacgacctatacgcgccacaacttaatacgacaaacgaaaacctgcgttggcaataataatagtttgtgttactctgctactaagaaggctcctccgcagaggattcttggtggtgcacgtggcagtagagtgattcttgctgccagccctcccaccgaagacgctctagttgccactgaccctctcaccaagcaggatcttgtcgattatcttgcctctggttgcaagcccaaggataaatggagaataggtactgaacatgagaagtttggttttgagtttggaaccttgcgtcctatgaagtatgaccaaatagcagaattgctgaatggcattgctgagagatttgactgggataaagtaatggaaggtgacaaaattattggactcaaacaggggaagcagagcatatcattggagcctggtggtcagtttgaacttagtggagctcctcttgaaaccttgcatcagacttgtgctgaagttaatttgcacctttatcaggttaaagctgttgccgaggaaatgggaattggatttttggggattggtttccagccaaagtggggaatcaaagacatacctataatgccaaagggaagatacgacattatgaggaattacatgcctaaagttggctctcttgggcttgacatgatgttcaggacatgcactgtacaggtcaatctggactttagttctgaagctgacatgatcaggaaatttcgtgcaggtcttgctttgcagccaatagcaacggctctttttgcaaattcaccctttaaagagggaaagccaaatggttttttcagtatgagaagccatatttggactgatactgacaaggatcgcacaggcatgctgccttttgtttttgatgactcttttgggtttcagcagtatgttgattatgcacttgatgttcctatgtattttgtctatcggaaacacagatatatcgactgtactggaaagaccttcagggacttcttggctggaagacttccttgtattcctggtgaattaccaactctcaatgattgggaaaatcacttgacaactatatttcctgaggtcaggctgaagagatatttggagatgagaggtgctgatggagggccttggagaaggttatgtgctttaccagcattttgggtagggttattgtacgatgaagtttctctacaaagtgttttggatatgacagctgattggactccagaagaaagacaaatgctaaggaataaggttcctgtaactggtttgaagacaccattccgagacggtttgctgaagcatgttgctgaagatgttctaaagttggcaaaggatggcttggaaagaagaggcttcaaggaatcaggatttttgaatgaggttgccgaggtggttagaacaggtgtcactccagccgagaggcttttggaattgtatcatggaaagtgggagcaatccgtcctggtggtctctcatttgagcttcatttag

SEQ ID NO: 175 - DNA, 2754 bp, Glycine max
  source 1..2754: mol_type = other DNA
atggcgaaatcggggaataagaatatgggatcaaccgcaaaagttcacccttccaatgatcccgaactggatcccgattcttacgccctggagaagttcagactctacgaaaccagagcgaggttttacctaatcggaagtgatcggaacaagaggttcttccgggtattgaaaatcgatcgatcagaggcgtgggatctgaatatcagccaagaccccgtgctgtattctccccaggaaatcaagagcttgctccagcggattgctgaaggtaacagggccactggcggcctcacttttgtcgctaaggttttcgggattgctggctgcatcaagtttctcgagtcctactatttgattctcgttactaagcgcagacaaattggttctatttgtggccatgcaatctatagcatcaaggagagccaattgagaaccattccacatgtttcaattcaatctgatttagctcactctaagactgagctcaggtacaaaaagcttttgtctagtgtcgatttgaccaaggattttttctttagttacaattaccctataatgcaaagtttgcaaaagaacgtctcctccggctccagtcaagaggaagggatgtcatatgataacatttttgtttggaatgcgtatttgacacaagcaatcagatcaagatgcaataacaccatttggaccgtagctttagttcatggtcattttaggcagattcgtctatcaatttttgggagggacttcagtgtttccttgatttctagacgatcgagacattttgcagggacacgggaaggattgaaggaggaggagcaacaaaccaggtacttgaaaaggggtgtgaatgatcgggggagggttgctaatgatgttgagacagagcagattgtccttgatgaagaatcaggctcttgcaagggaaagatgagttcagttgtgcaaatgcgtggatcaataccgcttttctggtcacaagaagcatcaagatttagccccaagcctgacataattttgcagaggtatgatccaacataccaagcaacaaaattgcattttgaagacctagctaagagatatggcaatccaattattgttcttaatttgattaagacagttgagaagcggcctcgagaaatgatgctgaggcgtgaatttgcaaatgctgttgggtatctgaaccaaattctaccagtagagaaccatcttagatttattcactgggattttcacaagtttgcaaagagcaagtctgccaatgttttggcagttttaggaggtgtagcaagtgaagcgcttgatttaactgggttttactacagtggtaaaaccagcatcattaagagagccaacaagagcaatagaacaagcacagggagggatacttcagtgagagatctgagagctagttctggggatcttgtgaggattgggaacagtaatgaaatgctaaattctgtggttaatcaagacaaagagactgacatgaaccatcagaataaaaaagataattttggtagtgatgcaccacattttcagagtggagttctccgtaccaactgcattgactgcttggaccgtacaaatgttgcccagtatgcttatggtcttcaagctttaggacgccagctccatgcaatgggtttgactgatgttccaaaagtggatcctgatagtagtattgctgcagctctaatggatatgtatcaaagtatgggagatgctcttgcccagcaatacggtggctctgctgctcacaatactgtgtttccagagaggcagggaaagtggaaagcaacaacacaatcaagagagtttttaaaatccataaaacgatactatagcaatgcttacacagatggtgaaaaacaagatgcaataaacttgtttttaggttacttccaaccacaggaagggaaacctgctctctgggagctggattcagattattatctccatgtatctgggattggggatgatctaattcctgagaagtgttctgaaccaaatatcagtccttctggaagaggtggaatggtattcatgcctataccagcttgtagagatgacttctctcgcataaagttgacatcatttgacatgttaattgaaaagacttgtagtaaaataaaaaatgtaagactatgctgtgagcctgatcagagaccaggtggggtttctggaaacagtggtgtagcacctgatgcagctgagatacagctcaaaagcccaaattggcttttcggccagagaaagtatgaagagggttcctctgctgcaaaagttgcttctcgtgaatctggcgttgaaggatctcatgctaatggcttttgtgacttgaattggctttcctctggcaatgatatgaatgaagaggatgttttccaaaggtaccttacaatgacctcaacaaatgaggccaacggttggtatggaggtagtctcctcggcgatcaagatgaaagcagtgagatatataaacattatgctgagttatgtcagggacctgccttggaacttttccaaaatgaccctgagaaggagcaacactatgcagatgctttaagcacgagttcatatgaaattgttaatgatgctgtcgttgcagcagaaatggaagcagctctaaaggagtatgaccaagttggtgctgaccttgggattattcccaaatcttgtaaattctatgttgatgatccgagctggctgacgagatggttaactggggacgaaaaagtaccaaggatatga

SEQ ID NO: 176 - DNA, 708 bp, Glycine max
  source 1..708: mol_type = other DNA
atgggggatagtcagtattcgttttctctcaccacatttagcccttctggaaagcttgttcagattgagcatgctttgacggctgtcgggtctggacaaacgtctttgggaattaaagctgcaaatggagttgtcattgctaccgagaagaaattgccatctatattggttgatgaagcatcagtccagaaaattcagttattaacaccaaatataggtgtcgtatatagtggcatgggtcctgattttcgagttttggttcggaaaagcaggaaacaagccgagcagtatcaccgtttatataaagaaccaattcctgtcactcaacttgtcagggaagttgccgccgtaatgcaggaatttactcaatctggtggtgttaggccttttggagtgtccctgctagttgctgggtttgatgataacgggccacaattatatcaggtggatccatcaggttcatatttttcttggaaagcttctgctatggggaaaaatgtttctaatgcaaagacgtttcttgagaagaggtacacagatgatatggagcttgatgatgcagtccacacagcaatattgactctgaaggaagggtttgagggacagatctcaggaaaaaacattgaaattggcataattggagctgataaaaagttcagggtattgacaccagctgaaattgacgactatttggctgaagtagaataa

SEQ ID NO: 177 - DNA, 1836 bp, Glycine max
  source 1..1836: mol_type = other DNA
atggctgaaccatcatctaatgttgcaacaaggaaaaataggacagaccctggttggaagtattgtcatccacttgtggaaggggatacaaacaccattgtttgtaattactgtggaaaaatcacccagggaagaataacgagagccaaagaacacttgattgggaagtcgagtaacgttgcatcttgcaagaaaactccaccagatgtagtcgaagagctcaagggatatatggctaacaaaaaaagtggggccacttacaatagtactggtggtggtaatatgccaaatataagagattttgaatttagtgaaccaattggatgtgatgaaagtgaagatgagtttgaggactctagcaatgctactgctttggccgcaaagacaaagtgtgcatcaatatattgctcgctttggtaccaagctggtttgtcattcaacctcattaaattgaaaagctttgagaatatggttgcagtcattggtcaatatgggccacgtttgcccattccaagctatcatgatatcagagttccactcctgcaaaaggaagttgaatacactgaaaatttgatgaaaggccatagggagcaatgggtcaaatgcatcattaattttttggttaactctcaagctggtacaatgtttttgaagtctgttgatgactctgattttgtgaagacaggtgaaaagttttttgagttgcttgatgctattgtggaggaagttggagaagagaacgttgttcaagttgtaactgataatgggagcaactatgtcttagcgggtaagttgttaaaggacaaaacgaaacatatttattggaatccttgtgcagcccattgtattgatttgatgcttgaagacattgggaagcttcccttgataaggaagacaattagaagggcaattaatccagttgggtttatctatgtccattctagtaccttaagcttgttgagaaattgtacaaacaagagggaattggtgagacatgctattactagatttgtcacttcttatctaaccttagaatggttccacaaagagaaagttaatattagaaagatgtttatttctgatgaatggatcttaagcaagttatctaaggagcctaaggggaaagaagctgcaaaggtagtgcttatgccttctttttggaatagtgtggtttacactcttaaagtcatgactccacttgtcaaagtgcttcgtcttctggatggtgaaaggaaatcaaccatgggctatatttatgaagcaaaggacaaggcaaaagaaacaactataagtctttcaacaacaacgaaagcaactgcctacttcttaaattcagagttattttatgacaacactgatttggagtttgattttgaggtcaccaatggattgtttgatagcattaagaagttggttccacaatttgatgtgcaacagaaaattctaactgagttgcatctttacaagattggtgctgaacactttggttcttacatattgtggcaaatgtttgggtcacaaactccaaatttgcaaaagctagctattaaaattttgagtttgacttgcagtgcttcaggatatgaaagaaattggagtgtatttgagcaaattcattccaaaaaaagaaataggcttgagcacaagaagttgcatgatttggtgtttgtcaaatacaaccaacaattgaagcaaagatataatgcaagagatgaaattgatccaatttctcttaatgattttgatgcgtctgatggagaagagggagagggaaatgctccactaccttatgataacaatgaagaggattatgttgggattggagaagatgattag

SEQ ID NO: 178 - DNA, 948 bp, Glycine max
  source 1..948: mol_type = other DNA
atgtcgaacgatattggattcttcgacctcaacacgggcgccaagatcccttccgtcggcttgggaacatggcagtccgatcctggcctcgtcgctcaagccgtcgctgcggccatcaaggttggttaccgccacattgattgtgctcaaatctatggcaatgagaaagagattggctctatgttgaagaagctgtttgaggagggtgtagtgaagcgtgaggatttgtggattacatctaaactatggaatactgatcatgctccagaagacgtaccactggcattggacaggaccttgaaagatttgcagcttgattatgtagatttgtatcttatccactggccgacttcaatgaagaaaggatcagttggctttaatcctgaaaatcttgtgcaaccaaacatacctaacacatggaaggcaatggaagcactctatgactccggcaaggctcgggctataggtgtaagcaatttttccaccaagaagctggcagatttgctggcaattgctcgtgttcctcctgctgtcaaccaggtggagtgtcatccttcatggcagcaggacaagctgcaggctttctgcaattccaaaggagtccacctcactggatattcacctttgggttcccctggaacaacctattttaaaagtgatgtccttaagcacccaattataaatatggttgcggagaaactaggcaagactcctgcacaagtagctcttcgctggggactgcaaatgggccatagtgttcttcccaagagtactaacgaaacacggataaaggaaaattttgatgtttctggctggtctatacctgaggacttccttgctaaattttctgagattcaacaggcaagattactcaggggtaccacatttgtccatgaaacttacggtgcctacaaatctgtcgaggaattatgggatggtgaaatctaa

SEQ ID NO: 179 - DNA, 2703 bp, Glycine max
  source 1..2703: mol_type = other DNA
atgtttggcgggggcggcgtgagcgtgagaatggaggaagaggaagatgaagtgggttggtgttcttctgattcgggcgacggcgacggcgacgacgatatcgaagaacggaaatctcagaatgtggatgccttgctcaggggcaacctcgtggtgaagagacaatcgttgctaccgcgacttctatcagtatcgcaaggagcggctgtttgtagaaagcccttcaaacctccatgctctaaatctcatgcttccgcttccgcttacaatcaagaccttactcgtaagctttccgctcgcaaaaggtttgttccctggggttcttcctctcctattcctattcctattcctagatctgccttagacgactccaaattcaatgttgctgttgctgttgctgatgttgccgaggagctcaagccctctttgcctcccgggattgatcctctcgtattgtggcaccctcaagactccgaggatgctaatgctaattttaccaccataacagttgatccgttgcttgttcgttaccttcgtccacatcagagagaaggggttcagttcatgtttgattgtgtctcaggcctgtctaccacaccaaatatacacggatgcattttggcagatgatatgggtttggggaagacattgcagtccatcacattactatatactctactttctcaagggtttgatggcaaaccaatggttcgaaaggccatcattgtgactcctaccagccttgtcagcaattgggaagctgaaatcaaaaagtgggttggagaaagagttcctcttgttgctttatgtgaaagcacaagggaagatgtcatctccgggattgacaattttacaagtcccaaaagcaatttacaggtgctcattgtttcatatgagacatttcgaatgcattcgtcaaagtttagctctactgactcatgtgaccttctcatatgtgatgaggctcacagactgaaaaatgatcagacgataacaaaccgggcattggcagctctcccctgcaaacgtagaattttgttgtcaggaactcccttgcaaaatgacttagaagaattctttgcaatggttaactttactaatcctggaatattgggtgatattgcacattttcgacgttactatgaggcaccaattatttgtggaagagaacctgctgccactgcggaagagaagaagctaggtgctgaacagtctgctgaattaagtgtcaatgttaaccggttcatattaagaaggactaatgcattgttatcaaatcacttaccacctaagatagttgaagttgtttgctgcaagttgactccactgcagtcagaactatataaacattttatacaatccaaaaatgttaagcgggcaattactgaagaactgaagcaatcaaagattttggcctacataacagctctcaaaaagttgtgcaatcatccaaagcttatctatgataccatacgaagtgggagtccaggaacttcaggttttgaggactgtatccgattcttccctcctgaaatgttatctggaaggtctggctcatggacgggagggcatggagcatgggttgagctatcagggaaaatgcatgtcttagcaagattacttgctcatttacgtcagagaacaaatgaccgcattgtccttgtctcaaactacactcagactcttgatctttttgctcaattgtgtcgggaacgaaggtaccctcacttgaggcttgacggctctacatcaatcagtaaaaggcagaagttggtcaactgctttaatgatccatccaaggacgaatttgtttttctcttaagcagcaaggctggtggttgtggactcaatttgattggtggaaatcgacttgtcttgttcgatcctgattggaatccagcaaatgataaacaagctgcagcaagagtatggagggatggacagaagaaaagagtttatatttatagatttttgagtgccggaacaatagaagaaaaggtctaccagcgtcagatgtcaaaagaagggctgcaaaaggtcattcagcaggagcaaaccgatagccttgtggcacagggtaacttgctttcaacagagaatcttcgtgatttgtttacttttcatgagaacatcaagtcagagattcatgaaaacatgcaatgcagtcggtgtcaaacatttgatggccctcgaagcactgaagcccagtcaacaataacagatagtgaatctgatgaagaaacctctgatattggtggatttgcagaaattgctggctgcttacagaatttaaaaagatcagaaaagcaggtagggagcccattggaagaggatttaggaagttggggccatcattttttcccaacttctgtaccagatgctatacttcaggcttcagccggtgatgaggtgacttttgtcttcacaaaccaagttaacggacgactggtacccgttgaatctattatgagtcccaagctacagcaaaaggatccaaaaaaggaattactcaagtcaaagcagaacggtaaacaaaagcccactcctttttctttacataacagactaccattgcagtctgcttctgtcgggattactaaaaacatctccatgaatgtggcatttaagccccagtactctcttgtaagtaaagtattgccacagaaaagatcatgtcctgctaatatcaatgatgataattga

SEQ ID NO: 180 - DNA, 1674 bp, Glycine max
  source 1..1674: mol_type = other DNA
atgtattctcattgggttcgattgatgcttctcattgcttgcattcttccagctttggttgaatgcagagagaggcactacaaatttcatgtggtgtctaaacaaactaatagattgtgttcaagcaaatctattgtcaccattaatggcaagttcccgggacccactctttacgccagggaagacgatacagtgttggtcaaagtcatcaaccaagtcaatcacaacgttaccatccattggcatggtgtgaggcaattgagaactggttgggctgatggacctgcatatgtcacacagtgcccaattcaaccagggcaaacctatgtatacaatttcacactcacagggcaaagaggaacacttttgtatcatgcacatgttaattggctaaggtcaactctctatggtgccttggttatcttgccaaagagaggtgtgccttatcctttcccaaaaccagatgatgagttggttgtggtattaggagaatggtggaaatctgatacagaagctgtaatcaatgaagcactcaaatcaggattggcaccaaatgtctcagatgctcatactattaatggccttccagggacagttaccaattgttctacacaggatgtttacaacctgcccgtggagagtggaaagacctaccttttgagaattgtcaatgctgcactcaatgaggagctattcttcaaaattgctggccacaagctcactgtggtagaagttgatgccacatatgtaaagccattcaagattgaaacaattgtgatagcccctggccaaaccaccaatgtgcttctaaatgcaaaccaaaaatctggcaagtacttggtagcagcttctccattcatggatgctcctgttgctgtggataacttgactgccacagcaacattgcactacaccggcactcttgctgccacccccacaattctcaccaccccacctcccaaaaatgctacccaaattgccaacaacttcatcagctctcttagaggccttaactccaagaaataccctgtcaatgtcccattaacagttgatcactcacttatcttcactgttgggttgggaattaacccttgtccatcttgcaaagctgcaaatgggagcagggtggtggctgctatcaacaatgttacatttattatgccaaccattgccttacttcaagcacattatttcaacatcaaaggggtgttcaccactgatttccctgccaatccaccccatgttttcaactactcagggcctggaccagccaatttgaacaccgaaaccggcacaaaggtttacaggctaccattcaacgccacagttcaggtggtgctacaagacacaggaatcattgcacctgagaaccacccagttcatcttcatgggttcaatttctttgttgttggaagaggagtaggcaatttcaaccccaaaattgaccctaagaactttaaccttgttgatcctgttgaaaggaacactattggagtaccagctggagggtggactgcatttagattcagagcagataatccaggagtttggttcatgcactgccacttggaggtacacacaacatggggactaaagatggcctttttggtggacaatggcaaaggtcctaagcaatcagtgatacccccaccaaaagatcttcccaaatgctaa

SEQ ID NO: 181 - DNA, 3441 bp, Glycine max
  source 1..3441: mol_type = other DNA
atgttggatagtgttctttccatcagagcaaagattgtagaatatattgtgggtccaattttacaccatgcaaaatacttgtgttgtttcaacaactttgctgtgaatctcccaaatgccaaaagagaattggaattgaccctagagcaagtgaaattacgagtaagagaagccactattaggattgaaaaaattgaaccaactgttgaagaatggctagaagaagttgagaaagttttagcagaagtgcagattctcgaaggaagagttttgaaagtgacaaagagtagtttcataaggcaatgtcgatattttcttgcaaaagaaatggtaagaaaaataggacaaatgaatcagctcaaatgtaacaagttagagcccttttctaggagtattaaccttccagacatgaagtactattcttccaaagattttgttctttccaattcaacagaatcaacttacaacaagcttttggaaacattaaaggataaaaatgttagcataattggactagttggaatagaaggctcgggaagaactactttggcaaatgaggtgggtaagaaagctgagaagttaaaactttttgagaaggttgtcatgacaacagtctctcaaaatctcaatatcataagcatccaagaacaaattgctgataagttgggtttcaaattagaggaagaatctgaggaaagtagagcaaaaacattatcacagagtttaagggagggcacaacacttctaatcttggatgatgtatgggaaaagctaaactttgaagatgtaggtattcctctcaatgaaaacaacaagagttgtgtaatactcctaacaactcaaagtagagaaatatgcacttctatgcaatgccaaagtataattgagctcaatcgtttaaccaacgaggaatcttggattttgttcaaattgtatgccaacataactgatgactctgcagatgctttgaaaagtgtggcaaaaaatattgttgatgaatgcgaaggattcctaatttcaattgtgactttgggaagcacactgaagaagaaaagtttaggagattggaagtcagcattgaaaagactacaagattccaaaccattggttattacaaaaggcctaaaaattcctcatgtatgtcttcaattaagttatgataacttgacagatgagttaaccaagtcgttgcttttgttgtgttctatctttccaaaggaccatgaaattgatttggaagatttatttcgatttggaaggggactgggactaactaagacttctgaaacaatggaaaaatcaaggagggagattgaaatagccgttaacatcctaaaggattcttgtttgttgttgaaagttagtaataaagaaagggtgaaaatgcatgacatggttcgtgatgtagccttgttgatggcatctgaaaggggtcaagcaatgttggcaagtactgcaatggatctaagaatgttggtagaggatgaaacactaaaagataagagagcaatatccttatgggatttgaaaaatggtcaacttcctaatgataatcaattgaattgtccaacactagaaattctcttgcttcattctccaaaggctggctttgaagtatctaatttgtgtcttgaaaggttgaaagttcttaaaatactgtcattcttaacatgtggctatacgtggaagttgcctcaatttagtccatcacagtacattttatcattgccacaatcaattgagtcattaaaaaatcttcaaactctgtgcttgagaggctataagctaggtgacatctcaattttggaaagtctacaagcactcgagattcttgacttgcgtggttcttatcttgaagaattgcctaatggaattgtagagttgaagaaattgaagcttttggatttgtacaattgttggattgagaaaaataatgcttacgaggttgtaggacgtttgcaactagaggaattgtattttcatttattctcgtataaagaagacattccacataatgtttctttctcaagattgcagaggtatgttattgtactagatcatagaccatattccttccacctaaagacagaaattatggaagagcatagaccatctagagctttgtacataaatggtttgaatgcatctactcaaaggtttatatcattaccaatcatggatctatttttaagagcagagtaccttcatttgaagcatctgaagggaggttacaaaaatcttatcccatccatggatcaacaaggcatgaatcagttgattgccttagtcctcgaatattctttagatatagaatacctctttgatagtactatgattaccaccaaagatgtttttttgtccaaacttgttactttaaggctaaatggcatgcatggccttcaagaagtgtttcatgaccagttttctctatgctctctggaaaatttacaagaactaatcatagaaaattgtgcacaattgtacagcatatcctttccaaggtactcaaatctatgcagtatcaaggtcttaagaatatacaattgcccagtcctaacttctctcttcatgccatccattgtcaaaactctagtgctgctggaggttctaaaaatatctgagtgccataaattgaagtacataatagaagaagtcaaagaagggactattaacagacaaaatcatgcatctatgacgctcccaaaattaagtatcattgatattgaaggatgtgaaaggttgaaatatatattgcctgtgtgttttctgggagagctagttagtttgcaaaggatgagcattcgaaaatgtagcgagttgatgtatgtatttggaaatgaaaaggagcatgatctttcagcgtaccaacaccagagtacataccattgcttgtatccaaatttgcttaatttggatactttcaagttggattccttaccaaatcttgttgacttttggcctgattactatcgtccgcgtttactaaatttgaaagagggtcgatgcactgagtgtcccggactgtccaattcagcgcgtaaggtggtgatttatttaaacttgcgccaagacataactgcaacggataaggtgggtaaagtctcctctcataggacacaacaggataccactataggcaagaccatcagacgtcttaagggaattttagatcgtgctgagatgacttcatcgactcaagaatcaattgctccttcttcatccaacaatgcctatgaggatatagctagagctcaacaacaagttgcagaagctcaaagggatgcatcaattgctcgacaagaaactactgttgtaagtcatctgcttgagcaaatgatggaaaagttggcagtaaatgagcagataggattacaaaagagggcccatgagcacgtgggactacagaggggaacccagcaagagccagaggaagatggataa

SEQ ID NO: 182 - DNA, 642 bp, Glycine max
  source 1..642: mol_type = other DNA
atgattaatttgaggaagcttgttcagaggtcactgtctttagaagcacttcgcaacttggtgcctcatattgaagccttagccttatctgccatgaactcttggggtggtgatggccaagtcattaacacatttaaggaaatgaaaatgttttcttttgaagttggaatcctcacagtttttggccatctagagccgcgtttgagagaggagctaaagaagaattaccggattgtggacaatggttacaaatcctttccaatgtgcatcccaagaacacaataccaaaaggcactattggctagaaggagacttagcaagattatatgtgacataatttgtgagaggaaggagaagaagttgtttgaaagggatctcttgagctgcttattgaattggaaaggtgaagggggagaagtgttatctgatgaccaaattgcagataacattattggggtgctttttgctgctcaggacaccacagctagtgccatgacatgggttgtaaagtacctgcatgatgaaccgaaacttctggagtctgtaaaggctgagcagaaggcaattcacaagtctaatgaaggtaacctaccactgagttgggatcagactagaaacatgcgaattactcacaagacctga

SEQ ID NO: 183 - DNA, 264 bp, Glycine max
  source 1..264: mol_type = other DNA
atggccgaagaatttgtgaatgactttgttcatcaaaatggcatcactgtcataatccacaatccccccaaaggtgatggggagaaaaagaccaaaatttggataatattagttgttgtggtggggctaatatgccttggaatccatatatttctggtatggaggtttaagagaaaacctaaagcaatttcatcagcttttggttacaacaacaatagcgaaatactagtcattgatctaacgaggagtacagacttatcataa

SEQ ID NO: 184 - DNA, 3753 bp, Glycine max
  source 1..3753: mol_type = other DNA
gcaggttacaaagatattggaaatcctatttggcaatgcaagcaatgtaaagcaaagatgtggtatgatgagaggattaacaaagataaacagacaaaaaatccaaagttttcattatgttgtggtgatggtaaaatacaactgccaatactacatgatgctccgcaacccttgtggcagctactctttgacagcagagactcacaagtaaagaaattccaacaaaacatacgtttatacaacctaatgtttgcattcacatctctcggtgtcaaagtagacacatcctataatactggaagaggtcctcctacattgcgcatacatggtcaatcgcatcaccttattggaagcctcctacctataccggataactcaccaaagtttgcacaactatatatttatgacacggagaacgaagtcaacaataggctttcacaataccctataaaaaataatgttgatgaagacattataattggtatcaagaatatgctagatacccacaatccatatgcacaaaaattcagaatggcaagggataaactagactcttctgcagtgtgtgacctgaaactaaaactcattagtgacagacaaactgatggcagattatataatttgccaaatgcttctgaagtggctgctttaattgttggtgatgagcatacaacaaataacaaagatatcatcattgagaagcaaacaggcatgctacaaagaataaatgaacttcatcctacatatttacctttgcaatatcctcttttgtacccacacggtgaagatggctacagaccaaacattcttcataacatcatccgcatagccatgctgcaaagaggaacaaagatgcaatcaagggataatgaagcacaaacaatacttcattcaagaagattgtttcaccaatgggttgttgatgggtattgtatgattgagtctcaaaaactaaactatgttagacaacatcaacaagaactccgagttgccaagtacatcaatttaaattactgtaataatcagcccttaacccaaggtaatgaaaaaggtaagagaattatactaccaagttcgttcgttgttagtcagagatatatggaacaattgtattttgataggatggcaatttgtgcccatgttggatttcctgatctatttttgacattaacgtgtaatccagtatggccagaaatacaacgacaagttgaaaagtcaaatcttacagctcatgattgccctgatgttatgtcacgggtattcaaaataaaactaaaccagttgatgcatgatcttaagagtggacatgtatttggtcccattcttgcatacattgacaacataatttttgctgaaataccaaacaaggacacatatccagaattgtatcaaattgtctccaaccacatgatgcatggtccatgtggactagctaataaaagggcaccatatatggccaatggcaagtgtttcaggtttttcccaaaaaagtttcagccagccacaattgttgaccaagatggttttcctgtttatagaagaagagacaccagacggattgtgcaaaagcagggtgttcatctcgataatcaatttgttgtcccatatagtccacatttgctactcaaatatagaacacacctaaatttggaatggtgcaatcaaagtacctcaataaaatatttgtttaagtacatcaacaaaggatctgatcggattacagctgctattgtcaatgaccaaaaccaggatggcacacacaatcaggttcatgatgaaattaaacattatcttgactgtcggtatgtgtcgactcctgaagcatgttggaagatttttccattcccaatgcatgggcatgcatcggcagttgaacgtctttatttccacctagaaaatcaacagcctgtttactggaaagatagtcaagaaattggcacagtactggctaagagtacaatcaaagaatcaatgttcacagcttggatggattctaataaaatataccatcatggacgatatcttacttatgctgaatatgtgtccaaatttgtttatgatgcacgaaaaagatgctggaaaccaaggaaacaaggaaatactattggcaggctgatttgggtgcccccttccagtggagagttattctacatgaggatgatgctttgctctgctaaaggatcacaatgttacgaagatattagaacagtagaaaatgttgtctatcatacattcagagaagcatgctttgcaaaaggttttctaggaaaagcaaacacttggggaactccacactatcttaagaagttatttgtgaagctgctatttatgaataccatggataggccagaatatgtgtggaaacaaacttggcaatggatgacagatgatattgtatttaatcatagaagacaagattttccttcaatgccatacccactaggatatgccgccaacacgcaccaaaataatctcatctacaatgaattgacttacgacagagacatattggtcgccgaatttgacaaatgctaccagtcgctaacagatgagcaggcttctatttttaataagattatgcatgtggttgcaactcaatcaggtggagtttattttctatatggatatggtggcacatgcaagacatttcttcaagtgggattgcctggcggtagaacaacacattctaagtttgctatttctgtccctgcaacaaaaaattctacatgcaatattcatcaagggactccaatgtgtcacagatacagtattgaggcccttgacaaaagtttacaagacatcatgcacaacagcaatccttttggaggaaaggtcattgtttttggtggtgatttccgtcaaattctacctgttgtgccaagaggtaatcgttctgacattgtctatgcaactttaaattcatcttacatttggaaccattgtcaaattctaaagctgactaaaaaaatgcgatttcaatcaaatcctactgaccattccaacttggatgaactaaaacaattttttgagtggttgctagacataggtgatgcaacatatccagacttattacacaactacaacaatggcgatttcttgcaaaaaagagttgtccttgcctctacaaaagatgttgttgataaaataaatgactatgtcctgtctttgattcctggtgaggagaaagagtattgtagtgctgattctattgacaaatcagatgaactactcaatcctgcatttggagtactaacagttgaatttctaaactcattgaagacatcaggcataccaaatcacaagctcataatcaaggttggtatgcctaccatcctactacgaaatctggaccaagctgatgggttatgcaacagaactaggcttattgttacaagacttggttcaagtgttgttgaagtggagattattactggacccaacataggccatagaacatacattccaagaatgaatctttctccctctgattcaccatggccattcaagctaattagaagacaatttccattcatggtttcatttgcaatgacaattaacaaatctcagggacaatcgttggcacatgttggattgtatttgccaaccccagttttttctcacggccagttatatgttgcactttcacgagttcaaagcaaaaaagggctccacattcttattcatgataatcaaggcactcccaaaaatactaccattaatgtagtatacaaagaagttttttcaaacttataa

SEQ ID NO: 185 - DNA, 1947 bp, Glycine max
  source 1..1947: mol_type = other DNA
atgcctccaaatttgttgcctatcgttgttacatctattagaacaaggatcgaagaagggttttggatgaaaagtgatcgaagtagtctgctgcaggcagctgctattggttgcttgacattagctttgtctacttcaccatcttctgctcaaataaggaagatgctctatgatgaggtttcttcaggttatattgtgactgaaaagaagtctggtgttctttctacgttatttgaatattcaatgcaatggagttgcccaaccatttgccttgaggcccttcaggcacttaaagctgtatcccacaattatcctaacatagtgtctgcttgttgggaaaaagtttctgctattgtgcatggcttccttagtactgtgtgccttgaagctccttccaggcagtcaagtgatcatgttggtagtccttcttcatttaataatgaaaaagttctgatcactgctattaaggttctggatgagggtcttcgggcagtctctggatttcaaggaacagaagatctttcagatgataaattgatggatattccttttgcatctgattgcattaggatgaagaaagtgtcatctgctccatcatatgagctagagtgcaaagatgatgttatagtcaactttgaatcttgtggatcaggaagtcagcagtggtgtgaggcaattgagaagcatatgcctcttattttgtgtcactcttcagcaatggtgagagctgcatcagttacttgttttgcgggtatgacttcttctgtattcatttgtttttctaaagagaaacaggacttcatattgtcttctttagtgcatgctgctgtacatgataatgtgccatcggtgagatctgctgcttgtcgagccattggtatcatttcatgttttccacaagtgtgtcagagtgcagaggtacttgacaagtttatccatgctgttgagattaacactcgcgatgccctaatctcagttaggataacggcttcatgggcattggcaaatatatgtgatgctatttgtcactctgatagaatactcccctatgggcaaatgggttcaaactccaatacccaggtgattgtatcattgagtgaatgtgctttgcatcttactaaggatggagacaaggtcaaatctaatgctgtaagggctcttgggtatatttcaagaatcttaaaatcttcaacatcaaaattccaaggcacgtcagcgggtcaccatgaccgcatgactgatgcgtatctcaatagtgagaatctaatggtgtgtcaacagaattgtgcatcagattctctgcaagatttgaataggctagagagaatagttcatgcatttatttcttgcattactactgggaatgttaaggtccagtggaatgtttgtcatgctctaggcaacttattcctcaacgagacattaaggctgcaagatatggattggactccagttgtgtttggtgttcttctgcaactattacgtaattcatcaaattttaaaatcaggatacaagctgcagcagcattggctgtgccaatgtcaatgcaagattatggcctatccttttcagagattgtgcaatctgtagagcatgtaatggagaatattgacgatgaccaaatttctgggccatcaaatttcaaatacagggtttcgttacaaaagcagcttaccttgacaatgttacacattctacgctttacctcaagcacaaatgatcaaaacctgaaagattttctagtgaagaaagcatcgatccttgaagattggttcaagggattatgctcatctggcgagggcatgctcgatgttcaagataagtgcattgcagaccgaaagagggttttgatatctggtgcgctacagtcgttgattgaagtatacaaagaaaaacaacaggatgcgatagctcagaagtttgaggaattgaagaacaatatgtaa

SEQ ID NO: 186 - DNA, 255 bp, Glycine max
  source 1..255: mol_type = other DNA
atggaaaaaagtttgtctcctagaagcaaaacaaacggagggtggttttcatgttgggggcgtctcaaattgaagcttccatggtcgaagagaacaagcacttataaacccattggtggttttaactatgaccctttaagctatgctcaaaactttgacgagggatgggaggaggatgatgaagaatctttacatcgcgggttctctgctagatatgcagcaccttcctcacataataaattgctcaagcaatga

SEQ ID NO: 187 - DNA, 1530 bp, Glycine max
  source 1..1530: mol_type = other DNA
atggatttgaagattcaacatattaaatgggtggggaatatttaccagaagtttgaagcagtgtgtcaggaggtggatgatattgtaggccaggatgcagttaaatatcttgagaatcaggtccaaaatgttggagacagtgtgaaaaagttctattctggagttgtacatgagctacttccttttcctacctcggctgattcaaaatatgaatctcattcagtggctttgacaaataatattggctttccagttgagtcagttgttggtcataaagataataataaaaaaagggatgaggaaaatcccaccaataatgttatcaagtcattacaggaatctagtgccattgatattgctaacaatcagcaagttggtgttcctatcaagcacaaacttattgatgaaacttgttcagattccctggaggtggaagattcttacataactcaggaagaggtcggtgatgattcaagagaaacttccggggctaagaaagaaaagttgaatacaagcattgaagaggtttctgttgagtctgtgcctaaatcgatgaacttgatgtctcttagagagaaagaatcccttgaattccctatacacagtgaatcttattctgattcttctgacagcgggtgtgaagattcaatcgcaaaaaaggataacatagatgtgactgtggaacaaaactcatgtttggttgttgaaaaaaatgctatgaattcatccacctcagaagtgttgagttcccagtctcttgacggggaagaatcaattaaagtttccttgttcagtgaatcatctgatgctgttgatgaggatacccatgacatactagctgaagtttcacctgatgcttctgtgtcaagcgaaagacctattatcacaatgacagaacctttatgctccagaaacttcataacttctgacagtctatattcaaagtcactcggaagctaccccttggaaattgaatcttgcaaaaacaattcaggtgatgctactctgtgtatttcagatagttccatgatgcatatatgttgtgaatcatccccccatgtagctcgtcaaatcatggaatcccaggatggacttgctttctctggctactgccaatctctggaatcaaatgataaatcactgttcagctctgttgaatccagtttggaagacattgatttgaatgatgatccaaagcttgaggaaaactgtgtatttgtggacgatagtgaactctatgcagtctcttgtagagcccaaaagcttagatcatataagaaaagaattctggatgcattttcttcaaagaagaggttatcaaaggagtatgaacagctagcaatatggtatggagacaccgatattgagccaaaacaaggattttcacaaacttcattgccatttatctccagaacgtacatggactcgaagaatgtgcaagttcagcgtgcttctgaaactgagtgggagctgctatatgcatataggatttcattccatgcatcagggactccaggtaggcaccagtga

SEQ ID NO: 188 - DNA, 1914 bp, Glycine max
  source 1..1914: mol_type = other DNA
atggaatcaagcatggatggaagatcctctccccatttgctgatgcttctttgctttgcttcatttcttcaaatatgtcactcagcatcccaactgaagagctacattgtctacactggcaatagcatgaatgatgaggcttctgcgttgactctttattcaagcatgctacaagaagttgcagacagcaatgctgagcccaagttagtacaacaccacttcaagcgtagtttcagtggctttgttgctatgctaacggaggaggaggccgatagaatggctagacatgatagagttgtagctgtctttcctaataaaaagaagcaactccatacaacaagatcatgggattttattggctttccactacaagcaaatagagcaccagctgaaagtgatgtcatcattgcagtacttgactctggaatttggcccgaatctgagagcttcaatgacaaaggattcggtccaccacctagtaaatggaagggcacctgccaaacttctaaaaatttcacatgcaataataaaatcattggggctaagatttacaaagctgatggattttttagcgatgatgatccaaaatctgtgagggatatagatggtcatggcactcatgtagcatcaacagcagctgggaacccagttagcacagctagcatgttaggccttggtcagggaacagcaagaggtggcgcgacaaaagcacgcattgctgtgtataaagtgtgttggtttgatggttgttctgatgcagacatacttgctgcatttgatgatgcaattgcagatggggttgacataataacagtttctcttggagggttcagtgatgaaagctactttagagatgtaatagccattggagcatttcatgctgtgagaaatggagcattaacagtaacttcagcaggaaacggtggtccaagaccttcctccctatccaacttttcgccttggtcaattactgtggctgctagtaccatagacaggaagtttgttaccaaggttgaattaggtaacaaaatcacctatgagggtacctcaataaatacatttgatctcaagggagaattgtatcctataatttatggtggagatgcaccaaacaaaggcgtaggcatagatggatcgtcatcaaggttttgcttcagcggttcgttggacaaaaagttagttcatggtaaaattgttctctgtgatagcagaagccaagtgtcaggcccttttgatgctggtgctgttggggcattggtacaaggtcaaggttttagagatattcctctctcttttccattgcctggatcttaccttgccttgcaggatggtgtctctgtatatgactacataaactctacaaggactccaactgcaaccatatttaagactgatgagacaaaagatacaatagctcctgttgtggcctctttctcttcaaggggcccaaacattgttacacctgaaattctcaagccggatttagtggctcccggagtttcgattctagctagttggtctccagtttcccctccttctgacattgaaggtgacaacagaacattgaatttcaatataatctcaggaacttcaatggcttgtccacatgtttctggagccgcagcgtatgttaaatcatttcatccaacatggtctcctgctgctattcgttcagctctaatgacaacagctaaacaacttagtccaaagactaaccttcaggcagaatttgcatacggctcaggccaaattgatccttccaaggctgtgtaccctggtttagtatatgatgctggtgaaatagactatgtaaggtttttatgtggacaaggctatagtacaaggacttacaactcatcacaggagataacagtagctgccctgaaacaaagaatggttcagcaagggatctaa

SEQ ID NO: 189 - DNA, 996 bp, Glycine max
  source 1..996: mol_type = other DNA
atggaaatcttgtctcgtctacccgtgaaacctttgatccaattcaagtatgtgtgcaagggatggaactccctgatgtcagatccctatttcatcaaattgcaccttagcaaatttgctgcgaaggacgatttggaacaccttcaactgatgaaaaatgtctgcctcggatccatccctgaaatccacatggaatcgtgtgatgtaagttcgttattccattccctacaaattgaaacgttcttgttcgatttcgaaaacatgccaggttaccatctggtcggttcatgtaatgggttgcactgtggggttagcgaaataccagaaggataccgtgtttgtttctggaacaaggcgacaagggtgatatccagagaatcgccaacgctgtctttttccctgggcattggtcgtagaaaaatgtttgggtttggctatgatccgtcaagtgacaaatacaaggttgtagcaattgcattgactatgctctcacttgacgtatttgaaaagactgagatgaaagtttatggcgcgggtgacagtagttggagaaaccttaaaggttttcctgttctttggactttacctaaagttgatggagtgtatctgagtggaacccttaattgggttgttattaagggaaaagaaaccattcattctgaaatcgtaattatttatgttgacctggagaaggagacttgcagatcactgtttcttcccgacgatttttgcttttttgatacaaatattggagtatttagagactcgctgtgcgtttggcaagatagcaacacccatcttggcttgtggcagatgaggaagtttggagatgacaagtcttggattcaattaataaattttagttatttacatcttaatattcgtccttatgaagaaaaatcgatgattttaccattgtgcatgtctaacaatcgagacttcttcatgctgaaattcactagaaatgctgatgatgaataccaaacaattctgtaa

SEQ ID NO: 190 - DNA, 2208 bp, Glycine max
  source 1..2208: mol_type = other DNA
atgagtgaggaagaaccagcgaatgaagtttctaagattgattcaaatggaaagagcttgcagaagataattttggatgaaattactgagaaagaggatttggaaactgatggaggaaaagtcttggagaacaatggaatcaaagagttgaaagaaaatattgtaaaagaaattgaagataagaaaactgatagtgtggaaaaggtagaagaggacaagaaagttggtgttgtggaagaagcaaaagaggataagaaagaagatggggtgaaagaagcaaaagaggataagaaagaagacggggtgaaagaagcaaaagaggataagaaagaaggggtggaagaagcaacagaggataagaaagaagatggggtggaagaagtaaacgaggataggaaagaagatggggtgggagaagtaaaagaggataaggaagttgatggtgtgtcatgtgtgaaagaagttgaggaggatggggaagttgatggtgtgaaaaaagtagaagataaaaaaggtgatgagttgaaagaaatagaagaggataagaaagatgatcatatatctgaaaatgataaaatggatgaagacactgagattaaggaaacaatggaaggtgaaccagaaaatggaaaagaggaatctgagaaaccagtagtagatgctatggaagtggagggtggtattaaggagaaggaggagagcaaagaaaatgagaaagttgaagtggtgatggaggaggatgagaaggatgaagatgaggataaggataatattgataagtcaaaagaagagaaggcagaagatagtaagggtgagaaaggatcaaaaaaacgtgggagagggaagatcaatggggagaaagtgaaagaaaaaagaaaagaactgaagaaaacagagccaaggactcctactattgatcgccctgtgcgggaaaggaaatcagttgagaggttggtagcatcaattgataaagatgcaaccaaagaatttcacattcaaaagggccgtggtacacctttaaaagatataccaaatgtggcatttaaattatctagaaggaagactgatgataccttcaaattgctccatacaattctctttggaaggagagggaaggcagttgaaatcaagagtaatatatcaaggttttctggttttgtgtggcgtgacaatgaggaaaagcaaatgattaaagtaaaagaaaaacttgacaagtgtaataaagagaagttactggaattctgtgatgtgcttgacataacaattaacaaggcaacaacgaggaaggaagatattattgctaagcttatagactttttagttgcccctcatgcaaccacaactgtgttacttgcagaaaaagagaagcccagtaagggaacaaagcgcaagcgtgttgtaaaacggggttcatcaagatctgggacaacttcaagacgttctgtgaagaggcagaagaaaaacgaagattctacagtagcgcggagaaagagtgcatctgacacagatgagtcagaggaagacaagaaagatgaagaaaacgaggaggaaaatgaaattggtgttgctgacaaatctgaggatgaaacaccagagaaatctgaaagtgaagacaaaagtgattctgggagtgaatctgaagatataaaagaaaagaaaaaaccttctaaaacatcatccacaaagaaagaatctgctaagaaaagtaaaaatgaaaaaattacagttcccaataaatctcgctcaccaccaaaaagagcacctaagaaaccatcatccaatctctcaaagtctgatgaagacagtgatgaaagtccaaaggtcttttcaaggaaaaagaaaaatgagaaaggaggaaagcaaaagacggcaacaccaactaaatctgcctccaaagagaaaactgcagaaaaggttactagaggaaaaggcaaaaagaaagagaagtcgagccccagtgacaatcagttgcgtgatgcgatatgtgaaattcttaaagaagttaacttcaatacggccacatttaccgacattctgaagaaacttgctaaacaatttgatatggatctcactccgagaaaggcatctataaaatccatgattcaggaagagctgacaaaactagctgatgaagcagatgacgaggacagagaagaggatgctgagaaagatgaagccccatctacaggccaagaggttgaaggctga

SEQ ID NO: 191 - DNA, 336 bp, Glycine max
  source 1..336: mol_type = other DNA
atggagttttgccccagttgtggtaacatgctacagtatgaattgccttatatgggtcgcccttcaagatttttttgttctgcttgcccatatgtttgccacattgagaatagggttgagatcaaaagaaagcaaaggttggtgagaaaagagatagagcctatattctccgaggatgacatgacaaatgcaccatcaactgaagcgacgtgcccattttgcggtcatggtaaagctgctttcaaagaatttcagactcgatcggctgatgagccagcaactctattttataagtgcttgaacaatgactgtaaaaaacaatggcgtgaaggttga

SEQ ID NO: 192 - DNA, 174 bp, Glycine max
  source 1..174: mol_type = other DNA
atggttcaccgcaacaattcctccttcttcgacaccacctctttccccttgtaccttcctcacaacatcgtggctctttgtttgccctctttcctttgtgcttcgctattgatgagatcaacacaacaaagatattggaatctccatcatgggttgatcattgataaagcttga

SEQ ID NO: 193 - DNA, 2532 bp, Glycine max
  source 1..2532: mol_type = other DNA
atgtcaggctatttagatattgggtatccaataatgcagtgtaaacaatgccaagcaatgatgtggtatggagaaagaatgcttaaacataggcatggtaccaatccaaagttcagtttgtgttgtggaaatgacaaggtccaacttccattattacaaacaccacctccattgttagcaaaacttgatgactttattaacgatggaagaggaccgccaacaatttgtatccaaggccaaccatgtcataggattgggagtctattaccaatgcccagaaaagaatcaaaattcacacaattgctacaattcaaacccatggaagcgcatacagatttacattcaaggagactattccaacaatttagtgttgatggctataccatggttgaatcagagagactttatttcataagaagtatccaatcaaaattaagggtcgacaaatattgtgatttgcgtgaaacatctaatcatggtgctactcaagggtcaaacagaggcaagcgtgccattttaccttctacatttgttggtagtcgtaggtacatggatcaattatacttcgatggtattatcatattcatgtacaccattgaattccaaaaaagaggtatgccatatgcacacttactcctatttttgcatccattgagcaagtatccaacgcctaaagatatcgacaagattatatgtgctgagataccatcaaagaatgaacaaaatgaactttactcgctggtccaggcacacatgatacatggttcgtgtggcatagccaaccaatcatcaccctgcatgaaggatggaaattataggtgttggaaaccatgcaaaaaagggttcacacttggcaggttaatttggatttcacctacccttggagaattatattacctaaggatgatgttgccagtgtgcaaagggcctacctgttatgaagatataagaacaatggaaaatattgtgtatcctacatttagagatgcttgttttgcattaggctttcttcaagatgataaggaatatgttgaagcaattaaagaagcgaaagattggggtacaagtcatcacttgagaaaactttttgttacaatgctaatcacaagttccattaataaaccagagcaggtttggaaacaaatatggcattggttagctgatgatattctatattctttgcaactgaatgatgatgaattgaaaaatttaacttcattggagattgagaaatttttgcgtgcgagtggaaagatcttaaaggactatccaccaatatcatgcatagctgcaaaattgtcaagttatataggaaattgtttgatacacttggaactacattttgatcttgctgaattagatgctgaattcaaaatattattctctgctcttactaccctaagagcaaagaaaaaaattgttttggtagttgcatcaagtggaatttcatctttattactacctagtggaagaacaacacattcaaagtttaggattcttctaccaactttagatgactcagtttgtaatataacccatgggagtgacttagatgagttgcttaaagttacaagtttgattatatgggatgaagcaccaatggtgcacaaattctgttttgaagcactaaacaaaaccttaaatgatatcatgaaaattagtgatgatagtgacactgttttcagaggaaaaatagttgttttttgtcaaattcaaacactacacatgcaactggagttacaaactttgcaaaatgaatccttcaaattggagatggaaattccaaaagaattgtttatcctgaattacactgatccaattgatgttatagttaaatgtaaataccctaatcgaaggcaacattacaaggattcagagtttctacaatccagggctattttagcatcaactaatgaaactgttgaccatattaatgattatgtattatctttgatctcagagagagaatacctaagctcagatttaattgaaaaagctaaaacaatagaaagtgaaaggtttagcacaataactatggaatttctaaactctttgtcaacctctggtcttccaaatcataagattaaggtcaaggttggctcaccaataatgttgttaagaaacttagatcaaaatgaaggcctatgtaatggtactagactaattatcacaagttttgccaaccatataattgaagctaaaatcatgacaggcaaaggccaaggcaacaaagtgtacattcctaggaggtcaatgtctccatcacaatcaccttggcctttcaaactcataagaaggcaattcccaataattgtctcttatgcaatgaccataaatgaatcacaaggacaatcattagcaagtgttgggttatatctacctagactagtatttagtcatggccagttgtatgtagcattttctagggtgcaaatgacaaatggactaaaagtaataatacaagataaagataaaatgccctcaagtacaacaactaatgtagttttcaaggaggtgttccagaacttataa

SEQ ID NO: 194 - DNA, 1290 bp, Glycine max
  source 1..1290: mol_type = other DNA
atggatccagcgttacccattgtagcgttatcgcttctcttgggtgccatcatcgcatttctctttttcagcacctactaccggaagcggcaatccgaaatccaatctatcgccgatcccaatcccaaacccttatcctccaaagcccctcccaagaagcctctctccaaaccccattcctccgacaaggatcaaaacaagcgacatcatccgttggatttgaataccttgaagggtcatggtgatgcagtgacagggatatgcttctcccccgatggacgaaatttggcaacagcttgtgctgatggagttgtgagagtatttaagttggatgatgcttcaagtaaaagtttcaagtttctgaggattaatttgccagccggtggtcatccaacagcagttgcattttctgatgatgcatcctccattgttgtggcgtcttatactctgtccggctgttcattatacatgtatggagaagagaaacctaaaacctctgaagataaaccacaaacaaagcttcctctcccagaaattaagtgggagcatcacaaagttcatgataagaaatcaataattactatgtttggagcctccgcaacttatggcactgcggatggaagtacaattgttgcctcatgttcagaagggactgacatcattctttggcatggaaaaactgggaagagtgttgggcatgttgatacaaatcaattgaaaaataacatggctactatatcacccaatggtcgttttattgcagcagcagcatttactgcagacgtgaagatctgggagattgtatatgctaaggatggatctgtgaaggaagttttaaatgttatgcagcttaaggggcataagagtgctgtgacttggttatgctttatgccaaactctgagcaaataatcactgcatccaaggatggttcaatgagactatggaatatcaatgttcgttatcatcttgatgaggatccgaagacgctgaaggtgttcctaattcctcttcatgattcttccggtactacactgcactatgaccgcctgagtgtttcccctgatggaaaaattttggctgcgacccatggctccacactgcagtggttatgtgttgagactggaaaggttctggatacagctgaaaaagcccatgatagtgacatttcatgcatttcatgggctcctaaacccattccaatgggaaatgaacaagttttggttttagccacagccagtgctgacaagaaagtgaagttgtgggcatctccatctatcccttcatcatga

SEQ ID NO: 195 - DNA, 3177 bp, Glycine max
  source 1..3177: mol_type = other DNA
atggctctgcgatcattctcctatgacgtgttcctcagcttccgaggggaagatactcgttatggtttcactggcaatctctacaatgtcctccgggaaaggggaattcacaccttcattgatgacgaggagctccagaaaggggatgaaatcaccacagcacttgaggaggcaattgagaagtccaagattttcatcatcgtgctctctgaaaactacgcatattcctccttttgcttaaacgaactcactcacatccttaactttactgaggggaagaatgatccgttggttttgccagttttttataaagtgaatccttcatatgtccgacaccacagaggtagttatggagaagcactggctaatcatgaaaagaagttgaactctaataacatggagaaacttgagacgtggaagatggctctgcgccaagtgtctaacatttctggccatcatctccaacatgatggtaataaatatgaatacaagttcattaaggagatagttgaatcggtgtctagcaagtttaatcgtgatcatttagatgttccgaatgtcctggttgggctagagtcaccggtgcgacaagtaaagtcgcttctggatgttggacgtgatgatgtcgtccacatggtggggatccatggactcgccggagtgggtaaaacaacacttgctgtcgcagtctataattccattgctgatcattttgaatcttcttgctttctagaaaatgtgagagaaactaccaataagaaggggttagaagatctccaaagtgcctttctttctaaaacagctggagaaattaagttaacaaattggagagaaggaattaccataataaagtgtaagctcaagcaaaagaaggttcttctgattctagatgatgttgatgaacataaacagttacaagcgattattggcagccctgattggtttggtcgtggtagtagagtcatcatcaccactcgagacgaacatttgttagcgcttcacaatgttaaaataacatataaggtgagagagttgaatgagaaacatgctcttcaattacttactcacaaggcttttgagttggaaaaagaagttgatccaagttaccatgatattttgaatcgagcgataacttatgcttcaggcctcccattagctctagaagtaataggttccaacttacttgaaaaaagtatagaagaatgggaatctgctctagatggatatgaaagaattcctgataaaaagatctatgacatacttaaagtaagctatgatgctttgaatgaagatgagaaaaatatttttcttgacattgcttgttgcttcaaagcttataaattggaagagctccaagatatactttatgctcattatggtcattgcatgaaatatcatattggggtgttggttaaaaaatctctgataaacattcatgggtcatgggattataaggtcatgagattgcatgacttgatagaagacatgggtaaagaaattgtccgaagagaatcaccaacaaatcctgggaaacgtagcaggttatggtcccatgaggatataaatcaggttttacaagaaaataaggggactagtaagattgaaatcatatgtatgaatttttcctcatttggagaagaagtagaatgggatggagatgccttcaagaagatgaaaaatctcaaaacacttatcatcaagagtgattgtttttccgaaggtcccaagcatcttcctaatactttaagagtattggaatggtggagatgtccttcacaggattggccacataattttaacccaaagcaacttgctatatgcaagttacccgatagtagctttacatcagtcgggttggccccattatttgaaaagaggctcgtgaatttgacaagtttaattttggacgagtgtgatagtttaacagagataccagatgtatcttgtctctcaaatttggaaaatttgtcatttagaaagtgtcggaatttatttacaattcaccattcagttggtttattggaaaagcttaaaatcttggatgctgaatgttgcccagagcttaagagttttccaccgttgaagttgacctctcttgaaaggtttgaactttggtattgtgtcagtctcgagagttttcctgaaatattaggaaaaatggaaaatataacacaactttgcttgtatgaatgtcccataacaaaactcccaccttcatttcgaaatcttactcggcttcgatccttaagtcttggacaccatcaccaaactgagcagttaatggactttgatgctgccaccctcatttcgaacatctgcatgatgccagaactggatggtatttcagctgacaatttgcaatggaggctattgcctgaggatgttttgaaattgacctcagtcgtgtgttcaagcgttcaatctcttactttgaaactgtcagatgagcttcttccgctatttctctcatgttttgtaaatgtgattgatttagagctatcagggagtgaattcacagttattcccgaatgcatcaaagaatgccgctttttatctacccttactttggatcgttgcgatcgtcttcaagaaattagagggattcctccaaacttgaaaactttctctgcaatggattccccagccttgacctcctcaagcataagcatgttgctgaatcaggaactgcatgaggctggagacaccgactttagtttgccaagagtacagattccacagtggtttgagcacaagaatccgggacggccaattcgtttctggttccgtaacgatttcccagccatagttgcttgcattgctaagtcagattttcagggagttttcgattatccagatctcagcgtgttcattaatggaagggaacataaacattacggtcgtacccctgtccttgaaaaaccttgtacagttctttttcatcttctaatagaagatgatttagatgtatcactgttagagaacgaatggaaccgtgcagagattgtatgttatggttcatgggacgaatgcggaatccatgtattgaaagagctaagtagcatggaggatattcgattcactgatccttttagaaaagaaaaatttgtagttcagaggttgcggttcgggaaaaagcaaaggttagcaagagtgaagttgttgagacacaatttgtag

SEQ ID NO: 196 - DNA, 3312 bp, Glycine max
  source 1..3312: mol_type = other DNA
atggctgcaacaacacgttcccgtgcatccatctacgatgtgttcctaaacttcagagggggagacacacgctatggtttcactggcaatctgtacagggctctttgtgacaagggaattcataccttctttgacgaaaagaagcttcacagaggagaggaaataacacctgcacttttgaaggcaattcaagagtccaggattgctattactgtgctttctaaaaactatgcttcttcctcattttgtttagatgaacttgtaaccatccttcactgcaagagtgaagggctgttggttataccggtcttttacaacgtagatccttctgatgtcagacaccagaaaggtagttatggagtagaaatggctaagcatcagaagaggttcaaagctaagaaggagaagctgcagaaatggaggatcgctttgaaacaagtagctgacttgtgtggatatcatttcaaagatggagatgcatatgaatacaagtttatccagagtattgttgagcaggtttctagggagattaatcgtgctcctttacatgttgcggattatccagttggtctagggtcacaagtgatagaggtaaggaagcttttggatgttggatcccatgatgttgtccacattatagggatccatggaatgggcgggttaggaaaaacaacacttgctctggcagtttataatttgattgctctccattttgatgaatcctgttttcttcaaaatgtgagagaagaatcaaataaacatgggttaaaacaccttcaaagcatccttctttcaaaattacttggggagaaggacatcaccttaacaagttggcaagaaggagcttcaatgatacaacataggctccaacgaaagaaggttctcttgattttagacgatgttgataagcgcgagcaattaaaggctattgttggaagacctgattggtttggtcccggtagcagagtcatcattaccactcgggacaaacatctgctaaaatatcatgaggttgaaagaacctatgaggtgaaggttttaaatcagagtgctgctcttcaattgcttaagtggaatgcttttaaaagagaaaaaattgatccaagttatgaggacgtcttgaatcgtgtagtaacttatgcttctggccttccattggctttggaagtcataggttccaacttgtttggaaagactgtagcagaatgggaatctgctatggaacattataaaagaattcccagtgatgaaatcctagagattctgaaagtaagctttgatgctttgggggaagaacaaaagaatgtttttcttgacattgcttgttgcttcagagggtataaatggacagaggttgatgatatactccgtgctctttatggtaactgcaagaagcatcatattggggtgttggttgaaaaatctctcataaagcttaactgttatggtactgatactgttgaaatgcacgacttgattcaggacatggctagagaaattgagcggaagagatcaccacaagagccagggaagtgcaagagattatggttaccaaaagatataattcaagtttttaaagacaacaccggaactagtaaaattgaaatcatatgcctggattcctccatatctgacaaagaagaaacagtggaatggaatgaaaacgccttcatgaagatggaaaaccttaaaatacttattattagaaatgacaaattttccaaaggtcccaattattttccagaaggtttgagagtactggaatggcacagatatccttcaaattgtttaccatctaactttcatccgaacaatcttgttatatgcaagttacctgacagttgtatgacgtcgtttgagttccatggcccatcgaagaagttcgggcatctaacagttttgaagtttgacaactgcaaatttttaacacagatacctgatgtatctgatctcccaaatttgagggaactttcatttgaagagtgtgagagtttagttgcagttgatgactcaattggttttctgaataaacttaaaaaattgagtgcgtatggttgcagcaagcttaagagttttccgcctctcaacttgacctctcttcaaacacttgaactttctcaatgttccagtcttgaatattttccagaaataataggagagatggaaaacataaagcatctttttttgtatggccttcccataaaagaattgtcattttcatttcaaaatcttattggactccgttggttaaccctgaggagctgtggaattgttaagttaccatgtagcttagcgatgatgcccgaactgtttgaattccatatggaatattgcaacaggtggcaatgggtagaatcggaagaaggtgaaaaaaaagtgggctcaataccatcttcaaaggcacatcggttttcggccaaggattgcaatctatgtgatgatttttttttaacaggtttcaagacctttgctcgtgtaggacatttaaatctgtcggggaataatttcacaatccttcctgaattcttcaaagaattgcaattattaagatcacttatggtgagtgattgcgagcatcttcaggaaattagaggtcttccaccaaacttagagtatttcgatgcaagaaactgtgcatccttgacttcctcgagtaaaaacatgcttttaaatcagaaactgcatgaggctggaggaaccaattttatgtttacaggaacaagtataccagagtggttcgatcagcaaagcagtggaccttcaagttctttctggtttcgtaataaattccctgccaaacttctttgtcttcttattgcacctgtgtctactgggattgttgtccttaatccaaaggtgttcatcaatggcaaatttcaagaaattcggccctattttggaagacacgagataaaaagtaggttgaacttggatcatacatatatctttgatctccaagcgtctgctttcataaataataatcggtttgaagaaatggctagggaaaaggaatggaaccatgtggaggttagatatcaaagtgtgttagcctatgaaaaagaaaagagagaagaaggtgtcttagacttagagagctcaatcattaaggcaagtggaatccatatattcaaagaaagcagtatggaggaagatattcgatttgatgatccttatctcagcagttctgcatcagaaagcccatcgttgctacaaaccatagcctttgggaaacgcaattccatagcttttttcttatttttttcatgttttttattttattattttggtttcagttgtcaaaaattcacttaa

SEQ ID NO: 197 - DNA, 1272 bp, Glycine max
  source 1..1272: mol_type = other DNA
atggcgacggctgaagatgctccagtgctccctgaggacctcatagtggaaattctgtcatgggttgaagtgaagaatctcatgcgattcaggtgcgtttccaagtcctggaattccctcatcttcaatcccacattcattaaattgcaccttcaaaggtcctcccaaaacatccacatcctgttaacatttgaccaagattcatcgaacccctacccgtaccatgatgataactacatctccgtcgttgccgcaccctgctctatacagcgtttacttgagaacccatcatccactatttataatatagttcattttctcgaggctcagtcaacatcatcatcgtccactatttattttgatgtttgttaccgatttaagcacacttatttgttcttgggtgtatgcaatgggttggtttgcttgcttgattgtttgtatgaggatgaatttgaggaatactgggtccgattttggaacccggccactagggccatgtccgcagattcgccccacttgcgtgtccattcgagcaattataaacttggagacattgctgtgaagcatgcgtttgggtacgatgattcgagtgacacttacaaggtgttggcgatcctttttaacgttaaatcacaagattgggagctgagagttcactgcatgggtgatgacactggttggagaaatgttttgacttgttctgcttttcccattttgcaacaagtttatgggcagtttgtgagtggcactcttaactggctagcacttgacaattcgtcgggttctgatcattatcaatgggaaactgtcactgtcgatcagttagtcattttttcctatgatctgaagaacgagacatacagttatttgtcgatgcctgatggtctttctgaaatctctcttgacgagccttatcttggggttttgaatggctgcctttgtctttctcatgatcacaggcgaaccaatcttgttgtttggctaatgagagagtttggagctgaaaagtcttggactcagttgctgaatgtaagctatcatcatcttcaagttctagattttccaccttgtccagtggtacctttgtgcaagtctgaaaatgacgacgtcctgttgctggaagactatggaggtggtgcagaatttgttctggttgataagagagataatagtatagaccgcatggaaggtttcaacaatggattatcgtctttctctgcatttgtctctcatgattatgttcaaagtttggttttgccatatcgaaattaa

SEQ ID NO: 198 - DNA, 2661 bp, Glycine max
  source 1..2661: mol_type = other DNA
atgggagctagtcgaaagcttcagggcgagattgatcgtgttctcaagaaggttcaggaaggcgtcgaagtcttcgatagcatctggaacaaggtttacgacaccgataatgccaaccagaaggagaagttcgaggctgacctgaagaaggagattaagaagctgcagaggtacagggaccagattaagacatggattcagtccagtgagatcaaagacaagaaggttagtgcctcatatgagcaggctctggtggatgcacgcaagattattgagcgtgaaatggaaagattcaagatctgtgagaaggagacaaaaacaaaagcattctccaaagaaggcttaggtcaacagcctaaaactgatcctagagagaaggctaaatcagagacaagggattggttgaacaatgtggttggggagttagaaaatcaaatcgataactttgaagctgagcttgagggactttctgtcaagaaaggaaagaacaggccacccagattgacacatctagaaacatcaattactcggcacaaggctcatataaagaaatgtgaatttatcttgaggctactagataatgatgaactaagtccagaggaggttaatgatgtcaaggatttcctggatgactatgtagaccgtaatcaggaggactttgaagaatttagtgatgttgatgagctatatagttcattacccttagacaaggtggagtcaattgaagatattgttacaattccccctggtcttgctaaggtggcacctgtgcttagcttgaagccttctgtggtagcatcagcttcagcatcacaaacatctgagcaagctgatgatacagcttcccaggatagcaattctgatattgttgcaggaactccacctcctaaaagtagtatagttagtcctactactacaacaccagcagggaatttttctactcctgtttctatgaatgttcccatgcctaacttgtcgagtccaccagccattgcgtcagccatgcctggttcaaattctgttcggagttccttggagattagtagtcctgtcaatgcttcatcttttgtaaatcagtctagcactatgaaggaggaagagattaatagtttccctggccaaagaccatctccatcactttctgatgttgcacttgtaaggaacatcagcagaaacagcttatcaaatcaagcaacaaataacatacctcttgtttctggcaatatggtttccagcaatgggccccttggttcagtcccttcagcatctgaaataaccaagagaaacatattggtagttgatgatagactcggaagcaatgggatggtgcagcctcttgtatcccctttaagtaatagaatgatcatgcctcaggttgcaaggcctaatgatggaacttcctcagttgactctagtagtgtcaacgaagctgcaactgtatctgggagagttttctccccttctgctgttcctggcatgcaatggagacctggaagccccttccagaatcagaatgatgtgggtcagattcgtggaagaactgaaatagctccagatcaaagggaaaggtatttgcagaagttgcagcaagtgcagcaacaagggcaaagtgcccttcttaatatgccatcttttgtaggagggaattctaagcagttttctgctcagcaacaaaatcctctcttacagcagttcaattctcaaggctcatctgttgcttctcaatctagcgtgggacttggagtccagtcaccaggtcttagtggtattgcttccacctcattaccgcagccacccaattctgtccattccccatctagccaacaatcattgctattagttgtttccaaagatgcagatgttggaaattctaaggttgacgagccacaactacatatttttcctgatgattcggggactgaatctactgctagtactgggattggcaaaaatttcgtgaatgaagatgaattgaaatccacatatgccgtagattctcctacaggagtacctgcctctcttccagagcctgctcagacttctagagatattgatttgtttcctggccaacctttacaaccaaatcagcgtagtggtaaccttggtgttataggaagaagtttgactgaccttggagctgttggtgacaacttcagtgcatcaactgctaattctggcggagttcgtgatcaattatataatttgcagatgcttgaggcagctcacctcaaacttccacaacctaaggactcagaacgtcctaggacctatactcctaaacaccctacgataacacctcctagttttcctcaagtacaggctcctattgttaataatcctgctttttgggagagagtaggtattgaacaatatggcactgataccctgttctttgcattttactatcaacagaacacttaccaacagtatatggctgcaaaagagctaaagaagcagtcttggagatatcaccggaagtataacacatggtttcaacggcatgaagagccaaaagttgcaacagatgaatatgagcaaggaacatatgtgtactttgatttccatattgcaaatgatgatctgcaacatgggtggtgtcaaagaatcaagaccgacttcacctttgaatataattatcttgaagatgagcctattgtctaa

SEQ ID NO: 199 - DNA, 1536 bp, Glycine max
  source 1..1536: mol_type = other DNA
atgtacttggaaatctcctctctaagtctatgtatacatgtccaatgtcgtgggagtcaggaaaagttgattccctcatgggatgaagcgatagagaatggtttgcgagaattggggtggtcggggatggcgcaatgcatccttgtgtccattgctatgtgctttgatgcacagcagtccttcatggcaatctactccgacgactacccaacatggcactgcacagagacagcatgcacctcatcacgttctgacatatgcaagcttccaaagtcctcttgggcatgggatggaccctcttctaagacaatcatatcacagtttggccttgaatgtgcaggcagcttcatcacaggtttacctcaatcttctttcttcgccggttgccttatcggttcctttcttcttgcctccttggctgatacatctcttggaagaaagaacctcctcttgctgtcttgtttatcaatgtccatatcttctatcttcattgtcttctccaccaatgtttggatctactccgcctttaaattcctcatcgggttttggcgttcatccatcggaacatgtgttctggtgttgctgactgagaaagtgagcactgagtggaggttcacagtgggaattgtcgaatatttctgcttcacattggggtatatgatcttgcctggaatagcttatataaacaagaattcttcatggaaaactctctatgtgtggacatccattcctgccattttttactctatcattgcttacctttttgttaccgagtctccaaggtggcttctcatgcaaggtcgagagcaagaagccatggcaatgcttaacggagtttcttcagtagaaaatggtgctaatttaactgctagcctgctgaaagttcctgctgctaaacaaaagtcttcaattttccaactttactcatcaatagcagagttgtttgagagaagttgggctcttaaacggatggtggccattatggtgcttggtcttgggattggaatggtgtattttggcatgcctttggccgtcgggaacttaggatttgacatttacttggcagttgtgcttaatgccttgatggaaataccttcttgtgtagtcacctacttcttggaaaactacagaagaaagccatctattcttgcattttcagtagcaagtgggatatgctgtgtattgtgtgtggtcgtaggcagtgggcagcaggtagctaaggtggggatatcattggtagccttttttagtgctgtcacggcttataatgtgtttctcatttacattatagagttgtttcccaccagcgtaaggaacaccacaacctcattggtgaggcaagcaactgtatttggtaacatattcatcccatttttgatatctgcaggaaggaaaaatgatattttctcctatggcgtatttggagtagttataatatcatcctgtttgactttagtctgtttgccagagaccacaggaatagcactttgtgataccatggatcaacaagagaaaaagcacagcttatctgtgtaa

SEQ ID NO: 200 - DNA, 960 bp, Glycine max
  source 1..960: mol_type = other DNA
atgaaacacctcctgcaattcgtgtctaccacctccaatctcaccagctttgcagtgtttcagaagcatttgaatgccaagacattggtttgtccaactgtgaaagcaatggggtctaaaagacccttttcaaattcatcaaaaccctcgtcaccatttgttgaggacaagaagaatgtgaagcttaaagggttggaagccagccttggttccaagaagcttgttgttgaggagaatgatgttaatagctattcgattgagcttgagagattaagaaatgacccaacaatagtagacacggtgactgttcaggaactccggaaaaccttgaagagatttaacgtccctgccaaaggtcgtaaagatgatattttgtctgcctggaagagtttcgtgggcagtaacatgtgtgaactagattctcatgcacaagaaaaaaaatggccatggatttcttctgaaaatgcatctgtagaagtggaggctaaaaaagtattggatgaagaccatattgaaaatgtcaatgaaaatcccgagatatctgaacttaaccaggctaagagaaggttaaaacaatcagaatctgagagaaaaactatcaaagtgacaacaaagaagaaagtttcattgaaatcagacgaggattcagattttaagccttccagggcaaagagaaaagtatcttcagatgttgctagcattgttgtacagtcagaggaaatcagtacagctactattcaaactgaaccatggacagttcttgcccacaagaagcctcaaaaaggttggattgcttataatcctagaactatgagacccccacctcttgctcaggatacaaaatttgtcaagcttttgtcatggaatgccaatggattgagagcattgctaaaattagaaggattctcagcacttcaacttgcccaaagggaagactttgatgtattgtgtttgcaatag

SEQ ID NO: 201 - DNA, 1194 bp, Glycine max
  source 1..1194: mol_type = other DNA
atgtcccacagaaacgtggtcacctggacggccatgatctggcaacaaccatatgagtgcatggagtgtgtttcctcaaatgctcagagatggggtgaaccccactgctttcaccatatcatcttttttgaaggcctctgtaagggtttgaaagctcttccatgtggccagttagttcatgctttggccttcaagattggtgtccaaggatcatccatatatattgataatgcacttatggatatgtatgccacttgttgcaataggatggatcatgcaaggatggttttcgatgatatcacaaccaagactgatgatgaaggagcgttaagtctcttcagcttctcaattgcagctaaagcttgtgcatcaattggatcgggtattttgggcaagcaggtaaatgctgcagtggttaagcatggatttgagtccaaccttcctgtcatgaattccatactggacatgtactgcaagtgtcattgtgcatctgaggaaaaacaattattctctgaaatgactcataaagataccatcacatggaataccttgatagctggatttgaagcattggattccagagagtctctctgcatattttctgcagtaggtgcatgtgctaacttagcagttctgtattgtgggcaacagcttcatggtgtaattcaaaatgctatgcacaaatttgtctcctggacttcaatgatgattgaatatggggatcacggatacggtaaagaggctgttgagttattcaatgaaatcgtcagatcaggtattaaaccagataagatggtatttatggcagttctaagtgcttgtagccatactggactagtggacgaagggatgagatattttatattaatgaccagctattatgatgtaacccctgatatagaggtatatgaatgtgtggttgatttgcttggacgtgctggaagagtaaaggaagcttatcaactaatagagagtatgccatttaatccaaatgagtctatctgggcagcccttcttggagcttgtaaagtgcataaacaacccactctggatatgaagccaaatagtgcaggaacttatgcgttgttatcaaatatttatgctgctgaaggaaactgggctgattttgccagctcaacgaagttgagaagtattaaaaataaaagtgagtcggggaggagtggattgaattga

SEQ ID NO: 202 - DNA, 309 bp, Glycine max
  source 1..309: mol_type = other DNA
atgattcataaggataaattcccttatgaaaacactaatcaaatccttgcttcctgttttttcattctctttgtgactagaaaagatgtgaaggctacctgcggaatacctgaaatagcttttgcaacatttgagaacatggaatatggagaagattacatgaagcctgatacagagacatacaattgggttattcaagcttatactagagctgaatcttatgacagagtacaagatgttgctgtgttacttggcatgatggttgaggatcacaaacgtatacagccaaatgcgaagacccatgcgtaa

SEQ ID NO: 203 - DNA, 213 bp, Glycine max
  source 1..213: mol_type = other DNA
atgtccctaataaatatagtcctaattaatgctatggatgtttccttagttgtcaatagtggcactgtagtgccaaagcagtgtggagtgacactgggccgttacaaggaggccatagcatctcagtttagttgtgcggctgtcacaactgctatcacggagtggccgaaatcacggtggagcaggtgctttgtggacgctacattgaaataa

SEQ ID NO: 204 - DNA, 2859 bp, Glycine max
  source 1..2859: mol_type = other DNA
atggttagcggcaacatttttcattgtagaaaaaactcgtggcctccgcaagagtatatcagtaaatccactttgcagctgtttgattatgatagttctgctccccctgaacaagcttggagaaggagattaaacagccatgccaatcttcttaaagaatttagagtaacttttacggaagctataaaaatggttcgattaggtatacgcatttggtcatatgttagggaagaggcttctcatggaaggaaagcgcctattgatcctttcactcgagaaagttgcaagccatctgcatctcaaggagttccacttggagggatggggagtggcagcatatcaagaggatttagaggcgagttccgacaatggcaaattattcccagcttatgtgaagcatcaccagtcatgtcaaatcagttctctatttttataagtagagagggaggaaagaaaaaatttgcatcagtcttggctcctggccagcatgaaggtttaggatctagcaggaaacctgatgatcagggcatatcatcgtggggatggaatctaagtggccagcactcaacctaccatgctttgtttccaagagcttggacagtttatgatggtgagccagatccagaactaaaaatatcgtgtcggcagatatcgccgttcataccacataattatagagaaagcagtctacctgctgctgttttcgtttatacgttggtgaatactggtaaggaaagagctaaagtcagccttctgtttacttgggcgaattccattggtggaagctcacactcgtcaggagatcatgtgaatgaaccattcaaagctgaagatggagtctctggtgtacttctatatcacaagacagcgaaaggaaaccctcctgttacctttgccattgctgcatgtgagacacagaatgttaatgtttctgttttgccaagttttgggctgtctgaagaaagtagtatgactgcaaaacacatgtggagtaaaatggtgaaggatgggcaatttgaccaggagaatttcaattctggacctagcatgccctcatcacctggagagacactttgtgctgctgttgcagcttccacatgggttgaaccccatggaaaatgcaccgttgcatttagtcttgcatggtcatctccaaaagtaaagttcgtgaaaggtagcactttcaacaggagatatacaaaattttacgggacttctgagaaagctgcggcagacttggcccatgatgcattgacacattacaatcggtgggaagaagagattgagaaatggcagaatcctgttcttaaggatgaggccctacctgaatggtacaaatttacattatttaatgagctgtactttcttgttgctggaggaacaatttggattgactctcctgtgctatcttcgaatatgcggaatgaccaggatcgagtaagggagttagaaagtgcagttgtgaaagaaactgaagataaaatgagcgaccgaaaaaggacagttgtggagagtacaacggatagtacctatgattctgctgttattacaggacatgatcgtgcagatgaaaaattgtatgaagatgatgatgatgttggaagattcttgtacttggaaggagtggaatatatcatgtggtgtacatatgatgtgcacttctatgcctcatttgcccttcttgagctctttcctaggattgaattaaatatacagcgtgactttgctagagctgtcttgtgtgaagatggaagaaaagtaaagtttctggcagagggaaattggggcattcgcaaggtttatggagcggtgccacatgatttggggacacatgatccatggcatgaaatgaatgcctacaacatccatgatactagcaagtggaaggacctgaacccaaaatttgttcttcaggtgtatcgagattttgctaccacaggtgatttgcaatttggagtagatgtgtggcctgctgtccgtgctgcaatggagtacatggaacaatttgatagagatggtgatggtcttattgagaatgatgggttccctgatcaaacatacgatacatggacagtccatggtgtgagcacttactgtggttgtctttggcttgctgctctacaagctgcagctgtaatggcccttgaactaggtgacagagaatttgcagaaacatgtaaaaggaagtttttgaaggctaagccagcatttgaagaaaaattgtggaatggtacgtattttaactatgacagtggatcaagcggtaacagtaaatccattcaagcagatcaattggctgggcagtggtatacagcatcctcagggctgccctccctttttgaggattctaaaatcaaaagtgctcttcggaaggtttatgatttcaatgtaatgaaagttaaaggaggcagaatgggtgctgtaaatggcatgcatcccaatggtaaggtggatgagacctgtatgcagtctcgagaagtatggacaggtgtgacctatggtcttgctgctacaatgatacacgcgggaatggaagaagaggccttcacaactgctgagggaatatttctagcaggctggtcagaagatggatatgggtactggtttcagacaccagaggcatggacaatggatgggcactacaggtcccttatgtatatgaggcccctagctatttggggcatgcaatatgcaataaatcggcccaaggcaattctagaggcccctaaaatcaatatcatggacagaatccacttatctcctgttattggaggatactctcataatgaaacaggtgtgaggaagattgcaacaaaagcaggatgctttagcaattctgtatttaattgtgcttgctga

SEQ ID NO: 205 - DNA, 1674 bp, Glycine max
  source 1..1674: mol_type = other DNA
aacgttctgctgttgagaaacaacttaaatttgagtccagatattgctgaagtctcacaagagaaattgctttcattggttgctgaaagattaattgattcaaatagtagtgtgaatggcaaagatgcaggctatgttgaaaatcaacaacagaacattgctgatgcaattgacttactccctaggcttgctactggaattgatgtaaatataaaatttaggagaatagctgattttgaattcactcgagagtgtgccatatttgatctgctggacattcctttgtatcatggctggatggttgatcctcaggattatgacactgcgaatgcaattggatcaaagtcctataatgcccttatgggagagcttgtctctcttgaaacactaaacatgaatgtccatcatgaaaataatccagaagattgtgttgattttgtggctgcaacaactgctactcttggagttccttctcccagtctttcaaaagccaggtcgtttgattattcttctcattctatttctgatcatatacaaagaaaaggtgatctagaagaagaggcagagttgttgagagtcttgaaaatgtccgaggctgaaaatgatcctgttgtaggtcacataaatggaggagaaatttctgtcagtatggatagaaatatgtgtgatgaggaggttataaatacggattctggagataaattaggaaatagcactggtgctgaatctcttgacctgaataatgtgattgagaacaaccttgatgcattggttcagaatgagagtgaagatatcccatatcctgaaaaacactctgtctctttgtttgagtgccgtgcagatgtttctggaggagatggaaaagtccatgatcaatccactcctacaactatagatcatgaagttgtagatgaatctcatggacctgatgccacagtattatcattctcatcccctggccatacaaattcagattcatctagtgtcagatatcatcaaactgatgtttctggagcattgacttcatgtgttcaagggagtgagcccatatatgaaggagaggaatgtgtgttggatacaagaactggaaattttgaggatcgtgaacttgtttatgaaggtgaggtggtacttgcagaacagtctgacaaaaacaccttagctgctcctgatctaagagctaaggatgaacttactccagaacaaggtgaattgatcaagagtttcttgaggaataacgccagccagttgacattttatggtcttttctgtttacaatatgggcttaaagagcgtgaatacaccatgttcaagtttgagggtgagctctatcttctagccacggaacaaggttacataaatcaacctgatctggtctgggaaaaactgaatgaggtcaatggtgatacattgtttttgaccagtaatttcaaggaattcaaggtagaaaaccatgaaagtagcacttgggatgagaacaatgctctgaccagcactgttgactatcttgccagcatagatagtgcaacacatgcaagcttagatatcaattctgatctacaattagcaatagccttgcaacaacaagagtttgagcaacagccaccacgccaggataattcacagcagcaatcatccattagcggtagctctagactggtcacaggtcccaag

SEQ ID NO: 206 - DNA, 873 bp, Glycine max
  source 1..873: mol_type = other DNA
atgggatttagaattagcagttcctcctcttcatcatgcttttgtttgcaacgtgcgcttcttcctccgttaacagcgatagccacttctactcgtgccttgaggaggagtaattgcataccaagttgttcagtatcttatcaaaagtttgttgagtttgcattggatgaaaccaaacgccacactcacttgatcccttcgcctttacaggaaaagttcagtttcatgaattccaaggatgttaaaggaagttttagtatgctatcatttgaagctgcaaaaattaggcttctacgaagtttgatcattcagacagaaacaatgcaggttttggattttactgtctttccaaaagcagaatatgatgtccccatattttgtgctaactttttcacctctgctaaaacaaacattattgtgttggaccttaaccctgtgcatgatatcatcaatcagtatgagtacaaggagaagtactttaaaagcttaattcctcttggccttaaatatgctgaggtatggttggaattgatatgcaaagcagttaaagagacagatgaatctcagattttccacaatctcgaagcacaacatagatatctgacatggagagctgaaaaggatccaggacgaggtgttttgaagaagctgattggtgacacacttgccaaggatatgttgagaagctttctctttaatggagtcgatgaacttggaagcaaaacattcaatgattattttccacagtactgctgtcaagagggaaatctaaataaaaaaggcaatattattgggaagtcctttgaaaatcgcccttggaatgctagaggagaatttattggatccttcagctggattgacgttgatgtgtag

SEQ ID NO: 207 - DNA, 2103 bp, Glycine max
  source 1..2103: mol_type = other DNA
atggcttcggagactgagacgttcgcttttcaggccgagatcaaccagctcctcagtctaataatcaacaccttctacagcaacaaggagatctttcttcgtgagctcatcagcaatgcctctgacgctttggacaagattcgatttgagagtcttacagacaagagcaagcttgatgctcagccagagttgttcattcacattattcctgacaagaccaacaacacattgtctatcattgacagtggtattggcatgactaaggctgatttggtgaataacctcggtactattgcaaggtctgggaccaaggaattcatggaagccctggctgctggtgctgacgtgagcatgattgggcagttcggtgttggtttctactcggcttatcttgtagctgataaggttattgttaccacaaagcacaatgatgatgaacagtatgtctgggagtcccatgccggtggctcatttactgtgacaagagatacatctggtgagaaccttggtaggggaacaaagataactttgtttcttaaggaagatcaacttgagtatcttgaggaacgccgtctgaaggatttgataaagaagcattctgaattcattagctatccaatttctctctggattgagaagactactgaaaaggagatttctgatgatgaggatgaggaagagaagaaggatgaggagggtaaagttgaagatgtggatgaagataaggagaaggaggagaagaaaaagaagaccattaaggaggtatctcacgagtggtcattggtgaacaagcagaagcctatttggatgaggaaacctgaagagattacgaaagaagaatatgctgctttctacaagagtcttaccaatgattgggaagagcatttggctgttaagcacttttctgttgagggtcagctggagtttaaggctgtcctctttatccccaagagggcaccttttgatctctttgacaccaggaagaagcctaacaacatcaaactgtatgtccgccgtgtcttcatcatggacaactgtgaggagttgatgcctgaatatcttagctttgttaagggtattgtggattctgaggatcttcctctcaacatttctagagaaatgctgcagcagaacaagatcctgaaagtcatcaggaagaacttggtcaagaagtgcattgagatgttctttgaaattgctgaaaacaaggaagactataacaagttttatgaagccttctctaagaacctgaaacttggtattcatgaggattctcagaacaagacaaagctagctgaattgctcaggtatcactccactaagagtggtgatgagatgaccagcctcaaggactatgttaccaggatgaaggaaggacagaatgacatctactacattactggtgaaagcaagaaagctgtcgagaattcccccttccttgaaaagcttaagaagaagggatacgaggttctctacatggttgatgctattgatgagtatgctgttggccagcttaaggaatttgagggaaagaagttggtctctgctaccaaggaaggcctaaaacttgatgagagcgaagacgagaagaaaaagaaggaagaactaaaggataaatttgagggtctttgccatgtgatcaaggatgtgttgggtgacaaggtggagaaagttgtggtgtctgatcgtgttgtggattctccttgctgtctggtgacaggtgaatatgggtggacagccaacatggaaaggattatgaaagcacaggcattgagggacagcagcatggctggatacatgtcaagcaagaagacgatggagattaaccctgagaaccccatcatggaggagctgaggaagcgagcagatgctgataagaatgacaagtctgtgaaggatctcgtgctcttgctctttgagactgctcttctaacttctgggttcagccttgatgatcccaacactttcggtaacaggattcacaggatgctgaagcttggactgagcattgatgaagatgctggtgaagcagatgctgacatgcctcctcttgaggacgctgatgcagatgctgagggtagcaagatggaagaagttgattaa

SEQ ID NO: 208 - DNA, 1869 bp, Glycine max
  source 1..1869: mol_type = other DNA
atgctctctcgctctctcagacctcgtcacatactctcctccctccgttcattaccctttaccctaaacccaaacccaaaccctaaccctaaccctaaccgaacccctccaatccgagcctccacacaccgtcccttctcttcttcatggctgtccgaacctggaaacccaatcatccaatggccctcactacccccaccaaaacccactccgaacccaaacccaaacccaaacctaaaccccacacccgaccctccctctcccaacccaaacgctctctccgtcatctccaacctcttcgccgacccttccctctcgcccggcccggccctccacgccgagctcgaccgggctgggatcgagccggacccggcccttctgctcgccgtgttcgatcgcttcggctcgtcgcccaagctgctgcactcgctcttcctctgggcccagacgcggcccgccttcaggcccggccccaagctctttgacgccgtagtcaacgcgctcgcgaaggcgagggagtttgacgccgcgtggaaacttgttctccatcacgccgagaaagacggggaagaagaaggagaaaaagaaaggttggtttccgttggaaccttcgctatcatgattcggcgctacgctcgtgcaggtatgtccaaacttgcaattagaacatatgagtttgccacaaataacaagtcaattgtggattctggctcagaaatgagtttattggagatattgatggattcactttgtaaagaagggtctgtgagggaagcttcagagtatttcctttggaaaaaggagttggacttaagttgggttccttctattcgggtttataacataatgttaaatggatggtttcgattgcggaaactcaagcagggagagagactttgggcagagatgaaggagaatatgagacccactgttgtgacatatggtacccttgtggaaggatattgtcgaatgcgccgtgtcgaaaaggcacttgaaatggttggtgatatgaccaaagaaggaattgcaccaaatgcaattgtgtataatccaataatcgatgcactggccgaagctggaagattcaaagaggcattgggtatgttggaacgttttcatgttttggaaataggccctactgattcaacatacaattctctggtaaaggggttttgtaaggcaggagatcttgtaggtgctagtaagattcttaaaatgatgataagtaggggtttcctcccaagtgctaccacttataactacttttttaggtacttttcaagatgtagaaaaatcgaggaggggatgaacctgtatacaaagttgattcaatctggttataccccagatcggttaacttaccatcttctggtgaagatgttatgtgaagaggagaagttggacttggcagttcaagttagcaaggaaatgaggcataatggatatgacatggacttggctacgagtaccatgttagttcacttgctttgcaaagtgcgtaggttggaagaggcttttgtggaatttgaggacatgatacggaggggtatagttcctcagtatctgactttccagagaatgaaagctgatttaaagaaacaaggtatgactgaaatggctcaaaaactttgcaagttgatgtcttctgttccttattctccgaatttgccaaatacttatggtgaagtcagagaagatgcatatgcacgaagaaaatctataattcggaaagccaaagcattttctgatatgctgaaggactgtaaggaccctagtgaacttcgtaagcatagaagttcatcagaaaatactgtctcaagcacaaacagtttgatagaggatattgagagaaaaagaaatacaggatga

SEQ ID NO: 209 - DNA, 2898 bp, Glycine max
  source 1..2898: mol_type = other DNA
atgccacttgctgaattgtatgatcctgctagaggttatcttgtgaatgatacatgtgttgttgaggctgacatttctgtgcgtaaagatatggattggtcatatgattcaaaaaaggaaactggttatgttggactgaaaaaccaaggagctacatgttatatgaattctctgcttcaaacattgtaccacatttcttatttccgaaaggctgtgtaccatatgcccacgactgaaaatgatatgccatcagggagcatccctctggcattacaaagtttgttttacaaattgcagtatagtgacactagtgtggcaacaaaagagttaacaaagtcttttggatgggatacatatgattcgttcatgcagcatgatgtccaagagcttaatagggttctttgtgaaaagctagaaggaaaaatgaagggaactattgtggaaggcactatacagcaattgtttgaaggacaccatatgaactatattgaatgcatcaatgtggattataaatcaacaagaaaagaatcattttatgatcttcagcttgatgtcaaaggctgtaaggatgtgtatgcttcttttgataagtatgtggaagtggaacgacttgagggtgataacaagtatcatgctgagaactatggtttacaggatgctaggaagggtgtgttgttcattgatttccctcctgttcttcagcttcagttaaaacggtttgagtatgattttacgcgagacactatggtaaagatcaatgaccgctatgagttccccttgcaactagatcttgatagggataatggcaaatatctatctcctgaagcagacagaagtattcgcaatttttatacacttcacagtgttttggttcacagtagtggggtgcatggcggacactactatgcttacattagaccaacactatcaaatcaatggtttaaatttgacgatgaacgagtatcaaaagaagatccaaagagggctttagaagaacagtatggtggtgaagaagagttacctcgtataaatcctggcttcaaccactctccttttaaatttacaaaacactcgaatgcatatatgcttgtctatgtacgtgagagtgacaaggataaaataatttgtaatgtggatgagaaggacattgctgaacaccttagaatgagattaaagaaagaacaagatgaaaaggagctgaagaggaaggagaaggctgaggctcacttgtacaccaccataaaggttgcttgtgatgaagatctacgtgaacagattggaaataatatactctttgatcttgtggattatgataaagtacgaagttttcgtgttcaaatacaaatgccttttatggttttcaaggaagaaattgccaaagagttcggtatacccatccagtatcaacgtttttggttgtgggcaaagcgccaaaacaacacatataggccaaatagagcactgacacctcaggaagaagcacaatcagttggactgctaagagaagtttccactaaagcaaataatgcagagctgaagttatttttggaactagaaatggggcaggatttgcgccctattcctcctcctgagaagtcaaaagagaatctcttgcttttctttaaactttatgaaccttcaaatgagaagcttcggtatgttgggcggctttttgtgaagagtagtgggaagccagaagatatattggtaaaactaaatgaaatggctggatatgctcctgatcaagatatagacatgttcgaggagataaaatttgtgcctaatgtcatgtgtgaacgggttgacaagaaatccacattctttgggagtcagcttgaggatggtgatattatttgcttccaaaagtccgtccaaactggaagtggagagcgatatcgctatccagatgttccttctttcttggaatatgtgcacaaccgtttggttgttcgctttaggactttggagaaaccaaaggaggatgaatttagtctggagctgacaaagcttgacacttatgacaatgttgtagaagaagttgctcaacatattggtttgagtgatccttctaaaattagactcacatctcataactgctactcccagcaacctaaaccacagtctatcaagtaccgaggaatggaacatttgtcagacatgctgattcactctaatcagacttctgatattctatactatgaagtattggatatccctctgccagaattgcaatgcctaaaaactcttaaaattgctttccatcatgataccaacgatgaagttgtgattcatactattagattaccgagacatagtactgtgtctgatgtaattaatgatcttaaatcaaaggtagatttatcacatcctgatgcagaacttagattgcttgaaattttctatcacaagatctataagattttccgtgtcagcgaaaagattgagaacattaatgatcaatattgtgctctacgagcagaggaggttcttgaagaagagaaaaaccttggcccgcatgatcggttgatccatgtttatcatttcttgaaagacacaactcaaaatcagcagcaagttcagaactttggacatccttttttattggttatccatgagggtgagacattaactgaagtcaaattgcgaatacagaaaaagttgcaagttccagatgaggagttttcaaagtggaagtttgcatttttgtcatttggtcgtcctgagtaccttcaagattcagatattgtttcagctcggtttcagaggagggatatctacggtgcatgggagcaatatctcggactggaacacactgacaatgcttcaaaaaggtcgaatgctgccaatcagatctttgctcctaactaccctccccccaaatggcaatggcaaaaatag

SEQ ID NO: 210 - RNA, 151 nt, Glycine max
  source 1..151: mol_type = other RNA
gacaccaaagatagggatattaaacgaatggaattagtatatggatgtaatataatgaaatagagccacttttgggttccctatgaaatgaggcatagaaaggagccactgtgaagaagttttacgagttacgaaggaaacttcgagttca

SEQ ID NO: 211 - DNA, 3248 bp, Glycine max
  source 1..3248: mol_type = other DNA
atatcttcaacatgattttctttttaaaactctatttgtagacatataaagtttttcaagataattttcgtttgttctaaaaaaattttctttttgttcttttagaaactaagaataatttatacatatttgtaagggttattacaattagtggtttgtttatacatccttttcttttttttttataagatagttataaaccagtaaaagtaggaagagtggatacaggtagtgtacacatgtttcaactaatcgaataatcagtatgtaaattcaacaacatcaattctataaagtaaataaaaaattgaaaaagataattgatttttttaaacaaattaataaccatttattaaaaaaaattattgaacaattatagaggattgtatgaaatttaattatcaagtttgttataaaaatttacaactgaagattcaatatcaaaaaaaaaatataaaataagattaaagtagaagaactatataaataactaaaaagagtattaaaataataaaaagatagcatatataaaaaattgatgttgatgaaaataatttgtaaattaatgttgaatatttttttagtaagttgtagatatagatataagatatattttacaaatttttcctttaaaggtgcactgttaaagatttcgtagatgaataaaacattttaattatagaatttaaacttgtattattagtaagttttgtattaattattaataaactggtaaattgaagtaggcattgatactgtgggccactaaactggtaaatttataaaataaaagtgttgaatataaaactaaaaatttttaattaaaagaattaaataaagatactctaaattttaaactttcatactaaaaaaaattacagataatttgaaattttcttatgaggcgtgtacactattatattaacaaacaatgttaacttataaatatattacactaatgacactcccattttttttcccttttttttgtacacattaaactgctttagaattcttattattcacataaaatgaggtggaaaattaaacaattaaactatacattaaatgattttacttactgtcaatagattatttgctaaatcatttataaaatatataaatattaataaacgtttatatatattttatattttttatacgtaaaaaatatttttaaacaaatatttattatttttatatttaattatatatttacttatcagtaagttttgaaaatgaaaagtttctaatttttaattaaaattaagttatttattaattgatattgaacttatatataaatataaaaattgcaattaatttttcaaactcacaatttattttattaaatttaactcacctattagctaaactttaataattagtaatactaattatcatacgtcatgtaatcttataaaataaacttgttagttaattttgttagattttatgttaatatgttcccttttcatcaaccgtttttttttttacgtgctataaagaaaatgttattaataaatttttagatcttttaaaataaaattcaactaactttacaacaaattattattaacaaatttaatgataggatgaattgtgtattaataaaaataaaacagtaaataaaatataggttttaatatcagatgtttagatattatctattaattttacaatacgaaactttaagtactaattttatttatattgaaaaaattttgtttatatataaagtttataattttttattatagtgttgttagatcaaaaaatttgtttctatttgctataattgattattaatttccttatttaaaaaattactaaatattaaagtctttaataaaccactaaaaaaatgattaaactactcagaacttagaactaactatcaaatgttaaagtttttggtaacatgacaaaataattaaactgggtgctaaattcgggtattattgtaccatataatcagactaggctactattttctttcttacccgagacctttatatgatggaagaagaccgtcgagactgtacagatagaggctcgaggcgctcttcgtggtaaccctcgatcttttagactgagactggtgtgttggagagttttgcaactttaagaagtgtaatcgacaataagttatgtagttcttttgtcgttatatagataaccgttttccacctacccaatacgaaaagtcacaacgtcaaccctgagaaccgtatcacgactactgggaaccaacggaggtactcttcgtagaactcctcgaagaacttataaaagcgaaacctgacaccacccaacgggaaccccaacgaagaagttaaccagatcctagaccaaacgtgtgtaaacaggatataaacccaggggtgtatcgtgacaaatgttatcctcgtcacgttacaccggctcaactaaacttttcacgaggaatactatgctatgttaatttttctccaggaagaaccgaactatttctgacaagactcaaacccggtggtaataaggtcagcgtccaaggcgaatcgtcgtaaaacggagttcaagtcgacctccgataagataccccataaccttgtcgatatcccctcgaaggaggaatgaaatagaggtcccgtcgtgcgaacagaccaccctctcatctacggtaccttcttaatctgtcgcttctattttctcagaactcagcttatttcacgaccaaagatagtgtgagcgttgtaaacttaaagaaatgataagatcacgaacgaagccaaggtttaggagataaactggaacggccgtagtacacacctgttaaaccgtaaggtaaaacctttaaaaaagaacgttggaactaacctttccgttaataattttgagtgtatgtctgctataagtattagagtcaaacgttgttagttgaagaactaacctaactcttacttaaataaacccaagagtcggtgtatggaccaaaacgtagacagaacggatctcactgacgatcagaggtacgttactttctattcatagactttcgtggggtaggtgaaaggggtttatttgtccccttttttaccctaaaaagaaaacgaagtcagaccttgtgacaacacaccgagtacgaatacttgaagaaacagttctatcacttacgttgaagggtctccatagacttctttgtcgtcctctgtgtcgaacgacgtaattgccttttcagatggggttgtctgagtctgcgtgttact

SEQ ID NO: 212 - DNA, 3362 bp, Glycine max
  source 1..3362: mol_type = other DNA
cctcgctccttacagtagttacagatgtcctttggattgacatgagtcttcttttttcctttaaacacctttttcttgatgttgttgttagagttagagactacaagaccagatccatttgattcttcagaatttccaagtgctttagatcgaagttcccttgagtataagctggatttcacttcctccatggtgacacattccttaccaacactaagagaattgacaaagctctcatatgatggtggtaaagaggctaacaaaatcacgacaacatcttcatcctctatcttaacatctatatctcttaattccatcaaaatagagttcagttcatcaagatgatccttaagagatgtaccttctttcatgtgtaaaaaaaatagttgccttttcaagaagagcttgttgcatattgacttggtcatatatagcttttccaacttgagccataagccacttgcaatttctttatttgcaacctcatataaaacttcattaggtaaagaaagcaagattagtgagtgagccttttcttcttgttccgcaagttctccaatctttaaaacagaggccttttcttttgattcagaagccactggtttcttcatagatgcagaagcaacaggagcccaaacacgttgttccttcaacaaagcacacatcttgatgtgccatagattgaagttgttttttccattgaatttctcaattctaattgtgtttgacataatttcttgagtgtccttgatcacaatgaagcaacgaaagataccagaaacaagactcacacaacttgatcttcgtattcaaacgagaatcttgaattcccttgatcgcaacttgagctcttgataccaatttgttatgaaagtgatgaagaacaaacaagaacagcagtagcaacaaaacaatatgtcatacacaaaattgcagtaaaatgaaagagagtaaagagagaaaagaacacatagaatttacgtggttcacccttaatcaggctacatccacgaggaggggcagaaagattttatttctgatgcaattcaaaaggaagtgcttcccaatacagaacaacccttccttatataagaaaaggatcagaaaaaacaaaacaaaacaaaataacagaattaactgccatatagattttagtcacaacctaacaacactattgatgaaattttcctaaaataaaaataagatagtgagagagaaatagtaatttataaagtagttatagaagtaaaataggacaaaaaaaggattttagccacaatccaacaacactattgataaaattttcctgaaataaaaataagatagtgagagagaaatagtgatttataaagtagttatagaagtaaaataggacaaaaaaatatgtataccaaaacttcatataatctctgcatatagttatagatatttttgaatttctctcgttttttttttctttacctctttctatttttcattcttacacccaaaaaatgagatctacatctaacattttccattttttttttgtgaagaaacattttcctttgtctccggcattcctccaccagcacatttttagccattggattaatccgatcactgagattaaaatttaaaaaaaaaaatattattgctggcctttggaagaatccaaaagacacattcggtgtgtcaattcaacatactttttgctactagaaaccagctcgtagggccctttaatttctaattcagacactcaaaccatgcgttcaactactattcttatccgagtccacacatagcattttatttttaaatcataatataagccatgtcatgtgccaaatttccttgaagctatttaatatttcaaaatatactacctcctttgttgaataaccttccatttcttagctgtcacattttgtcaatcaattaacaagtgcgtcttttttaatcgcatccctcttgtggctttccttccatttttgcaggagcaatttggcgggttgatcatgagacacgtggttattttttctttcaatttcttgctgctttgtgtcttcatttctgctataacaagcacaaaaagtgaagtatctagcacaaatgaagaacaatcattttcttaccatatcaaagctttggaattcatatggaaacatttgggctatcaacatgtgtggccagaaatggaatttagctggagaattgttgttggaaccttgattggaatcttgggagcagcatttggaagtgtagggggagttggtggtggtggcatctttgtgccaatgctaatcctcattattgggtttgatccaaaatcagcagttgctatttcaaagtgtatggtcacaggtgcagccatctcagcagtattcttctgcatgaagcaaaggcatcccacacttgatgaacccgttatcgactacgatttgatgttgctaatacaaccaaccctcatgcttggaatcagcataggagttattttgagtgtaatatttgctgattggatggtcacaattcttctaattattctatgtatagtgacatcaatcagggcattcttcatgggtgctgacacatggaaaaaggaaaccaaaatgaaagaggtgagcatccttggcaacatgtattggaaggagtttgtacttattttcattgtctggctcgcatttgtgaactatacagtttcctgttcagtcacatactggatacttattttgtcacagataccaattactgtaggattttatttgtaccaagcaagagccctatatcaggggagagctgcaggatctcaacacacacattggccattgcaccatctatttctagctagcatttgttctctgttagctggaattgttggtggacttcttggtacaggttctggatttgttatgggtcctttatttctagaagtgggaattgctccacaggtagcaagtgccacagccacttttggaatgatgtattcatcatctttatctgtcatacaatattacctgttgaatcgttttcctgttccttatggtaaaattaaacttaattcacattgtacccttctattatttctgaatggttttcaaaggcaacataaacttattcaagtaatttttatgcatgtttcaacctgttttgcagctctcttccttactcttgtggctgcaattgcagcattcctaggacagtatctcattgacaagcttgttaatatcttccaaagggcttctttaattatttttgtcttggccttcacaatatttgttagttcaattgcattaggtggagtcggcatatcaaacatgatcttgaagattcaaaggaatgaatacatgggatttgataatttttgcaggaatgatacatag

SEQ ID NO: 213 - DNA, 2449 bp, Glycine max
  misc_difference 432..531: note = n is a, c, g, or t
  misc_difference 1991: note = n = a sequence of between 3 and 13 nucleotides
  source 1..2449: mol_type = other DNA
ttaccctcgaaattcaatttactttatccactttagtcgatacttcctctatcacgttcaaaatagtacacttcgattgtaagattggttatatagttatatttatataaaatgactattttttagtaaggttgttggtacttggtgtgttttttagtctttaccggtgactgggcgaacgtcgggacatttgttccaaggtcgaagaagttgttgttgagctactcgacgtgcgcagaaggaacgtttgtggtattcgtttgtagttgcaatttttaaaaaaatagtttgggccctggaacaaagggcttggttggggaataaaaagaattggattttcttcgaattggattgttggaataattgcattggaccaaaagaaggctttcacttaaattacaaccagaagggctttgaccgttcacgttcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtcccgaattcccaagttagcttccgaagttagcttcggtgttccgacagatgtcccaagttacgaactttgactttgttgtttatgcaactcccgttcgctcttaccacttcccaagttagcttccagtgttccgacagatgtcccaagttacgagttcccaagttacgatccagagtcacgacacaagtttccttttctgttatgcaactcccttcgccaaattaaaagtaaaaaaaaacaaaattgagatgcagccaaaaatgacttttggccgcatttacaatatcggacattgtaagattccgccaaaaaatattggcggaatctaacgttcagcatcttacgtttaataaagtgtttttaatgtttttacggtggcaagttgaataatctcgccaaggatttattggctgcatttaaaccatagcatttttcggaaaaaaaatcatcactgaaagagggggttgtttagttttgaagtaaggaaaagaaactaaggcaatttaatggtaatttgtgtccatgaaagtgcggacacacaagtgttaaaaataaaaggacgttgtaaaaaacaattttaactatataaaaaagttatagagaatcataggcgtaatatattgtgtgttgtgttattgtttttacattattgtgtgttatacataaaactttaacttcatatagatattaagtgaaatcaataaagggaaatattattacacggttacggaacctacgtgtgtatgtaccattgtaagataaagtttttaataaaaaacaaaattttttaaaaattttttatgaaaagaaaatttttaaattataacttatttcactaaaaattttaacaaaattttaaacataagaaaaagacggatataaaagataacaaattatattaagtaaaataacttatgtattatgtactaattaaaattttttaatgtgaattaaaaaattactttaatacaaaaaattaaaaaaatcttttattattttaatattttttcgtacggatgtttaatcatttcaaccaaaattaaaaagatgtacagtgaaacaactaaattttgtattttaactgagatacatattaactagcttccaaaaagaaaaaattagattacacttttcacataaataataaatgaaattaaatcgacacaattatttaataattatgtatcagggcaatttaagttaatacgtttgaattttagtcagaatctaaaccatggttattctcaattaaacaatggttacttctctctcttccaatatcctctctcttgttacttaactttcaattaaacaatgtattttttcttaatgtatattaattagtaattatgtataatatgtggactttacctaaggatctttatttttttttatattttaagtacaattaacacaacgaagcgaaagagatatcgaacgaggagaaaatctattangaaggtgttcggatcgagcttcttccttctctattctcccgggataccttttacaccagtctttaggtattatctcaggctggtgttgatagcttaattaatagaagtacgtattcttatgatctaataggtcatattttctaactttttagtagagtttggagagaaaaagaaaagataacgttaaagacctaagaaaaagataacgttaaaaacctaataatatattgctaaaaagttgaaaggtaaaaggttttaaaaggtaaatatctttatctatctgatctttgctgtagagaatagagttactgtggtttctatccctataatttgcttaccttaatcatatacctacattatattactttatctcggtgaaaacccaagggatactttactccgtatctttcctcggtgacacttcttcaaaatgctcaatgcttcctttgaagctcaag

(19) United States
(12) Patent Application Publication
(10) Pub. No.: US 2025/0163452 A1
(43) Pub. Date: May 22, 2025

(54) DISCOVERY OF SOYBEAN CYST NEMATODE RESISTANCE GENES BASED ON EPIGENETIC ANALYSIS

(71) Applicant: UNIVERSITY OF TENNESSEE RESEARCH FOUNDATION, KNOXVILLE, TN (US)

(72) Inventors: TAREK ABDELFATTAH HEWEZI, KNOXVILLE, TN (US); ADITI RAMBANI, KNOXVILLE, TN (US); VINCENT R. PANTALONE, KNOXVILLE, TN (US); J. HOLLIS RICE, KNOXVILLE, TN (US)

(21) Appl. No.: 18/965,202

(22) Filed: Dec. 2, 2024

Related U.S. Application Data
(63) Continuation of application No. 17/278,346, filed on Mar. 22, 2021, now Pat. No. 12,157,896.
(60) Provisional application No. 62/860,451, filed on Jun. 12, 2019.
(60) Provisional application No. 62/737,383, filed on Sep. 27, 2018.

(51) Int. Cl. C12N 15/82; C07K 14/415

(52) U.S. Cl. CPC C12N 15/8285; C07K 14/415; C12N 15/8213

(57) ABSTRACT

The invention relates to genes which may be utilized to induce resistance to soybean cyst nematode (SCN). More specifically the present disclosure provides genes that, when inactivated or overexpressed in a plant, particularly, a soybean plant, can confer upon the plant resistance to SCN. Methods of using these genes to obtain plants, particularly, soybean plants, that are resistant to SCN are also provided.

### CROSS-REFERENCE TO RELATED APPLICATIONS

This application is a divisional of U.S. application Ser. No. 17/278,346, filed Mar. 22, 2021, now U.S. Pat. No. 12,157,896, which is the U.S. National Stage Application of International Patent Application No. PCT/US2019/053329, filed on Sep. 27, 2019, which claims the benefit of U.S. Provisional Ser. No. 62/737,383, filed Sep. 27, 2018, and U.S. Provisional Ser. No. 62/860,451, filed Jun. 12, 2019, the disclosures of which are hereby incorporated by reference in their entireties, including all figures, tables and amino acid or nucleic acid sequences.

The Sequence Listing for this application is labeled “SeqList-replace.xml” which was created on Feb. 20, 2025, and is 530,243 bytes. The Sequence Listing is incorporated herein by reference in its entirety.

### FIELD OF THE INVENTION

This disclosure relates to genes that may be overexpressed or inactivated in a plant to induce resistance in the plant to soybean cyst nematode (SCN). The present disclosure also relates to methods of identification of gene(s) that can confer upon a soybean plant resistance to SCN and the methods of overexpressing or inactivating the genes so identified to obtain soybean plants that are resistant to SCN.

### BACKGROUND OF THE INVENTION

DNA methylation is a consequential epigenetic change that impacts gene expression, transposon mobility, genomic stability and imprinting. Previous studies identified key enzymes that carry-out cytosine DNA methylation in various sequence contexts. After DNA replication cytosine methylation in CG and CHG is maintained through the activity of Methyltransferase1 (MET1) and Chromomethylase3 (CMT3), respectively. Also, a small portion of CHH methylation can be maintained by CMT2 following DNA replication, but the large majority of CHH methylation sites are reestablished de novo. De novo DNA methylation in CG and non-CG contexts is carried-out through the synchronized activity of the RNA-directed DNA methylation (RdDM) pathway and Domains Rearranged Methyltransferase 2. Failure to faithfully maintain DNA methylation status by the maintenance enzymes after fertilization may cause spontaneous DNA methylation polymorphisms referred to as epialleles. Epialleles can be induced by developmental and environmental stimuli as well as through transposon insertions and genome rearrangements that induce directed chromatin modifications.

DNA methylation patterns of Arabidopsis lines generated through single-seed descent for 30 generations with their ancestral lines revealed that spontaneous loss and gain of DNA methylation at individual cytosine sites occur at high levels mainly in the genic regions in the CG sequence context. Regions with continuous cytosine methylation polymorphisms were also found but at relatively lower rate. Studies of inheritance and stability of DNA methylation patterns in maize and soybean using populations of recombinant inbred lines (RILs) provided additional evidences for transgenerational inheritance of DNA methylation variants over several generations in the segregating populations.

Similar to spontaneously generated DNA methylation variants experimentally induced DNA methylation changes may result in generation of novel non-parental DNA methylation polymorphisms that are heritable. For example, DNA hypomethylation induced by mutation in the Arabidopsis DDM1 gene was stably transmitted over several generations. Similarly, ddm1-induced hypermethylation of BONSAI gene was consistently maintained in the ddm1 after recurrent self-pollination. More comprehensive studies of transgenerational inheritance of DNA methylation variants were conducted in Arabidopsis using epigenetic recombinant inbred lines known as epiRILs that were generated by crossing the hypomethylated mutants met1 or ddm1 with wild-type plants. Analysis of DNA methylation profiles in these epiRILs documented the transgenerational inheritance of parental DNA methylation patterns in addition to the presence of newly acquired non-parental methylation variants. Furthermore, phenotypic analysis of the epiRILs for various traits including plant growth, plant height, flowering time and response to biotic and abiotic stresses revealed high degree of heritability, indicating that epiallelic variations may contribute to the heritability of complex traits. Combining hypomethylated and normally methylated genomes in F1 plants triggers substantial reprogramming of plant methylomes that may result in novel and heritable epialleles. Therefore, interference with DNA methylation programs may spontaneously trigger heritable epigenetic variations that may be conditioned by cis- and/or trans-acting differences.

In soybean, SCN (Heterodera glycines) is a damaging pathogen, causing significant yield and quality losses. SCN induces vascular root cells to fuse and form a permanent feeding structure, the syncytium, essential for nematode development and maturity. Resistance to SCN is conferred by two main loci, Rhg1 (for resistance to H. glycines) and Rhg4, at chromosome 18 and 8, respectively. The Rhg1 locus contains three genes within a 31-kb repeat region that encode an α-SNAP protein, a putative amino acid transporter, and a wound-inducible protein. Increased expression of these three genes, mediated by high copy number, was found to contribute to SCN resistance in an additive manner. Rhg4 locus contains only one gene encoding serine hydroxymethyltransferase (GmSHMT08). SCN resistance in commercially available soybean cultivars is gained from two main sources that include plant introduction (PI) 88788 and Peking. (PI) 88788-derived resistance is mediated by high copy number of rhg1-b allele (7 to 10 copies), and cultivars produced from this source display slow degeneration of the nematode feeding site, leading to delayed arrest of nematode development. Peking-derived resistance requires both Rhg4 and rhg1-a alleles, and cultivars produced from this source display stronger and faster resistance response, leading to rapid arrest of nematode development at the infective juvenile stage. However, molecular mechanisms through which Rhg1 and Rhg4 mediate SCN resistance remain to be elucidated.

Serine hydroxymethyltransferases (SHMTs) are key enzymes involved in one-carbon metabolism, a housekeeping cellular function that supports various physiological processes including redox defense and DNA methylation. The anticipated function of Rhg4 (GmSHMT08) in redox defense is consistent with the activation of significant numbers of genes associated with oxidative stress, hypersensitive responses and programmed cell death in the syncytium formed in Peking. The potential implication of Rhg4 in modulating plant DNA methylation landscape is striking since recent studies indicated that level and pattern of plant DNA methylation are considerably modulated during cyst nematode infection.

Therefore, discovery of proteins and genes that modulate plant DNA methylation pattern to confer genetic resistance to cyst nematode infection is warranted.

### SUMMARY OF THE INVENTION

The instant invention pertains to the function of Rhg4 and Rhg1 loci in establishing DNA methylome landscapes of soybean roots during SCN infection. Highly homozygous near-isogenic lines (NILs) differing at Rhg4 and Rgh1 loci were analyzed to obtain insights into the role of GmSHMT08 and GmSNAP18 in reprograming soybean methylomes that may prime a plant's response to SCN parasitism.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Comparison of global DNA methylation levels between TN09-16 and TN09-29 over genes and TEs in various sequence contexts under non-infected conditions. Global DNA methylation levels over protein-coding genes (A-C) and TEs (D-F) in the CG, CHG, and CHH sequence contexts.

FIG. 2: Characterization of the differentially methylated regions between TN09-16 and TN09-29. A-C, Differentially methylated regions overlapping with protein-coding genes in the CG, CHG, and CHH sequence contexts. D-F, Differentially methylated regions overlapping with TEs in the CG, CHG, and CHH sequence contexts.

FIGS. 3A-3D: Functional classification of the differentially methylated and differentially expressed genes between TN09-16 and TN09-29. A and B: Gene Ontology enrichment analysis of the differentially methylated genes (A) and differentially expressed genes (B). C: Venn diagram demonstrating the overlap between differentially methylated genes and differentially expressed genes. D: Genome browser image showing DNA hypermethylation in the promoter and gene body regions of Glyma.09G133900 in TN09-16. The hypermethylation of Glyma.09G133900 was associated with gene downregulation. Promoter region, 1 kb upstream of ATG, is highlighted.

FIG. 4: Comparison of global DNA methylation levels between the isogenic lines at 5 day post SCN infection. A-F: Global DNA methylation levels over protein-coding genes (A-C) and TEs (D-F) in infected and non-infected root samples of TN09-16. G-L: Global DNA methylation levels over protein-coding genes (G-I) and TEs (J-L) in infected and non-infected root samples of TN09-29.

FIGS. 5A-5K: Specificity and magnitude of DNA methylation changes induced by SCN in the isogenic lines. A and B: Venn diagrams showing the numbers of DMRs overlapping with protein-coding genes and TEs in TN09-16 (A) and TN09-29 (B). C: Venn diagram showing 74 DMRs overlapping between those identified in TN09-16 and TN09-29. D and E: Gene Ontology enrichment analysis of the DMRs-associated genes identified in TN09-16 (D) and TN09-29 (E). F to K: Methylation level, direction, sequence contexts, and numbers of the DMRs overlapping with protein-coding genes (F-H) and TEs (I-K) identified in TN09-16 (blue lines) and TN09-29 (red lines) in response to infection by soybean cyst nematode.

FIGS. 6A-6C: Association between DNA methylation reprograming during the susceptible interaction and gene expression changes. A: Gene Ontology enrichment analysis of the differentially expressed genes identified in the TN09-016 in response to SCN infection. B and C: Venn diagrams showing the overlaps between the DMGs, DMTE-associated genes, and DEGs identified in TN09-29 (B) and TN09-16 (C).

FIGS. 7A-7D: Characterization of differential methylation patterns identified in the isogenic lines that were stably inherited from the parents. A: Heat map of 58 DMRs showing the methylation patterns in TN09-29 and TN09-16 that were inherited from the parental lines Fowler and Anand, respectively. B: Example of DMRs showing hypermethylation patterns in the susceptible line TN09-16 that were inherited from the susceptible parent Anand. In contrast, the resistant line TN09-29 and the resistant parent Fowler showed hypomethylation in these regions. C and D: Proportions for methylation contexts (C) and the overlapping genic features of the 58 DMRs (D).

FIGS. 8A-8F: Identification of novel non-parental DMRs specific to the susceptible isogenic line. A: Heat map of 56 DMRs showing methylation patterns specific to TN09-16. B: Example of a DMR overlapping with a protein coding gene (Glyma.08g157400) and showing methylation patterns specific to TN09-16. The region was hypermethylated in TN09-16 but hypomethylated in TN09-29 and the parental lines Fowler and Anand. C and D: Proportions for methylation contexts (C) and overlapping genic features of the identified 56 DMGs (D). E: Identification of 13 significantly differentially used exons/junctions in the RNA-seq data of TN09-16 versus TN09-29 under non-infected conditions. The mean normalized coverage of each gene was plotted against fold change values and 13 significantly differentially used exons/junctions (red dots) were identified using q value less than 0.1. F: Exon/junction expression profile for the Glyma. 17G149600 gene. The numbers of normalized sequencing read aligned to each exon or splice junction in TN09-16 (blue) and TN09-29 (red) were obtained from RNA-seq data under non-infected condition and were displayed as gene profile plot. A gene diagram showing the location of each exon (boxes) and the predicted junction sites (dashed lines) is included below the plot. One statistically significantly (q value=0.0072) used exon is highlighted in pink.

FIGS. 9A-9F: Identification of novel non-parental DMRs specific to the resistant isogenic line. A: Heat map of 102 DMRs showing methylation patterns specific to TN09-29. B: Example of a DMR overlapping with a protein coding gene (Glyma. 18g33500) and showing methylation patterns specific to TN09-29. The region was hypermethylated in TN09-29 but hypomethylated in TN09-16 and the parental lines Fowler and Anand. C and D: Proportions for methylation contexts (C) and overlapping genic features of the identified 102 DMrs (D). E. Identification of 11 genes overlapping between the 100 DMR-associated genes and the previously identified syncytium DEGs. F: Accession numbers of the 11 overlapping genes.

FIGS. 10A-10B: Identification of novel soybean cyst nematode resistance and susceptibility genes. A: Nematode susceptibility assays of transgenic soybean hairy root plants overexpressing two SCN resistant genes. The composite plants overexpressing a sulfite exporter TauE/SafE family protein (Glyma.14G142500, according to SEQ ID NO: 2), a SNARE associated Golgi protein family (Glyma. 11G004900, according to SEQ ID NO: 1), or the empty vector (control) were generated in the susceptible isogenic line TN09-016 and inoculated with 3000 eggs of SCN (race 3). B: Nematode susceptibility assays of transgenic soybean hairy root plants overexpressing two SCN susceptibility genes. The composite plants overexpressing an aspartate aminotransferase 5 (Glyma. 17G216000, according to SEQ ID NO: 3), a transcription regulator of the NOT2/NOT3/NOT5 family protein (Glyma.02G071300, according to SEQ ID NO: 4), or the empty vector were generated in the susceptible isogenic line TN09-016 and inoculated with 3000 eggs of SCN (race 3). The number of cysts per plant was counted 5 weeks after inoculation and used to calculate female index. Data are presented as mean value of 8 independent replicates±SE.

FIG. 11: Schematic representation of the two single nucleotide polymorphisms identified in the susceptible line TN09-16 leading to R130P and Y358N amino acid substitutions.

FIG. 12: Responses of TN09-16, TN09-29, and the parental lines Fowler and Anand to infection by the soybean cyst nematode race 3 (HG type 0). The number of cysts was determined five weeks post inoculation. Data from two experiments (highlighted in red and blue), each with at least 10 plants per line are shown. Each data point represents the mean of cyst numbers obtained from two plants.

FIG. 13: Mapping DMRs identified between TN09-16 and TN09-29 under non-infected conditions to various annotated features of protein-coding genes, including promoter, gene body, and 5′ and 3′UTRs.

FIG. 14: Mapping DMRs identified between TN09-16 and TN09-29 under non-infected conditions to various transposon families, including Helitron, TIR, LTR, and Line.

FIG. 15: Numbers and direction of the DMRs identified in TN09-16 (blue) and TN09-29 (red) in response to SCN infection and overlapping with protein-coding genes.

FIG. 16: Numbers and direction of the DMRs identified in TN09-16 (blue) and TN09-29 (red) in response to SCN infection and overlapping with various transposon families.

FIGS. 17A-17F: Comparison of global DNA methylation levels between the parental lines (Anand and fowler) over protein-coding genes (A to C) and TEs (D to F) in various sequence contexts under non-infected conditions.

FIG. 18: Nematode susceptibility assays of transgenic soybean hairy root plants overexpressing two SCN resistance genes (Glyma.11G004900 and Glyma.14G142500). The composite plants overexpressing Glyma.11G004900, Glyma.14G142500, or the empty vector (control) were generated in a soybean susceptible line and inoculated with 3000 eggs of SCN (race 3). The numbers of cysts per plant were counted 5 weeks after inoculation. Data are presented as mean±SE of 8 independent replicates.

FIG. 19: Nematode susceptibility assays of transgenic soybean hairy root plants overexpressing gma-miR5032. The composite plants overexpressing gma-miR5032 or the empty vector (control) were generated in a soybean susceptible line and inoculated with 3000 eggs of SCN (race 3). The numbers of cysts per plant were counted 5 weeks after inoculation. Data are presented as mean values of 10 independent replicates±SE.

FIGS. 20A-20B: Examples of genetic polymorphisms in the SCN resistance Glyma.11G004900 associated with soybean resistance against five SCN races. A: C/T polymorphism in promoter region at the position −1020 is associated with resistance against race 1, 2, 3, 5, and 14. For each race, the bar on the left corresponds to genotype having C at the position −1020 and the bar on the right corresponds to the genotype having T at the position −1020. B: CAACAT/C polymorphism in the promoter region at positions 1718/23 is associated with resistance against race 1, 2, 3, 5, and 14. For each race, the bar on the left corresponds to genotype having C at the position 1718/23 and the bar on the right corresponds to the genotype having the sequence “CAACAT” at that position.

FIGS. 21A-21B: Examples of genetic polymorphisms in the SCN resistance Glyma. 14G142500 associated with soybean resistance against three SCN races. A: A/AT polymorphism in promoter region at the position −482 is associated with resistance against races 3, 5, and 14. For each race, the bar on the left corresponds to genotype having A at the position −482 and the bar on the right corresponds to the genotype having AT that position. B: G/A polymorphism in the promoter region at the position −1101 is associated with resistance against race 3, 5, and 14. For each race, the bar on the left corresponds to genotype having A at the position −1101 and the bar on the right corresponds to the genotype having G at the position −1101.

FIGS. 22A-22B: Examples of genetic polymorphisms in the gma-miR5032 associated with soybean resistance against four SCN races. A: C/T polymorphism in promoter region at the position −905 is associated with resistance against race 2. For each race, the bar on the left corresponds to the genotype having C at the position −905 and the bar on the right corresponds to the genotype having T at the position −905. B: T/G polymorphism in the promoter region at position 2051 is associated with resistance against race 1, 2, 3, and 5. For each race, the bar on the left corresponds to genotype having G at the position −2051 and the bar on the right corresponds to the genotype having T at the position −2051.

### DETAILED DESCRIPTION OF THE INVENTION

The present invention relates to a novel and useful methods for introducing in a reliable and predictable manner SCN resistance into non-resistant soybean germplasm. The method involves the genetic-mapping of loci associated with SCN resistance based on epigenetic analysis of parent and progeny lines that are resistant or susceptible to SCN. The genetic loci identified to be associated with the SCN resistance can be overexpressed or inactivated in a plant to render it resistant to SCN.

Epigenetics can be defined as the biochemical modifications of DNA and associated proteins that regulate gene expression and chromosome structure and function, without changing DNA nucleotide sequences. DNA methylation, the most common epigenetic modifications, is the addition or removal of a methyl group (CH₃), mostly where cytosine bases occur repeatedly. In plants, DNA methylation occurs in symmetric (CG and CHG) and asymmetric (CHH) contexts where H refers to any nucleotide but G. The CG and CHG patterns are symmetric across the two DNA strands, which are believed to be important for the maintenance of methylation at these sites following DNA replication. DNA cytosine methylation, as the main epigenetic mark, controls gene expression networks and hence plays essential roles in different aspects of plant growth, development, and response to biotic stress (Zhang et al., 2010; He et al., 2011, Dowen et al. 2012). While DNA methylation has been initially reported to control various developmental processes in plants, recent studies revealed that this silencing pathway plays a key role in modulating plant defense responses during biotrophic interactions (Yu et al., 2013; Dowen et al. 2012; Luna et al., 2012). Recently, Dowen et al. (2012) provided a clear evidence of dynamic changes in DNA methylation in response to infection by the bacterial pathogen Pseudomonas syringae pv. tomato DC3000 (Pst). Using deep sequencing of bisulfite treated DNA, they found that differentially methylated regions (DMRs) are preferentially associated with genes involved in defense response, and that hypomethylation in DMRs is frequently accompanied by activation of the proximal genes, specifically those with defense response function. Similarly, another recent study indicated that DNA demethylation restricts the multiplication and vascular propagation of the Pst and, consequently some immune response genes, are repressed by DNA methylation (Yu et al., 2013). Chemical demethylation of the silenced resistance Xa21G gene in rice reestablished its resistance function against Xanthomomonas oryzae (Akimoto et al., 2007). Similarly, induced DNA hypomethylation at the NBS-LRR gene clusters by the tobacco mosaic virus was associated with increased genomic rearrangements at these genomic loci (Boyko et al., 2007). The expression difference between the resistant alleles of the Medicago truncatula REP1 gene, which confers resistance against the powdery mildew disease caused by the biotrophic fungus Erysiphe pisi, was found to be correlated with the methylation status at the promoter regions (Yang et al., 2013). In soybean, differential hypermethylation patterns at the genomic regions that contain multiple copies of SCN resistance gene Rhg1 have been recently identified (Cook et al., 2014). Collectively these results indicate that DNA methylation plays a crucial role in regulating the immune system in response to pathogen infection including cyst nematodes.

Epigenetic variation is when the phenotypic traits of an individual vary without altering the primary sequence of its DNA. This can occur through changes in the expression of particular genes via processes such as DNA methylation and chromatin remodeling, and by influencing the activity of RNA structures which regulate levels of gene expression.

Epigenetic changes in gene expression enable an individual to respond to changes in the environment and adjust the synthesis of proteins accordingly. It has become apparent that while many of the epigenetic modifications to the genome are reset during the process of meiosis, some epigenetic information can be transmitted between generations, so that the phenotypic traits of offspring are affected without altering the primary structure of the DNA. Thus offspring can inherit tolerance to a particular environmental condition before they have been exposed.

In this disclosure the term “isolated nucleic acid” molecule means a nucleic acid molecule that is separated from other nucleic acid molecules that are usually associated with the isolated nucleic acid molecule. Thus, an “isolated nucleic acid molecule” includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamine) containing restriction-digested genomic DNA, is not an “isolated nucleic acid”.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain or a microRNA (miRNA); it includes regions preceding and following the coding region (leader and trailer) involved in the transcription/translation of the gene product and the regulation of the transcription/translation, as well as intervening sequences (introns) between individual coding segments (exons).

In this application, the terms “polypeptide”, “peptide”, and “protein” are used interchangeably herein to refer to a polymer of amino acids. The terms apply to amino acid polymers in which one or more amino acid residues are artificial chemical mimetic of a corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used in herein, the terms “identical” or percent “identity”, in the context of describing two or more polynucleotide or amino acid sequences, refer to two or more sequences or subsequences that are the same or have a specified percentage of amino acid residues or nucleotides that are the same (for example, a variant protein used in the method of this invention has at least 80% sequence identity, preferably 85%, 90%, 91%, 92%, 93, 94%, 95%, 96%, 97%, 98%, 99%, or 100% identity, to a reference sequence), when compared and aligned for maximum correspondence over a comparison window, or designated region as measured using one of the following sequence comparison algorithms or by manual alignment and visual inspection. Such sequences are then said to be “substantially identical”. With regard to polynucleotide sequences, this definition also refers to the complement of a test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

In this disclosure the terms “stringent hybridization conditions” and “high stringency” refer to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993) and will be readily understood by those skilled in the art. Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5xSSC, and 1% SDS, incubating at 42° C., or, 5xSSC, 1% SDS, incubating at 65° C., with wash in 0.2xSSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1xSSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al.

As used herein, the singular forms “a”, “an” and “the” are intended to include the plural forms as well, unless the context clearly indicates otherwise. Further, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, include the phrases “consisting essentially of”, “consists essentially of”, “consisting”, and “consists”.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed.

In the present disclosure, ranges are stated in shorthand, to avoid having to set out at length and describe each and every value within the range. Any appropriate value within the range can be selected, where appropriate, as the upper value, lower value, or the terminus of the range. For example, a range of 1-10 represents the terminal values of 1 and 10, as well as the intermediate values of 2, 3, 4, 5, 6, 7, 8, 9, and all intermediate ranges encompassed within 1-10, such as 2-5, 2-8, and 7-10. Also, when ranges are used herein, combinations and sub-combinations of ranges (e.g., subranges within the disclosed range) and specific embodiments therein are intended to be explicitly included.

An endogenous nucleic acid is a nucleic acid that is naturally present in a cell. For example, a nucleic acid present in the genomic DNA of a cell is an endogenous nucleic acid.

An exogenous nucleic acid is any nucleic acid that is not naturally present in a cell. For example, a nucleic acid vector introduced into a cell constitutes an exogenous nucleic acid.

The subject invention provides for the use of “homologous nucleic acid sequences” or “homologs of nucleic acid sequences”. Homologs of nucleic acid sequences will be understood to mean any nucleotide sequence obtained by mutagenesis according to techniques well known to persons skilled in the art, and exhibiting modifications in relation to the parent sequences. For example, mutations in the regulatory and/or promoter sequences for the expression of a polypeptide that result in a modification of the level of expression of a polypeptide according to the invention provide for a “homolog of a nucleotide sequence”. Likewise, substitutions, deletions, or additions of nucleic acid to the polynucleotides of the invention provide for “homologs” of nucleotide sequences. In various embodiments, “homologs” of nucleic acid sequences have substantially the same biological activity as the corresponding reference gene, i.e., a gene homologous to a native gene would encode for a protein having the same biological activity as the corresponding protein encoded by the naturally occurring gene. Typically, a homolog of a gene shares a sequence identity with the gene of at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. These percentages are purely statistical and differences between two nucleic acid sequences can be distributed randomly and over the entire sequence length.

Likewise, the subject invention also provides for the use of “homologs of proteins”. Homologs of proteins will be understood to mean any proteins obtained by mutagenesis according to techniques well known to persons skilled in the art, and exhibiting modifications in relation to the parent protein. Such modifications in a protein sequence include substitutions, deletions, or additions of amino acids to produce homologs of proteins. In various embodiments, a homolog of a protein has substantially the same biological activity as the protein, i.e., a protein homolog of a native protein would have the same biological activity as the native protein. Typically, a homolog of a reference protein shares a sequence identity with the reference protein of at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. These percentages are purely statistical and differences between two nucleic acid sequences can be distributed randomly and over the entire sequence length.

Rhg4 is a major locus required for SCN resistance in certain soybean accessions. Rhg4 encodes a serine hydroxymethyltransferase (GmSHMT08), whose function in nematode resistance remains mostly unknown. A key role of GmSHMT08 in reprograming soybean methylome during SCN infection is demonstrated using a pair of highly homozygous NILs containing the resistant or susceptible Rhg4 allele. Under non-infected conditions, the genomes of the NILs were considerably differentially methylated. The substantial differences in DNA methylation patterns between the NILs were associated with differential gene expression that may prime their response to SCN parasitism. In response to SCN infection, the NILs exhibited contrasted DNA methylation patterns with the methylome of the susceptible line being more dynamic than that of the resistant line.

Analysis of the methylome landscapes mediated by the SCN resistance gene Rhg4 was facilitated by using highly genetically identical NILs differing in Rhg4 locus and showing opposite responses to SCN infection. A key role of Rhg4 in establishing the root methylomes of the isogenic lines was demonstrated by the finding that the genomes of the isogenic lines were substantially differentially methylated both under non-infected and SCN-infected conditions. Rhg4 is believed to be the key factor mediating these differences since no gene involved in DNA methylation machinery was identified as differentially expressed when the root transcriptome of the isogenic lines were compared. This disclosure also indicates that methylome differences in the isogenic lines were established de novo since only 59 genetic regions showing opposite methylation patterns in these lines were inherited from the parents.

Under non-infected conditions, a general trend was observed of increased global DNA methylation in the SCN susceptible line relative to the SCN resistant line, specifically in the CHG and CHH contexts over protein-coding genes and TEs. However, the resistant line exhibited increased DNA methylation over the body of protein-coding genes and TEs in the CG context. This finding suggests that global decrease in CHG and CHH methylation in the resistant line complemented with an increase of CG methylation. Unlike other plant species in which gene body methylation occurs exclusively at CG sites, a significant number of CHG-DMRs were observed in the body of protein-coding genes when the methylomes of the two isogenic lines were compared under non-infected conditions. This could be the results of high insertion rate of short TEs in the introns of these genes. However, only 32% of the body CHG-methylated genes possess DMTEs inside their transcribed regions. Another possibility is that CHG methylation in gene body may be linked to the activity of various histone demethylases that function in eliminating methylation at histone H3 lysine 9 (H3K9me). In Arabidopsis, loss-of-function of the H3K9 demethylase IBM1 (Increase in BONSAI Methylation 1) induced CHG methylation in the body of thousands of highly expressed genes. Thus, differential accumulation of H3K9me in the body of actively transcribed genes may be responsible for CMT3-mediated differential genic CHG methylation between the isogenic lines. If CHG-methylation in gene body was mechanistically associated with CG or CHH methylation was also examined. Interestingly, only 24 and 3% of body CHG-methylated genes showed differential methylation in CG or CHH contexts, respectively. This finding suggests that CHG methylation in transcribed regions is mostly independent of CG and CHH methylation. The isogenic lines also exhibited high level of differential DNA methylation in TEs, particularly LTR-type in the CHG context. This result may reflect differences in the transposition dynamics of Copia and Gypsy retrotransposons, which tend to be more vulnerable to DNA methylation than other types of TEs.

Rhg4-mediated methylome changes in the isogenic lines under non-infected conditions appeared to significantly impact the methylation patterns of several genes involved in epigenetic regulation. Various epigenetic components including DNA methylation, histone modification and siRNA accumulation are highly interconnected. Thus, differential methylation of key genes involved in these pathways may be a part of epigenetic feedback regulatory mechanisms that maintain epigenetic information. Maintenance of epigenetic information could contribute to priming of plant defense responses. The RNA-seq analysis of the NILs under non-infected conditions pointed into a possible difference in some type of defense priming between the lines. This possibility was further reinforced by a number of findings. First, the DEGs were highly enriched for genes involved in defense responses and biological processes associated with nematode susceptibility. Second, about one-third of the DEGs were previously shown to change expression in SCN-induced syncytium. Third, the DEGs included numerous marker genes of defense priming, including several WRKY transcription factors, ROS-related genes, and lipoxygenases. Arabidopsis hyper- and hypomethylated mutants developed opposite responses to pathogen infection that were dependent on the mutants' ability to prime salicylic acid-mediated defense responses and callose deposition. In this context, DNA methylation can prime defense responses in a way that alters chromatin structure to expedite gene transcription.

The role of GmSHMT08 in establishing the methylomes of the isogenic lines became more evident when the methylomes of isogenic lines were determined under SCN-infected conditions. Consistent with the role of SHMT enzymes in cellular methylation, the susceptible line, which contains a non-functional allele of Rhg4, exhibited reduced global methylation levels in both protein-coding regions and TEs, whereas the resistant line showed the opposite response of increased global methylation levels. This trend was observed in all three methylation contexts, suggesting that the SHMT enzymes may have significant impact the whole methylation pathway. Also, the methylome of the susceptible line is more dynamic than that of the resistant line in response to SCN infection. 50,040 DMRs were identified in the susceptible line compared to only 5,080 DMRs in the resistant line. This dynamic can be explained by the many cellular processes that accompany syncytium formation and development during the susceptible interaction compared to localized cell death that occurs during the resistant interaction. In addition to the dramatic differences between the isogenic lines in term of methylation level and direction, differential DNA methylation patterns within protein-coding genes and TEs demonstrated remarkable level of specificity as only 74 DMRs were found common to both lines.

A low overlap was observed between the DEGs and the DMGs in the resistant line during SCN infection. This may be due to the dilution of localized gene expression changes at the infection sites by using whole roots as analyzed here. Because DNA methylation can precede gene expression changes during cyst nematode infections, the DMGs may associate with gene transcription at later stage of infection. Alternatively, DNA methylation patterns may regulate the steady-state expression of these genes, preventing their induction or repression during the resistant response. A role of DNA methylation as a secondary stabilizer of gene expression has been recently proposed. Also, DNA methylation function mutually with other epigenetic modification and hence, one can anticipate that methylation status of these genes may necessitate additional epigenetic marks to influence gene transcription to the level of significance.

In contrast to the resistant interaction, DNA methylation reprograming during the susceptible interaction seem to directly impact gene transcription levels. A set of 147 differentially expressed genes (DEGs) and differentially methylated genes (DMGs) was identified. Differential DNA methylation seems to impact cellular functions that are directly modulated by cyst nematode effectors. For example, genes involved in pectin demethylesterification and polyamine oxidation, which are targeted by the cyst nematode effectors cellulose binding protein and 10A06, respectively, were among the 147 DEGs/DMGs. The potential effect of DNA methylation on gene transcription was obvious in many situations as in the case of an adaptin family protein gene, which was hypomethylated in the promoter, gene body, and 3′UTR in various sequence contexts and highly induced in response to SCN infection. Adaptin family proteins are involved in intracellular protein trafficking and thus hypomethylation-mediated upregulation of this gene may facilitate assimilate flow to the syncytium. Another example of the impact of DNA methylation is the downregulation of cycling DOF factor 2 (CDF2), which was hypomethylated in gene body in the CG context, but was hypermethylated in the promoter region in the CHH context. In Arabidopsis, CDF2 has been reported to regulate the expression of a number of miRNA genes at both transcriptional and posttranscriptional levels by direct binding to miRNA promoters or through modulation of DCL1-mediated processing of primary miRNA transcripts. Thus, reprogramming of DNA methylation patterns may function in concert with other epigenetic pathways during SCN parasitism.

Increased activity of metabolism pathways is known to play central role in successful nematode parasitism. This disclosure demonstrates that DNA methylation contributes to the regulation of the transcriptional activity of several key genes specifically associated with the metabolic processes of carbohydrate, glucan and malate, presumably to maintain metabolite levels at an active physiological status compatible with nematode feeding and development. Associations between hyper- and hypomethylation and significant changes in gene expression were also observed for several genes involved in the biogenesis of primary and secondary cell walls, organization of actin and microtubules, defense responses, and signal transduction. Thus, cyst nematode-induced differential methylation during the susceptible interaction appears to regulate similar cellular processes in various plant species.

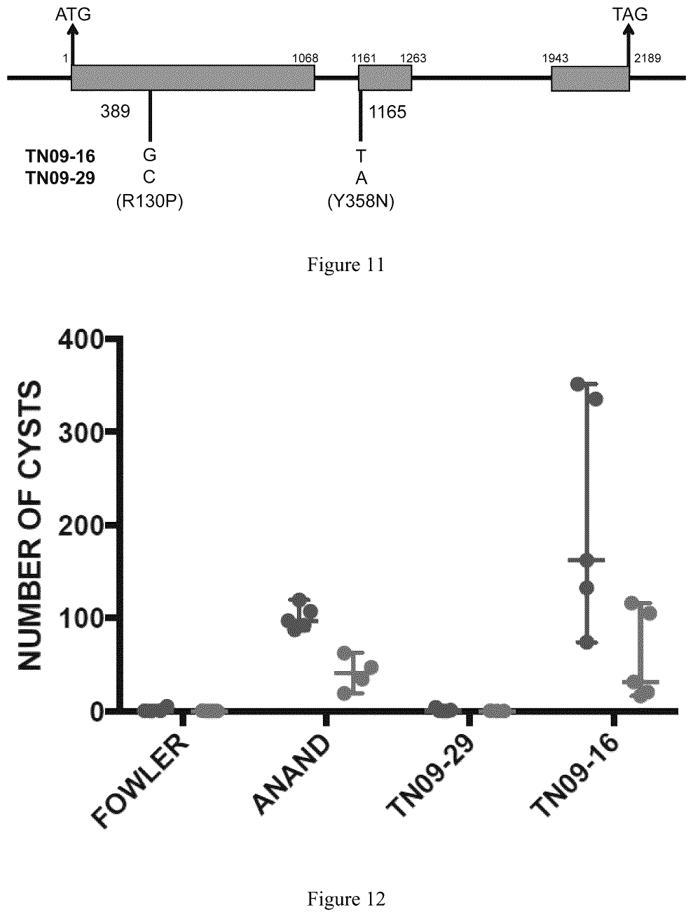

Two near isogenic lines (NILs), TN09-16 and TN09-29, respectively containing the susceptible and resistant allele of Rhg4, were generated. These NILs are highly homozygous recombinant inbred lines derived from individual F13 generation single plants from a cross between the SCN-resistant variety Fowler and the SCN-susceptible variety Anand. Homozygosity level of these NILs was estimated to be 0.9998 based on the number of inbreeding generations after the creation of F1. SCN resistance in the parental line Fowler was acquired from the Plant Introduction (PI) 437654. PI 437654, which exhibit ‘Peking-type’ resistance, has been shown to carry the SCN resistance gene Rhg4 and three copies of rhg1a. Initially, Simple Sequence Repeat (SSR) markers associated with rhg1 (Satt309) and Rhg4 (Satt162 and Satt632) were used to test potential genetic differences between these two NILs at rhg1 and Rhg1 loci. The resistant line TN09-29 inherited the two resistant alleles from its Hartwig ancestry. The susceptible line TN09-016, however, did not inherit the Rhg4 allele but did receive the Rhg1 resistant allele. The SCN resistance genes soluble NSF attachment proteins (GmSNAP 18) and the serine hydroxymethyltransferase (GmSHMT08) at the rhg1 and Rhg4 loci, respectively, were cloned and sequenced from the isogenic lines. While the nucleotide sequences of GmSNAP18 were identical in both lines, GmSHMT08 showed two single nucleotide polymorphisms (SNPs), leading to R130P and Y358N amino acid substitutions between the TN09-29 and TN09-16 (FIG. 11). These two amino acid substitutions were previously reported to establish the difference between the resistant and susceptible alleles of Rhg4.

The methylome analysis of the parental lines and the two isogenic lines revealed 56 and 102 genetic regions that exhibit novel non-parental methylation patterns unique to TN09-16 and TN09-29, respectively. Gain or loss of DNA methylation in these regions seem to occur to a similar extent with preference observed for CG and CHG contexts. Gain of DNA methylation appears to be introduced in the isogenic lines, whether environmentally or genetically, during the 13 generations of breeding and was faithfully maintained through the activity of MET1 and CMT3. In contrast, loss of DNA methylation in certain genic regions could be the result of absence of corrective DNA methylation mechanisms that can restore DNA methylation. Failure to reestablish DNA methylation in specific genic regions may be due to the loss of the repressive histone mark H3K9me2, which has been shown to be directly or indirectly linked to the activity of MET1 and CMT3-mediated DNA methylation in CG and non-CG contexts.

The non-parental methylation patterns in these genic regions could be induced by transposons located in proximity to these DMR-associated genes, specifically those transposons that are prone to DNA methylation change under infected conditions. 12 of the 56 DMRs found to be unique to the susceptible line TN09-16 were located within 2 Kb of differentially methylated TEs identified in TN09-16 in response to SCN infection. Previously, it has been shown that TEs can induce heritable epialleles by bringing neighboring genes under their own regulation. Whether these 56 DMRs are particularly vulnerable to DNA methylation changes under SCN infected conditions was examined. Interestingly, 18 of these DMRs were found to be among those identified in TN09-16 in response to SCN infection. Similarly, 20 of the 102 DMRs that were unique to TN09-29 were among those showing differential methylation under SCN infection. Together, these findings suggest that the non-parental methylation patterns occur in regions that are vulnerable to methylation changes and that some introduced variations in DNA methylation pattern can be inherited and stably transmitted to offspring. A small portion of stress-induced DNA methylation changes can be faithfully transmitted to next generations. However, the association of these regions with genes previously shown to change expression in the syncytium highlight sheds lights into a role of these regions in SCN parasitism of soybean.

Thus, comparing the methylomes of the parental lines with that of the NILs resulted in the identification of heritable as well as novel non-parental differentially methylated regions overlapping with genes related to SCN parasitism of soybean. Thus, the disclosure provides the genes involved in biochemical basis of Rhg4 function in SCN resistance.

The disclosure provides that the genes listed in Table 1 (SEQ ID NOs: 1-210) provide resistance or susceptibility to a plant cell or a plant, particularly, a soybean plant cell or a soybean plant. For example, overexpressing in a plant cell or a plant, particularly, a soybean plant cell or a soybean plant, one or more genes comprising a protein coding sequence selected from SEQ ID NOs: 1-209 or homologs thereof renders the plant cell or the plant, particularly, the soybean plant cell or the soybean plant, resistance to SCN. Similarly, overexpressing in a plant cell or a plant, particularly, a soybean plant cell or a soybean plant, the gene encoding the miRNA having the sequence of SEQ ID NO: 210 (gma-miR5032) or a homolog thereof renders the plant cell or the plant, particularly, the soybean plant cell or the soybean plant, resistance to SCN. Also, inactivating in a plant cell or a plant, particularly, a soybean plant cell or a soybean plant, one or more genes comprising a protein coding sequence selected from SEQ ID NOs: 1-209 or homologs thereof renders the plant cell or the plant, particularly, the soybean plant cell or the soybean plant, resistance to SCN.

TABLE 1
SEQ ID NOs: 1 to 210 providing candidate
genes that confer SCN resistance
when overexpressed or inactivated.

| | SEQ |
|---|---|
| | ID |
| Glyma ID No. | NO: |
| Glyma.11G004900 | 1 |
| Glyma.14G142500 | 2 |
| Glyma.17G216000 | 3 |
| Glyma.09G171300 | 4 |
| Glyma.10G083800 | 5 |
| Glyma.U040800 | 6 |
| Glyma.17G017300 | 7 |
| Glyma.17G223100 | 8 |
| Glyma.09G094000 | 9 |
| Glyma.09G198400 | 10 |
| Glyma.09G123300 | 11 |
| Glyma.09G132300 | 12 |
| Glyma.09G133900 | 13 |
| Glyma.09G129600 | 14 |
| Glyma.04G157200 | 15 |
| Glyma.04G191800 | 16 |
| Glyma.04G036000 | 17 |
| Glyma.07G168900 | 18 |
| Glyma.15G015100 | 19 |
| Glyma.13G228800 | 20 |
| Glyma.13G234600 | 21 |
| Glyma.13G288000 | 22 |
| Glyma.13G005900 | 23 |
| Glyma.13G320100 | 24 |
| Glyma.13G339600 | 25 |
| Glyma.19G044300 | 26 |
| Glyma.19G136000 | 27 |
| Glyma.03G131000 | 28 |
| Glyma.03G085300 | 29 |
| Glyma.03G129100 | 30 |
| Glyma.20G052100 | 31 |
| Glyma.05G093400 | 32 |
| Glyma.05G132100 | 33 |
| Glyma.05G203000 | 34 |
| Glyma.05G092300 | 35 |
| Glyma.11G092400 | 36 |
| Glyma.14G103300 | 37 |
| Glyma.08G078200 | 38 |
| Glyma.08G028400 | 39 |
| Glyma.08G060200 | 40 |
| Glyma.08G314100 | 41 |
| Glyma.08G100300 | 42 |
| Glyma.01G154500 | 43 |
| Glyma.12G060400 | 44 |
| Glyma.12G121100 | 45 |
| Glyma.06G251700 | 46 |
| Glyma.06G228100 | 47 |
| Glyma.06G148300 | 48 |
| Glyma.18G168000 | 49 |
| Glyma.16G088900 | 50 |
| Glyma.16G065300 | 51 |
| Glyma.04g041900 | 52 |
| Glyma.10g104900 | 53 |
| Glyma.12g081900 | 54 |
| Glyma.14g021300 | 55 |
| Glyma.17g091700 | 56 |
| Glyma.20g126100 | 57 |
| Glyma.20g134300 | 58 |
| Glyma.10G067400 | 59 |
| Glyma.10G289000 | 60 |
| Glyma.17G193800 | 61 |
| Glyma.17G214800 | 62 |
| Glyma.17G057300 | 63 |
| Glyma.09G002300 | 64 |
| Glyma.04G174100 | 65 |
| Glyma.04G238800 | 66 |
| Glyma.04G149700 | 67 |
| Glyma.04G095300 | 68 |
| Glyma.04G029400 | 69 |
| Glyma.04G033600 | 70 |
| Glyma.04G241200 | 71 |
| Glyma.07G132300 | 72 |
| Glyma.07G118800 | 73 |
| Glyma.15G204600 | 74 |
| Glyma.15G219000 | 75 |
| Glyma.13G010200 | 76 |
| Glyma.13G137000 | 77 |
| Glyma.02G236000 | 78 |
| Glyma.02G181200 | 79 |
| Glyma.19G130000 | 80 |
| Glyma.03G240800 | 81 |
| Glyma.03G100000 | 82 |
| Glyma.03G243400 | 83 |
| Glyma.03G205600 | 84 |
| Glyma.20G219600 | 85 |
| Glyma.20G237500 | 86 |
| Glyma.20G212100 | 87 |
| Glyma.05G142200 | 88 |
| Glyma.11G164700 | 89 |
| Glyma.11G181300 | 90 |
| Glyma.11G229600 | 91 |
| Glyma.14G047400 | 92 |
| Glyma.08G269900 | 93 |
| Glyma.08G238200 | 94 |
| Glyma.08G237500 | 95 |
| Glyma.08G226000 | 96 |
| Glyma.08G079300 | 97 |
| Glyma.08G168000 | 98 |
| Glyma.01G060200 | 99 |
| Glyma.01G008900 | 100 |
| Glyma.01G218100 | 101 |
| Glyma.12G173100 | 102 |
| Glyma.06G233300 | 103 |
| Glyma.18G279400 | 104 |
| Glyma.18G073400 | 105 |
| Glyma.16G088200 | 106 |
| Glyma.16G174500 | 107 |
| Glyma.16G164500 | 108 |
| Glyma.19g104100 | 109 |
| Glyma.20g189700 | 110 |
| Glyma.10G098700 | 111 |
| Glyma.10G028800 | 112 |
| Glyma.10G238200 | 113 |
| Glyma.10G066100 | 114 |
| Glyma.10G076500 | 115 |
| Glyma.10G073100 | 116 |
| Glyma.17G008600 | 117 |
| Glyma.17G032500 | 118 |
| Glyma.17G135500 | 119 |
| Glyma.17G049500 | 120 |
| Glyma.17G012400 | 121 |
| Glyma.17G038800 | 122 |
| Glyma.09G159500 | 123 |
| Glyma.09G198900 | 124 |
| Glyma.09G190500 | 125 |
| Glyma.04G220100 | 126 |
| Glyma.04G221900 | 127 |
| Glyma.04G136700 | 128 |
| Glyma.04G177000 | 129 |
| Glyma.04G094900 | 130 |
| Glyma.04G140800 | 131 |
| Glyma.04G149400 | 132 |
| Glyma.04G111200 | 133 |
| Glyma.04G245700 | 134 |
| Glyma.07G243600 | 135 |
| Glyma.07G188300 | 136 |
| Glyma.07G000600 | 137 |
| Glyma.07G156900 | 138 |
| Glyma.07G166000 | 139 |
| Glyma.15G136000 | 140 |
| Glyma.15G125100 | 141 |
| Glyma.13G355400 | 142 |
| Glyma.13G056100 | 143 |
| Glyma.02G093000 | 144 |
| Glyma.02G129200 | 145 |
| Glyma.02G221300 | 146 |
| Glyma.02G164300 | 147 |
| Glyma.19G018100 | 148 |
| Glyma.19G153500 | 149 |
| Glyma.19G023000 | 150 |
| Glyma.19G164800 | 151 |
| Glyma.19G036100 | 152 |
| Glyma.03G058300 | 153 |
| Glyma.03G034500 | 154 |
| Glyma.03G107700 | 155 |
| Glyma.03G064800 | 156 |
| Glyma.05G185300 | 157 |
| Glyma.05G098000 | 158 |
| Glyma.05G003500 | 159 |
| Glyma.05G084500 | 160 |
| Glyma.05G100700 | 161 |
| Glyma.05G035100 | 162 |
| Glyma.05G092600 | 163 |
| Glyma.05G012700 | 164 |
| Glyma.11G125600 | 165 |
| Glyma.14G193900 | 166 |
| Glyma.14G098600 | 167 |
| Glyma.14G122700 | 168 |
| Glyma.14G138300 | 169 |
| Glyma.14G065500 | 170 |
| Glyma.14G104300 | 171 |
| Glyma.14G193600 | 172 |
| Glyma.08G190600 | 173 |
| Glyma.08G014300 | 174 |
| Glyma.08G238800 | 175 |
| Glyma.08G167600 | 176 |
| Glyma.08G258600 | 177 |
| Glyma.01G102900 | 178 |
| Glyma.01G244700 | 179 |
| Glyma.01G112600 | 180 |
| Glyma.12G136700 | 181 |
| Glyma.12G146400 | 182 |
| Glyma.06G286900 | 183 |
| Glyma.06G196500 | 184 |
| Glyma.06G180700 | 185 |
| Glyma.06G092500 | 186 |
| Glyma.06G231200 | 187 |
| Glyma.18G033500 | 188 |
| Glyma.18G168500 | 189 |
| Glyma.18G077700 | 190 |
| Glyma.18G179600 | 191 |
| Glyma.18G066700 | 192 |
| Glyma.18G131100 | 193 |
| Glyma.18G220700 | 194 |
| Glyma.16G135200 | 195 |
| Glyma.16G214100 | 196 |
| Glyma.02g041600 | 197 |
| Glyma.02g071300 | 198 |
| Glyma.09g001700 | 199 |
| Glyma.10g105800 | 200 |
| Glyma.10g145700 | 201 |
| Glyma.10g168000 | 202 |
| Glyma.10g181600 | 203 |
| Glyma.10g192400 | 204 |
| Glyma.11g000100 | 205 |
| Glyma.14g009100 | 206 |
| Glyma.14g011600 | 207 |
| Glyma.17g205100 | 208 |
| Glyma.20g071200 | 209 |
| gma-miR5032 | 210 |

Accordingly, certain embodiments of the invention provide a method of producing an SCN resistant plant cell or a plant comprising overexpressing in the plant one or more genes comprising a protein coding sequence selected from SEQ ID NOs: 1-209 or homologs thereof. Further embodiments of the invention provide a method of producing an SCN resistant plant cell or a plant comprising overexpressing in the plant cell or the plant a gene encoding the miRNA having the sequence of SEQ ID NO: 210 (miR5032) or a homolog thereof. In preferred embodiments, the plant cell or a plant is a soybean plant cell or soybean plant.

As used herein, the term “overexpressing a gene” or grammatical variations thereof refer to a condition in a genetically modified plant cell or a genetically modified plant wherein the gene encodes for a protein or an miRNA at a level higher than the parent plant cell or the plant without the genetic modification. Thus, a parent plant cell or a parent plant is genetically modified to produce a modified plant cell or modified plant that expresses a gene to produce a protein or an miRNA at a higher level compared to the parent plant cell or parent plant.

Typically, overexpressing a gene in a plant cell or a plant comprises introducing into the plant cell or a plant, a nucleic acid construct comprising the gene. The nucleic acid construct is designed to induce the expression of the protein or the miRNA encoded by the gene. Methods of producing and introducing various nucleic acid constructs comprising genes of interest into a plant cell or a plant to overexpress the genes are well known to a person of ordinary skill in the art and such embodiments are within the purview of the invention. Certain such embodiments are identified below.

A gene is referred to as “operably linked” when it is placed into a functional relationship with another DNA segment (for example, a promoter that is operably linked to any one of SEQ ID NOs: 1-209 or homologs thereof or a sequence encoding miR5032 of SEQ ID NO: 210 or a homolog thereof). However, enhancers need not be contiguous with the coding sequences whose transcription they control. Linking is accomplished by ligation at convenient restriction sites or at adapters or linkers inserted in lieu thereof. The expression cassette can include one or more enhancers in addition to the promoter. By “enhancer” is intended a cis-acting sequence that increases the utilization of a promoter. Such enhancers can be native to a gene or from a heterologous gene. Further, it is recognized that some promoters can contain one or more native, enhancers or enhancer-like elements. An example of one such enhancer is the 35S enhancer, which can be a single enhancer, or duplicated. See for example, McPherson et al, U.S. Pat. No. 5,322,938, which is hereby incorporated by reference in its entirety.

The promoter for driving expression of the genes of interest may be selected based on a number of criteria including, but not limited to, what the desired use is for the operably linked polynucleotide, what location in the plant is expression of the gene of interest desired, and at what level is expression of gene of interest desired or whether it needs to be controlled in another spatial or temporal manner. In one aspect, a promoter that directs expression to particular tissue may be desirable. When referring to a promoter that directs expression to a particular tissue is meant to include promoters referred to as tissue specific or tissue preferred. Included within the scope of the invention are promoters that express highly in the plant tissue, express more in the plant tissue than in other plant tissue, or express exclusively in the plant tissue. For example, “seed-specific” promoters may be employed to drive expression. Specific-seed promoters include those promoters active during seed development, promoters active during seed germination, and/or that are expressed only in the seed. Seed-specific promoters, such as annexin, P34, beta-phaseolin, alpha subunit of beta-conglycinin, oleosin, zein, napin promoters have been identified in many plant species such as maize, wheat, rice and barley. See U.S. Pat. Nos. 7,157,629, 7,129,089, and 7,109,392. Such seed-preferred promoters further include, but are not limited to, Cim1 (cytokinin-induced message); cZ19B1 (maize 19 kDa zein); and milps (myo-inositol-1-phosphate synthase); (see WO 00/11177, herein incorporated by reference). The 27 kDa gamma-zein promoter is a preferred endosperm-specific promoter. The maize globulin-1 and oleosin promoters are preferred embryo-specific promoters. For dicots, seed-specific promoters include, but are not limited to, bean beta phaseolin, napin, beta-conglycinin, soybean lectin, cruciferin, and the like. For monocots, seed-specific promoters include, but are not limited to, promoters of the 15 kDa beta-zein, 22 kDa alpha-zein, 27 kDa gamma-zein, waxy, shrunken 1, shrunken 2, globulin 1, an LtpI, an Ltp2, and oleosin genes. See also WO 00/12733, where seed-preferred promoters from end1 and end2 genes are disclosed; herein incorporated by reference. Each of these aforementioned references is hereby incorporated by reference in its entirety, particularly as it relates to the promoters disclosed within the reference.

In preferred embodiments, a promoter used in the present invention is a promoter for soybean ubiquitin promoters, for example, the promoters for soybean ubiquitin B (UBB)/ubiquitin C (UBC) gene. Certain examples of soybean ubiquitin promoters that could be used in the present invention are described in United States patent application publication numbers 20140053296 and 20100186119. Each of these publications is incorporated by reference in its entirety, particularly, the sequence listing.

The promoters useful in the present invention can also include constitutive, inducible or tissue-specific (preferred) promoters that are operably linked to a gene comprising a protein coding sequence of any one of SEQ ID NOs: 1-209 or homologs thereof or a sequence encoding miR5032 or a homolog thereof and are heterologous to the nucleic acid sequences to which they are operably linked. In other words, the promoters are not those found operably linked to a gene comprising a protein coding sequence of SEQ ID NOs: 1-209 or homologs thereof or a sequence encoding miR5032 or a homolog thereof in their native context within a plant, such as a soybean plant. Constitutive promoters, generally, are active in most or all tissues of a plant; inducible promoters, which generally are inactive or exhibit a low basal level of expression, and can be induced to a relatively high activity upon contact of cells with an appropriate inducing agent; tissue-specific (or tissue-preferred) promoters, which generally are expressed in only one or a few particular cell types (e.g., root cells); and developmental-or stage-specific promoters, which are active only during a defined period during the growth or development of a plant. Often promoters can be modified, if necessary, to vary the expression level. Certain embodiments comprise promoters exogenous to the species being manipulated (e.g. a soybean plant).

Non-limiting examples of root-specific promoters (a subset of tissue-specific promoters) include root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664). Each of these aforementioned references is hereby incorporated by reference in its entirety, particularly as it relates to the promoters disclosed within the reference.

Exemplary constitutive promoters include the 35S cauliflower mosaic virus (CaMV) promoter (Odell et al. (1985) Nature 313:810-812), the maize ubiquitin promoter (Christensen et al. (1989) Plant Mol. Biol. 12:619-632 and Christensen et al. (1992) Plant Mol. Biol. 18:675-689); the core promoter of the Rsyn7 promoter and other constitutive promoters disclosed in WO 99/43838 and U.S. Pat. No. 6,072,050; rice actin (McElroy et al. (1990) Plant Cell 2:163-171); pEMU (Last et al. (1991) Theor. Appl. Genet. 81:581-588); MAS (Velten et al. (1984) EMBO J. 3:2723-2730); ALS promoter (U.S. Pat. No. 5,659,026); rice actin promoter (U.S. Pat. No. 5,641,876; WO 00/70067), maize histone promoter (Brignon et al., Plant Mol Bio 22(6):1007-1015 (1993); Rasco-Gaunt et al., Plant Cell Rep. 21(6):569-576 (2003)) and the like. Other constitutive promoters include, for example, those described in U.S. Pat. Nos. 5,608,144 and 6,177,611, and PCT publication WO 03/102198. Each of these aforementioned references is hereby incorporated by reference in its entirety, particularly as it relates to the promoters disclosed within the reference.

An inducible promoter/regulatory element is one that is capable of directly or indirectly activating transcription of a gene comprising a protein coding sequence of one or more of SEQ ID NOs: 1-209 or a sequence encoding miR5032 or a homolog thereof in response to an inducer. The inducer can be a chemical agent such as a protein, metabolite, growth regulator, herbicide or phenolic compound; or a physiological stress, such as that imposed directly by heat, cold, salt, or toxic elements, or indirectly through the action of a pathogen or disease agent such as a virus; or other biological or physical agent or environmental condition. A plant cell containing an inducible promoter/regulatory element may be exposed to an inducer by externally applying the inducer to the cell or plant such as by spraying, watering, heating or similar methods. An inducing agent useful for inducing expression from an inducible promoter is selected based on the particular inducible regulatory element. In response to exposure to an inducing agent, transcription from the inducible regulatory element generally is initiated de novo or is increased above a basal or constitutive level of expression.

Any inducible promoter/regulatory element can be used in the instant invention (See Ward et al., Plant Mol. Biol. 22: 361-366, 1993). Non-limiting examples of such promoters/regulatory elements include: a metallothionein regulatory element, a copper-inducible regulatory element, or a tetracycline-inducible regulatory element, the transcription from which can be effected in response to divalent metal ions, copper or tetracycline, respectively (Furst et al., Cell 55:705-717, 1988; Mett et al., Proc. Natl. Acad. Sci., USA 90:4567-4571, 1993; Gatz et al., Plant J. 2:397-404, 1992; Roder et al., Mol. Gen. Genet. 243:32-38, 1994). Inducible promoters/regulatory elements also include an ecdysone regulatory element or a glucocorticoid regulatory element, the transcription from which can be effected in response to ecdysone or other steroid (Christopherson et al., Proc. Natl. Acad. Sci., USA 89:6314-6318, 1992; Schena et al., Proc. Natl. Acad. Sci., USA 88:10421-10425, 1991; U.S. Pat. No. 6,504,082); a cold responsive regulatory element or a heat shock regulatory element, the transcription of which can be effected in response to exposure to cold or heat, respectively (Takahashi et al., Plant Physiol. 99:383-390, 1992); the promoter of the alcohol dehydrogenase gene (Gerlach et al., PNAS USA 79:2981-2985 (1982); Walker et al., PNAS 84(19):6624-6628 (1987)), inducible by anaerobic conditions; and the light-inducible promoter derived from the pea rbcS gene or pea psaDb gene (Yamamoto et al. (1997) Plant J. 12(2):255-265); a light-inducible regulatory element (Feinbaum et al., Mol. Gen. Genet. 226:449, 1991; Lam and Chua, Science 248:471, 1990; Matsuoka et al. (1993) Proc. Natl. Acad. Sci. USA 90(20):9586-9590; Orozco et al. (1993) Plant Mol. Bio. 23(6):1129-1138), a plant hormone inducible regulatory element (Yamaguchi-Shinozaki et al., Plant Mol. Biol. 15:905, 1990; Kares et al., Plant Mol. Biol. 15:225, 1990), and the like. An inducible promoter/regulatory element also can be the promoter of the maize In2-1 or In2-2 gene, which responds to benzenesulfonamide herbicide safeners (Hershey et al., Mol. Gen. Gene. 227:229-237, 1991; Gatz et al., Mol. Gen. Genet. 243:32-38, 1994), and the Tet repressor of transposon Tn10 (Gatz et al., Mol. Gen. Genet. 227:229-237, 1991). Stress inducible promoters include salt/water stress-inducible promoters such as P5CS (Zang et al. (1997) Plant Sciences 129:81-89); cold-inducible promoters, such as, cor15a (Hajela et al. (1990) Plant Physiol. 93:1246-1252), cor15b (Wilhelm et al. (1993) Plant Mol Biol 23:1073-1077), wsc120 (Ouellet et al. (1998) FEBS Lett. 423-324-328), ci7 (Kirch et al. (1997) Plant Mol Biol. 33:897-909), ci21A (Schneider et al. (1997) Plant Physiol. 113:335-45); drought-inducible promoters, such as, Trg-31 (Chaudhary et al (1996) Plant Mol. Biol. 30:1247-57), rd29 (Kasuga et al. (1999) Nature Biotechnology 18:287-291); osmotic inducible promoters, such as Rab17 (Vilardell et al. (1991) Plant Mol. Biol. 17:985-93) and osmotin (Raghothama et al. (1993) Plant Mol Biol 23:1117-28); and heat inducible promoters, such as heat shock proteins (Barros et al. (1992) Plant Mol. 19:665-75; Marrs et al. (1993) Dev. Genet. 14:27-41), smHSP (Waters et al. (1996) J. Experimental Botany 47:325-338), and the heat-shock inducible element from the parsley ubiquitin promoter (WO 03/102198). Other stress-inducible promoters include rip2 (U.S. Pat. No. 5,332,808 and U.S. Publication No. 2003/0217393) and rd29a (Yamaguchi-Shinozaki et al. (1993) Mol. Gen. Genetics 236:331-340). Certain promoters are inducible by wounding, including the Agrobacterium pmas promoter (Guevara-Garcia et al. (1993) Plant J. 4(3):495-505) and the Agrobacterium ORF13 promoter (Hansen et al., (1997) Mol. Gen. Genet. 254(3):337-343). Each of these aforementioned references is hereby incorporated by reference in its entirety, particularly as it relates to the promoters disclosed within the reference.

Overexpression of a gene comprising a protein coding sequence selected from SEQ ID NOs: 1-209 or homologs thereof or a sequence encoding miR5032 or a homolog thereof can also be achieved by one or one or more mutations in the endogenous promoter of the gene, wherein the one or more mutations increase the expression of the gene. For a particular gene, a skilled artisan can identify one or more mutations that would increase the expression of the gene and such embodiments are within the purview of the invention.

Certain preferred embodiments of the invention provide a method of producing a plant cell or a plant that is resistant to SCN, the method comprising overexpressing in the plant cell or the plant a gene comprising a protein coding sequence of a sulfite exporter TauE/SafE protein and/or overexpressing a gene comprising a protein coding sequence of a SNARE associated Golgi protein. The overexpressed gene encoding the SNARE associated Golgi protein can comprise a protein coding sequence of Glyma.11G004900, according to SEQ ID NO: 1, or a homolog thereof. The overexpressed gene encoding the sulfite exporter TauE/SafE protein can comprise a protein coding sequence of Glyma. 14G142500, according to SEQ ID NO: 2, or a homolog thereof.

Additional embodiments of the invention also provide a plant cell comprising an overexpressed gene comprising a protein coding sequence selected from SEQ ID NOs: 1-209 or homologs thereof or a sequence encoding miR5032 or a homolog thereof. The plant cell or the plant can be a soybean plant cell or soybean plant.

In preferred embodiments, the plant cell or the plant overexpresses a gene encoding a sulfite exporter TauE/SafE protein and/or a gene encoding a SNARE associated Golgi protein. The overexpressed gene encoding the SNARE associated Golgi protein can comprise a protein coding sequence of Glyma.11G004900, according to SEQ ID NO: 1, or a homolog thereof. The overexpressed gene encoding the sulfite exporter TauE/SafE protein can comprise a protein coding sequence of Glyma.14G142500, according to SEQ ID NO: 2, or a homolog thereof. The plant cell or a plant can also comprise overexpression of a gene encoding the SNARE associated Golgi protein of the protein coding sequence of Glyma.11G004900, according to SEQ ID NO: 1, or a homolog thereof and overexpression of a gene encoding the sulfite exporter TauE/SafE protein of the protein coding sequence of Glyma.14G142500, according to SEQ ID NO: 2, or a homolog thereof.

In further embodiments, the plant cell or the plant overexpresses a gene encoding a sequence encoding miR5032 or a homolog thereof. The plant cell or a plant can also further comprise overexpression of a gene comprising a protein coding sequence of SEQ ID NO: 1 or a homolog thereof and/or a gene comprising a protein coding sequence of SEQ ID NO: 2 or a homolog thereof. In addition to these modifications, the plant cell or a plant can also further comprise an inactivation of a gene comprising a protein coding sequence of Glyma.17G216000, according to SEQ ID NO: 3, or a homolog thereof and an inactivation of a gene comprising a protein coding sequence of Glyma.02G071300, according to SEQ ID NO: 4, or a homolog thereof.

Further embodiments of the invention provide a method of producing an SCN resistant plant cell or a plant comprising inactivating in the plant one or more genes comprising a protein coding sequence selected from SEQ ID NOs: 1-209 or homologs thereof. In preferred embodiments, the plant cell or a plant is a soybean plant cell or soybean plant.

Typically, inactivating a gene in a plant cell or a plant comprises introducing into the gene one or more mutations that inhibit or abolish the expression of an active protein from the gene. Mutations in a gene that inhibit or abolish the expression of a protein from the gene can be achieved either by deleting the entire coding region of the gene or a portion of the coding region of the gene, by introducing a frame shift mutation within the coding region of the gene, by introducing a missense mutation, insertion of sequences that disrupt the activity of the protein encoded by the gene, by introducing a stop codon or any combination of the aforementioned gene mutations. Inactivating a gene can also be performed by using molecular markers or other traditional breeding methods to integrate activated or inhibited genes in any soybean germplasm. Further, overexpressing one or more genes can be performed by introducing and/or expressing the one or more genes under soybean endogenous promoters and/or any exogenous promoters.

Methods of inactivating a gene of interest in a plant cell or a plant to inhibit or abolish the expression of an active protein from the gene are well known to a person of ordinary skill in the art and such embodiments are within the purview of the invention. Certain such embodiments are identified below.

Sanagala et al. (2017), Journal of Genetic Engineering and Biotechnology; 15(2):317-321, describe several methods of inactivating a gene, for example, by implementing homologous recombination, zinc finger nucleases, Transcription activator-like effector nucleases (TALENs), and the clustered regularly interspaced short palindromic repeats/CRISPR-associated (CRISPR/Cas) system. The Sanagala et al. reference is incorporated herein by reference in its entirety.

In preferred embodiments, inactivating a gene of interest is performed using the CRISPR/Cas system. An example of such system to inactivate genes in a plant cell or a plant is provided by Ordon et al. (2017), The Plant Journal; 89:155-168. The Ordon et al. reference is incorporated herein by reference in its entirety.

Typically, a CRISPR/Cas system mediated inactivation of a gene involves the use of a guide RNA targeted to a gene of interest. A DNA oligomer targeted to a gene of interest can be transcribed into single guide RNA (sgRNA). sgRNA guides the Cas9 DNA endonuclease to the gene of interest by sgRNA hybridization to the target site. The endonuclease Cas9 makes a double strand break 3 bp upstream of Palindromic Adjacent Motif (PAM). The DNA breakage engages the repair mechanism, such as homologous recombination (HR) or the non-homologous end joining (NHEJ) mechanism. The NHEJ mechanism is a major double strand break repair pathway in plants and is known to be error prone. NHEJ DNA repair process introduces errors in the DNA repair, which causes irreversible mutations at the gene of interest. The chances of errors in DNA repair can be increased by providing multiple sgRNA. Based on the sequence of a gene comprising a protein coding sequence selected from SEQ ID NOs: 1-209 or homologs thereof, a person of ordinary skill in the art can design and perform inactivation of the gene using the CRISPR/Cas system and such embodiments are within the purview of the invention.

Methods of inactivating a gene of interest in a plant cell or a plant to inhibit or abolish the expression of an active protein from the gene also include introduction into the plant cell or the plant one or more inhibitory oligonucleotides, such as small interfering RNA (siRNA) or short hairpin RNAs (shRNA). Methods of producing and introducing inhibitory RNA are also well known to a person of ordinary skill in the art and such embodiments are within the purview of the invention.

Certain preferred embodiments of the invention provide a method of producing a plant cell or a plant that is resistant to SCN, the method comprising inactivating in the plant cell or the plant a gene encoding an aspartate aminotransferase protein and/or a gene encoding a transcription regulator of the NOT2/NOT3/NOT5 protein. The inactivated gene encoding the aspartate aminotransferase protein can comprise a protein coding sequence of Glyma.17G216000, according to SEQ ID NO: 3, or a homolog thereof. The inactivated gene encoding the transcription regulator of the NOT2/NOT3/NOT5 can comprise a protein coding sequence of Glyma.02G071300, according to SEQ ID NO: 4, or a homolog thereof. A gene comprising a protein coding sequence of Glyma.17G216000, according to SEQ ID NO: 3, or a homolog thereof and a gene comprising a protein coding sequence of Glyma.02G071300, according to SEQ ID NO: 4, or a homolog thereof can also be inactivated.

Additional embodiments of the invention also provide a plant cell comprising an inactivated gene comprising a protein coding sequence selected from SEQ ID NOs: 1-209 or homologs thereof. The plant cell or the plant can be a soybean plant cell or soybean plant.

In preferred embodiments, the plant cell or the plant comprises inactivation of a gene encoding an aspartate aminotransferase protein and/or inactivation of a gene encoding a transcription regulator of the NOT2/NOT3/NOT5 protein. The inactivated gene encoding the aspartate aminotransferase protein can comprise a protein coding sequence of Glyma.17G216000, according to SEQ ID NO: 3, or a homolog thereof. The inactivated gene encoding the transcription regulator of the NOT2/NOT3/NOT5 can comprise a protein coding sequence of Glyma.02G071300, according to SEQ ID NO: 4, or a homolog thereof. A plant cell or plant can also contain an inactivation of a gene comprising a protein coding sequence of Glyma.17G216000, according to SEQ ID NO: 3, or a homolog thereof and an inactivation of a gene comprising a protein coding sequence of Glyma.02G071300, according to SEQ ID NO: 4, or a homolog thereof.

In the methods of producing a plant cell described herein the plant cell can be in a plant part, for example, a seed, endosperm, ovule or pollen. The plant can be a soybean plant.

Further embodiments of the invention provide methods for identifying a gene that induces SCN resistance in a plant cell or a plant when overexpressed or inactivated, the method comprising the steps of: (a) overexpressing a gene in a plant cell or a plant, compared to the expression in a plant cell or a plant known to be susceptible to SCN, or inactivating a gene in a plant cell or a plant known to be resistant to SCN, (b) testing the SCN resistance in the plant cell or the plant comprising the overexpressed or inactivated gene, and (c) identifying the gene that induces resistance in the plant cell or the plant when overexpressed or inactivated.

In preferred embodiments, an overexpressed or inactivated gene comprises a protein coding sequence selected from SEQ ID NOs: 1-209 or homologs thereof or a sequence encoding miR5032 or a homolog thereof. In some embodiments, one or more genes comprising protein coding sequences selected from SEQ ID NOs: 1-209 or homologs thereof or a sequence encoding miR5032 or a homolog thereof are overexpressed and/or one or more genes comprising protein coding sequences selected from SEQ ID NOs: 1-209 or homologs thereof are inactivated.

The plant cell or the plant can be a soybean plant cell or a soybean plant.

The details discussed above for overexpressing and inactivating genes in a plant cell or a plant are also applicable to the methods of identifying a gene that induces SCN resistance in a plant cell or a plant when overexpressed or inactivated and such embodiments are within the purview of the invention.

Modifications to the promoter regions of the genes comprising protein coding sequences selected from SEQ ID NOs: 1 to 209 or a sequence encoding miR5032 or a homolog thereof can affect the SCN susceptibility of a plant. To identify genomic changes that render soybean plants resistant to SCN infection, ninety nine completely sequenced soybean accessions were phenotyped against five races of SCN (race 1, 2, 3, 5, and 14). The DNA sequences of the promoter regions of the genes Glyma.11G004900, Glyma.14G142500, and gma-miR5032 were retrieved from these 99 soybean accessions and analyzed for potential genetic differences and polymorphisms linked with resistance against five SCN races. Certain nucleotide polymorphisms associated with resistance to SCN infection are provided in Tables 5-8.

In a polymorphism as described in Tables 5-8, the nucleotide to the left of “/” describes the genomic sequence in an SCN susceptible plant and the nucleotide to the right of “/” describes the genomic sequence in an SCN resistant plant. For example, a single nucleotide polymorphism (SNP) A/T at position 500 indicates that a soybean plant having A at the 500^{th} position would be susceptible to SCN infection and a soybean plant having T at the 500^{th} position would be resistant to SCN infection.

Also, in a polymorphism as described in Tables 5-8, the nucleotide position can be described relative to the transcription start site (TSS) for the miRNA and ATG start codon for the protein. For example, a nucleotide at position −100 corresponds to the 100^{th} nucleotide upstream of the TSS or the ATG codon. Particularly, for SEQ ID NO: 211, TAC codon at positions 2001-2003 corresponds to the ATG codon on the opposite strand, which encodes the protein. For SEQ ID NO: 212, the protein coding sequence begins with the ATG codon at positions 2001-2003. For SEQ ID NO: 213, TSS for the miRNA is at the position 2300.

Thirteen polymorphisms in the promoter region and one polymorphism in the gene body (K/P) of the gene Glyma.11G004900 were identified to be associated with soybean resistance against one or more SCN races (Table 5). The sequence of the Glyma.11G004900 gene, including the promoter region and the protein coding region is provided in SEQ ID NO: 211. In the SEQ ID NO: 211, the corresponding nucleotides are on the negative strand, i.e., on the strand that does not encode the protein. Therefore, in the promoter sequence of the coding strand as provided in SEQ ID NO: 211, the corresponding polymorphisms contain the nucleotides that are complementary to the nucleotides indicated in Table 5.

The sequence of SEQ ID NO: 211 corresponds to the sequence having the relevant polymorphisms as found in the SCN susceptible plants. Therefore, when the nucleotides at the relevant positions of the susceptible genotype are replaced with the nucleotides of the resistant genotype as shown in Table 5, an SCN susceptible plant is converted in to an SCN resistant plant.

Ten polymorphisms in the promoter region of the gene Glyma.14G142500 were identified (Table 6). Six of these polymorphisms were found to be statistically significantly associated with resistance against SCN race 3, 5, and 14 (Table 6).

Seven polymorphisms in the promoter region of the gene encoding for miR5032 were identified (Table 7). Three polymorphisms were statistically linked to SCN resistance. The “insert” provided in Table 7 at the −309 position (“n” at the 1991^{st} position of SEQ ID NO: 213) represents a sequence of between 3 and 13 nucleotides.

Table 8 provides a list of polymorphisms from Tables 5-7 that are associated with susceptibility/resistance against an SCN infection. Accordingly, certain embodiments of the invention provide a method of producing an SCN resistant plant cell or a plant comprising one or more polymorphisms from Table 8. In preferred embodiments, the plant cell or a plant is a soybean plant cell or soybean plant.

The methods disclosed herein for producing an SCN resistant plant includes editing a genome to replace one or more nucleotides in an SCN susceptible plant cell or a plant with one or more nucleotides that confer resistant to an SCN infection.

Several techniques for editing a genome to replace one or more nucleotide with different one or more nucleotides are known in the art and are within the purview of the invention. Such techniques include homologous recombination using sequences that flank a genomic site to replace a native sequence with a non-native sequence. Additional such techniques include genome editing using CRISPR-Cas9 system, Zinc Finger based nuclease (ZFN) system, Transcription Activator-Like Effector Nucleases (TALEN) system, viral systems, such as recombinant adeno-associated viruses (rAAV), and transposons. Details of using these systems are known in the art and a person of ordinary skill in the art can design appropriate machinery to edit a genome and such embodiments are within the purview of this invention. Additional techniques of genome editing are also known in the art and such embodiments are within the purview of the invention.

Further embodiments of the invention provide an isolated nucleic acid sequence comprising a sequence of SEQ ID NO: 211, 212 or 213 or a fragment thereof. As discussed above, one or more nucleotides in SEQ ID NO: 211, 212 or 213 contain polymorphisms as provided in Tables 5-8, some of which are associated with susceptibility or resistance to an SCN infection. Accordingly, preferred embodiments of the invention provide an isolated nucleic acid sequence comprising SEQ ID NO: 211, 212 or 213 or a fragment thereof, wherein the sequence comprises one or more polymorphisms that are associated with resistance to SCN infection.

The fragment of SEQ ID NO: 211, 212 or 213 can be a fragment from the promoter region or the coding region.

For SEQ ID NO: 211, the promoter region is from the nucleotide at position 1 to the nucleotide at position 2000.

For SEQ ID NO: 212, the promoter region is from the nucleotide at position 1 to the nucleotide at position 2000.

For SEQ ID NO: 213, the promoter region is from the nucleotide at position 1 to the nucleotide at position 2298.

Further embodiments of the invention provide a combination of one or more probes that are suitable for predicting susceptibility or resistance of a plant cell or a plant to an SCN infection. The one or more probes are designed to determine in a plant cell or a plant the genomic sequence corresponding to SEQ ID NO: 211, 212 or 213, particularly, the genomic sequence corresponding to one or more polymorphisms described in Tables 5-8. A person of ordinary skill in the art can design one or more probes to identify in a genomic sequence one or more polymorphisms from Tables 5-8.

A genomic sequence can be detected using techniques known in the art, for example, using a labeled probe complementary to a sequence of a polymorphism. For example, a polymorphism can be detected based a labeled probe specific for that polymorphism.

The term “label” refers to a molecule detectable by spectroscopic, photochemical, biochemical, immunochemical, chemical, or other physical means. For example, useful labels include fluorescent dyes (fluorophores), fluorescent quenchers, luminescent agents, electron-dense reagents, biotin, digoxigenin, ³²P and other isotopes or other molecules that can be made detectable, e.g., by incorporating into an oligonucleotide. The term includes combinations of labeling agents, e.g., a combination of fluorophores each providing a unique detectable signature, e.g., at a particular wavelength or combination of wavelengths.

Exemplary fluorophores include, but are not limited to, Alexa dyes (e.g., Alexa 350, Alexa 430, Alexa 488, etc.), AMCA, BODIPY 630/650, BODIPY 650/665, BODIPY-FL, BODIPY-R6G, BODIPY-TMR, BODIPY-TRX, Cascade Blue, Cy2, Cy3, Cy5, Cy5.5, Cy7, Cy7.5, Dylight dyes (Dylight405, Dylight488, Dylight549, Dylight550, Dylight 649, Dylight680, Dylight750, Dylight800), 6-FAM, fluorescein, FITC, HEX, 6-JOE, Oregon Green 488, Oregon Green 500, Oregon Green 514, Pacific Blue, REG, Rhodamine Green, Rhodamine Red, ROX, R-Phycoerythrin (R-PE), Starbright Blue Dyes (e.g., Starbright Blue 520, Starbright Blue 700), TAMRA, TET, Tetramethylrhodamine, Texas Red, and TRITC.

Accordingly, methods of determining susceptibility or resistance of a plant cell or a plant to an SCN infection are also envisioned. Such methods comprise determining in a plant cell or a plant the genomic sequences corresponding one to more polymorphisms provided in Tables 5-8 and determining, based on the genomic sequences, whether a plant cell or a plant is resistant or susceptible to an SCN infection.

As discussed above, comparing the methylomes of the parental lines that are resistant or susceptible to SCN with that of the NILs that are resistant or susceptible to SCN resulted in the identification of heritable as well as novel non-parental differentially methylated regions overlapping with genes related to SCN parasitism of soybean. Thus, the disclosure provides the genes involved in biochemical basis of Rhg4 function in SCN resistance. Such comparisons can also be used to identify genes involved in conferring traits other than SCN resistance.

Accordingly, certain embodiments of the invention provide a method of identifying one or more genes that confer a trait to a plant when expressed or inactivated, the method comprising the steps of:
a) providing two NILs, one of the two NILs having one or more genetic loci known to confer the trait and the other NIL not having the one or more genetic loci known to confer the trait;
b) analyzing the methylation status of a plurality of genetic loci within the genomes of the two NILS,
c) comparing the methylation status of the plurality of genetic loci between the two NILs to identify a plurality of genetic loci that are differentially methylated between the two NILS,
d) comparing the mRNA expression profiles of the two NILs and identifying a plurality of mRNAs that are differentially expressed between the two NILS,
e) identifying a plurality of genetic loci that is differentially methylated between the two NILs and that encodes differentially expressed mRNAs,
f) expressing or inactivating in a plant cell each of a plurality of genes located in the plurality of genetic loci that is differentially methylated between the two NILs and that encodes differentially expressed mRNAs; and
g) identifying one or more genes that confer the trait to the plant when expressed or inactivated.

Additional genes that confer a trait to a plant when expressed or inactivated can be identified by further steps of:
h) comparing the methylation status of the plurality of genetic loci between the two NILs and the two parent lines to identify a plurality of genetic loci that are differentially methylated in one of the two NILs compared to the other NIL and the two parent lines,
i) identifying a plurality of genes located in the plurality of genetic loci that are differentially methylated in one of the two NILs compared to the other NIL and the two parent lines,
j) expressing or inactivating in a plant cell each of the plurality of genes identified in step i), and
k) identifying one or more genes from the genes expressed or inactivated step i) that impart the trait to the plant when expressed or inactivated.

As used herein, the phrase “a gene confers a trait” or grammatical variations thereof indicates that a plant containing the gene exhibits the trait and an otherwise genetically identical plant lacking the gene does not exhibit the trait. Thus, in two otherwise genetically identical plants, the presence of a trait in the plant containing the gene and the absence of the trait in the plant not containing the gene is attributed to the presence or the absence of the gene.

The differentially methylated regions can be located in the protein coding regions or transposon elements. The differential methylation can be differential CG-methylation, differential CHG-methylation, or differential CHH-methylation.

In preferred embodiments, the two NILs are derived from a parent line having the trait and a parent line not having the trait by repeated back-crossing.

In preferred embodiments of the invention, the plant is a soybean plant and the trait is resistance to SCN. Also, the one or more genetic loci known to confer the SCN resistance encodes a serine hydroxymethyltransferase, an α-SNAP protein, a putative amino acid transporter, a wound-inducible protein, or a combination thereof.

Exemplary embodiments of such methods are described in Examples 1 to 9 below. A skilled artisan can implement such methods to identify genes that confer a trait of interest in a plant and such embodiments are within the purview of the invention.

### Materials and Methods

### Developing Near-Isogenic Soybean Lines Differing in GmSHMT08 Gene

Two NILs, TN09-16 and TN09-29, which exhibit susceptible and resistant responses, respectively, to SCN HG type 0 (race 3) were developed. These NILs are highly homozygous recombinant inbred lines derived from individual F13 generation single plants from the cross ‘Fowler’×‘Anand’. Anand was developed from the cross Holladay×Hartwig by the Missouri Agricultural Experiment Station and released in 1999 (Anand et al., 2001). Fowler was developed from the cross Hartwig×Holladay by the USDA-ARS at Jackson, TN, and released in 1999 (Young LD. 2001). Hartwig derived its resistance from Plant Introduction (PI) 437654. Simple sequence repeat (SSR) markers associated with rhg1 (Satt309) and Rhg4 (Satt162 and Satt632) were initially used to examine the genetic differences between these two NILs. In addition, the SCN resistance genes soluble NSF attachment proteins (GmSNAP18) and the serine hydroxymethyltransferase (GmSHMT08) at the rhg1-b and RHg4 loci, respectively, were amplified, ligated into pGMT-easy vector (Promega), and sequenced to further confirm the genetic differences between the NILs at GmSHMT08 locus.

### Nematode Inoculation and Collection of Root Tissues

Soybean seeds of TN09-16 and TN09-29 were washed for 30 minutes under running water and then soaked in 10% bleach for 10 minutes. After this surface sterilization the seeds were washed again for 30 minutes to remove bleach remnants. The seeds were then germinated on wet germination paper in dark at 26° C. for three days. Healthy looking 3 day old seedlings were selected for nematode inoculation. Freshly hatched second stage juveniles (J2s) of SCN HG type 0 (race 3) were surface-sterilized and then suspended in 0.1% sterile agarose solution at a concentration of approximately 500 J2s per 100 μL. Each seedling was inoculated with about 3000 J2s, by spreading the nematodes across the whole root of a seedling. Control plants were set up in exact same way, except mock inoculations were performed using 0.1% (w/v) Control and inoculated plants were arranged in replicates, each agarose per seedling. containing at least six plants and maintained in a controlled plant growth chamber at 26° C. with 16-h light/8-h dark conditions as previously described (Rambani et al., 2015). Five days post SCN-inoculation, roots tissues were collected from both inoculated and non-inoculated soybean roots in three biologically independent replicates resulting in a total of 12 samples. Successful infection of each replicate was confirmed by examining one-fourth of the infected seedlings using acid fuschin stain. The two parental lines (Fowler and Anand) were only mock-inoculated in the same experimental settings and a total of 6 biologically independent samples were similarly collected five days later. DNA and RNA were isolated from each sample and used construct methylC-seq and RNA-seq library.

### Preparation of MethylC-Seq Libraries

Genomic DNA of the infected and non-infected root samples was extracted using DNeasy Plant Mini Kit (Qiagen). Whole genome methylC-seq libraries were constructed as per protocol from Illumina TruSeq Library Prep kit (Illumina, San Diego, CA) with slight modifications of the bisulfite treatment. Briefly, about 2 μg of genomic DNA (gDNA) in addition to unmethylated lambda DNA were fragmented using Bioruptor (Diagenode Inc. USA, Denville, NJ) and then spiked with unmethylated fragmented lambda DNA (Promega, Madison, WI) that constitutes up to 2% of total concentration. Fragment size distribution of sheared DNA was verified using the Agilent Bioanalyzer 1000 DNA chip (Agilent Technologies, Santa Clara, CA). DNA fragments were then ligated to cytosine-methylated adapters (provided by Illumina) and then subjected to sodium bisulfite treatment using MethylCode™ Bisulfite Conversion Kit (Invitrogen, Grand Island, NY). DNA fragments between 400 and 500 bp were selected on the Pippin Prep system (Sage Sciences) and enriched by 10 cycles of PCR as recently described (Rambani et al., 2015). PCR products were then purified using Agencourt AMPure XP beads (Beckman Coulter, Inc., Brea, CA) and subsequently enriched using 5 additional PCR cycles according to Illumina's protocol. The PCR products were purified once more and library size distribution was examined using the Agilent Bioanalyzer 1000 DNA chip. Finally, the libraries were quantified and sequenced using Illumina HiSeq 2500 platform.

### Identification of DMRs and Overlapping Genomic Regions

Sequencing adapters were trimmed from bisulfite sequencing reads (BS reads) and low quality reads below Phred threshold of 33 were removed using Trimmomatic. Then high quality paired-end reads were aligned to the soybean reference genome (Wm82.a2.v1) using Bismark with default parameters. Alignment files generated by Bismark were analyzed by the R bioconductor package methylKit to identify differentially methylated cytosines. Methylation status at each cytosine covered by at least 10 reads in the CG, CHG and CHH sequence contexts were calculated. A non-overlapping sliding window of 200 bp overall the 20 soybean chromosomes was used to identify DMRs with methylation difference of at least 50%. Significance of differentially hyper- and hypo-DMRs was determined using q-value less 1%. DMRs were mapped to various genic regions including prompter (1 kb upstream of the transcription start site), 5′ and 3′ untranslated regions (UTRs), and gene body (transcribed region) using Bioconductor package rtracklayer in a custom R script. Methylation cytosine report files generated by Bismark were used to visualize global methylation levels over protein-coding genes and TEs using ViewBS package (see world-wide-website: github.com/xie186/ViewBS).

The most recent assembly of soybean genome (Wm82.a2.v1) was released without annotation of TEs. Thus, sequences of previously annotated and known TEs in soybean assembly were obtained from SoyTEdb. The TE sequences were used to mask the new soybean assembly using RepeatMasker and output file with TE coordinates in the new assembly was created. Then, overlaps of DMRs with TEs belonging to various families were reported. BED tools were used to calculate the distance from DMR-associated TEs to the nearest gene.

### RNA Library Preparation and Transcriptome Analysis

mRNA was isolated using magnetic mRNA isolation kit (NEB). NEBnext mRNA library prep master mix (NEB) was used to build libraries following manufacturer's protocol. RNA-seq libraries were sequenced on Illumina Hiseq 2500 platform. Quality of paired-ended reads was verified with FastQC (version 0.11.4) (see world-wide-website: bioinformatics.babraham.ac.uk/projects/fastqc/). Adapter sequences and low-quality reads were removed using Trimmomatic (version 0.35). Qualified reads were then mapped to the soybean reference genome (Wm82.a2.v1) using TOPHAT v.2.0.13 with default parameters. Reads mapped to multiple loci were discarded and numbers of uniquely mapped reads per gene were determined using HTSeq. Counts generated by HTSeq were used to determine differentially expressed genes using the R bioconductor package edgeR. Genes with false discovery rate less than 0.1 or 0.05 were considered significantly differentially expressed. Separate count files were generated using the python-based package QoRTs for counting sequencing reads spanning exons for every gene. The count files were used with the bioconductor R package JunctionSeq to determine differentially spliced transcripts.

### GO Terms Enrichment Analysis

GO terms enrichment analysis of differentially methylated genes and differentially expressed genes were determined using soybase tools and AgriGO database. Statistically significant enriched GO terms were calculated using Fisher's exact test and Bonferroni multi-test adjustment with a q value less than 0.05. GO terms were clustered based on semantic similarity to other GO terms in Uniprot database using REVIGO.

### Generation of Transgenic Hairy Roots and Nematode Infection Assay

The coding sequences of four soybean genes were amplified from root cDNA and cloned under the control of a soybean ubiquitin promoter and the RuBisCO small subunit terminator in the binary vector pG2RNAi2, which contains the superfolded green fluorescent protein (sGFP) to facilitate identification of transgenic hairy roots. All constructs were confirmed by sequencing. Each of the four constructs as well GFP-only control vector were transformed into Agrobacterium rhizogenes strain K599 and used for generation of transgenic soybean hairy roots Transgenic hairy roots overexpressing these constructs were generated in seven-day-old seedlings of the susceptible isogenic line TN09-016 using the method previously described by Kereszt et al. (2007). Three weeks after A. rhizogenes inoculation, the transgenic hairy roots (3 per plant) were selected using an epifluorescent microscope (Olympus, model SZX12) equipped with GFP filter. Transgenic hairy roots expressing the empty vector containing only the GFP marker gene were used as control. In all cases, non-transgenic hairy roots as well as the main root system were removed. The composite transgenic plants were then planted in 656 cm³ cone-tainers (6.4 cm×25.4 cm) containing steam-sterilized sand mixed with top soil (3:1) and arranged in a randomized complete block design. Two days after planting, each composite plant was inoculated with about 3000 eggs of SCN race 3 (HG Type 0). The plants were maintained under controlled growth conditions (16-h day length and 26° C.) in Percival reach-in plant growth chambers. Five weeks after inoculation, the cysts were extracted from each plant separately and counted under a stereoscope. The number of cysts was used to calculate the female index as a percentage of the average number of cysts determined on the test lines relative to those determined on the control line. Nematode susceptibility assays of the isogenic lines and their parents were conducted in the greenhouse using SCN HG type 0 (race 3). Seeds of each line were planted in pots (2 seeds per pot) containing soil:sand (1:1) mixture and organized in a randomized complete block design. Each pot was inoculated with approximately 4,000 eggs at seeding. Approximately 5 weeks after planting the cysts were blasted off the roots and counted under the microscope. Statistically significant differences between the lines were calculated using t-tests with P value<0.001.

The sequences of the genes identified by the Glyma IDs provided throughout this disclosure can be found in the soybean genome database (see the website: soybase.org) and the sequences associated with the Glyma IDs within the soybean genome database are hereby incorporated by reference in their entireties.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1—Developing Near-Isogenic Soybean Lines Differing at the SCN Resistance Gene Rhg4

Two near isogenic lines (NILs), TN09-16 and TN09-29, respectively containing the susceptible and resistant allele of RHg4, were generated. These NILs are highly homozygous recombinant inbred lines derived from individual F13 generation single plants from a cross between the SCN-resistant variety Fowler and the SCN-susceptible variety Anand. Homozygosity level of these NILs was estimated to be 0.9998 based on the number of inbreeding generations after the creation of F1. SCN resistance in the parental line Fowler was acquired from the Plant Introduction (PI) 437654. PI 437654, which exhibit ‘Peking-type’ resistance, has been shown to carry the SCN resistance gene Rhg4 and three copies of rhg1a. Initially, Simple Sequence Repeat (SSR) markers associated with rhg1 (Satt309) and Rhg4 (Satt162 and Satt632) were used to test potential genetic differences between these two NILS at rhg1 and Rhg4 loci. The resistant line TN09-29 inherited the two resistant alleles from its Hartwig ancestry. The susceptible line TN09-016, however, did not inherit the Rhg4 allele but did receive the rhg/resistant allele. The SCN resistance genes soluble NSF attachment proteins (GmSNAP18) and the serine hydroxymethyltransferase (GmSHMT08) at the rhg1 and Rhg4 loci, respectively, were cloned and sequenced from the isogenic lines. While the nucleotide sequences of GmSNAP18 were identical in both lines, GmSHMT08 showed two single nucleotide polymorphisms (SNPs), leading to R130P and Y358N amino acid substitutions between the TN09-29 and TN09-16 (FIG. 11). These two amino acid substitutions were previously reported to establish the difference between the resistant and susceptible alleles of Rhg4.

Nematode susceptibility assays of the parental lines and the NILs were conducted against SCN HG Type 0 (race 3). The parental line Anand showed very high level of SCN susceptibility with more than 100 cysts per plants. In contrast, Fowler showed high level of resistance with only one or two cysts in the inoculated plants (FIG. 12). Similarly, the NILs showed completely opposite responses to SCN infection. TN09-16 showed very high level of susceptibility, whereas TN09-29 showed complete resistance to SCN (FIGS. 12). TN09-16 and TN09-29 were also evaluated for SCN resistance in the USDA Southern Uniform Soybean Test program. TN09-29 showed strong resistance to race 2 (HG Type 1.2.5.7), race 3, and race 5 (HG Type 2.5.7) with the highest possible resistant score ratings (1, 1, 1). In contrast, TN09-16 showed high level of susceptibility rated at scores of 4, 4, and 4, for SCN races 2, 3, and 5, respectively. Thus, Rhg4 is required for complete resistance against SCN in the PI 437654-derived germplasms.

### Example 2—the Genomes of the Near Isogenic Lines are Substantially Differentially Methylated

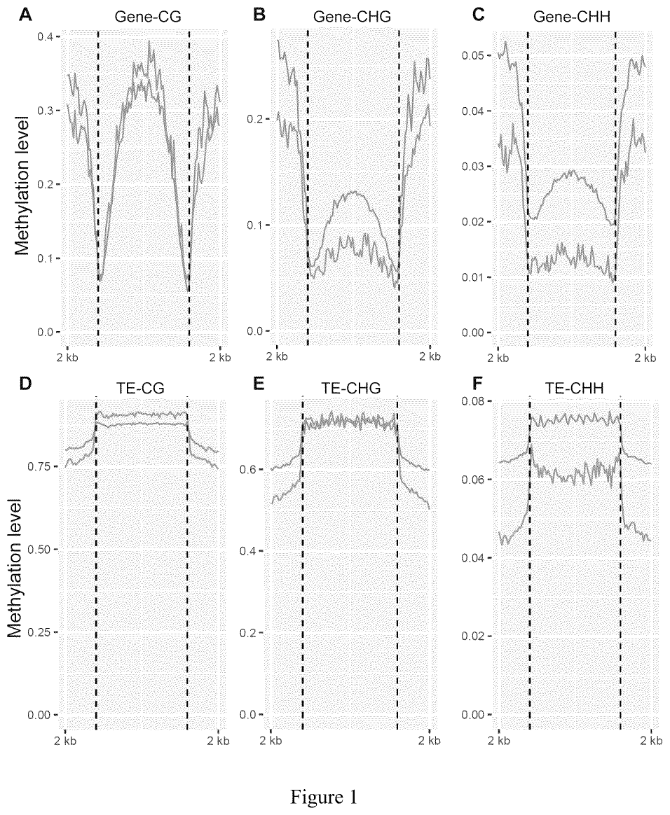

Taking into consideration the function of GmSHMT08 in cellular methylation, its potential impact on the methylome of the NILs under non-infected conditions was examined. Seeds of the NILs TN09-16 and TN09-29 were germinated and root tissues of non-infected one-week-old seedlings were collected in three biological independent samples. DNA were isolated from these six samples and used to prepare methylC-seq libraries. The libraries were sequenced using Illumina HiSeq 2500 sequencing platform. 1.190E+09 100-bp reads were obtained for the susceptible TN09-16 line and 1.159E+09 reads were obtained for the resistant TN09-29 line, providing more than 100× coverage of the soybean genomes. Bisulfite conversion rate was estimated using the λ phage genome and found to be greater than 99.7%. Differentially methylated cytosines in the CG, CHH and CHG sequence contexts were considered for downstream analyses only if they covered by at least 10 high-quality reads. The global methylation levels were compared between TN09-16 and TN09-29 over genes and transposable elements (TEs) in the CG, CHG, and CHH contexts. Interestingly, differences in the global methylation levels between the two lines were detected in gene body as well as upstream and downstream regions in all sequence contexts (FIGS. 1A-1C). The susceptible line TN09-16 showed higher methylation levels than the resistant TN09-29 line over gene body and the flanking regions in the CHG and CHH contexts (FIGS. 1B and 1C). In the CG context, TN09-16 showed higher methylation level in the flanking regions but lower methylation in gene body (FIG. 1A). Differences in the global methylation levels between the two lines were also observed in TEs (FIGS. 1D-1F). TN09-16 showed higher methylation levels than TN09-29 in the TE flaking regions in all sequence contexts (FIGS. 1D-1F). Over the body of TEs, the global methylation levels of TN09-16 and TN09-29 in the CHG context were comparable (FIG. 1E). In CG and CHH, however, the differences between the two lines were noticeable. TN09-16 showed higher CHH-methylation and lower CG-methylation than TN09-29 (FIGS. 1D and 1F). These analyses indicate that the genomes of these NILs are considerably differentially methylated.

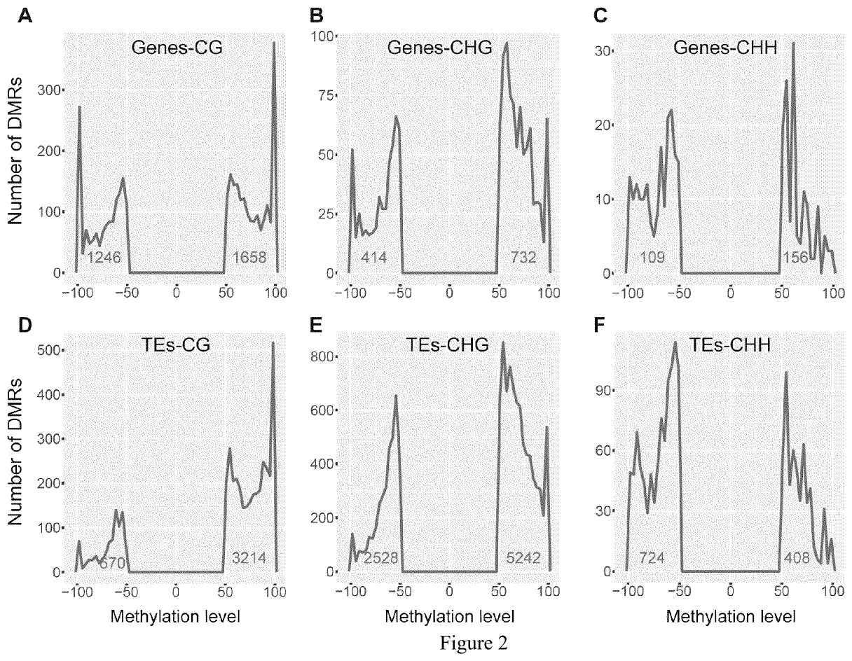

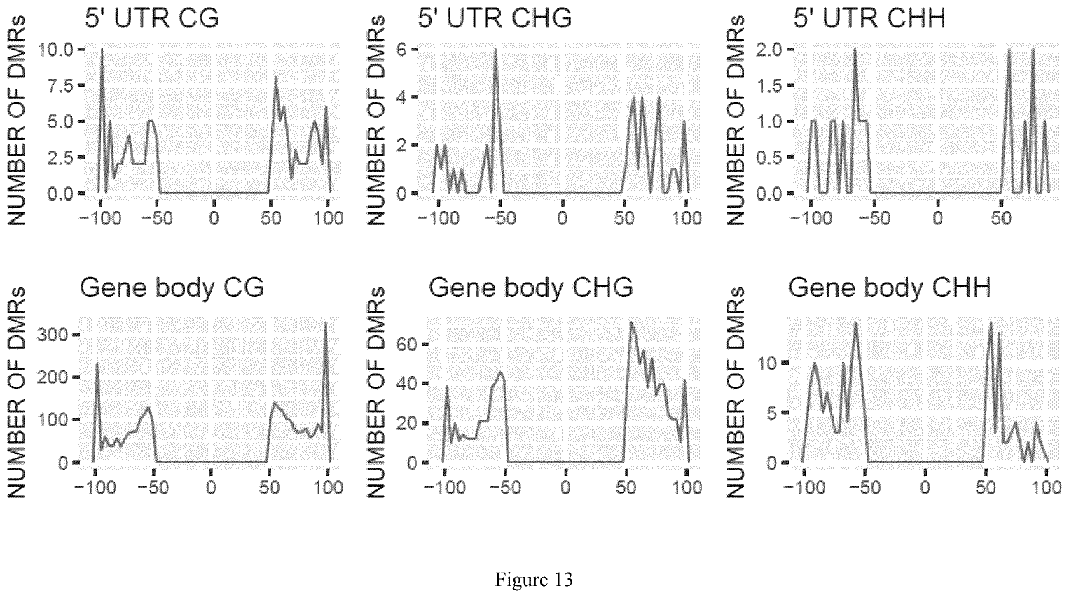

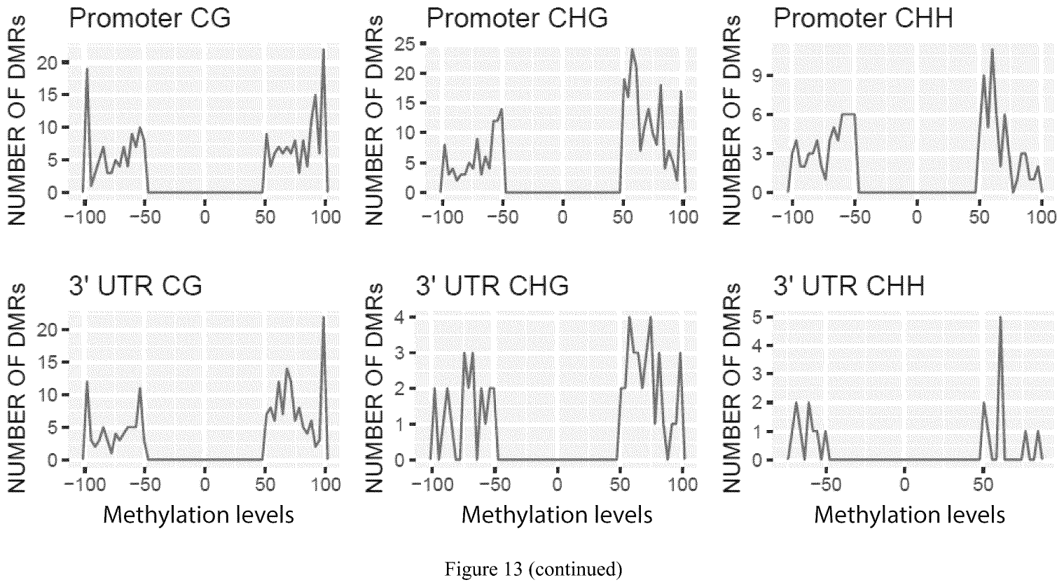

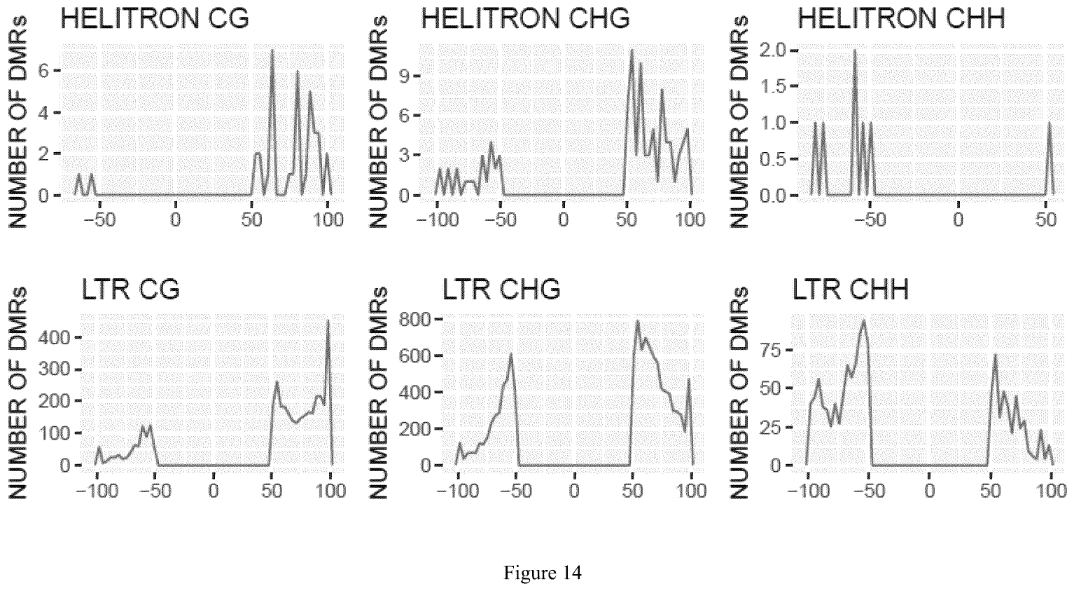

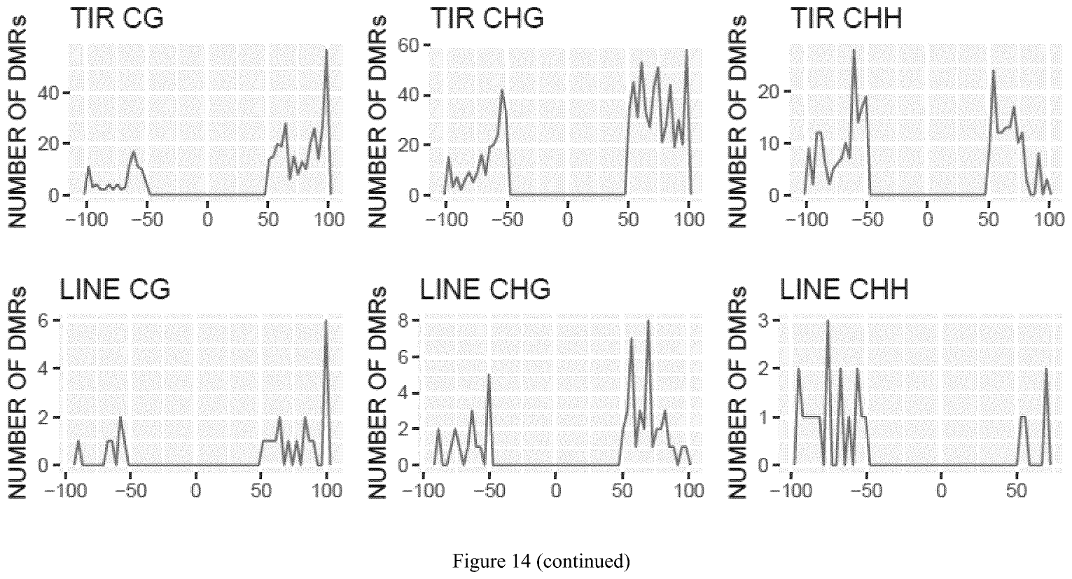

To identify genomic regions with the most significant methylation differences, the genomes of these two lines were divided into 200-bp non-overlapping bins and then differentially methylated regions (DMRs) with at least 50% methylation differences were identified using a false discovery rate (FDR) of 0.01. Using these stringent criteria, 21,852 unique DMRs between TN09-16 and TN09-29 were identified. Of these 4,180 and 11,211 DMRs overlapped with protein-coding genes and TEs, respectively (FIG. 2). The remaining DMRs are located in unannotated regions of the genome. As shown in FIG. 2, the majority of these DMRs occurred in the CHG and CG contexts, with approximately two-thirds of these regions being hypermethylated in the susceptible line TN09-16 compared with the resistant line TN09-29. When the 4,180 DMRs were mapped to various annotated features of protein-coding genes CG-DMRs were found to occur mostly in gene body and to a much lesser extent in gene promoters and 5′ and 3′ untranslated regions (UTRs) (FIG. 13). Notably, 70% of CHG-DMRs were found in gene body (FIG. 13). In contrast to CG- and CHG-DMRs, the number of CHH-DMRs overlapping with gene features was relatively small (FIG. 11). TE-associated DMRs were mapped to various transposon families. Remarkably, 88% of these DMRs were associated with long terminal repeat (LTR) retrotransposons (FIG. 14). DMRs overlapping with the DNA transposons Helitron and TIR constituted about 10% of the total number of TE-associated DMRs (FIG. 14).

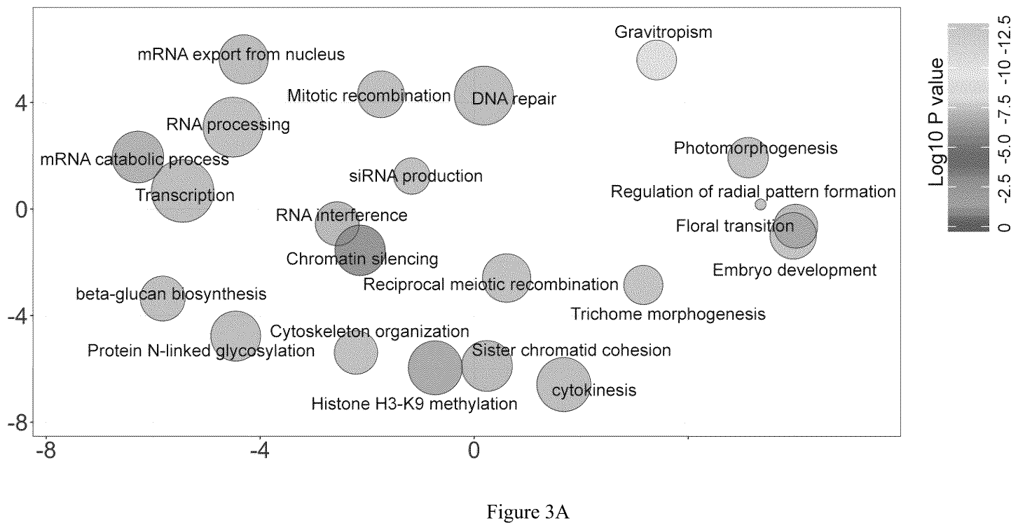

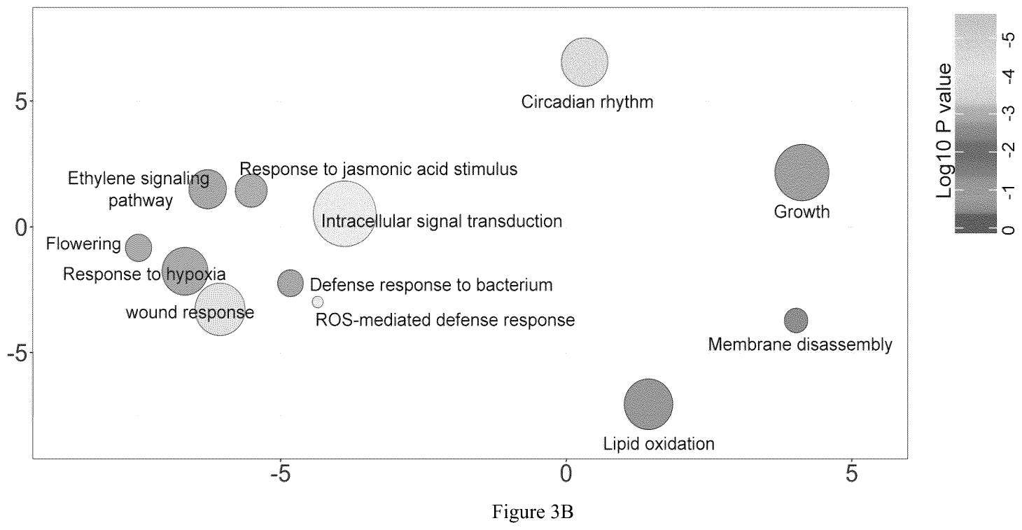

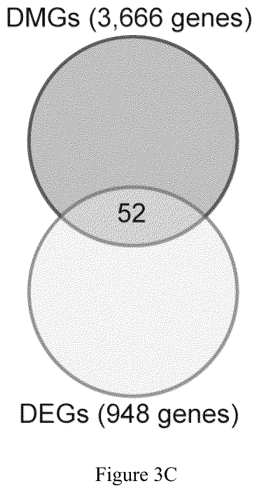

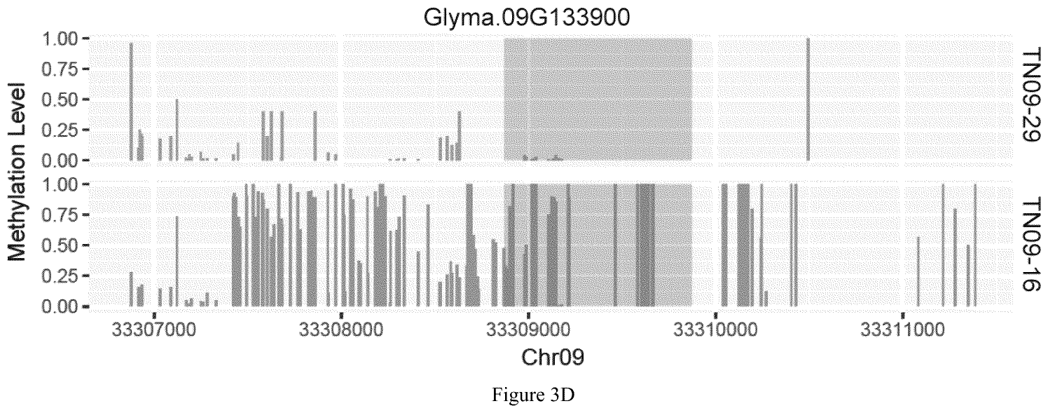

Gene Ontology (GO) enrichment analysis of the 3,666 differentially methylated genes (DMGs) between TN09-16 and TN09-29 revealed overrepresentation of genes involved in various biological processes (FIG. 3A). Of note is that ontologies with functions related to chromatin silencing, RNA interference, DNA repair, production of siRNAs, and histone H3-K9 methylation were significantly overrepresented. Thus, the enrichment of genes related to epigenetic modifications among the DMGs may explain the remarkable methylome differences between these two NILs.

### Example 3—Differential DNA Methylation Between the Isogenic Lines may Prime Their Responses to SCN Infection

To examine the degree to which cytosine methylation impacts gene expression in the isogenic lines, RNA-seq libraries were generated from the same root samples used for DNA methylation analysis. 948 differentially expressed genes (DEGs) were identified between TN09-16 and TN09-29 at a FDR of 0.05. GO term analysis revealed statistically significant enrichment for categories related to wounding response, defense response, membrane disassembly, and intracellular signal transduction (FIG. 3B). These findings suggest that differences in priming response between TN09-16 and TN09-29 may contribute to their contrasted response to SCN infection. To examine this suggestion the 948 DEGs were compared with a list of previously identified syncytium DEGs (6,903) and 267 genes were identified to be common between these two gene lists. This significant overlap (28.16%, x2 =38.91, P=1.81E-08) further supports that differences in priming response between TN09-16 and TN09-29 may contribute to their contrasted response to SCN infection. The 948-gene list was next compared with the 3,666 DMGs to determine if there is a significant enrichment for the DMGs among the DEGs. Fifty two genes were common to both gene lists, revealing a significant enrichment (5.48%, x2 =27.76, P=4.07E-06) for the DMGs among the DEGs (FIG. 3C). The methylation patterns of these 52 genes seem to impact their expression levels. For example, promoter and gene body hypermethylation of Glyma.09G133900 in TN09-16 was associated with a significant gene downregulation (FIG. 3D). In addition, 9 DEGs were identified that are located with 2 kb from differentially methylated TEs. Together, these data indicate that under non-infected conditions differential DNA methylation contributes to differential gene expression between the NILs that may prime their responses to SCN parasitism.

### EXAMPLE 4—the Susceptible and Resistant Isogenic Lines Exhibit Contrasted DNA Methylation Patterns in Response to SCN Infection

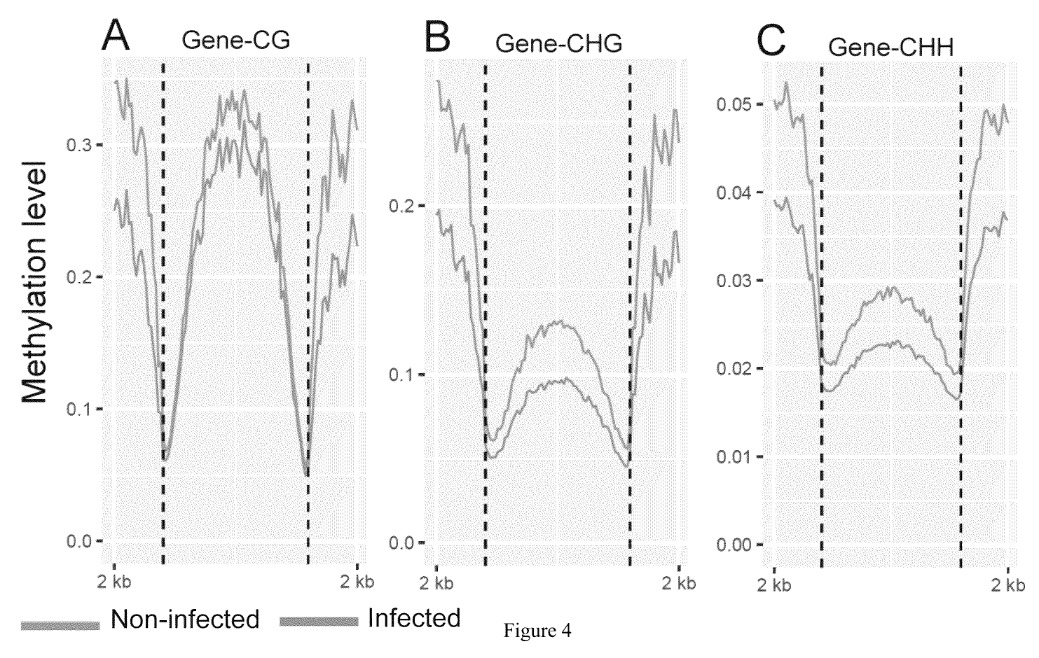

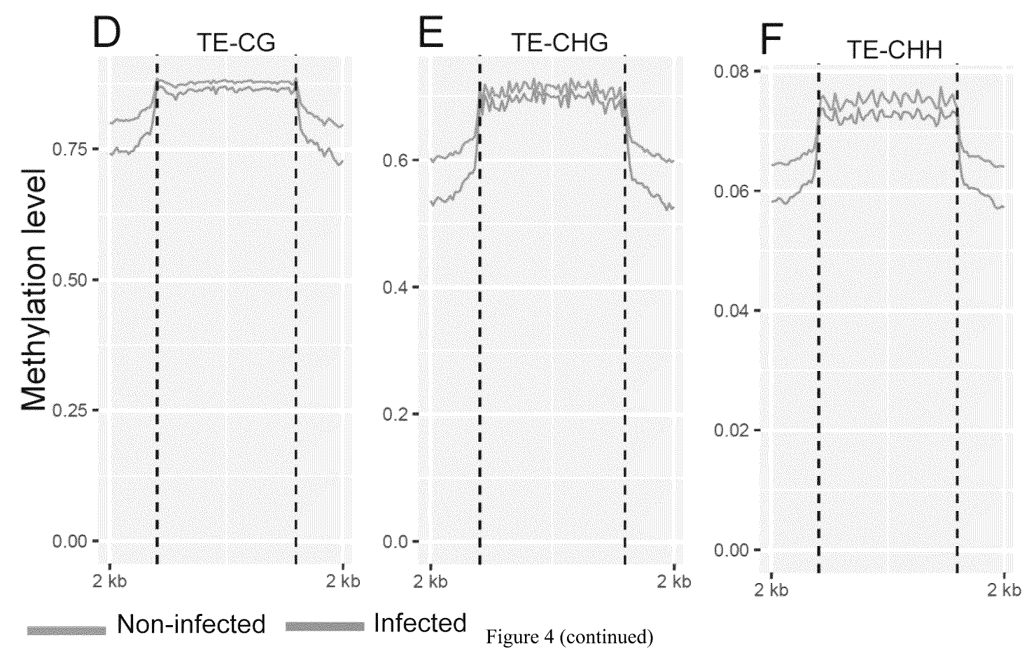

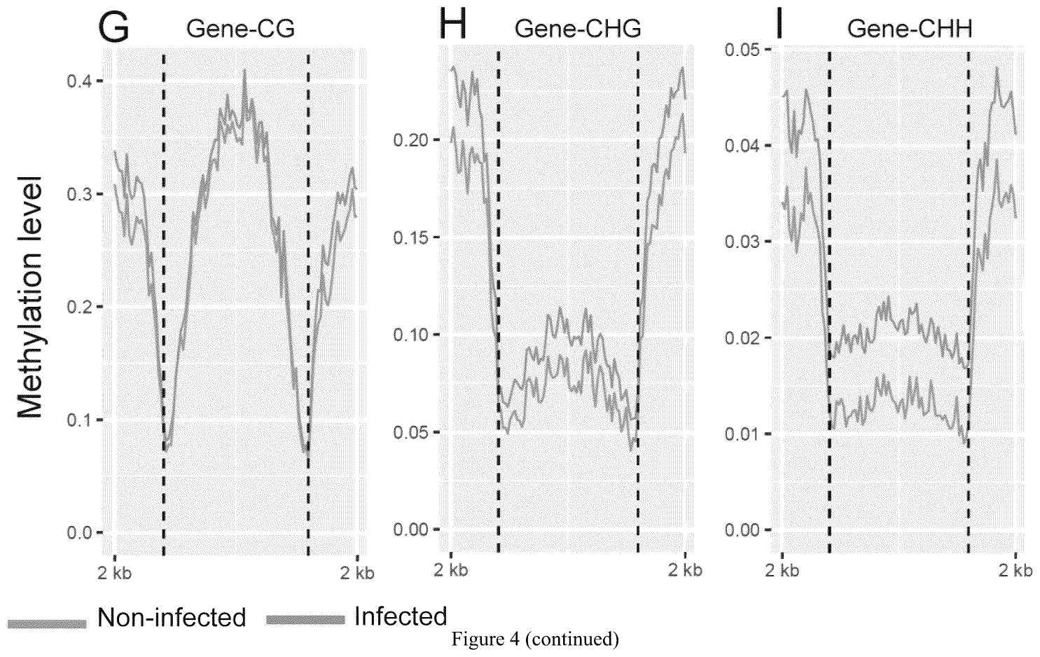

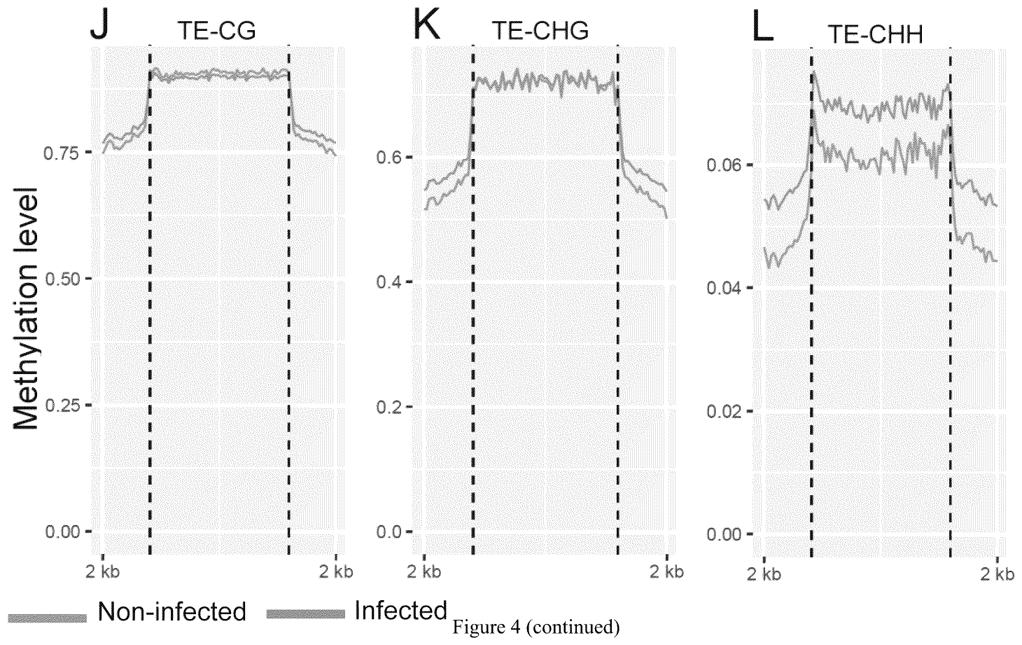

Whether the methylomes of the TN09-16 and TN09-29 are similarly altered in response to SCN (race 3) infection was examined. MethylC-seq libraries were constructed from SCN-infected roots at 5 day post infection (dpi) and compared with control samples. Both infected and non-infected libraries were prepared from root tissues collected from the same experiments at the same time. Differentially methylated cytosines were identified as described above and global methylation levels were compared between infected and non-infected samples over genes and TEs in all sequence contexts. In response to SCN infection, the susceptible line TN09-16 showed reduced methylation levels over protein-coding genes in all sequence contexts compared with non-infected control (FIGS. 4A-4C). In sharp contrast, the SCN-infected samples of the resistant line TN09-29 showed increased methylation levels over protein-coding genes in all sequence contexts compared with non-infected control (FIGS. 4G-4I).

Differences in global methylation patterns over TEs in response to SCN infection were also observed between the NILs. Infected TN09-16 samples showed reduced methylation levels over the body of TEs and flaking regions in all sequence contexts in comparison with the non-infected control samples (FIGS. 4D-4F). On the contrary, infected TN09-29 samples showed increased methylation level over the body of TEs and flaking regions in CHH context relative to non-infected samples (FIG. 4L). In the CG and CHG contexts, however, increased methylation levels in the TN09-29-infected samples were observed only over the TE flaking regions (FIGS. 4J and 4K). These analyses indicate that the NILs exhibit contrasted DNA methylation patterns in response to SCN infection with hypomethylation being more predominant in the susceptible line, whereas hypermethylation being more predominant in the resistant line.

### Example 5—DNA Methylation Patterns Associated With the Susceptible and Resistant Responses are Highly Specific

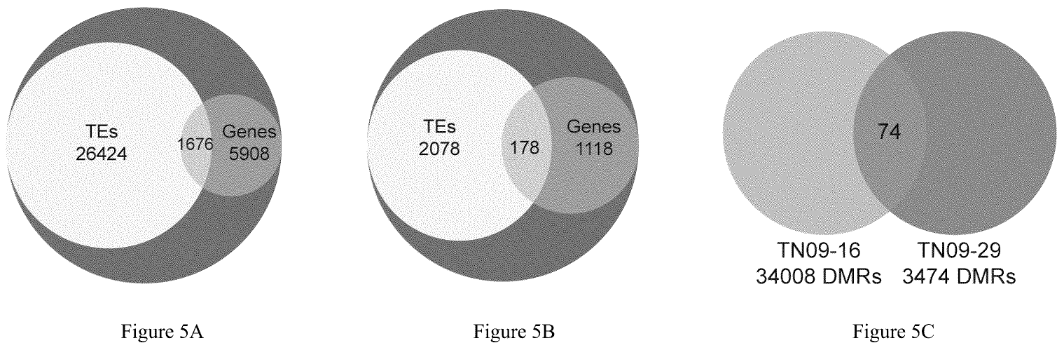

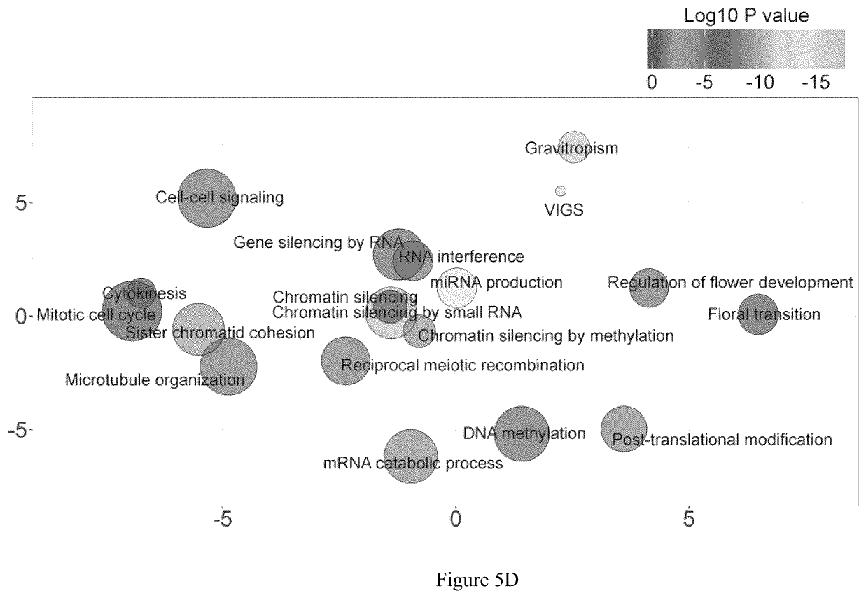

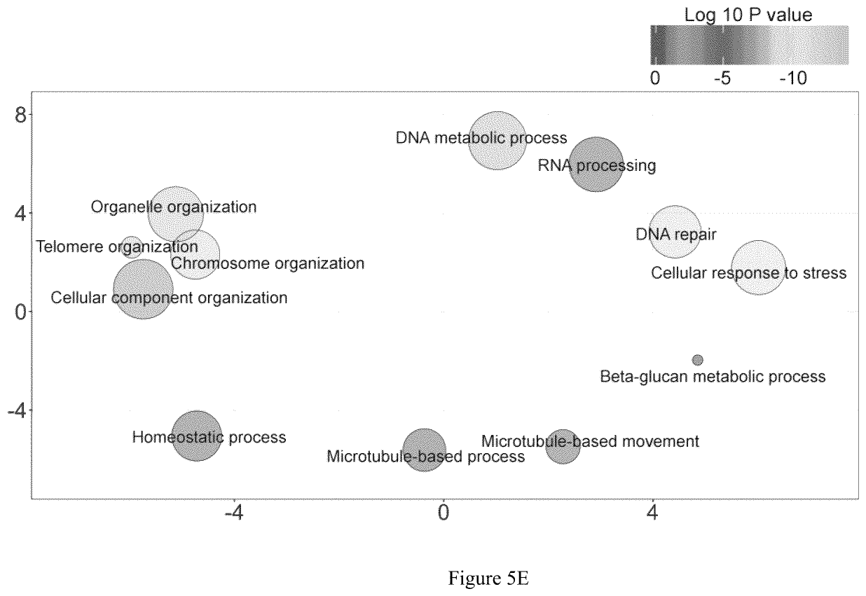

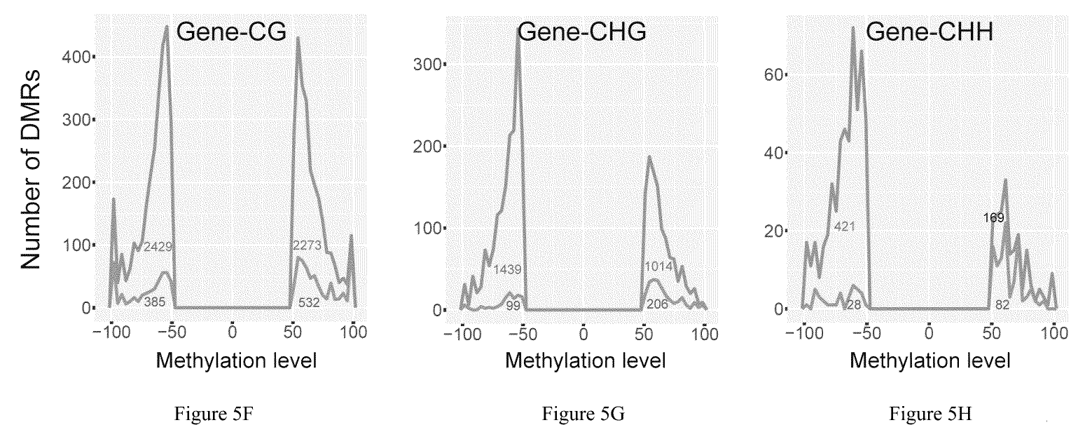

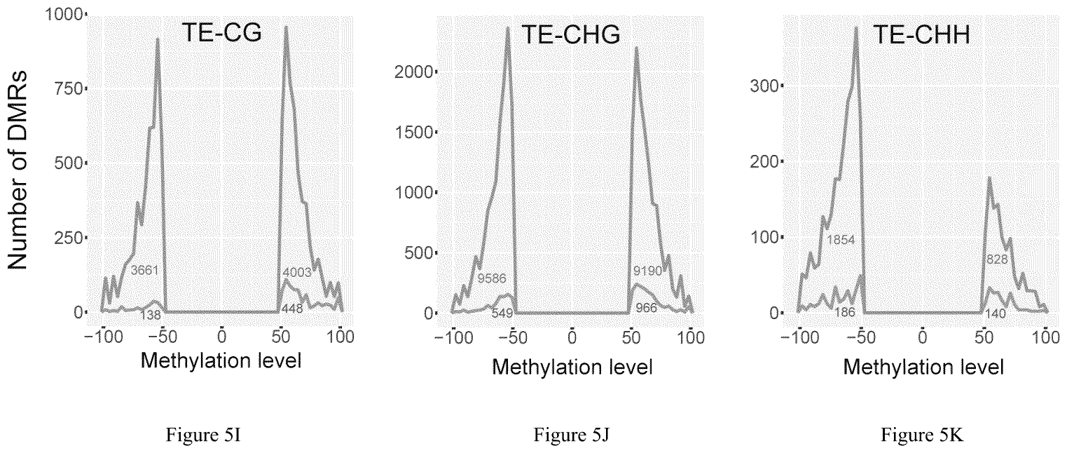

To localize the genome-wide DNA methylation profiles induced by SCN in both lines DMRs were identified as indicated above and mapped to the annotated protein-coding genes and TEs. In response to SCN infection 50,040 DMRs were identified in TN09-016. Of these, 7,585 (15.16%) overlapped with protein-coding genes, and 28,100 (56.16%) overlapped with TEs (FIG. 5A). Of the 28,100 TE-associated DMRs 1,676 (5.96%) were located in genes. Notably, the number of DMRs in TN09-29 was dramatically lower (FIG. 5B). A total number of 5,080 DMRs were identified in the TN09-29-infected samples compared with controls (FIG. 5B). Of these, 1,296 (25.51%) overlapped with protein-coding genes, and 2,356 (46.38%) overlapped with TEs (FIG. 5B). Of the 2,356 TE-associated DMRs 178 (7.55%) were located in genes. When the DMRs overlapping with protein-coding genes or TEs were compared between the two lines, only 74 DMRs were common (FIG. 5C), indicating that DNA methylation patterns associated with the susceptible and resistant responses are highly specific. This indication was further supported by GO term enrichment analysis showing the association of the DMGs in TN09-16 and TN09-29 with different GO biological process categories (FIGS. 5D and 5E).

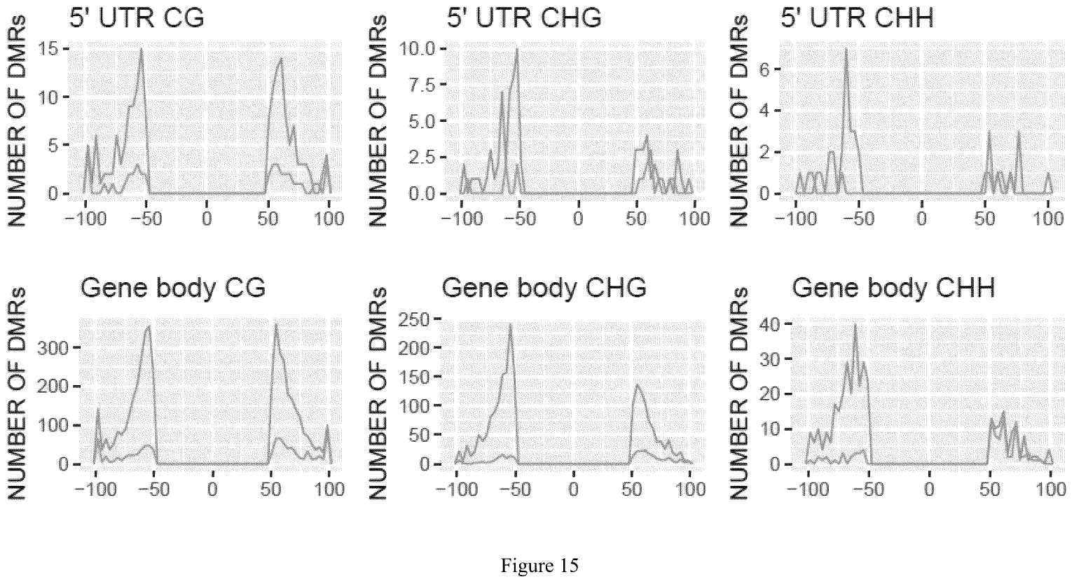

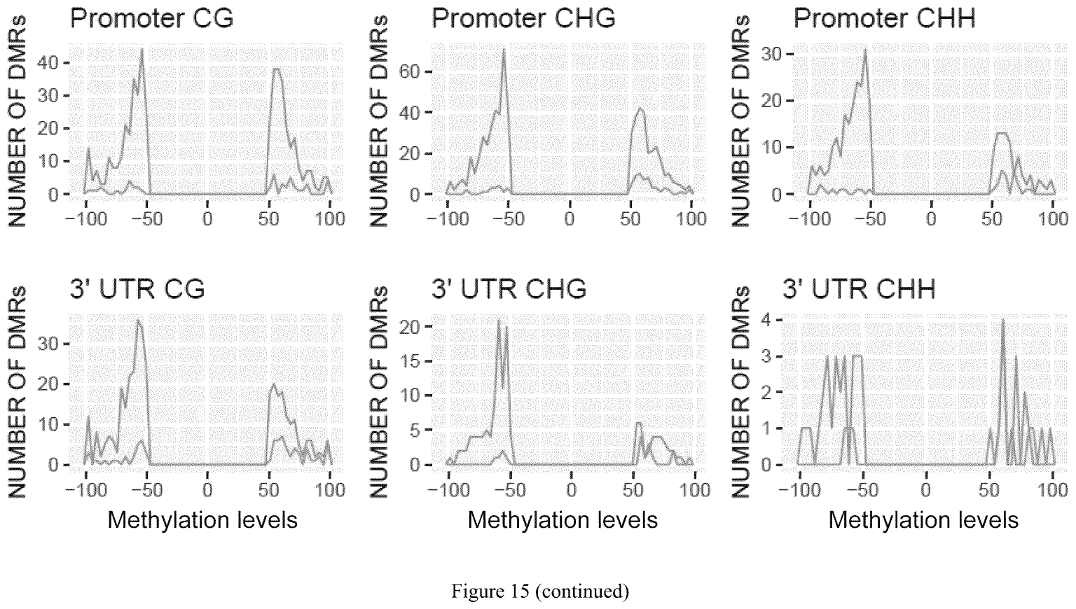

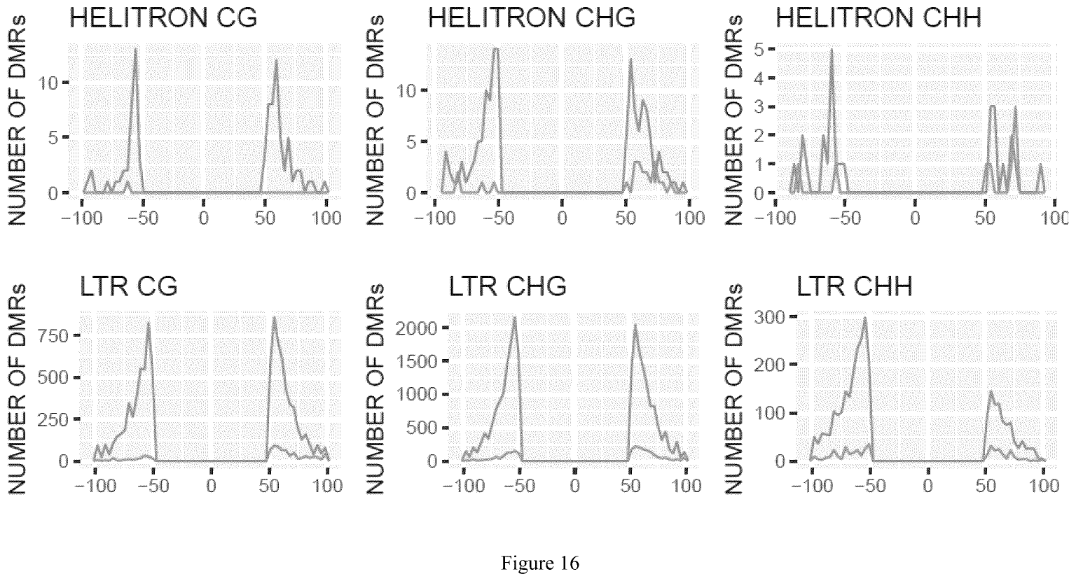

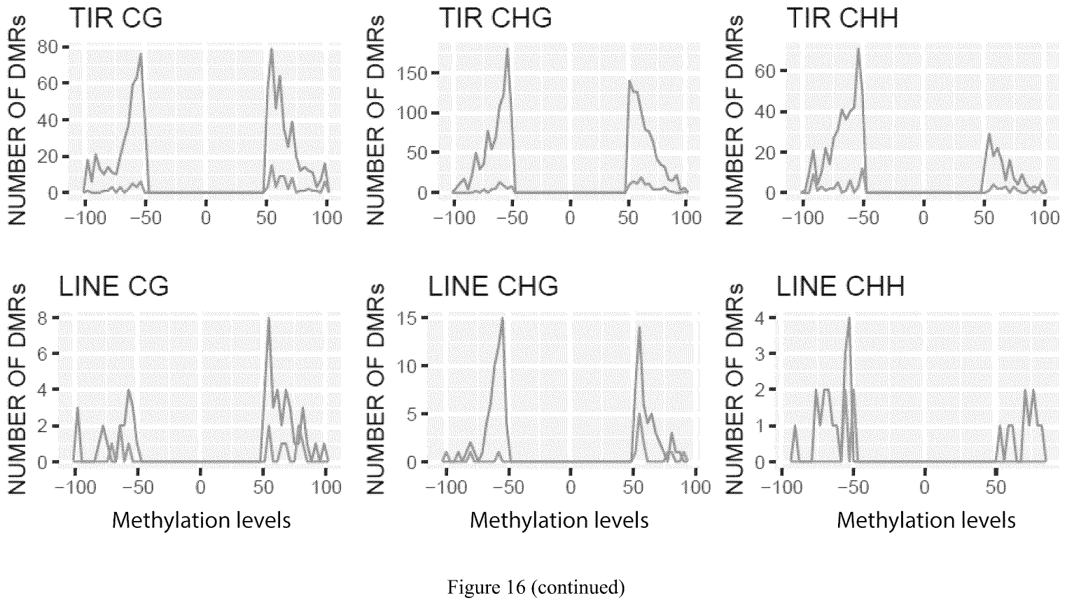

To better understand the methylation differences between TN09-16 and TN09-29 in response to SCN infection, the methylation level, direction, and sequence contexts of the DMRs were examined. The numbers of hyper- and hypo-DMRs associated with protein-coding genes and TEs in the CG, CHG and CHH contexts are shown in FIGS. 5F-5K. With the exception of the CG-DMRs overlapping with TEs (FIG. 5I) the numbers of hypo-DMRs in various sequence contexts in TN09-16 were higher than hyper-DMRs. In TN09-29, however, the opposite trend was observed with hyper-DMRs being more predominant than hypo-DMRs in all cases except CHH-DMRs associated with TEs (FIG. 5K). This trend is also evident when hyper- and hypo-DMRs overlapping with various annotated features of protein-coding genes (FIG. 15) and transposon families (FIG. 16) were compared within each line. Together, these data indicate that DNA methylation reprograming occurs prominently during the susceptible interaction and to a much lesser extent during the resistant interaction.

### Example 6—DNA Methylation Reprograming During the Susceptible Interaction Contributes to Gene Expression Changes

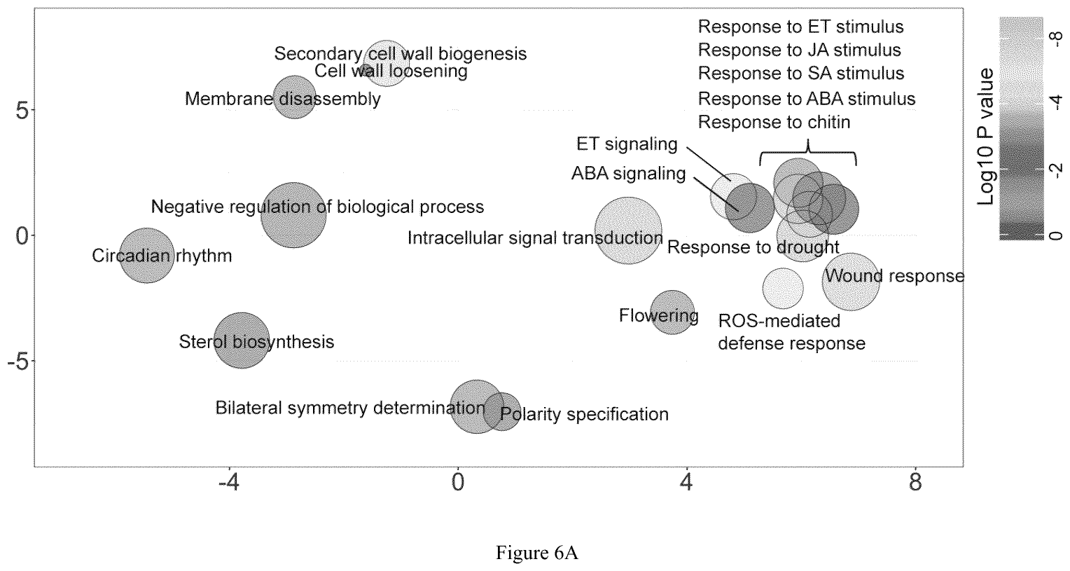

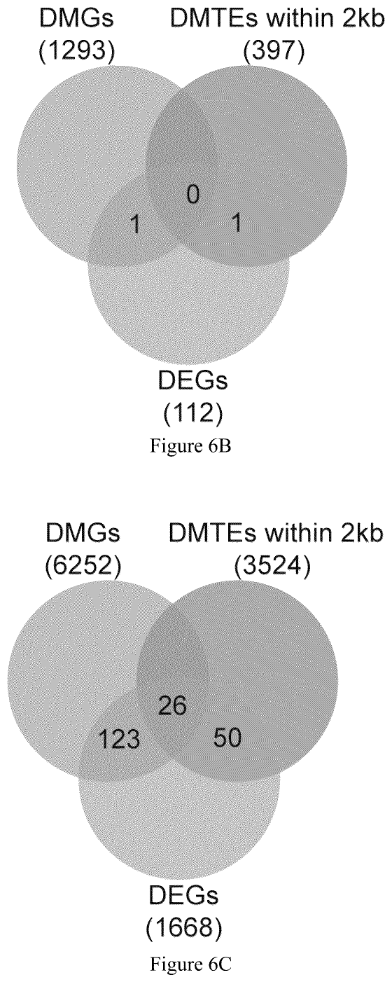

Gene expression changes were further studied in the TN09-16 and TN09-29 in response to SCN using RNA-seq approach. The RNA libraries were prepared from the same tissue samples used for DNA methylation analysis to facilitate examining the potential link between DNA methylation and transcriptome changes. Because of the heterogeneity nature of SCN-infected roots a less stringent P value cutoff of <0.05 and a FDR<0.1 to identify DEGs was used. 1,668 and 112 DEGs in TN09-16 and TN09-29, respectively, were identified at 5 d post SCN infection. The low number of the DEGs identified in the resistant lines may reflect the localized response to SCN infection compared with the susceptible line in which localized and systemic responses may occur both in the developing syncytium as well as in cells far from the infection sites. GO analysis revealed a significant enrichment of three biological process terms associated with plant responses to oxidative stress, chemical stimulus, and oxidation reduction among the TN09-29 DEGs. These results are consistent with the previous reports associating oxidoreductase activity and oxidative stress response with Peking-type resistance, and support the potential function of GmSHMT08 in redox defense. Among the TN09-16 DEGs genes a significant enrichment of biological process terms corresponding to plant response to stimulus and signaling of various phytohormones was noted, including ethylene, salicylic acid, jasmonic acid, and abscisic acid (FIG. 6A). Genes involved in ROS-mediated defense response, secondary cell wall biogenesis, cell wall loosening, membrane disassembly, and cellular responses to wounding and chitin were also overrepresented among the TN09-16 DEGs (FIG. 6A).

Next, the potential association between DNA methylation and gene expression changes was determined. The 112 DEGs and the 1293 DMGs identified in the TN09-29 upon SCN infection were compared and only one gene (Glyma.04G180400) was common between the two gene lists (FIG. 6B). Also, whether differentially methylated TEs are located within 2 kb upstream or downstream of the 112 DEGs was examined. Only one gene (Glyma.06G102000) was found to contain a differentially methylated TE 1.813 kb from its transcriptional start site (TSS) (FIG. 6B). These results imply that differential DNA methylation doesn't seem to directly impact gene transcription during the resistant interaction. Similarly, the 1668 DEGs and the 6252 DMGs identified in the TN09-16 after SCN infection were compared and 123 genes were common between the two gene lists (FIG. 6C), implying that DMGs are statistically significantly enriched among the DEGs (7.37%, x2 =140.3, P=3.27E-30). Furthermore, 50 DEGs were identified containing differentially methylated TEs in their gene body or promoters, 2 kb upstream of the TSS, resulting in a unique list of 147 differentially expressed DMGs (FIG. 6C). Of these 147 genes, 47 have been previously shown to change expression in the SCN-induced syncytium, providing additional support for the involvement of these genes in plant-SCN interaction. Thus, unlike the resistant interaction, DNA methylation reprograming during the susceptible interaction may directly contribute to gene expression changes.

### Example 7—Identification of Stably Inherited DMRs Potentially Associated With SCN Infection

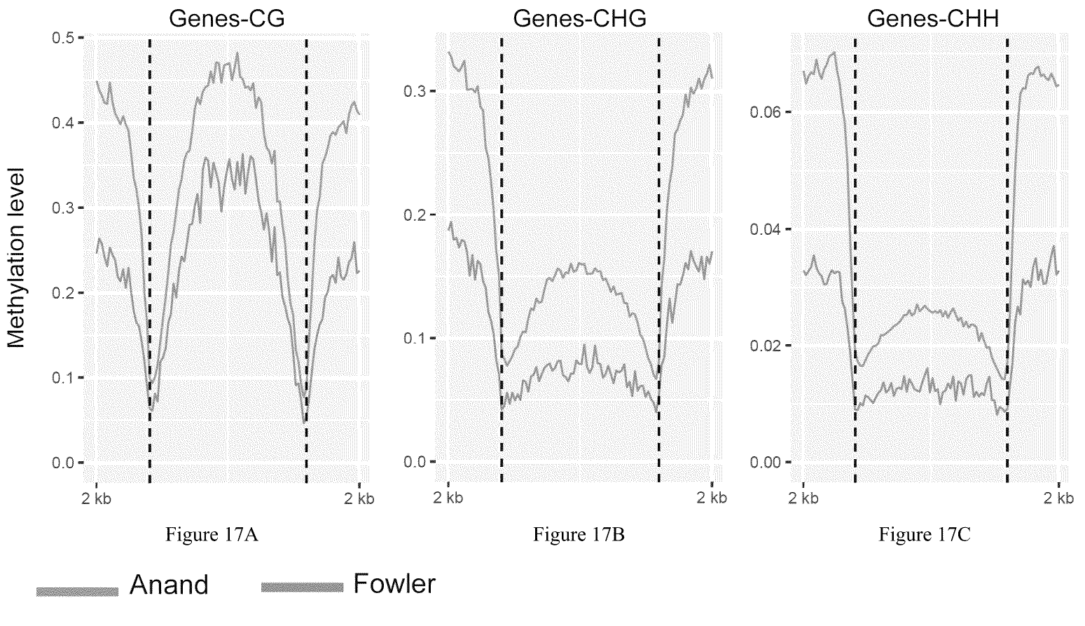

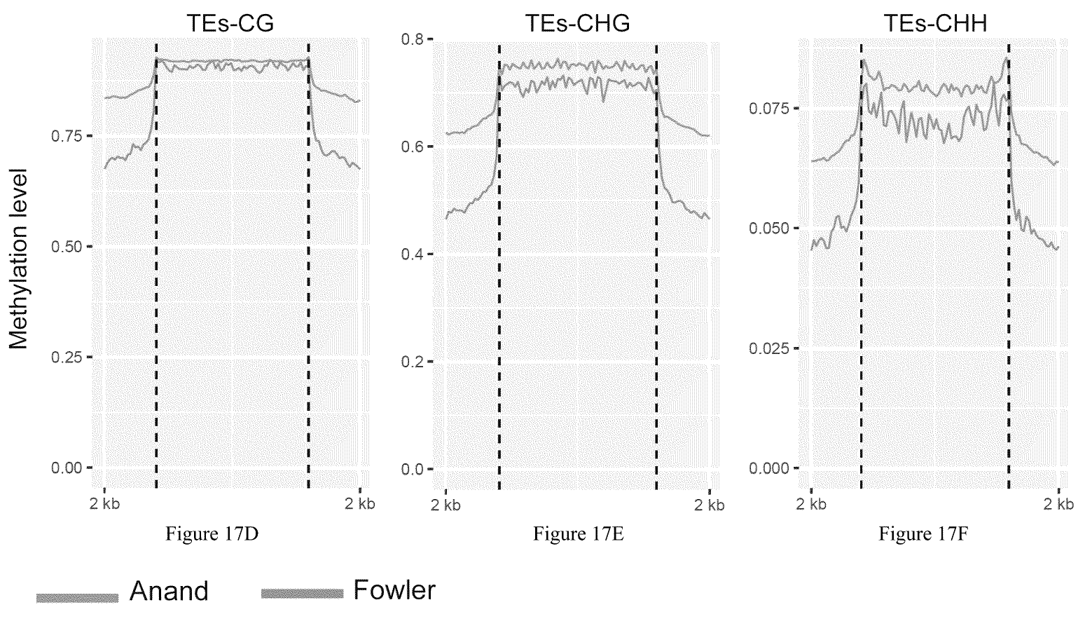

To identify stably inherited DMRs in the genic regions with potential association with SCN resistance/susceptibility differential methylation was searched in the isogenic lines that are inherited from the parents. In other words, DMRs with the exact genomic coordinates that are hypermethylated in the susceptible parent (Anand) and the susceptible line TN09-16 but hypomethylated in the resistant parent (Fowler) and the resistant line TN09-29, and vice versa (hypomethylated in Anand and TN09-16 but hypermethylated in Fowler and TN09-29) were pursued. Therefore, methylC-seq libraries were generated from the two parental lines Fowler and Anand using non-infected root tissues collected from the same experimental settings described above. Differentially methylated cytosines were identified and global methylation levels over genes and TEs in all sequence contexts were compared between the parental lines. Interestingly, the susceptible parent (Anand) showed higher methylation levels than the resistant parent (Fowler) over genes and TEs in all sequence contexts (FIG. 17). A significant number of DMRs between the parental lines were also detected. Of the 45,603 DMRs detected, 7,000 mapped to protein-coding genes and 21,667 mapped to TEs. Consistent with global methylation patterns, the majority (65.70%) of DMRs were hypermethylated in the susceptible parent (Anand) compared with the resistant parent (Fowler). These DNA methylation patterns are consistent with our results mentioned above and showing increased global methylation levels and hyper-DMRs in the susceptible line TN09-16 compared to the resistant line TN09-29.

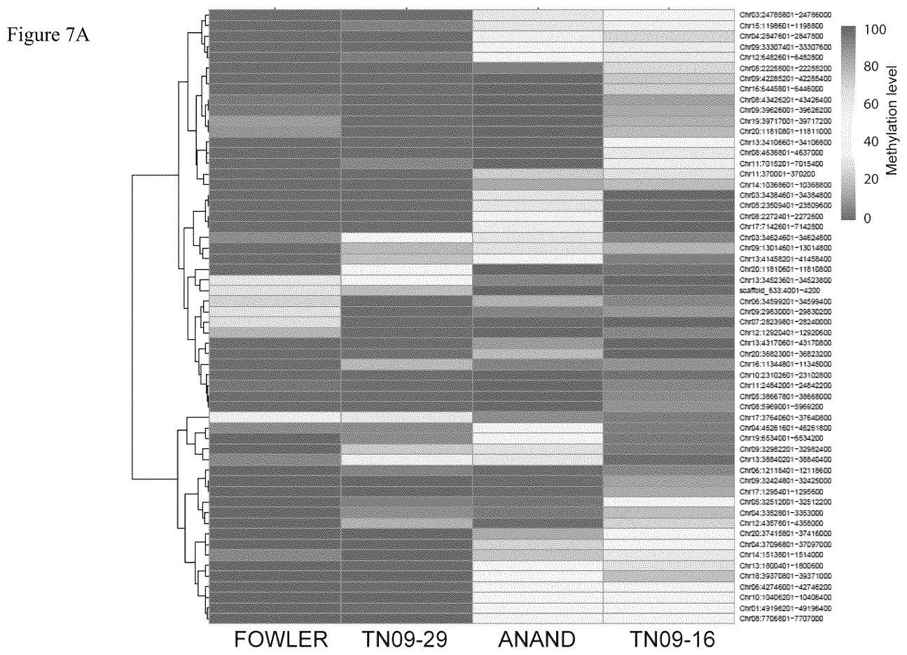

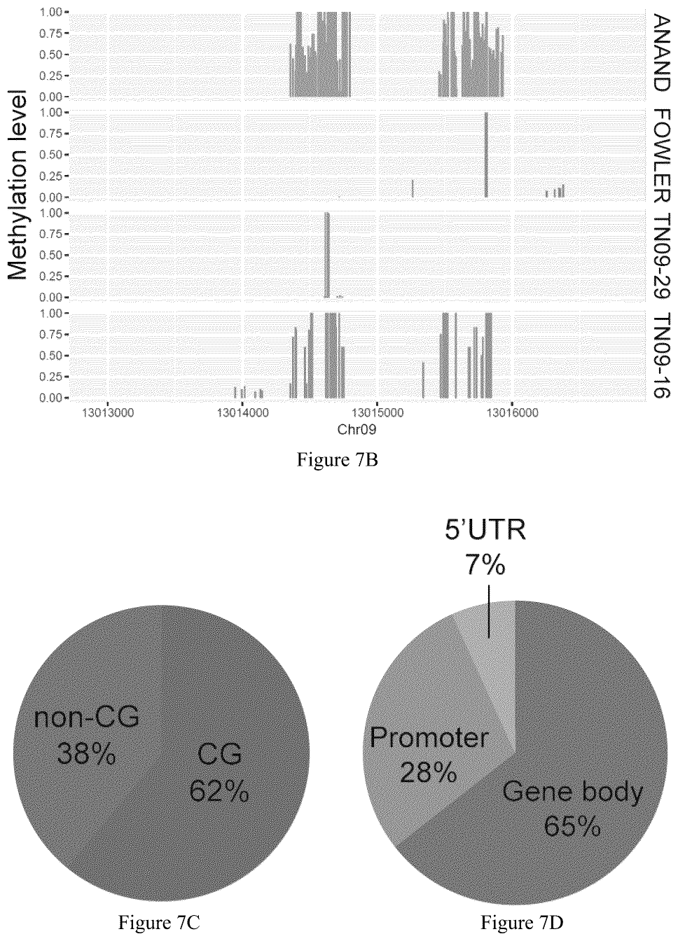

The methylomes of the parental lines and that of the isogenic lines were then compared. 59 DMRs in the isogenic lines were identified with differential methylation patterns that were inherited from the parents (FIG. 7A). As shown in FIG. 7A, the 58 DMRs are grouped into two main clusters. The first cluster contains 38 regions that were hypomethylated in Fowler and TN09-29 but hypermethylated in Anand and TN09-016. An example of these regions is highlighted in FIG. 7B. The second cluster contains 22 regions that were hypermethylated in Fowler and TN09-29 but hypomethylated in Anand and TN09-16 (FIG. 7A). These regions showed differential methylation in the CG (36) and non-CG contexts (22) and are located in the gene body (38), promoter (16) and 5′UTR (4) (FIGS. 7C and 7D). These 58 DMRs overlapped with 57 unique protein-coding genes, four of them were previously reported as differentially expressed in soybean syncytium (Table 2).

TABLE 2
57 protein-coding genes associated with DMRs in isogenic lines

| | | | |
|---|---|---|---|
| Glyma.10G083800 | Glyma.06G251700 | Glyma.11G004900 | Glyma.16G065300 |
| Glyma.01G154500 | Glyma.07G168900 | Glyma.11G092400 | Glyma.16G088900 |
| Glyma.03G085300 | Glyma.08G028400 | Glyma.11G181300 | Glyma.17G017300 |
| Glyma.03G129100 | Glyma.08G060200 | Glyma.12G060400 | Glyma.17g091700 |
| Glyma.03G131000 | Glyma.08G078200 | Glyma.12g081900 | Glyma.17G223100 |
| Glyma.04G036000 | Glyma.08G100300 | Glyma.12G121100 | Glyma.18G168000 |
| Glyma.04g041900 | Glyma.08G314100 | Glyma.13G005900 | Glyma.19G044300 |
| Glyma.04G157200 | Glyma.09G094000 | Glyma.13G228800 | Glyma.19G136000 |
| Glyma.04G191800 | Glyma.09G123300 | Glyma.13G234600 | Glyma.20G052100 |
| Glyma.05G092300 | Glyma.09G129600 | Glyma.13G288000 | Glyma.20g126100 |
| Glyma.05G093400 | Glyma.09G132300 | Glyma.13G320100 | Glyma.20g134300 |
| Glyma.05G132100 | Glyma.09G133900 | Glyma.13G339600 | Glyma.U040800 |
| Glyma.05G203000 | Glyma.09G171300 | Glyma.14g021300 | |
| Glyma.06G148300 | Glyma.09G198400 | Glyma.14G103300 | |
| Glyma.06G228100 | Glyma.10g104900 | Glyma.15G015100 | |

### Example 8—Identification of Novel Non-Parental DMRs Specific to the Isogenic Lines

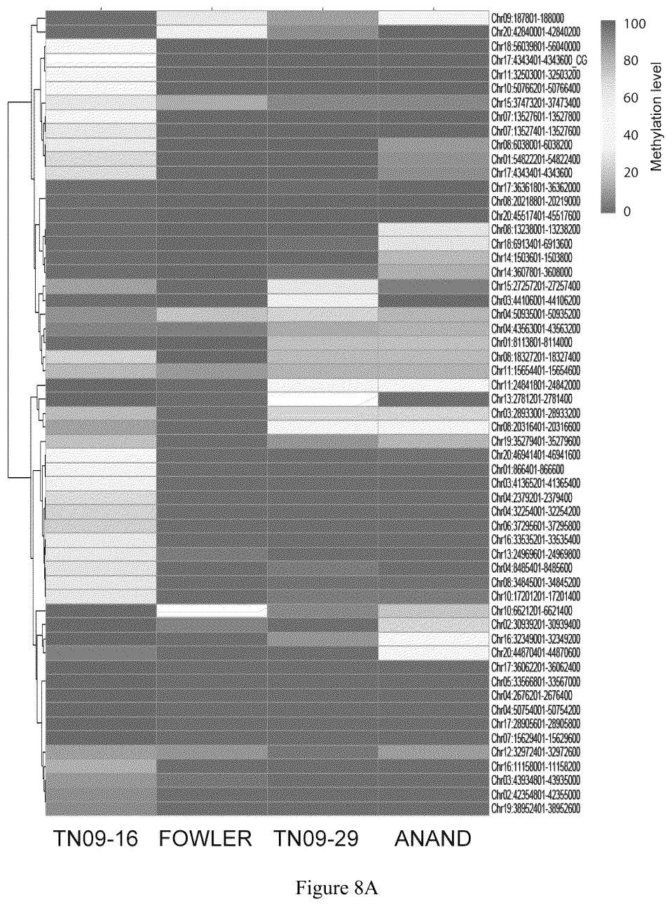

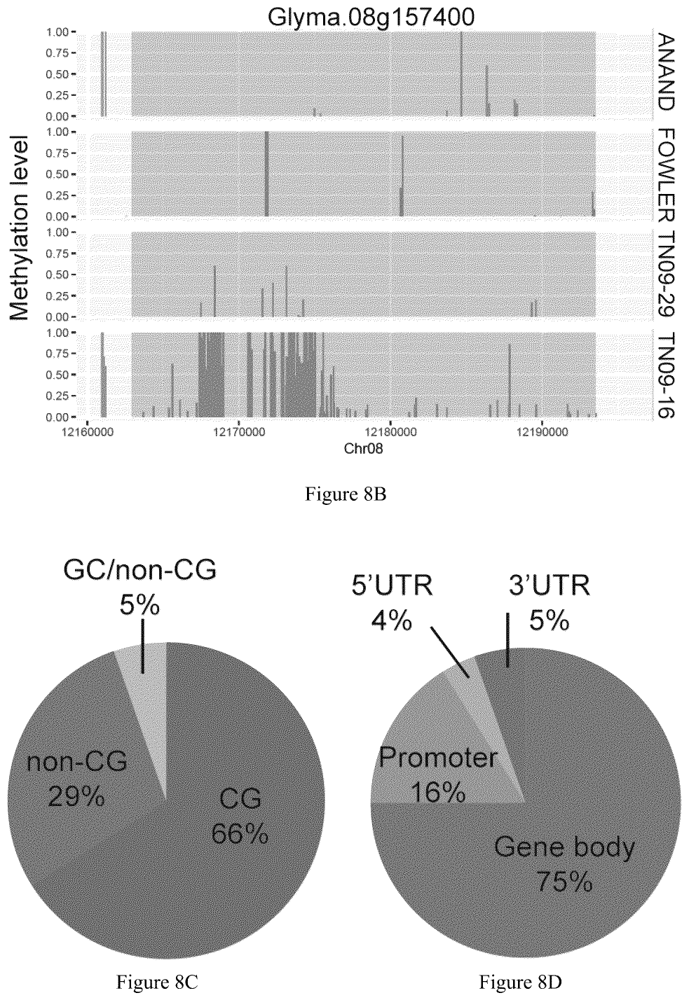

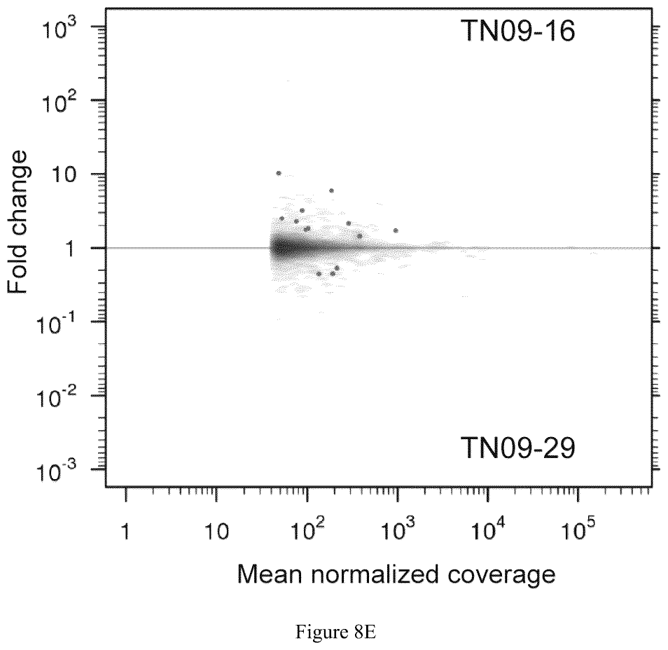

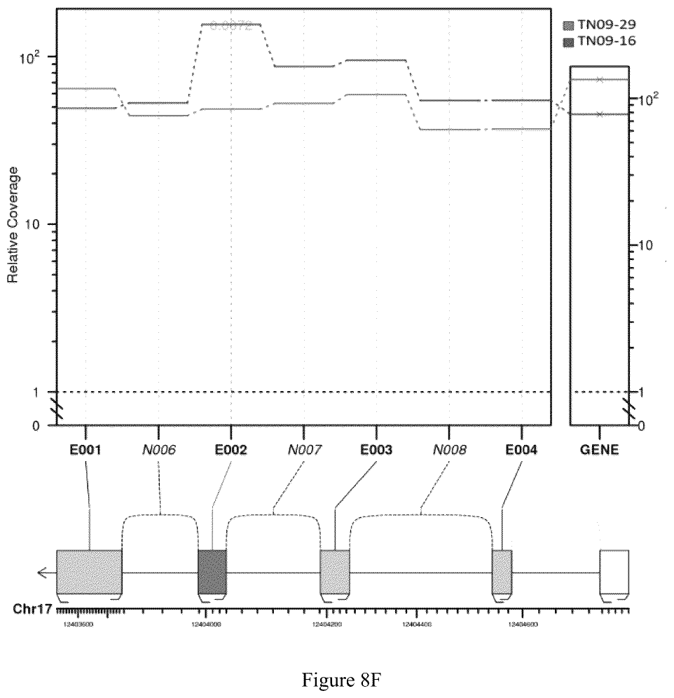

These analyses were extended by comparing the methylomes of the parental lines and the isogenic lines under non-infected conditions to identify novel non-parental DMRs unique to TN09-16 or TN09-29. Interestingly, 56 DMRs unique to TN09-16 were identified. Gain or loss of DNA methylation in these regions occurred to a similar extent and was opposite of that detected in the parental lines and TN09-29 (FIG. 8A). An example of these regions is provided in FIG. 8B. The opposite methylation patterns were detected in CG (37), non-CG (16), and both CG and non-CG contexts (3) (FIG. 8C). The DMRs were located in gene body (42), gene promoters (9), and UTRs (5) (FIG. 8D). The 56 DMRs overlapped with 55 protein-coding genes (Table 3). This gene list included 9 of the previously identified syncytium DEGs, implying a role in the susceptible soybean-SCN interaction. A homolog of Arabidopsis mRNA splicing factor was also identified among this gene list. This finding prompted examination of RNA-seq data for differential usage of exon and exon-exon junctions using JunctionSeq package. Using a FDR cut-off of 0.1, 12 alternatively spliced genes with 13 differentially used exons/junctions were identified when the transcriptomes of TN09-16 and TN09-29 were compared under non-infected conditions (FIG. 8E). Of these exons/junctions 10 were significantly highly used in TN09-16 as in the case of Glyma17G149600 (FIG. 8F), whereas the remaining 3 were significantly highly used in TN09-29. Interestingly, 4 out of these 12 differentially spliced genes are among the syncytium DEGs, suggesting that novel non-parental hypomethylation of a splicing factor-encoding gene in TN09-16 may affect alternative splicing of syncytium DEGs. Because DNA methylation can impact splicing efficiency, these 12 genes were intersected with 3,666 genes showing differential DNA methylation between TN09-16 and TN09-29. None of these genes was common to both lists, indicating that alternative splicing of these genes occurred independently of their methylation patterns.

TABLE 3
55 protein-coding genes associated with DMRs unique to TN09-16

| | | | |
|---|---|---|---|
| Glyma.10G067400 | Glyma.04G238800 | Glyma.11G164700 | Glyma.17G214800 |
| Glyma.01G008900 | Glyma.04G241200 | Glyma.11G181300 | Glyma.17G216000 |
| Glyma.01G060200 | Glyma.05G142200 | Glyma.11G229600 | Glyma.18G073400 |
| Glyma.01G218100 | Glyma.06G233300 | Glyma.12G173100 | Glyma.18G279400 |
| Glyma.02G181200 | Glyma.07G118800 | Glyma.13G010200 | Glyma.19g104100 |
| Glyma.02G236000 | Glyma.07G132300 | Glyma.13G137000 | Glyma.19G130000 |
| Glyma.03G100000 | Glyma.08G079300 | Glyma.14g021300 | Glyma.20g189700 |
| Glyma.03G205600 | Glyma.08G168000 | Glyma.14G047400 | Glyma.20G212100 |
| Glyma.03G240800 | Glyma.08G226000 | Glyma.15G204600 | Glyma.20G219600 |
| Glyma.03G243400 | Glyma.08G237500 | Glyma.15G219000 | Glyma.20G237500 |
| Glyma.04G029400 | Glyma.08G238200 | Glyma.16G088200 | |
| Glyma.04G033600 | Glyma.08G269900 | Glyma.16G164500 | |
| Glyma.04G095300 | Glyma.09G002300 | Glyma.16G174500 | |
| Glyma.04G149700 | Glyma.10G098700 | Glyma.17G057300 | |
| Glyma.04G174100 | Glyma.10G289000 | Glyma.17G193800 | |

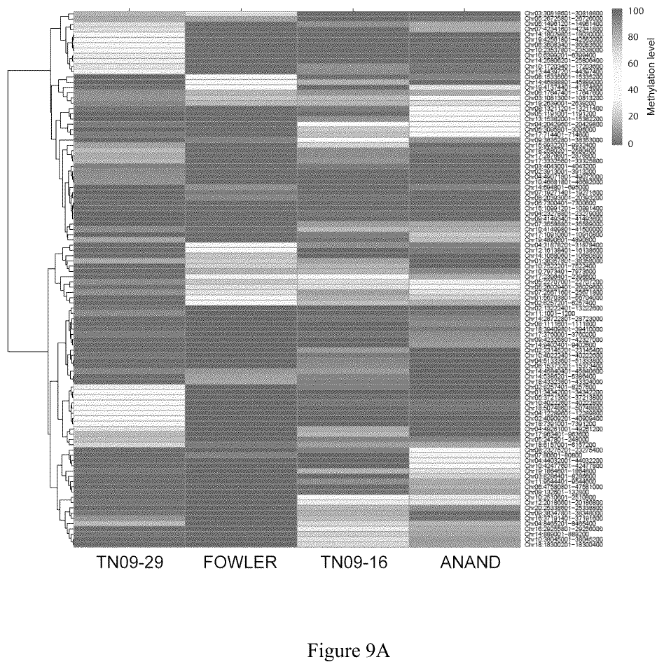

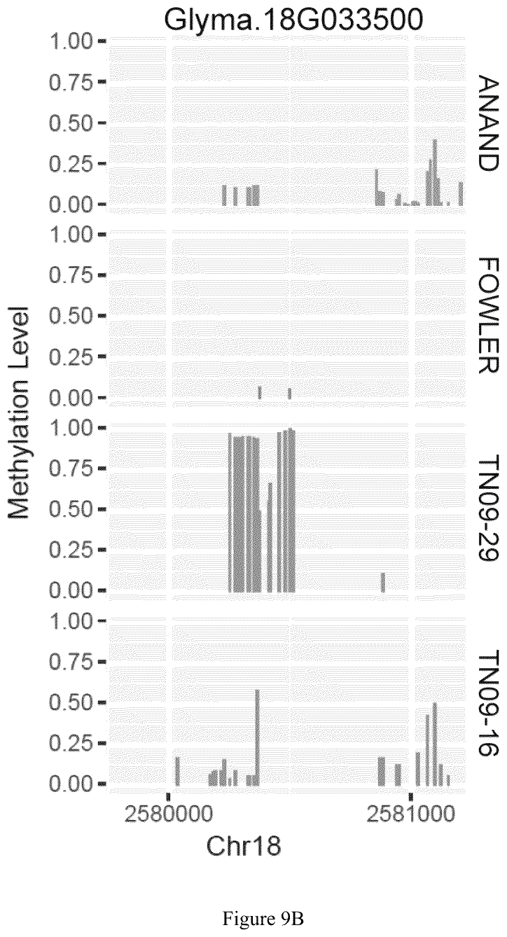

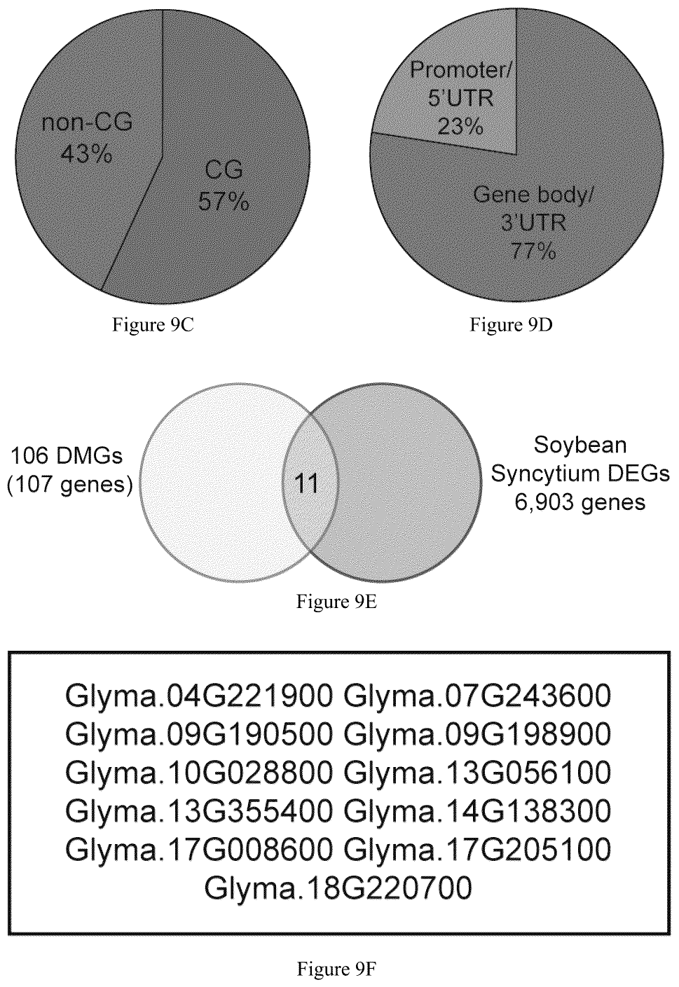

Similarly, the methylomes of the parental lines and the isogenic lines were compared under non-infected conditions to identify novel non-parental DMRs unique to TN09-29. Interestingly, 102 DMRs specific to TN09-29 were identified (FIG. 9A). Hyper- and hypomethylation in these 102 regions occurred to a similar extent and was opposite of that detected in the parental lines and TN09-16 (FIG. 9A). An example of hypermethylated region specific to TN09-29 is provided in FIG. 9B. Differential methylation of these DMRs was found in CG (58) and non-CG (44) (FIG. 9C). The opposite methylation patterns were detected in gene promoter/5′UTR (23) and gene body/3′UTR (79) (FIG. 9D). The 102 DMRs overlapped with 100 protein-coding genes (Table 4), 11 of them previously reported to change expression in SCN-induced syncytium (FIGS. 9E and 9F). Together, these data suggest that novel non-parental DMRs specific to the isogenic lines may impact gene expression in the nematode feeding sites.

TABLE 4
100 protein-coding genes associated with DMRs unique to TN09-29

| | | | |
|---|---|---|---|
| Glyma.10G098700 | Glyma.05G035100 | Glyma.10G028800 | Glyma.14G193900 |
| Glyma.01G102900 | Glyma.05G084500 | Glyma.10G066100 | Glyma.15G125100 |
| Glyma.01G112600 | Glyma.05G092600 | Glyma.10G073100 | Glyma.15G136000 |
| Glyma.01G244700 | Glyma.05G098000 | Glyma.10G076500 | Glyma.16G135200 |
| Glyma.02g041600 | Glyma.05G100700 | Glyma.10g105800 | Glyma.16G214100 |
| Glyma.02g071300 | Glyma.05G185300 | Glyma.10g145700 | Glyma.17G008600 |
| Glyma.02G093000 | Glyma.06G092500 | Glyma.10g168000 | Glyma.17G012400 |
| Glyma.02G129200 | Glyma.06G180700 | Glyma.10g181600 | Glyma.17G032500 |
| Glyma.02G164300 | Glyma.06G196500 | Glyma.10g192400 | Glyma.17G038800 |
| Glyma.02G221300 | Glyma.06G231200 | Glyma.10G238200 | Glyma.17G049500 |
| Glyma.03G034500 | Glyma.06G286900 | Glyma.11g000100 | Glyma.17G135500 |
| Glyma.03G058300 | Glyma.07G000600 | Glyma.11G125600 | Glyma.17g205100 |
| Glyma.03G064800 | Glyma.07G156900 | Glyma.12G136700 | Glyma.18G033500 |
| Glyma.03G107700 | Glyma.07G166000 | Glyma.12G146400 | Glyma.18G066700 |
| Glyma.04G094900 | Glyma.07G188300 | Glyma.13G056100 | Glyma.18G077700 |
| Glyma.04G111200 | Glyma.07G243600 | Glyma.13G355400 | Glyma.18G131100 |
| Glyma.04G136700 | Glyma.08G014300 | Glyma.14g009100 | Glyma.18G168500 |
| Glyma.04G140800 | Glyma.08G167600 | Glyma.14g011600 | Glyma.18G179600 |
| Glyma.04G149400 | Glyma.08G190600 | Glyma.14G065500 | Glyma.18G220700 |
| Glyma.04G177000 | Glyma.08G238800 | Glyma.14G098600 | Glyma.19G018100 |
| Glyma.04G220100 | Glyma.08G258600 | Glyma.14G104300 | Glyma.19G023000 |
| Glyma.04G221900 | Glyma.09g001700 | Glyma.14G122700 | Glyma.19G036100 |
| Glyma.04G245700 | Glyma.09G159500 | Glyma.14G138300 | Glyma.19G153500 |
| Glyma.05G003500 | Glyma.09G190500 | Glyma.14G142500 | Glyma.19G164800 |
| Glyma.05G012700 | Glyma.09G198900 | Glyma.14G193600 | Glyma.20g071200 |

### Example 9—Genes With Inherited or Introduced Differential Methylation are of Biological Significance

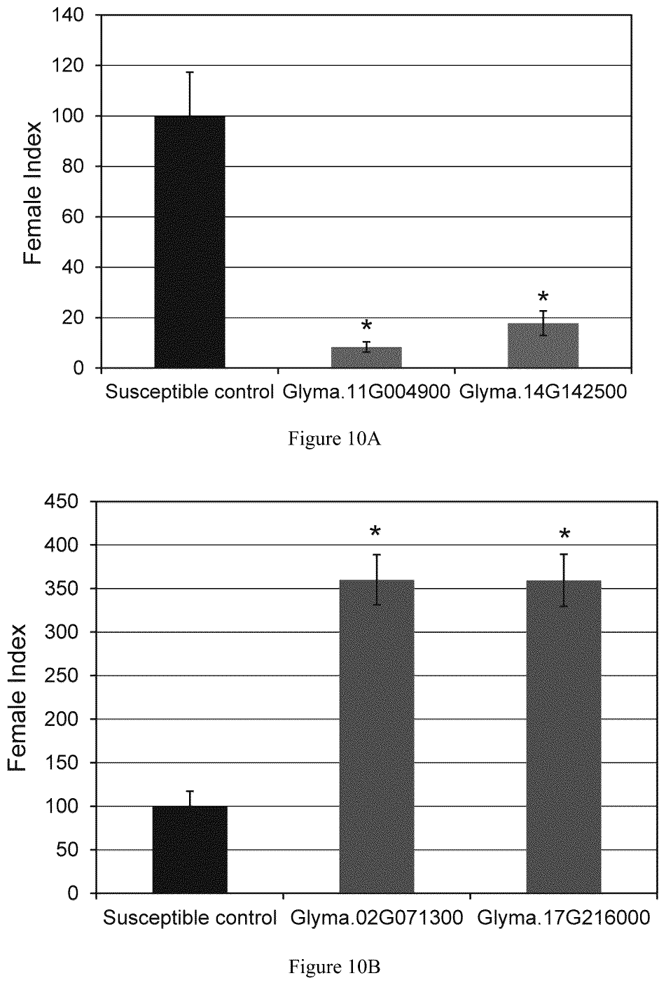

It was examined whether the genes with inherited or introduced differential methylation are of biological significance and impact plant response to SCN. Four genes coding for a sulfite exporter TauE/SafE family protein (Glyma.14G142500), a SNARE associated Golgi protein family (Glyma.11G004900), an aspartate aminotransferase 5 (Glyma.17G216000), and a transcription regulator of the NOT2/NOT3/NOT5 family protein (Glyma.02G071300) were overexpressed in the susceptible isogenic line TN09-016 using soybean transgenic hairy root system. The composite transgenic plants were assayed for SCN (race 3) response. Interestingly, overexpression of SNARE associated Golgi protein family was able to complement the RHg4 susceptible allele coffering very high level of resistance with female index of 8% compared with the control (FIG. 10A). Overexpression of the aspartate aminotransferase also produced very high level of resistance resulting in a female index of less than 20% (FIG. 10A). Equally important, overexpression of the aspartate aminotransferase 5 and the NOT2/NOT3/NOT5 family transcription regulator dramatically increased plant susceptibility to SCN resulting in a female index of more than 350% (FIG. 10B). These results indicate that genes associated with stably inherited or introduced DNA methylation are bona fide candidate genes for enhancing plant resistance to SCN.

### Example 10—Discovery of SCN Resistance Genes Based on Epigenetic Analysis

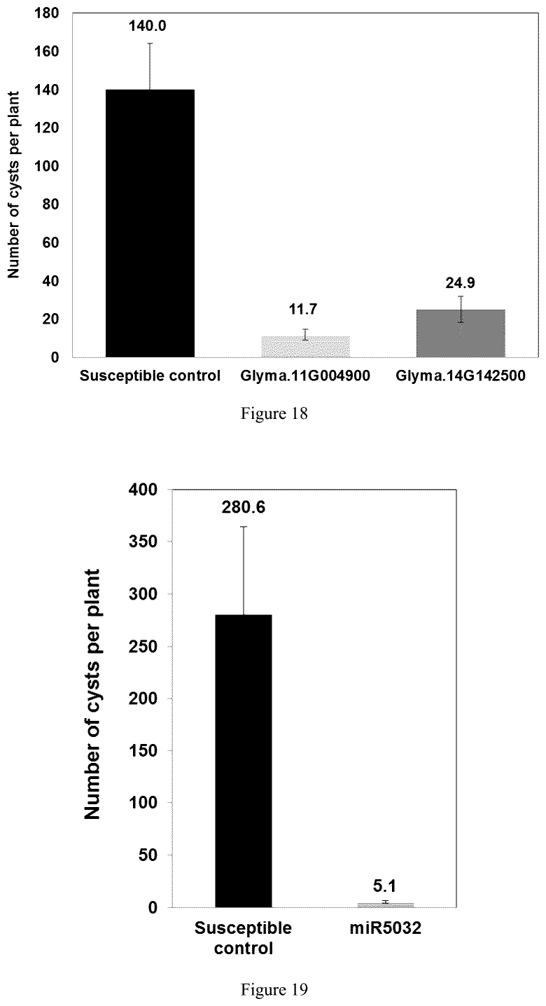

A novel epigenetic analysis-based approach was used to identify a SNARE associated Golgi protein family (Glyma.11G004900) and a sulfite exporter TauE/SafE family protein (Glyma.14G142500) as SCN resistance genes. The functional roles of these genes in SCN resistance were confirmed using transgenic soybean hairy root system. These genes were overexpressed in the susceptible isogenic line TN09-016 using transgenic hairy root system. The composite plants were assayed for SCN (race 3) response. Overexpression of the coding sequences of SNARE associated Golgi protein family was able to complement the Rhg4 susceptible allele coffering very high level of resistance with an average of cyst of 11.7 compared with 140.0 found in the susceptible control (FIG. 18). Similarly, overexpression of the coding sequences of the sulfite exporter TauE/SafE family protein produced very high level of resistance of about 80% (FIG. 18).

### Example 11—Discovery of SCN Resistance miRNA Gene Based on Epigenetic Analysis

A novel epigenetic analysis—based approach was used to identify miRNA5032 as an SCN resistance gene. The functional roles of miRNA5032 in SCN resistance were confirmed using transgenic soybean hairy root system. miRNA5032 was overexpressed in the susceptible isogenic line TN09-016 using transgenic hairy root system. The composite plants were assayed for SCN (race 3) response. Overexpression of the primary transcript of the gma-miR5032 produced extreme resistance against SCN race 3 compared with the susceptible control. An average of 5.1 cysts per plant was counted on the plants containing overexpression of miRNA5032 compared to 280.6 cysts per plant recorded in the susceptible control (FIG. 19).

Thus, certain embodiments of the disclosure provide the gene encoding miR5032 provides SCN resistance to a plant cell or a plant, particularly, a soybean plant cell or a soybean plant. For example, overexpressing in a plant cell or a plant, particularly, a soybean plant cell or a soybean plant, a gene encoding an miRNA having the sequence of SEQ ID NO: 210 or homologs thereof renders the plant cell or the plant, particularly, the soybean plant cell or the soybean plant, resistance to SCN. An example of a gene encoding an miRNA having the sequence of SEQ ID NO: 210 is provided by the gene comprising the sequence of SEQ ID NO: 213.

### Example 12—Identification of Genetic Polymorphisms in the Three Major SCN Resistance Genes Associated With Soybean Resistance Against Five SCN Races

Ninety nine completely sequenced soybean accessions were phenotyped against five races of SCN (race 1, 2, 3, 5, and 14). The DNA sequences of the promoter regions of these three SCN resistance genes in addition to the coding sequences of the Glyma.11G004900 and Glyma.14G142500 were retrieved from these 99 soybean accessions and analyzed for potential genetic differences and polymorphisms linked with resistance against five SCN races. Genetic polymorphisms in the promoter and/or coding sequences of these three genes were identified. Statistical analysis revealed significant association between the identified genetic polymorphisms and soybean resistance against one or more races.

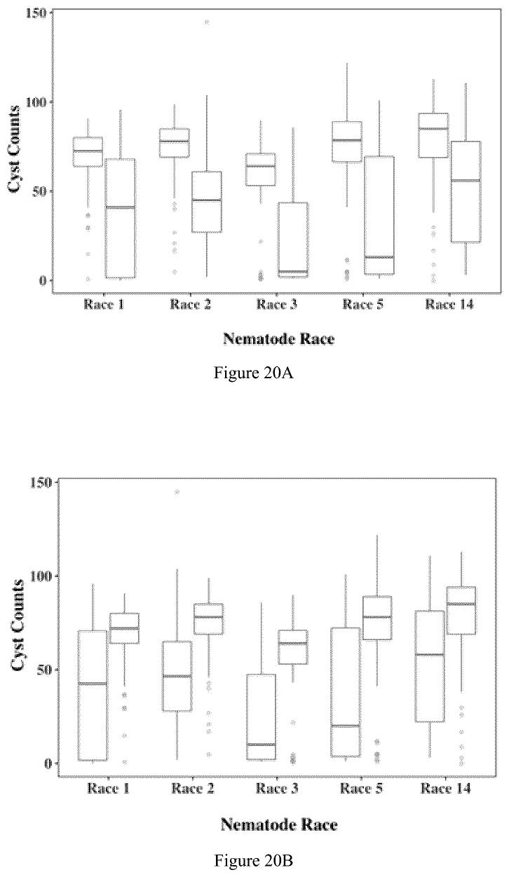

For Glyma.11G004900, fourteen single nucleotide polymorphism (SNPs) were identified to be associated with SCN resistance; one in the gene body (K/P), and 13 in the promoter region (Table 5). All of these fourteen SNPs were found to be statistically significantly associated with resistance against all the five SCN races (race 1, 2, 3, 5, and 14) (Table 5). As shown in FIGS. 20A and 20B, the C/T and CAACAT/C changes in the promoter of this gene at positions 1020 and 1718/23 upstream of the translation start codon ATG were associated with resistance against all the five SCN races tested.

TABLE 5
Genomic locations and P values of the genetic polymorphisms identified in
the SCN resistance gene Glyma.11G004900.

| Genomic | | | | | | |
|---|---|---|---|---|---|---|
| location | Polymorphism | P value | | | | |
| relative to | Susceptibility/ | SCN | SCN | SCN | SCN | SCN |
| ATG | Resistance | Race 1 | Race 2 | Race 3 | Race 5 | Race 14 |
| Gene | K/R | 0.00012 | 2.911E−07 | 3.552E−06 | 2.662E−06 | 0.00000 |
| body, 355 | | | | | | |
| Promoter, | G/T | 0.00022 | 3.069E−06 | 2.368E−06 | 5.232E−06 | 0.00002 |
| −209 | | | | | | |
| Promoter, | A/T | 0.00022 | 0.00039 | 0.000018 | 0.00007 | 0.00024 |
| −384 | | | | | | |
| Promoter, | A/G | 0.00022 | 0.00001 | 2.499E−06 | 0.00002 | 0.00003 |
| −460 | | | | | | |
| Promoter, | T/A | 0.00022 | 0.00165 | 0.00003 | 0.00171 | 0.00059 |
| −823 | | | | | | |
| Promoter, | TA/T | 0.00022 | 0.00000 | 0.00000 | 0.00002 | 0.00004 |
| −828/29 | | | | | | |
| Promoter, | C/A | 0.00043 | 0.00150 | 0.00001 | 0.00024 | 0.00035 |
| −866 | | | | | | |
| Promoter, | G/GTT | 0.00061 | 0.00022 | 0.00002 | 0.00030 | 0.00023 |
| −1011 | | | | | | |
| Promoter, | C/T | 0.00006 | 0.00036 | 1.339E−06 | 0.00005 | 0.00020 |
| −1020 | | | | | | |
| Promoter, | C/A | 0.00044 | 0.00061 | 4.592E−06 | 0.00012 | 0.00038 |
| −1155 | | | | | | |
| Promoter, | T/C | 0.00031 | 0.00033 | 2.303E−06 | 0.00006 | 0.00022 |
| −1434 | | | | | | |
| Promoter, | CAACAT/C | 0.00030 | 0.00039 | 2.757E−06 | 0.00008 | 0.00035 |
| −1718/23 | | | | | | |
| Promoter, | T/C | 0.00203 | 0.00021 | 0.000014 | 0.00081 | 0.00029 |
| −1910 | | | | | | |
| Promoter, | C/T | 0.00031 | 0.00033 | 2.303E−06 | 0.00006 | 0.00022 |
| −1969 | | | | | | |

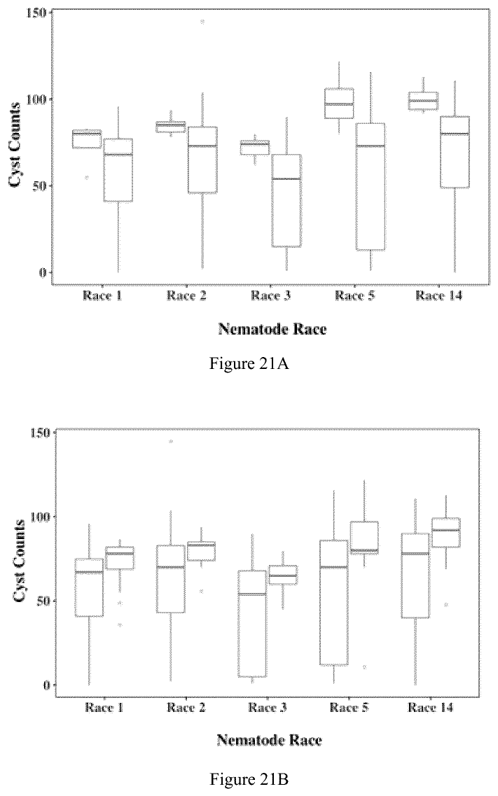

Similarly, for Glyma.14G142500, ten SNPs were identified in the promoter region (Table 6). Six of these ten SNPs were found to be statistically significantly associated with resistance against SCN race 3, 5, and 14 (Table 6). These SNPs are located at positions 166, 383, 387, 482, 1101, and 1103 upstream of the ATG. FIGS. 21A and 21B show that A/AT and G/A polymorphisms in the promoter of this gene at positions 482 and 1101, respectively were associated with resistance against SCN race 3, 5, and 14.

TABLE 6
Genomic locations and P values of the genetic polymorphisms identified in
the SCN resistance gene Glyma.14G142500.

| Genomic | | | | | | |
|---|---|---|---|---|---|---|
| location | Polymorphism | P value | | | | |
| relative to | Susceptibility/ | SCN | SCN | SCN | SCN | SCN |
| ATG | Resistance | Race 1 | Race 2 | Race 3 | Race 5 | Race 14 |
| Promoter, −1587 | T/G | 0.14590 | 0.13690 | 0.64240 | 0.66710 | 0.29470 |
| Promoter, −1578 | G/A | 0.26320 | 0.17040 | 0.08625 | 0.10850 | 0.06291 |
| Promoter, −1103 | C/T | 0.16770 | 0.12800 | 0.01725* | 0.02008* | 0.01309* |
| Promoter, −1101 | G/A | 0.16770 | 0.12800 | 0.01725* | 0.02008* | 0.01309* |
| Promoter, −1092 | A/G | 0.14590 | 0.13690 | 0.64240 | 0.66710 | 0.29470 |
| Promoter, −556 | G/GT | 0.10790 | 0.27220 | 0.64240 | 0.66710 | 0.29470 |
| Promoter, −482 | A/AT | 0.39120 | 0.14810 | 0.01642* | 0.00493* | 0.00204* |
| Promoter, −387 | A/AT | 0.39120 | 0.14810 | 0.01642* | 0.00493* | 0.00204* |
| Promoter, −383 | A/T | 0.39120 | 0.14810 | 0.01642* | 0.00493* | 0.00204* |
| Promoter, −166 | C/A | 0.39120 | 0.14810 | 0.01642* | 0.00493* | 0.00204* |

*indicates statistically significant increase in SCN resistance.

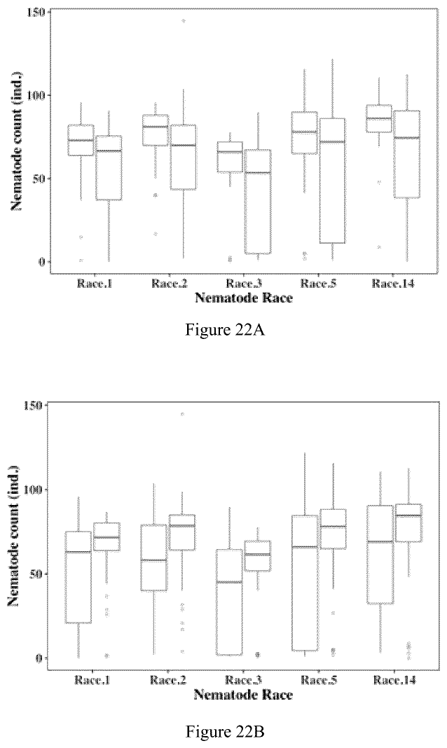

For gma-miR5032, seven SNPs were identified in the promoter region (Table 7). Only three SNPs were statistically linked to SCN resistance. The C/T and A/C changes in the promoter of this microRNA gene at positions 905 and 1259 upstream of the transcription start site (TSS) were associated with resistance against race 2 (FIG. 22A). Importantly, the T/G change in the promoter of this gene at position 2051 upstream of the transcription start site was associated with resistance against SCN races 1, 2, 3 and 5 (FIG. 22B).

TABLE 7
Genomic locations and P values of the genetic polymorphisms identified in
the gma-miR5032.

| Genomic | Polymorphism | P value | | | | |
|---|---|---|---|---|---|---|
| location relative | Susceptibility/ | SCN | SCN | SCN | SCN | SCN |
| to TSS | Resistance | Race 1 | Race 2 | Race 3 | Race 5 | Race 14 |
| Promoter, −127 | C/G | 0.55600 | 0.54630 | 0.15510 | 0.97860 | 0.24670 |
| Promoter, −309 | Insert/A | 0.27480 | 0.18970 | 0.11880 | 0.40080 | 0.93000 |
| Promoter, −905 | C/T | 0.08572 | 0.02218 | 0.12120 | 0.13340 | 0.06569 |
| Promoter, −1259 | A/C | 0.08062 | 0.02061 | 0.17240 | 0.08697 | 0.06886 |
| Promoter, −1503 | G/A | 0.13470 | 0.05441 | 0.48540 | 0.34600 | 0.27790 |
| Promoter, −1526/27 | CA/C | 0.44120 | 0.13120 | 0.38180 | 0.52580 | 0.48150 |
| Promoter, −2051 | T/G | 0.03660 | 0.00455 | 0.01562 | 0.01090 | 0.08924 |

TABLE 8
The list of polymorphisms from Tables 5-7
that are associated with
susceptibility/resistance against an SCN infection.

| | |
|---|---|
| | Polymorphism |
| Genomic location relative to | Susceptibility/ |
| start site for transcription | Resistance |

Glyma. 11G004900 (SEQ ID NO: 211)

| | |
|---|---|
| Gene body, 355 | K/R |
| Promoter, −209 | G/T |
| Promoter, −384 | A/T |
| Promoter, −460 | A/G |
| Promoter, -823 | T/A |
| Promoter, −828/29 | TA/T |
| Promoter, 866 | C/A |
| Promoter, −1011 | G/GTT |
| Promoter, −1020 | C/T |
| Promoter, −1155 | C/A |
| Promoter, −1434 | T/C |
| Promoter, −1718/23 | CAACAT/C |
| Promoter, −1910 | T/C |
| Promoter, −1969 | C/T |

Glyma. 14G142500 (SEQ ID NO: 212)

| | |
|---|---|
| Promoter, −1103 | C/T |
| Promoter, −1101 | G/A |
| Promoter, −482 | A/AT |
| Promoter, −387 | A/AT |
| Promoter, −383 | A/T |
| Promoter, −166 | C/A |

gma-miR5032 (SEQ ID NO: 211)

| | |
|---|---|
| Promoter, −905 | C/T |
| Promoter, −1259 | A/C |
| Promoter, −2051 | T/G |

### REFERENCES

1. Akalin A, Kormaksson M, Li S, Garrett-Bakelman F E, Figueroa M E, Melnick A, Mason CE (2012) methylKit: a comprehensive R package for the analysis of genome-wide DNA methylation profiles. Genome Biol 13: R87.
2. Anand S C, Newman T, Fisher J. 2001. Registration of ‘Anand’ soybean. Crop Sci 41:919-920.
3. Anders S, Pyl P T, Huber W (2015) HTSeq—a Python framework to work with high-throughput sequencing data. Bioinformatics 31: 166-169.
4. Becker, C., Hagmann, J., Müller, J., Koenig, D., Stegle, O., Borgwardt, K. and Weigel, D., 2011. Spontaneous epigenetic variation in the Arabidopsis thaliana methylome. Nature, 480(7376), p.245.
5. Bernatavichute Y V, Zhang X, Cokus S, Pellegrini M, Jacobsen S E (2008) Genome-wide association of histone H3 lysine nine methylation with CHG DNA methylation in Arabidopsis thaliana. PLoS One 3: e3156.
6. Boehm, M. and Bonifacino, J. S., 2001. Adaptins the final recount. Molecular biology of the cell, 12(10), pp. 2907-2920.
7. Bolger A M, Lohse M, Usadel B (2014) Trimmomatic: a flexible trimmer for Illumina sequence data. Bioinformatics 30: 2114-2120.
8. Brucker E, Carlson S, Wright E, Niblack T, Diers B (2005) Rhg1 alleles from soybean PI 437654 and PI 88788 respond differentially to isolates of Heterodera glycines in the greenhouse. Theor Appl Genet 111: 44-49.
9. Concibido V C, Diers B W, Arelli P R. 2004. A decade of QTL mapping for cyst nematode resistance in soybean. Crop Sci 44:1121-1131.
10. Cook D E, Bayless A M, Wang K, Guo X L, Song Q J, Jiang J M, et al. 2014. Distinct copy number, coding sequence, and locus methylation patterns underlie Rhg1-mediated soybean resistance to soybean cyst nematode. Plant Physiol 165:630-647.
11. Cook, D. E., Lee, T. G., Guo, X., Melito, S., Wang, K., Bayless, A., Wang, J., Hughes, T. J., Willis, D. K., Clemente, T. E., Diers, B. W., Jiang, J., Hudson, M. E., and Bent, A. F. 2012. Copy number variation of multiple genes at Rhg1 mediates nematode resistance in soybean. Science 338:1206-1209.
12. Daykin M E., Hussey R S. 1985. Staining and histopathological techniques in nematology. In K R Barker, C C Carter, J N Sasser, eds, An Advanced treatise on Meloidogyne, Vol 2. North Carolina State University Graphics, Raleigh, NC, 39-48.
13. Du J, et al. (2012) Dual binding of chromomethylase domains to H3K9me2-containing nucleosomes directs DNA methylation in plants. Cell; 151:167-180.
14. Du Z, Zhou X, Ling Y, Zhang Z, Su Z (2010) agriGO: a GO analysis toolkit for the agricultural community. Nucleic Acids Res 38: W64-W70.
15. Du, J., Johnson, L. M., Jacobsen, S. E. and Patel, D. J., 2015. DNA methylation pathways and their crosstalk with histone methylation. Nature reviews. Molecular cell biology, 16(9), pp. 519-532.
16. Eichten, S. R., Briskine, R., Song, J., Li, Q., Swanson-Wagner, R., Hermanson, P. J., Waters, A. J., Starr, E., West, P. T., Tiffin, P. and Myers, C. L., 2013. Epigenetic and genetic influences on DNA methylation variation in maize populations. The Plant Cell, 25:2783-2797.
17. Gillen A M, Shelton G W (ed.) (2012) Uniform soybean tests, southern states, 2011. USDA-ARS, Stoneville, MS. Available at ars.usda.gov/ARSUserFiles/60661000/UniformSoybeanTests/2011SoyBook.pdf.

18. Hartley, S. W. and Mullikin, J. C., 2015. QoRTs: a comprehensive toolset for quality control and data processing of RNA-Seq experiments. BMC bioinformatics, 16:224.
19. Hartley, S. W. and Mullikin, J. C., 2016. Detection and visualization of differential splicing in RNA-Seq data with JunctionSeq. Nucleic acids research, 44(15), pp. e127-e127.
20. He, X. J., Chen, T. and Zhu, J. K., 2011. Regulation and function of DNA methylation in plants and animals. Cell research, 21(3), p. 442.
21. Hewezi T, Lane T, Piya S, Rambani A, Rice J H, Staton M. (2017) Cyst nematode parasitism induces dynamic changes in the root epigenome. Plant Physiology. 174:405-420.
22. Hewezi, T., Howe, P., Maier, T. R., Hussey, R. S., Mitchum, M. G., Davis, E. L., Baum, T. J. (2008). Cellulose binding protein from the parasitic nematode Heterodera schachtii interacts with Arabidopsis pectin methylesterase: cooperative cell wall modification during parasitism. Plant Cell 20: 3080-3093.
23. Hewezi, T., Howe, P. J., Maier, T. R., Hussey, R. S., Mitchum, M. G., Davis, E. L., Baum, T. J. (2010). Arabidopsis spermidine synthase is targeted by an effector protein of the cyst nematode Heterodera schachtii. Plant Physiol. 152: 968-984.
24. Hofmann J, El Ashry AelN, Anwar S, Erban A, Kopka J, Grundler F (2010) Metabolic profiling reveals local and systemic responses of host plants to nematode parasitism. Plant J 62: 1058-1071.
25. Hollister, J. D. & Gaut, B. S. Epigenetic silencing of transposable elements: a trade-off between reduced transposition and deleterious effects on neighboring gene expression. Genome Res. 19, 1419-1428 (2009).
26. Holoch, D. and Moazed, D., 2015. RNA-mediated epigenetic regulation of gene expression. Nature Reviews Genetics, 16(2), pp. 71-84.
27. Inagaki S, Miura-Kamio A, Nakamura Y, Lu F, Cui X, Cao X, Kimura H, Saze H, Kakutani T. (2010) Autocatalytic differentiation of epigenetic modifications within the Arabidopsis genome. EMBO J. 29:3496-506.
28. Inagaki, S., Miura-Kamio, A., Nakamura, Y., Lu, F., Cui, X., Cao, X., Kimura, H., Saze, H. and Kakutani, T., 2010. Autocatalytic differentiation of epigenetic modifications within the Arabidopsis genome. The EMBO Journal, 29:3496-3506.
29. Johannes F, et al. (2009) Assessing the impact of transgenerational epigenetic variation on complex traits. PLoS Genet 5(6):e1000530.
30. Kakutani T, Munakata K, Richards E J, Hirochika H (1999) Meiotically and mitotically stable inheritance of DNA hypomethylation induced by ddm1 mutation of Arabidopsis thaliana. Genetics 151(2):831-838.

31. Kazi S, Shultz J, Afzal J, Hashmi R, Jasim M, Bond J, et al. 2010. Iso-lines and inbred-lines confirmed loci that underlie resistance from cultivar ‘Hartwig’ to three soybean cyst nematode populations. Theor Appl Genet 120:633-644.
32. Kim, Y. H., Kim, K. S., and Riggs, R. D. 2010. Differential subcellular responses in resistance soybeans infected with soybean cyst nematode races. Plant Pathol. J. 26:154-158.
33. Klink V P, Hosseini P, Matsye P, Alkharouf N W, Matthews B F (2009) A gene expression analysis of syncytia laser microdissected from the roots of the Glycine max (soybean) genotype PI 548402 (Peking) undergoing a resistant reaction after infection by Heterodera glycines (soybean cyst nematode). Plant Mol Biol 71: 525-567.
34. Klink, V. P., Hosseini, P., Matsye, P. D., Alkharouf, N. W., and Matthews, B. F. 2011. Differences in gene expression amplitude overlie a conserved transcriptomic program occurring between the rapid and potent localized resistant reaction at the syncytium of the Glycine max genotype Peking (PI 548402) as compared to the prolonged and potent resistant reaction of PI 88788. Plant Mol. Biol. 75:141-165.
35. Klink, V. P., Overall, C. C., Alkharouf, N. W., MacDonald, M. H., and Matthews, B. F. 2007. A time-course comparative microarray analysis of an incompatible and compatible response by Glycine max (soybean) to Heterodera glycines (soybean cyst nematode) infection. Planta 226:1423-1447.
36. Krueger F, Andrews S R (2011) Bismark: a flexible aligner and methylation caller for Bisulfite-Seq applications. Bioinformatics 27: 1571-1572.
37. Lawrence M, Gentleman R, Carey V (2009) rtracklayer: an R package for interfacing with genome browsers. Bioinformatics 25: 1841-1842.
38. Li X, et al. (2012) Single-base resolution maps of cultivated and wild rice methylomes and regulatory roles of DNA methylation in plant gene expression. BMC Genomics 13:300.
39. Lister R, O'Malley R C, Tonti-Filippini J, Gregory B D, Berry C C, Millar A H, Ecker J R (2008) Highly integrated single-base resolution maps of the epigenome in Arabidopsis. Cell 133: 523-536.
40. Liu, S., Kandoth, P. K., Warren, S. D., Yeckel, G., Heinz, R., Alden, J., Yang, C., Jamai, A., El-Mellouki, T., Juvale, P. S., Hill, J., Baum, T. J., Cianzio, S., Whitham, S. A., Korkin, D., Mitchum, M. G., and Meksem, K. 2012. A soybean cyst nematode resistance gene points to a new mechanism of plant resistance to pathogens. Nature 492:256-260.
41. Locasale, J. W., 2013. Serine, glycine and the one-carbon cycle: cancer metabolism in full circle. Nature reviews. Cancer, 13(8), p. 572.
42. López Sánchez A, Stassen J H, Furci L, Smith L M, Ton J. (2016) The role of DNA (de)methylation in immune responsiveness of Arabidopsis. Plant J. 88: 361-374.
43. Mahalingam, R., and Skorupska, H. T. 1996. Cytological expression of early response to infection by Heterodera glycines Ichinohe in resistant PI 437654 soybean. Genome 39:986-998. 10.1139/g96-123.
44. Martinez-Medina A, Flors V, Heil M, Mauch-Mani B, Pieterse C M, Pozo M J, Ton J, van Dam N M, Conrath U. (2016). Recognizing Plant Defense Priming. Trends Plant Sci. 10:818-822.
45. Matzke M A, Mosher R A (2014) RNA-directed DNA methylation: An epigenetic pathway of increasing complexity. Nat Rev Genet 15(6):394-408.
46. Mauch-Mani B, Baccelli I, Luna E, Flors V (2017). Defense Priming: An Adaptive Part of Induced Resistance. Annu Rev Plant Biol;68:485-512.
47. Meksem, K., Pantazopoulos, P., Nijiti, V. N., Hyten, D., Arelli, P. R., and Lightfoot, D. A. 2001. ‘Forrest’ resistance to the soybean cyst nematode is bigenic: Saturation mapping of the Rhg1 and Rhg4 loci. Theor. Appl. Genet. 103:710-717.
48. Mitchum, M .G., 2016. Soybean resistance to the soybean cyst nematode Heterodera glycines: an update. Phytopathology, 106(12), pp. 1444-1450.
49. Miura A, Nakamura M, Inagaki S, Kobayashi A, Saze H, Kakutani T (2009) An Arabidopsis jmjC domain protein protects transcribed genes from DNA methylation at CHG sites. EMBO J 28: 1078-1086.
50. Mosher R. A et al. Uniparental expression of PolIV-dependent siRNAs in developing endosperm of Arabidopsis. Nature 460, 283 (2009). doi:10.1038/nature08084 pmid:19494814.
51. Niederhuth, C. E. and Schmitz, R. J., 2017. Putting DNA methylation in context: from genomes to gene expression in plants. Biochimica et Biophysica Acta (BBA)-Gene Regulatory Mechanisms, 1860(1), pp. 149-156.
52. Quinlan, A. R. and Hall, I. M., 2010. BEDTools: a flexible suite of utilities for comparing genomic features. Bioinformatics, 26: 841-842.
53. Rambani A, Rice J H, Liu J, Lane T, Ranjan P, Mazarei M, Pantalone V, Stewart C N Jr, Staton M, Hewezi T. (2015) The methylome of soybean roots during the compatible interaction with the soybean cyst nematode. Plant Physiology. 168:1364-1377.
54. Regulski, M., Lu, Z., Kendall, J., Donoghue, M. T., Reinders, J., Llaca, V., Deschamps, S., Smith, A., Levy, D., McCombie, W. R. and Tingey, S., 2013. The maize methylome influences mRNA splice sites and reveals widespread paramutation-like switches guided by small RNA. Genome research, 23:1651-1662.
55. Reinders J, et al. (2009) Compromised stability of DNA methylation and transposon immobilization in mosaic Arabidopsis epigenomes. Genes Dev 23(8):939-950.
56. Rigal, M., Becker, C., Pélissier, T., Pogorelcnik, R., Devos, J., Ikeda, Y., Weigel, D. and Mathieu, O., 2016. Epigenome confrontation triggers immediate reprogramming of DNA methylation and transposon silencing in Arabidopsis thaliana F1 epihybrids. Proceedings of the National Academy of Sciences, 113(14), pp. E2083-E2092.
57. Robinson M D, McCarthy D J, Smyth G K (2010) edgeR: A Bioconductor package for differential expression analysis of digital gene expression data. Bioinformatics 26: 139-140.
58. Saze H, Kakutani T (2007) Heritable epigenetic mutation of a transposon-flanked Arabidopsis gene due to lack of the chromatin-remodeling factor DDM1. EMBO J 26(15):3641-3652.
59. Saze H, Shiraishi A, Miura A, Kakutani T (2008) Control of genic DNA methylation by a jmjC domain-containing protein in Arabidopsis thaliana. Science 319: 462-465.
60. Saze, H., Tsugane, K., Kanno, T. and Nishimura, T., 2012. DNA methylation in plants: relationship to small RNAs and histone modifications, and functions in transposon inactivation. Plant and Cell Physiology, 53(5), pp. 766-784.
61. Schmitz, R. J., He, Y., Valdés-López, O., Khan, S. M., Joshi, T., Urich, M. A., Nery, J. R., Diers, B., Xu, D., Stacey, G. and Ecker, J. R., 2013. Epigenome-wide inheritance of cytosine methylation variants in a recombinant inbred population. Genome research, 23(10), pp. 1663-1674.
62. Schmitz, R. J., Schultz, M. D., Lewsey, M. G., O'Malley, R. C., Urich, M. A., Libiger, O., Schork, N. J. and Ecker, J. R., 2011. Transgenerational epigenetic instability is a source of novel methylation variants. Science, 334(6054), pp. 369-373.
63. Slotkin, R. K. and Martienssen, R., 2007. Transposable elements and the epigenetic regulation of the genome. Nature reviews. Genetics, 8: 272-285.
64. Slotkin, R. K., Vaughn, M., Borges, F., Tanurdžić, M., Becker, J. D., Feijó, J. A. and Martienssen, R. A., 2009. Epigenetic reprogramming and small RNA silencing of transposable elements in pollen. Cell, 136: 461-472.
65. Smit, A F A, Hubley, R & Green, P. RepeatMasker Open-4.0. 2013-2015, world-wide website: repeatmasker.org.
66. Smith, Z. D. and Meissner, A., 2013. DNA methylation: roles in mammalian development. Nature Reviews Genetics, 14(3), pp. 204-220.
67. Springer, N. M. and Schmitz, R. J., 2017. Exploiting induced and natural epigenetic variation for crop improvement. Nature Reviews Genetics. 18(9):563-575.
68. Sun Z, Guo T, Liu Y, Liu Q, Fang Y (2015) The Roles of Arabidopsis CDF2 in Transcriptional and Posttranscriptional Regulation of Primary MicroRNAs. PLoS Genet 11(10): e1005598.
69. Supek, F., Bošnjak, M., Škunca, N. and Šmuc, T., 2011. REVIGO summarizes and visualizes long lists of gene ontology terms. PloS ONE 6, e21800.
70. Taudt, A., Colome-Tatche, M. & Johannes, F. Genetic sources of population epigenomic variation. Nat. Rev. Genet. 17, 319-332 (2016).
71. To, T. K., Saze, H. and Kakutani, T., 2015. DNA Methylation within Transcribed Regions. Plant physiology, 168(4), p. 1219.
72. Trapnell, C., Pachter, L. and Salzberg, S. L., 2009. TopHat: discovering splice junctions with RNA-Seq. Bioinformatics, 25:1105-1111.
73. Wang, H., Beyene, G., Zhai, J., Feng, S., Fahlgren, N., Taylor, N. J., Bart, R., Carrington, J. C., Jacobsen, S. E. and Ausin, I., 2015. CG gene body DNA methylation changes and evolution of duplicated genes in cassava. Proceedings of the National Academy of Sciences, 112(44), pp. 13729-13734.
74. Wang, X., Hu, L., Wang, X., Li, N., Xu, C., Gong, L. and Liu, B., 2016. DNA methylation affects gene alternative splicing in plants: an example from rice. Molecular plant, 9(2), pp. 305-307.
75. Wibowo A, Becker C, Marconi G, Durr J, Price J, Hagmann J, Papareddy R, Putra H, Kageyama J, Becker J, Weigel D, Gutierrez-Marcos J. (2016) Hyperosmotic stress memory in Arabidopsis is mediated by distinct epigenetically labile sites in the genome and is restricted in the male germline by DNA glycosylase activity. ELife 5, e13546.
76. Young L D. (2001). Registration of ‘Fowler’ soybean. Crop Sci 41:257.
77. Zilberman, D., Coleman-Derr, D., Ballinger, T. & Henikoff, S. Histone H2A.Z and DNA methylation are mutually antagonistic chromatin marks. Nature 456, 125-129 (2008).

What is claimed is:

1. A plant cell comprising an overexpressed or inactivated gene, wherein the overexpressed or inactivated gene comprises a protein coding sequence and/or its homologue selected from SEQ ID NOs: 3 to 209 or homologs thereof.

2. The plant cell of claim 1, wherein the plant cell comprises:
i) in the endogenous promoter of the gene one or more mutations that increase the expression of the gene;
ii) a heterologous nucleic acid comprising the gene;
iii) one or more mutations in the endogenous promoter of the gene that inhibits or abolishes the expression of the gene;
iv) an inhibitory oligonucleotide that inhibits or abolishes the expression of the gene;
v) a deletion of the endogenous gene;
vi) the overexpressed or inactivated gene integrated into the germplasm of the plant cell or the plant; or
vii) the overexpressed gene under soybean endogenous promoter and/or an exogenous promoter.

3. The plant cell of claim 1, wherein a gene encoding an aspartate aminotransferase protein comprising the protein coding sequence of Glyma.17G216000 (SEQ ID NO: 3) is inactivated and/or a gene encoding a transcription regulator of the NOT2/NOT3/NOT5 protein comprising the protein coding sequence of Glyma.02G071300 (SEQ ID NO: 4) is inactivated.

4. The plant cell of claim 1, wherein the plant cell is in a plant part.

5. The plant cell of claim 4, wherein the plant part is a seed, endosperm, ovule or pollen.

6. The plant cell of claim 1, wherein the plant is a soybean plant or other cyst nematode-host plants.

7. A plant cell comprising an overexpressed gene, wherein the overexpressed gene encodes an miRNA comprising the sequence of SEQ ID NO: 210 or a homolog thereof.

8. The plant cell of claim 7, wherein the plant cell comprises:
i) in the endogenous promoter of the gene one or more mutations that increase the expression of the gene;
ii) a heterologous nucleic acid comprising the gene;
iii) the overexpressed gene integrated into the germplasm of the plant cell or the plant; and/or
iv) the overexpressed gene under soybean endogenous promoter and/or an exogenous promoter.

9. The plant cell of claim 7, wherein the plant cell is in a plant part.

10. The plant cell of claim 9, wherein the plant part is a seed, endosperm, ovule or pollen.

11. The plant cell of claim 9, wherein the plant is a soybean plant or other cyst nematode-host plants.

12. A plant cell or a plant comprising one or more genetic polymorphisms selected from the polymorphisms provided in Table 8, wherein the one or more genetic polymorphisms render the plant cell or the plant resist to an SCN infection.

13. A method of producing a plant cell or a plant of claim 12, the method comprising editing the genome of a plant cell or a plant that is susceptible to an SCN infection to replace one or more polymorphisms in an SCN susceptible plant cell or a plant with one or more polymorphisms that confer resistance to an SCN infection.

14. An isolated nucleic acid sequence comprising the sequence of SEQ ID NO: 211, 212 or 213 or a fragment thereof.

15. The isolated nucleic acid sequence of claim 14, said nucleic acid sequence comprising SEQ ID NO: 211, 212 or 213 or a fragment thereof, wherein the sequence comprises one or more polymorphisms that are associated with resistance to an SCN infection as provided in Table 8.
